SEQUENCE LISTING (31 sequences)

SEQ ID NO: 1 - DNA, 2512 bp, Homo sapiens
ctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 2 - protein, 393 aa, Homo sapiens [sequence omitted]

SEQ ID NO: 3 - DNA, 4768 bp, Homo sapiens
agttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa

SEQ ID NO: 4 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 5 - DNA, 1954 bp, Homo sapiens
aggagcagccggcgcggccgccagcgcggtgtagggggcaggcgcggatcccgccaccgccgcgcgctcggcccgccgactcccggcgccgccgccgccactgccgtcgccgccgccgcctgccgggactggagcgcgccgtccgccgcggacaagaccctggcctcaggccggagcagccccatcatgccgagggagcgcagggagcgggatgcgaaggagcgggacaccatgaaggaggacggcggcgcggagttctcggctcgctccaggaagaggaaggcaaacgtgaccgtttttttgcaggatccagatgaagaaatggccaaaatcgacaggacggcgagggaccagtgtgggagccagccttgggacaataatgcagtctgtgcagacccctgctccctgatccccacacctgacaaagaagatgatgaccgggtttacccaaactcaacgtgcaagcctcggattattgcaccatccagaggctccccgctgcctgtactgagctgggcaaatagagaggaagtctggaaaatcatgttaaacaaggaaaagacatacttaagggatcagcactttcttgagcaacaccctcttctgcagccaaaaatgcgagcaattcttctggattggttaatggaggtgtgtgaagtctataaacttcacagggagaccttttacttggcacaagatttctttgaccggtatatggcgacacaagaaaatgttgtaaaaactcttttacagcttattgggatttcatctttatttattgcagccaaacttgaggaaatctatcctccaaagttgcaccagtttgcgtatgtgacagatggagcttgttcaggagatgaaattctcaccatggaattaatgattatgaaggcccttaagtggcgtttaagtcccctgactattgtgtcctggctgaatgtatacatgcaggttgcatatctaaatgacttacatgaagtgctactgccgcagtatccccagcaaatctttatacagattgcagagctgttggatctctgtgtcctggatgttgactgccttgaatttccttatggtatacttgctgcttcggccttgtatcatttctcgtcatctgaattgatgcaaaaggtttcagggtatcagtggtgcgacatagagaactgtgtcaagtggatggttccatttgccatggttataagggagacggggagctcaaaactgaagcacttcaggggcgtcgctgatgaagatgcacacaacatacagacccacagagacagcttggatttgctggacaaagcccgagcaaagaaagccatgttgtctgaacaaaatagggcttctcctctccccagtgggctcctcaccccgccacagagcggtaagaagcagagcagcgggccggaaatggcgtgaccaccccatccttctccaccaaagacagttgcgcgcctgctccacgttctcttctgtctgttgcagcggaggcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactcccacaacaacaaaagcttgaagctgtggagggccacggtggcgtggctctcctcgcaggtgttctgggctccgttgtaccaagtggagcaggtggttgcgggcaagcgttgtgcagagcccatagccagctgggcagggggctgccctctccacattatcagttgacagtgtacaatgcctttgatgaactgttttgtaagtgctgctatatctatccattttttaataaagataatactgtttttgagaca

SEQ ID NO: 6 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 7 - DNA, 7088 bp, Homo sapiens
gctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttcca

SEQ ID NO: 8 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 9 - DNA, 11954 bp, Homo sapiens
agaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttagattgtgtcattaaatggaatgaggtctcttagtacagttattttgatgcagataattccttttagtttagctactattttaggggattttttttagaggtaactcactatgaaatagttctccttaatgcaaatatgttggttctgctatagttccatcctgttcaaaagtcaggatgaatatgaagagtggtgtttccttttgagcaattcttcatccttaagtcagcatgattataagaaaaatagaaccctcagtgtaactctaattcctttttactattccagtgtgatctctgaaattaaattacttcaactaaaaattcaaatactttaaatcagaagatttcatagttaatttattttttttttcaacaaaatggtcatccaaactcaaacttgagaaaatatcttgctttcaaattggcactgattctgcctgctttatttttagcgctatcacaggacccagagcctatgcccttttaaacttaccacaaaagcagaagattaattcaatttaagatgatactctcatttgttacgtcctttttttttttttttggagatggagtcttgctttgtcgcccatgctggagtgcagtggcatgatcctggctcactgcagcctccacttcccgggttcacgtaattctcccacctcaagcctccctagtagctgggattacagggacgcaccaccatgcccagctaatttttgcatttttagtagagactgggttttaccatgttggccaagctggtctcaaactcctgatgtcaggtgatccatctgcctcagcctcccaaagtgctgggattataggcgtgagccactgtgcccggccaatatttgttactttcttaggtttaatagagaaaagggataaaacatttctaactgggagttaattgcatggagaaggtcttaaatcagatgttttaatgccttaaatgtctgtataatatcatgttttcaaatctaattataaatacgtttaaagccaagaataaatcttttaaaaaattga

SEQ ID NO: 10 - protein, 3418 aa, Homo sapiens [sequence omitted]

SEQ ID NO: 11 - protein, 898 aa, Homo Sapiens
MSVTEEDLCHHMKVVVRVRPENTKEKAAGFHKVVHVVDKHILVFDPKQEEVSFFHGKKTTNQNVIKKQNKDLKFVFDAVFDETSTQSEVFEHTTKPILRSFLNGYNCTVLAYGATGAGKTHTMLGSADEPGVMYLTMLHLYKCMDEIKEEKICSTAVSYLEVYNEQIRDLLVNSGPLAVREDTQKGVVVHGLTLHQPKSSEEILHLLDNGNKNRTQHPTDMNATSSRSHAVFQIYLRQQDKTASINQNVRIAKMSLIDLAGSERASTSGAKGTRFVEGTNINRSLLALGNVINALADSKRKNQHIPYRNSKLTRLLKDSLGGNCQTIMIAAVSPSSVFYDDTYNTLKYANRAKDIKSSLKSNVLNVNNHITQYVKICNEQKAEILLLKEKLKAYEEQKAFTNENDQAKLMISNPQEKEIERFQEILNCLFQNREEIRQEYLKLEMLLKENELKSFYQQQCHKQIEMMCSEDKVEKATGKRDHRLAMLKTRRSYLEKRREEELKQFDENTNWLHRVEKEMGLLSQNGHIPKELKKDLHCHHLHLQNKDLKAQIRHMMDLACLQEQQHRQTEAVLNALLPTLRKQYCTLKEAGLSNAAFESDFKEIEHLVERKKVVVWADQTAEQPKQNDLPGISVLMTFPQLGPVQPIPCCSSSGGTNLVKIPTEKRTRRKLMPSPLKGQHTLKSPPSQSVQLNDSLSKELQPIVYTPEDCRKAFQNPSTVTLMKPSSFTTSFQAISSNINSDNCLKMLCEVAIPHNRRKECGQEDLDSTFTICEDIKSSKCKLPEQESLPNDNKDILQRLDPSSFSTKHSMPVPSMVPSYMAMTTAAKRKRKLTSSTSNSSLTADVNSGFAKRVRQDNSSEKHLQENKPTMEHKRNICKINPSMVRKFGRNISKGNLR

SEQ ID NO: 12 - DNA, 21 bp, Homo sapiens
atccgtctacagtaaccttaa

SEQ ID NO: 13 - DNA, 21 bp, Homo sapiens
caggtggaactaatctggtta

SEQ ID NO: 14 - DNA, 21 bp, Homo sapiens
caggaggacttggactctaca

SEQ ID NO: 15 - RNA, 19 nt, Homo sapiens
uaaauuacccgaacaagaa

SEQ ID NO: 16 - DNA, 21 bp, Homo sapiens
ctcgaagtgtaaattacccga

SEQ ID NO: 17 - RNA, 19 nt, Homo sapiens
ggauauaauugcacaguac

SEQ ID NO: 18 - DNA, 21 bp, Homo sapiens
gcagcuggauuucauaaagtt

SEQ ID NO: 19 - DNA, 21 bp, Homo sapiens
gccgataagatagaagatcaa

SEQ ID NO: 20 - DNA, 21 bp, Homo sapiens
ctcgggaagctggaaatataa

SEQ ID NO: 21 - RNA, 19 nt, Homo sapiens
ugguuuacaugucgacuaa

SEQ ID NO: 22 - RNA, 19 nt, Homo sapiens
ugguuuacaugucgacuaa

SEQ ID NO: 23 - RNA, 19 nt, Homo sapiens
ugguuuacauguuuucuga

SEQ ID NO: 24 - RNA, 19 nt, Homo sapiens
ugguuuacauguuuuccua

SEQ ID NO: 25 - DNA, 21 bp, Homo sapiens
aacgcagagttcgaccgttta

SEQ ID NO: 26 - DNA, 21 bp, Homo sapiens
aaggcgggtccggcagttttt

SEQ ID NO: 27 - DNA, 21 bp, Homo sapiens
aatgcgcttccctgtttttat

SEQ ID NO: 28 - DNA, 21 bp, Homo sapiens
aaccaccttgaacacgtattt

SEQ ID NO: 29 - DNA, 21 bp, Homo sapiens
aaggccacttgcgtcagattt

SEQ ID NO: 30 - DNA, 87562 bp, Homo sapiens
cactttaaaatctacatctttaatatatcaagtagcaagggacaatattttatttgtgatgactaaataatatttacaatgattttatgggcacatatatttacacttgtacattaaagtatttttgaaaaggaaagtcatatatatgattaaaataatgtatcaaaacataatagagaaccaaatacatcttgggccataattttaccacttctaaattcttatttttaaaaaataataaagataatttaactcactaccatttactaaaatgtcctagtcaacttgttgtggcatagtaaaactaagctgccaccatggtaacttacactcagggattttaaaaggcaacaaatatttaaaaaacttaaaagacaaccttttcttttgaaattttatttttttagaacacaaaagaagaaaacaaagagtttcatttttctgcttgctgaaagtacttgggtaaacttagctttaagatgggtcttctttcaaagattttaaatatatttttgaaagggtattgataaactttgaaaagcagatttgatcaacttcattttgcttggttttgaagtgatttatcttagatttccttttgaaatatttcttccaaattttctaaccatgcttggatttattttacagatgtttcttttatgttctagagaagaaataaaaagatgcataaatatggcataaatatcattcaaaaagttttcatcgttcaacttataaaaattatgaccatagaaagaaaaatttactcttgaaattcatagttgttagttaacataatttaaaatataacaacaacaaagagacattaatacctttggtttaaaagctgttcaaattcatattgcaaactaaagtttcagtgagtgcgtgcacatacacagacatcgggtgaagtcgctgaattaaatactatgttcattttctttctttctgactaataaaagaccatttctacaagcatttcttcattttctttgttgtatcaccattttttctcattccttggattatatttccccagggatttaaacttccatatttttccaattttaaaattatcatttgcatctagtacttcctaaagggctgatcttaatcctatagctcttttttcttttactgccacctctcaagtatgttctatttatgcccattcttctggttcttcattagctttcaccgtcattttctactcaacaaagtgttgttacaacagggtgccattgacttttttcaggattagtttaattgcttatccactttgttggccatattaggttttagttttagagggacatattcccttaacactctctgttatttctctaactgctaagttaacaggagttgcctttttccctactgatggtcctatgtttctttatttgtacattgttagttagaaaactggtttaatacatttttcacttgaacattataatgtatataaaatatctttccttacaaactaatacctctttggcaagcattgtggtttattatcatctcccacttttttttccccatagtagatatttttactcacatctctttatacatgaaatatttgatgaatttgtatcttcagataataagcatattagaaaaagccaagttctatgatttgagttttttttttttttgagacggagttttgctcagtcgcccaggctggagtgcagtggtgcgatctcggctcactgcaagctccgcctcccaggttcacgccattctcctgcctcagcttcccgagtagctgggactacagccgcctgccaccatgcccggctaattttttaaaatttttttggtagagacggggtttcaccatgttagccaggatggtctcgatctcctgacctcgtgatccacccgcctcagcctcccaaagtgctgggattacaggcgtgagccacggcgcccggccaatttgagagtttttataaatttattatttggctgtagaattatgattcttgaagaactttttaagaagatcagtggggcagtacacacctaaactaactacagatacacatttttaagcattattaaatacaacatgtcaaaccaaatgatcaaggtaaattttgtactttactaccttactgtctgatgtttcaatttgctgtctgatgcttcaatttccattgaacttaaggtaaacactggctactacaggaagcaggcatttaccaatatagtttgacaaagttactgaggaattttatattactctaatcaaagggcatcgacagaaaaaaactaacaatagctttcatgttatccttagaaaaattatatcatgagacacctttaaattgcatttgctataattatcagttcctatagagaacaatcaaactgtgtcttactgctctctgtattcatcacaggacatggtaaatgcctagtgaataaatagtttctgaatgaagtcttaggaactgtagtatcagagacagaggtctagatgttggggcctgaaaactggctggcatgatacaccactagtttaactgaagtggttttgttaccataaactcatgtcactcccatatgcggttgatggaatgtagtatctttcatactgatgggaaagatccatcagtatcttttccaacttgccaatgggagttacaatatgattgtaactagatacagactggaatctgtgtagtagttacacattgagttagaggccaaattattcaaaatattacgctaagagaaaaatttaaagtgggtatagtttatctgcaaaatacacttaattagtaacattaagtaatacatttagccaattatttcaattattatgagacagtagggataactaaaagggtgtagaaagtagagttttaagatctggttcttgagttttggctctaccaatataatatctgtgtgatcttgggtaaatgcttttgtgttttaaagtttccttatctataatatgtggataatcatacttgttctaccacatagtaaaggtgatgaataaagggagattcttatgaaaacacaataaaaaacgtaaagtactatatgtgtgttacagagtcataccattaaatatcaaattaaaagctgagacatacccattgttggtttgttttcttgtaagtgcttctcacttgaattatcttgtcgaacacgtttggcaaatccagaatttacgtctgcagttaacgaactgtttgatgtagaacttgagaggaaaagtttttaatttagttaagcattttttttatacctcaaagttctaatacatattgtacatgcgacaatgattaaagaaataaaacacaaaaatattaagttgtgaaatctcacagaagtggaggtactaaatacatttttggcccacagagaaatttagctgtgtgtgattttaaattggatattttatatttttattcaaattgaatgttatgattgtaggtgtatgttacaattctgtagcagaatattccagggtgctttgctggcagataagtaagcactattattcaaaatgtagagatcagtagcacatagtcacaagaggttaaaaaaaaaaaaaaaaaaaactaagctcatgttacaaatttggaccaagaatcctaatgtgaaaggtataaacaagtgagattgtttgaaaaaccttgttttaaaatgaaagcagggtaacaataaaaatctctaattattacaatcacagaaactgaaataaaaagaatgacctgttttcatttgctctagcttcaaaaatttaatcagtcttacactgctgatggttcaactttcttactcaatttgagttgccatattagtaaacacctattatggccttgtgttacatgatatacaagtaaacaaggcacaaaaatccctgtcctaaaaacttatagaatagacatacagtggagatgagaaaaaaataagataaatacatgaaataatataaggtaaagtaaaaatatagtgccaaggtttccataatacaaggagctatagattcttgagtgtagagtaagaaagaataatggaaattttgtggatgcaatgaatttgatctgggtcctgaaaaacagcaatccttcctagtcctaaagaggatcaatttttaacaggtaggattggggggggttgggtggaaaaagaacatccttccacgctgattcaaacaacctaagcttagggaaattctggctaataaagatactatatagtaatcccccttatcgacgggggggatatcttctaagatcgccagtggatgcctgcaaccgaggatagtatcaaaccggatatatactatgtattctcctacacatatctatgataaagtttaaattataaattaggcacagtaagagattaacaataataataaaatagagcaattataattataattattataaggttatgtgaatgtggtctcgctctctattaaaatatcttattgttctacaatcgcctgtttttgaactgcggttgaccatgggtaactgaaaccacagaaagcaaaacttgaataaggggtgactactcagtggaagtaatgaaaatgacacagtttaagaagataaaatcacaaatttgtatgcacaaatattatatcaactacagtagtccccacttcttaaatggtgataaattactatgattctgttttgcatattaaacacacttttgtatatatcagctgtccaaggcagaaattcttttttgatcatttggtgattttattttttcaaatataaagatcaatattattagttacttttgataaaacattgttttttcttaaatttatcccaaagttgtctttattatttcatagtacgattatgcattatggctagttccaggaccctactttttactcatagcatgttctttcttgaaaattaggcttttgaataaagccatttgattagaactcatatttctagaaatcaaatccaaccctaataaattgcgtatttatataaaagtaaaatatacccaaacatgtttacgatttagttaaatagtccactgatgaaatgtttaacatttatgttgtaggtcataaactacaaagacttaaaactgatgagcgcaagttcaaattatgttaaaggctatactgaaaaatccaaagacacttccagttgtgcacaaaacatattcccataactataaataaagttaagaattaaccttttgctcccttatataggtagaatgtttctactcaacataatacctcttctactattttatctgaaataatttctagttgaaataattagacatctttcaaaacaaaataaaagtgtatggcaaatatgaaactgctttggggtgctcagaagattgtggacaaacaaaaaaccaatcaaccaaacaaatacaaaacaaacaatccaaaactctgattaatataattgagaagtaaaagctaacattatgtatgcattatatttaaatatttgataggctaatcaatttagtaaaatagtgtgctagagaaatattaaataaattgttaagattaaaaatctaatatactcaattatttaaacaaatctgttgggtaggttttggaatatccagtgctttcaaactctaatgccattttatgcaaaataaatgaaatacatatatagagagcttccccttctttattttaaaaactatattagaatatatacacaactaaatatgtatataaagctgctaaatggaaaataccacagtttattactctgaagcgcatgtaactttaattggggaaaaagaagattaaaaaaagtaatcatgtacatttctagcattaatctcttttaaccttagttaatggcaggatctaattactggtaccagagggaatgtaatagaacaattaataggctataagaaatcaagttactttctttacatgtcctttaatattatttcacaatattagttgttcatgtggaagccctttaacttatctaaaatcactccaaatacacaattcacatacctggacatttcctgaaaaactatttgattttataatagctaagttcatatttaaaaattattcttatctataaacatttgtgacatcttagtaaacacaacatactaccagtgaaggacaagaaaactcactaaaattaattaatggaacaaatgacaaaactgacactatgcaaattagtgataaaagtagacgaaacagatgattgtggttagctaggctcactcataaagtgacagacttctgtatataaaccagaataattgtacaaatcaaatttgtaccttagcaaatataaaattttaaatgtcatagatgagacatttaggaaaaatccataaattttaaatttacacattccaagtctctgttatactttgtttaggagggacaaggacagatattaaattcacttatgagaagatactctatgtagcacaaataagtaggctacttgtaaattcgcttcctgttacttaaatgataacaggctcctggtggaaaatacatttaaaaagagtcattttaaggtcttctataaacttttctccccataatcacatagcaaagaaaaagaggtagaggaagtcagatccattttatatttattgagtatatttgtcattgcattcaatttataggatataaagaatgcatcatgaacttctagtcatataactcaatatatttaagatttttccttatctttagtttttagttgttttactactatatacctaggtatgtttctctttatattaacccggcttttggtttgcagtgctactcaaatccatggtttaatgtcttttgttagttttgaataagtattagccatcatctcaaatattacttttccctattctttctctcactcccttctgagacacaaaatacatattttctagacttttaaaaattgtgtcccatgtctctttcacttcttcccatatttttatcatactgcacatcagtttggatattttcctaccagtctatctttcagaaaaaattccaaaacctgaacaaatgaataataagtaacaaaatcaaagtcataataaagtctcccagcaaagaaaagcttggaatttgatggcttcactgctgatttttaccaaacatttaaaaaagaaccaataccaatcttactcaaactattctgaaaaacagaggcggaaggaatacttccaaaatcattatatgaggccagtattactctgataccaaaaccagacaaggacacatcaaaacaagaaaactccaggccaatatccctgatgaacacatgcacaaatccttaacaaaatactagcaaactgaatccaataacaaacctgaaagatcattcatcatggccaagtgggatttatcccagggatgcaagatggttcaacatatgcaaattaaccaatgtgatacattgtatcaatagaatgaagaacaaaaaccatatgatcatttcaatagatgctgaaaaagtatttgataaaattcaacatccctttatgataaaaactctcaccaagcggacttaatagacgtctacagaactctccacccacaatcaacagaatatacattcttttcagcaccacactgcacttattccaaaattgaccacatagttggaagtaaagcactcctcagcaaatataaaagaacagaaattagaacaatctgtctctcagaatacagtgcaatcaaactagaactcaggattaagacactcactcaaaaccactcaactacatggaaactgaacaacctgctcctgaatgactactgggtacataacgaaatgaaggcagaaataaagacgttctttgaaaccaacgagaacaaagacatgacataccagaatctctgggacacatttaaagcagtgtgtagagggaaatttatagcagtaaatgcccacaagagaaagcaggaaagatctaaaattgacaccctaacatcacaattaaaagaactagagaagcaagagcaaacacattaaaaagccagcagaaggcaagaaataactaagatcagagcagaactgaaggagatagagacgcaaaaaacccttcaaaaaatcaatgaatccaggagctggttttttgaaaagatcaacaaaattgatagactgctagcaagactaagaaagaggaaaagagagaagaatcaaatagacacaataaaaaaagataaaggggatatcaccaccgatcccacagaaatacaaactaccatcagagaatactacaaacacctctatgcaaataaactagaaaatctagaagaaatggataaattcctggacacatacaccctcccaagactaaaccaggacgaagttgaatctctgaatagaccaataataggctctgaaattgaggcaataattaatagcataccaaccgaaaaaagtccaggaccgggcggattcacagctgaattctaccagaggtacaaagaggagctggtaccattccttctgaaactatttcaatctatagaaaaagagggaatcctccctaactcattttatgagaccagcatcatcctgataccaaagcctggcagagacacaacaaaaaaagagaattttagaccaatatccctgaagaacattgatgcaaaaatcctcaataaactactggcaagcccaatccagcagcacatcaaaaagcttatccaccatgagcaagtgggcttcatccctgggatgcaaggctggttcaatatacgcaaatcaataaatgtaatccagcatatacacagaaccaaagacaaaaaccacatgattatctcaatagatgcagaaaaggcctttgacaaaattcaacagcccttcatgctaaaaactctcaataaattaggtattgatgggacgtatctcaaaataataagagctatctatgacaaacccacagccaatatcatactgaatgtgcaaaaactggaagcattccctttgaaaactggcacaagacagggatgccctctctcaccactcctactcaatatagtgtgggaagttctggccagggcaatgaggcaagagaaggaaataaaaggtattcaattaggaaaagaagaagtcaaattgtccctgtttgcagatgacatgattgtatatctagaaaaccccatcgtctcagcccaaaatctccttaagctgataagcaacttcagcaaagtctcaggatacaaaatcaatatacaaaaatcacaagcattcttatacaccaataacagacaaacagagagacaaatcatgagtgaactcccattcacaactgcttcaaagagaataaaatacctaggaatccaacttacaagggatgtgaaggacctcttcaaggagaactacaaaccactgctcaatgaaataaaagaggacacaaacaaatggaagaacattccatgctcatggataggaagaatcaatatcatgaaaatgtccatactgcccaaggtaatttatagattcagtgccatccccatcaagctaccaatgactttcttcacagaattggaaaagactactttaaagttcatatggaaccagaaaacagcccgcattgccaagacaatcctaaggcaaaagaacaaaactggaggcatcacgctacctgacttcaaactatactacaaggctacagtaaccaaaacagcctggtactggtaccaaaacagagatatagaccaatggaacagaacagagccctcagaaataataccatacatctacaaccatctgatctttgacaaatctgacaaaaacaagaaatggggaaaggattccccatttaataaatggtgctgggaaaactggctagccatatgtagaaagctgaaactggatcccttccttacaccttatacaaaaattaattcaagatggattaaagacttaaatgttagagctaaaaccataaaaaccccagaagaaaacctaggcaataccattcaggacataggcatgggtaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaattttcgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaaaaaatgctcatgatcactggccatcagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagacagtgtggcgatttctcaaggatctagaactagaaataccatttgacccagccatgtcgttactgggtatatacccaaaggattataaatcatgctgctataaagacacatgcacacgtatgtttattgcggcactattcacaatagcaaagacttgtacccaatccaaatgtccatcaatgatagactggattaagaagatgtggcacatatacaccatggaagactatgcagccataaaaaaggatgagttcatgtcctttgtagggacaggatgaagctgaaaaccatcattctcagcaaactatcgcaaggacaaaaaaccaaacaccgcatgttctcactcataggtgggaactgaacaatgagaacacatggacacaggaaggtgaacatcacacaccggggcctgttgtggggtagggggagcgcagagggaaagcattaggagatataccaaatgtaaataacgagttaatgggtgcagcacaccaacatggcacatgtatacatatgtaacaaacctgcacgttatgcacatgtaccgtggaacttaaagcataaaaaaaaaaaacaactctcaaaaaactgggtatagaaggaacatatctcaacacgataaaagccatacacgacagacccacagctggtactatcctgaatgcagaaaatctgaaattcttttctctaagttgtggaacatgacaaggatgcccactttcaccagtatttattatttaacatattactggaagtcctaagtagagcaatcagacaagagaaagaaaagaaagaaagaacatccaaattggaaaggaagaagtgaaattatccttgtttgcagatgacatgatcttatcatatatttggaaaaacgtagagtccacaaaaaactatgagaacctagaaacaaattcagtagagttccagaatacaaaatcaatatacaaaaatcagtagcatatctatatacaaacactgaaataatttgaaaaaaaaagtaatcccctttacaaaagctaaaaataaaataagtaggagttaaccaaagaagtaaaagagctccataatgaaaactataaaacactgatgtaagaaaagcagatacaaaaaaatagaaagatattccatgttcatggattggaagaatcaatattgttaaaatgtccacactactcacagtggtctacagaatcaatgcaatccctatcaaaataccaatgacactcttcatagaaatagaaaaaacaatcctaaaatttatatggaatcacaaaagacccagaatagccaaagctatcctgagcaaaaagatcaaaactggaggaatcacattacctgacttcaaattacactacagagctatagtaaccaaaaaagcacgggactggcataaaaccagacacacaaaccagtggaacagaatagagagccccaaaataaatctacatatttacaataaacttatttttgacaaagctgccaagaacatacattgggaaaaaagatagtctcttcaataaacagtgctggaaaactgtttatccatatgcagaagaatgaagctagatccctatctcttgcaatatacagaaatgaaatcaaaatggataaaaatttaaatttaagacctcaaactatgaaactgctaaaagaaaacattggggaaactctccaggacactggactggacaaagatttcttgagtaataccctacacagggaaccaaagcaaaaatggataaatgggatcacatcaagttaaaaagcttctgtaaagcaaaggaaacaatcaataaagtgaagagacaacccacagaattacagaaaatacctgcaaactatctatctgacaagggattaataaccataatacataaggagctcaaacaactctaggaaaaaaatctaatgatctgatttttaaaatgggcaaaagaatagacatttctcaaaagaagacataaatggcaaagaagcttatgaaaaggtactcaacatcactgattatcagaaaaatgtaaatcaaaactacaatgagatatcatctcaccccagttaaaatggctttttttccaaaagacagtcaacaacaaacactggcaaggatatggagaaaagggaacacttgtatactgttcatggggatgtaaattagtacaaccactataaagaaaagtttgaagagtcctcaaaaaactaaaaatagtgctaccatacgatccagcaatgcccctactagatacatgccaaaatgaaaggaaatcagtatactgaagagatatctgcactaccatgtttattgcaacactactcacaatagccaagatttggaagcaacctaagtgccaatcaacagatgaatagaaaaagaaaatatagtacctatacacaatagagtaccattcagccatgaaacaagaatgagaccctgtcatttgcaacagcatggatggaactgatggtaattatgttaagtgaaataagccaggtacagaaagataagcttggcatgttctcacttattttggggagctaaaaaataaattgaactcatggagatagagagtagaatgatggttaccagaggctgggaagggtttttcgggagattggggaggatggttaatggggggatgcttaatctaaatataacgaataagatctagtatttgatagcacaacagggtaactacagtcaacaataatgtattgtacatttaaaaaataactaagagtataattagattgtttataacacaaaggataaatggatgaatactccatttacactgatgtgattattacacattgtatgcttgtatcaaaatatcccatgcaccccctaatatatacacctactacgtacccacaaaaataaaaagtaaaaaacaaatccatctttcagttcactaatactctcttctgctgtatgtaatctgctgaattgatttattgagttcttcatctcagtaaattctatattttcagttctagaatatccacttgattaattttaatagaatctggttctttggggaagtatctatttgttgaaattattacagttaaaatttttaaattcctatctgataattccaatatctgcctcacgtatgaatcactgctcccatgatgtttttttctcttaatttttagtcatttggtcctgtctcctggcatactttgtaatttttgtttaagtgccaaacattgatgcagagcaaagataaatcaccttgatccaatcaagaattaagttggtctaaggtctgtttcagactttcaagaactggcctatttctaggttgccctactcctagtgaaacacccttcagggatctcaaataaaatcctaatgtgtttaacagggtcttttcttcatggtggtccctgaactccaatttttctgtgaaagcctcataagaacatcaaaactctgcttaatactttagcctcttagcagctactttcagctaggcatcttagcctctcattccacaaagctgagaacttggcgaataccttgaggacaaaacctgtacagaataattttgtttccctagctcagagtaagacagctgatagctctaagtttccattttctgttcggcctctatgccttatgccataaattggcaaatgctaaataaaaaacagaggcagagaaagtctagctgtctatagcatgtttctcttttctctgggatctcaggctctccatcctggctatcatggttgttctccattgccttcaagcagctgttggttctattttatgcactttttacaactgttcttgataggtagcttggtctgatacaagctacactgtcttaggcagaaataaaagtctcttcactcattatattttagtcatatatcttaatccattaaaataacataattctatgatggaagaaatttggtaatgtttaaaaaggataatgaagtaagtagaaataacgaaataagtaaaaattgtcaccccagaaaagttaaaattaagtagtttttgacagaaaacttttcaatttatttttaagtctgtaatatgaaatattttataaaatatcaagtgaaatacttgagtttttcaagatctccctttgataatataaatccagtaccccattcagagaaatttatcctaagtatgcttaaacatgaattgagagtctaattataaaaaataaaaaagcaatagcatataatatataaagattatcttatataactacagaaccaaaccagtttatgtttaatcataccttgttaatttccgtttccttttggcagcagtagtcattgccatgtaggatggcaccatgcttggtacaggcatagaatgcttagttgagaatgaagaaggatcaagcctgtatattaatagtgacaaataaaaaataataattttgatttgaactatgaagagaaaaggaagcaaatgtgtccataattagaaaaccatttggtattaatatataaagaaaaacattctgctatactgtaaagattcccttgcttgtcctggggtattcttttctctttataacacaggtaaagttgctatcaagagatttctgcattcccagtggcatagaatagtgaaggtatttttctttccatatttagattgggtatgattatgcagataaagtgagaaggatatgtttgcttggatgacacacattcaaccatactttaggggtatgaccatttcagagtaagttgaggtttgctaaacatcaatagaaagtgtgaaatttccttcagtgaaaatattaatgaataataaacatgataatcctccaacaatagcttgtataatccaccttcagagtaagaagaacaaactaaacatatcttaaggtctctcctagactaacgacggtcaactgacttaagatatcaaatttaagactttaaaatattttgttagtgaattaatattcagagagaaagataattaaaatttaatgaaaataaaacagactgttttataatcaaaatggcacacttggtttatgaaataaacctcaactaatagttgaaagtctatgttaaaaaaaaagaattggcaaatattatttttttgtaccgttgtaaaatgtctttgttatcatttggtagtgattcttgttcgggtaatttacacttcgagctcttgatgtcttcacatatagtaaatgtagagtccaagtcctcctgtccacattcttttcttctattatgagggatagctacttcacacaacattttcagacaattatcactgtttatgtttgagctgatagcctgaaaacttgtagtaaatgatgatggtttcattaaggttactgtagacggattttgaaaagcttttctacagtcttctggtgtatatacaataggctgaagttctttgctaagagaatcattgagctgcacactttgagatggtggagactttagagtatgctgtcctttcaagggagatggcattagttttctccgagttcttttttctgtaggaatcttaaccagattagttccacctgaagatgagcctattcaaaaaaataaaaaaagacactcgataatgtttcctagttttttaaataacccccattacttttaaaactaagaaaccctaataataaggtatttttatgccaattgataaaaatctgaaaaatataagttggaaaggaaaatatcacggtttactgctaaaaactttaaataaagataaactcattataaagaaattctataacaaaagaatatattgctaatatcaatattaatttataggtatagctttcttgagatattgggtaattaggaaacaaattatcaatcaattccaataaaattcacaatttctagctttcatgtttgatttgggtttaagatgaaaacagtattaaacttttataggcacagaggttaacacttgttccattgtaaaatattaccagtttattacttttgttttgccttgttttttagatttgcgggtacacgtacaggtttgttatatgagtatactgggtgatgctagggtttgggtgtcagtgaccagtttattactaattgagtggaagtctagtctaaatgaacaagaatggcaataaccagtaaaattctgggtgaatgaaagcattgtttctgttacagtgtcaggtatacagagttgaccctcagtagctgcagattctgcatctgtggattcaaccaaccagagactgaaaatattcacaaaacaaacaaacaaataaaccaaaacaattaaaaaaatatacaatataacaaccacttaatggcatttgtattgtattaggtattataagtaatctagagatgatttaaagtacacagaaggatacatgtaggctatatactatgccattttatgtaagggacttgagtatccatggattttggtatctgggagggttcctggaatcaacttgcctgtccccaggatattgagagaaaactgtagtcaattaagcattctagttaactgatttgtctcctgtatcatacataactcataggtttgtaaaaacaagatttaatactaaagataaatgaaataattcagagagttcagagtactttcaccctcatcactctggagaagtcagtctcccaaatgtcaagaggtcactaagtctgatgatctttctcagccttatctgataattcctgtagcttttcaaaattatatctgtactattctaacatctgtgttattctcttcttccatctagtaaatgtacacatttccctaaggagctgtacttgaccctctagttgccttgttctacacagtttctcatatttctaattatcctcttaccattaccttctctgtcacctgggttccaaattactagatttttttttctaccttttcccttgcattttccaatgtataccaattctgtcttttttatgcttatagttaaaacataattctgttcatgtcatccttctgattaaagactttttagtaatctttaaatagaaacaaaatatgctgatatttttagttgggtaattcttggtccttggtccaaaattaagaaacaaattgtttcattagaatttattaccacaatactagatttagtatttaatgtttaacatttaaaaacacaaacatctggtaaacaccttttattaatgccccaaataatgacatatgtaacaatacacttgatcaacttcagtgtgattatcttatatttttcaattaacttttttctactctacctacttcattctggtgaacttcatggtgtctgacatgaaatacctttgcttggagtgtacggtcaatggaagaaactattacgtgtatttccattcacgttgtctaatcatgaagtaaaattcccagttgatatttactttactgtataatactaacttaaagaacattgaaagcagaataagataagtgttatgcatttcaataaagcaattcaaactttcaaccaagttagaaaatacacttaaacaatcagcaacagtacaagcactattcttattatattagttgacaatatgctctgctttggctaggttgacaattaccctctaggaaataaaattcaaaaacagaagaaaaatatgatccctgttctactgaggaattttacttttctgagaactagataaatgagatccagatcttaaaataattttccactccctttaacatgacatttgtcaaattttccattaccagatatttttatactaatgctgcagagtaaagacagttacttttctccactgtgctgctaccaccaatttggtggctattaatagctggcagattaacttcttttcccatgagaggcagcaggggtataggggatagaaaactacatggagggtagaggactgaattgggagtcaggagacttggattctatttctggctccaccaacagctcaccaagtgaccttgggcaactcacttaagcagtgtgggcattaatatcccctagaaaaaagcggatattacctttttatatatgaataggattatatgataagggttcaaagaaaggctctagaaatgcaaatatgtttcaactgttaatattacaacttttacaaggagagttgtaatttactttccaatcatcaaaaacttactgttatctctaaaacaatatatttcaaacagtgcttcactttcagtttatcagatatccagtttatcaaggctgatgattaatataaaccacaggtattattgaaacaggtatatatgttctttgcaaatgcagaccttataataacattttaaactttaaaatacaatggaacatgaaatctcaagggacaagaattatagagtcattattttttaaaatacatacttagcaggtataatctctgggagtggcaactgggcaaattcccatcatggtggaccacattttccctactgctagtaaagagtgataaaaatcacagtcttctggtagaacactgaagaaatatccacgtgtcctctgggataatctaagcatacacccgacatcaaatttagtatgatcagactgactaacaatttgtcagtaaaaggaatagtggtgttacagtgatatatctttagctataatcctgctctcctggcagagtataagctgaaggaatagcatggagatgtggtagttaatgtggatttacatacaattgttttactgggcctagaagaagagatgctacagacacacaacatgtatatgatataaatctaaagagttcttttcattctctcaattattaagttcagataaaataaaagggcagaaggcagatggtcaattcagaactacttaggctgattagttgcttagaaagcttcaggagctaaccagagcaaaaacaaaatttaaaggtttaaataaaaatttaagtcaatcttctgtaactataaacataaaaggaaaatgacccaacttaatgaaaactaaaagcataaacaactaaattttactctgaatacaatttttataatatataaatacaattacataacaaataattattatcctttagctaagttaggtaataaagaactttatgtgggtatggcaagtggagagtgttgagttgggggatcagaaagttcaataggctataagaactgttggatatgttaggtctatgtctataaaagagatctgctgtacttttccattagaggaaaagggtgtacagtaaatttccagaagagcaggcatgggacagttgtagtttcccctgattcagcttgggaaaacactatctaacaatattccataattcttgtgcttatagagtagaagtgataagaaggaacttggaggcagattataattccttcagaaaggccccataaaatgagaaaatgaatcacaaagttgcaggctgtgccttgcagaacaagagaccatatagatgatgatagcccataaaataccctagcaacgtgggtgctacacagaagggaacaggaaattaaaaatgcattgttataaggcacgctcttgcaaactggaatagggaagactggcagtcacttgggaagacatatctctagggtctatctaaggaactagtccagtgtatcatatacttaaatataagaggtagtgggtagcaatgtatgttatggtttctgagtaattttgctatttccacaacatctcttgagactttaactcctcaacatattgtaagtgtgctccatttctattaccttgaatgattatgacccatgggatgtaagtgaataggactttttgaaatttatgcccaaataataacaggaagcataatgtcaaaaaatttaaaagaaaaaaatcactactactattttgaatccaggatcaacatatgttttatttatgaaaaaaagttgtagtcagctatggtacaaaagcaacaatctaaaaggagtacacctgcataaaaagaattcactgttcatcccacccaatatttattaagtacctaccataccatgctaggtacagtggtaaacaagacagttaagaatttacatttttgaggggagaaggcagatgacaataaacaaataaaataaattaggatgataaattaattgtaaagacaataaaacagagaaatggaatagacagtgatgggagataggatgttttaactatgatggctagaataacaattctaaggatggaatatgtgatctgagacttgaatggctaaggaggcagttctatgaagatctgggggaaaaggcagaaaaaatagcatgtgcagagatccaacggcaacaatgaatttgttgagctcgaggaaaagaggctagtttgattgaagtgctgtgagttagagggacaggcagaataagaggctagagagatcaaaaggggcaacttatgttaggatttatggacaagacaagggtttttatttattccaagtgtaaagggagatctgtggagggtttcaaatatggatataatgtgatctgattgatgtttaggaaaaagagtgatttgcttgctgtgtcaagaatggtcaagagagaaaacagaaaaatcggtaacaaggctattgctatagcttaagcaagaaatgacagcttgggtaagagtggtaggagcagtgatggagagatgtgagaagggtattataatatctatatatgtaattagctgccttataataaagagttcacagaaagtagtatatattaattctacataagtaaattaagagaaattgacaatgctccataaaaccatgaaccatttatgcatgtcattccctcaactttggcattcttttttttttgacatccccttccctctactcccactcatcaagcttgggatcctgtcctctattcaggagttctgatccatgtaactgtaggagtcctggaatctgcatttttccctttctagtttggacactcatagagacttgaggcagtagaaactctttggggtttgaggctgtgatttcttattcagagacgatgagtgttattattatgctgaggtaggggaaaggaaagaaaaggagtaaggataagtaggaaacagtcctagaacaaaagtagcagatagagacagtattgaaaatgaagttcacctcctaggaagttgtaccaaaggcaattaatgggtaagatttaagttggtagaatgatgttaaaaacagcatctctttttaggttaggcaccctactgtctctgcatattcatctctggacactcccaaacaattaaaacaggtttatagacagttccaggcatagacatactatgttttcagatacaaggtctaattgctcctcttataatattttgttagtatagcattcagttctttaaaaaggagccaatgcattttgatccatgtttaaggaagcttgaaaatgcaaaatgcaaacttgtaatgaatgagttttgataaaagaatatacatattagaatcttttctttttgatagggaaggaatttagaaagacactggctgcttgaagaatttctcactagaaacagtacttggttggcacacttgagaaatacccatagtacaaggaggcatttgaaggttaactgcaaaagacaattttaaccaaagtgataacctgaatagaggcatctagcgcccactcagattccctttaaaatgatcacatcattaaaaaaagggtaaacttgacctaatgttggtaatgggaaaacattatcatctgagtgccagaacattagaggactttctaaagagtattgtatagctcagactcttaagggaaggcatggaggagaatgacttatagtactccctatcctttacccttcagtgctatcccaaggagttaagtaaaagacactggcagatatcagtaaagggccttaaattagaaccagggatgatggggtaaggtgggagttggggagaatgagtgagacacagctatgcaatatagtaaaatggcaacatctgaacactaaaagtagcgctaagaggctctaaaattcagtcctggctcagttgtttcagtgcagaaaaaaaatattaaaaagtacccgatattatcaggcttgttcccatcactgttggctatatcacacatggatgagatactcttgggcaaaggggcagatttctagctgagactacctgtggctctggttccttccctacattccctgggctgttggatcagtggacaggaatatattttttccccttaattttacaggaaaaaacataattataataagcaaacagaaaactcactggacctataaaaagaaaatgtaaagaaatgacagaactggcatacaaactcagaccttttgctaatccagatgccaagttcttcaccaccacacaatgctgagcccctaccacagaagttaattaaatcacttaggcaatgactaaataactaaatgaacatgaacatccacaattacactgatatttaaaactatattttatagattatttgatgatataaaaaaatgttcattaatgaggtagtatacagtatgatcccaattttgacaaaacaaaaacaaaaacaaaaacaaaaaccaaatcaaagcgcacaaacctcctgagcaaaaatggggggaaaagtaatagaaaacagagaaggaaatataatccaattcatcaaaatgttaacacaggtttatagctcactgggaaaataatgaattattttattttcctttttatacatttgattattttatataataaatataatcctgctctgtaaaataataaatgaccaaaaaaaaaaagggggaataatgtttggggtggtgggtaggaagccaataaaaaatcagctctttcaaacctctgaagatctcaggaactgaaggcatgaggttgtctctgaaagcacagggtggagttaaaacaggagaattcttggaagtctgtataaggaagttggattcccagatctcccttaccccattaccatcacctacattgctaggtaactgctcctttgttaccctattaaaagacatgaagttttactctctggataggctgtagactggggccaacatgcacagctgacactaggtgcattaaataatttcttaataataaatatatgctgatttttctcccacttctctgtaatactggtagccaggcccatataccccctaaatggaaaatcaaaaagttctttgtaaatgaaacataaaaataaaaaagatggacatctgtaggttcccccagattgactagttttctggagataactataaatcagagctttgtttaataagtccaaccctagttttgtaatagttactcattaatagtttagcaaacatttttaaaatatttatatgatgctttaaggcatcctataaaaaactgaaagtatgcgtttcaggataaagtggcgagtgttctattcatctgttcctgcattatggctagagattcccgcttcaaaaagtttgtttactgtaagtccaaagacagacttaaaaaggaagttagacaaaaacattttgttgagactgccaagttttagaagcctctcaaaagacttttaagtattaaaaccacttttaaaattaactgtaaatgcatgcactgaaatcatggattagatggagaagaaatacaaatattttcatcaataacccaggttctaaaacaatgtaacatggattacaacagaagtatattaacattagtattatagagtattaaatttgctattaataaaaccacagctaaaaataaaaacaaaaacagtgaaatcataatgaaaacataattaatttgcatgacaaactacatttgctaagtggttataaaaattataaataaatatttggtgagtggttaagtggttaaaaaagtaaacaaagtttggatattcatctgccattcactatctctcaaatgtgtaggattttaagacatataattgatgaatggtagtgtaataaaagcaataaattgggcattttgattttgacaataaaacataggataatctgatgaagtacataaaaaagacttgaaaaaagtttgataaattgactgcctatacctcaagagaaacatctacaaatgtatttattggtggtattgctctttaagctacttaagtctacctataaggacaatcaatctacttcagtatttgcatctttcaaatatttcaattctccaggaagtctatctcacagatttggctgaggactgtgaaacgtttccagtaaaaagcagaaaaaaaggaaaaaaaaaaagagatgcttcatgtctgtttccctaaaattagttttaagaaatgtcagtatattcttacttctaataaatgttttaaataatattttatttaattataaaatatttccttaagtaggctctaaatgttacacatattaacatatgtataaatataatgtgaaattgcatactttatgtatatattatataacatacacatgtcacttcttacctgatcctgctaaaatacattagcaaaaccagagaagaaataaggtggcggtgttgtaagtgagctaattcttcatctctcatgattagaagttataacatctaaacactaatgaatcaagaaacagcagtatagcatactgtttagaaatattgtaggttgcaacctactcatctgacaaagggctaatatccagaatctacaatgaactcaaacaaatttacaagaaaaaaacaaccccatcaaaaagtgggtgaaggacatgaacagacacttctcaaaagaagacatttatgcagccaaaaaacacatgaaaaaatgctcatcatcactggccatcagagaaatgcaaatcaaaaccacaatgagataccatctcacaccagttagaatggcaatcattaaaaagtcaggaaacaacaggtgctggagaggatgtagagaaataggaacacttttacactgttggtgggactgtcaactagttcaaccattgtggaagtcagtgtggcgattcctcagggatctagaactagaagtaccatttgacccagccatcccattactgggtatatatacccaaaggactataaatcatgctgctataaagacacatgcacacgtatgtttattgcggcactattcacaatagcaaagacttggaaccaacccaaatgtccaacaatgatagactggattaagaaaatgtggcacatatacaccatggaagactatgcagccataaaaaatgatgagttcatgtcctttgtagggacatggatgaaattggaaatcatcattctcagtaaactattgcaagaacaaaaaaccaaacaccgcatattctcactcataggtgggaattgaacaatgagatcacatggacacaggaaggggaacatcacactctggggactgttgtggggtagggggaggggggagggatagcattgggagatatacctaatgctagatgacgagttagtgggtgcagcgcaccagcatggcacatgtatacatatgtaactaacctgcacaatgtgcacatgtaccctaaaacttaaagtataataataaaagaaaaaaaaaaagaaaaaaaatattgaaattaaatgaaaaaaaaaaaaagaaatattgtaaatactgtctggctgtggtggctcatgcctgtaatcccagcactttgggaggctgagataggtggatcaccggaggtcaggagtttaagaccagcctggtaaatgtggtgaaaccccttctctactaaaaatacaaaaaaattagccgggcctggtggtgggcgcctgtaatcccagctatttgggaggctgagggaggagaatcgcttgaacctgggatgcagaggttgcagtgagcctaggtcatgccattgcagtccagcctgggcaacaagagcaacacttcgtctcagaaaaaaaaaaaaaaaaaaaaaagagaaagaaatattgtaaatattgtaagtttgttatatataatcatccagcatatattccaatagaaaacagaaaaatccccagtaattttatatcttcctaaggatagttttaagaaatgttggaaatgtccttaaaacagttgtcaatgacaagtcaaatccaaataatttgaaaccaaatgcttggttttagtccatcgctacaagaaaaaaggaataaacttacacacaaatgtttcctaatagcaaaaactgaaaggctcaagcaagataaggccttacgtactttaactatgacatttctatgtcatgggaattatatagtttagcaatctatatctatttttcactgaatctctactttaaaagagtggtcctaactactggccaccaggaaagttacaatggggatcatcttatacaaacaaccagaatttagtactagatggttttagtgtttatttaaacttcattattttataatttgttgctcacagcaagaaaatgacattctcaaagtcatgatagcaattatatttgttgttgccagagttggagtcatgtttcttattttccaggtagaagtcaggtaaaaagtaatgcaagtaatacagagtaataacatatataataaacataatggagacttaaaaaaataaaactttcttcacagactcaaaaatatgaataggaggcaacatactacttcattgctcccaaaatgaaatgctatttcctattattactgaaaagagaaagtaggcaaataaaatatgaatatcttcataacttaaaattgggttacataaaaatcaaaaccaaccatgaataagagagaaaaaaatacatgtaacacatttggcaaaggatttagagctcgtgtatgtgtatgtatgtatgtctatgtatacatgtgtatataggttggctggtctgtctctgagagaaataattcttacaaaacaaaatggataagagcaaaataaggcaaaggaagaggcagttaattcagaacaaactataactagacaaaaagcacatgaaaaaatgttcaccttcaccttcattacccacttacaatggtagactacacttataatagttctgggggtggggaatctgacaatgcatatctaatgcctttaaagtatacatactgtttgattaagctattcttcttctaagggcttagcctaaggaaataattaggcaagttgcaaaataaatacacatgcacagacatttaatggggtgtcgcttgtaataataaagtgaaaacaaactaaatgttaattagcaggagattaggcaaatataatatgcagctattaaaaatgacttaaattcatattcgttgatagcaaaatgtgtctacagcaagtagttcagtgaaaaagaggctggttatcaactatatatatataatagattccactgttcttgttttaaaaaagcaaaccacaaatgtatgcttagaaaaaaagttcagtaggctattaaaactaaatactaatataagttaataaagtatatcttgtttcttttggcttcacttttttccaaattaaaataaacaaatatttattgctaagacagtaacaaggttaaaacactagtgaaaggaataaaaaacttaaaagccatgggaatacagatgaatatatgcaggcaaaggaaagatgatccattaaaaggaagatttaataaagaaggaagtatctgaattggactctcattgtggagaaagatttaggccaagaagtgggatggatgtagagaaaacaagcatttgaaattgatggaaaagcttggaaattgcagaggccagaaacagaaatcatgataacagaaggttgggatacaaggtgctggacaaaagtatgcctatagcttgacactggacttggaaagcctttgaagactcttctgagaggtctacactttattaggaagatcactttgttggcagtgtgaagtgtagactgcagtggaaagagacaagagaaattaaaactagtttagatgtcactatattattcatttcttatgccaaattttctaaaaatattttaaaataaattaagaaacatctttttctctctttgttttaaataaatctctaaaacataattgctaaatggcaattcatgtggtttcttaagtagagggggctttatatttatttttcagcttttggaaatatttcacagatggttcctttacataaaataaagagactagataatttatcactaatgggtttaaaatccctctcttaaaaatgggcagggcttttggggtcatcttttgatgtgccatatcatgtgttctgaatgattttacccagaggaagtaatttatcaaaagtttaagactgtaaacgttaagagatttatagagctgtatgttattgtaaaggaagaatgcggtatttagcaaacatgagtcataggctgcaaagtagggttcatgacagggagacattgatgaatgggattagatctaattgaggtttggtacaaaacgatggcaaggattcttcctttgcaagcaagcactcctgttaatatgtacgtgaggagaaattctgggaaactgtgctaaatcaaaattagttggcaaagttctaaagtgaaatcaaatattaatgtatacatttcaataaaaacttctaggctacaaatgattgatacaattagtgtactcatcataaatctggattctttttaacagccttatgactcatggaattcatagttgcaaatgatacaggtaaatgaattttaactatttataagatacgtaaacttatcttataagataaagtaccattaaattaaccaaatataagataaggtaccactaaattaaccaaactaataaacattttataatacacttatgagacactctcatggttagggatgtataatagttccctaatacatagggaactattgtcctaatacatagggaactattatagtcctttaaattaatccagcactttgaataataaatcaacccataaccttttgatattttttctgtgggaaatagcagatatcatgtttctctctcagtttcattttctcagtatttacacttaccaaagatggaaaagaagggacaatacaaagaaccttttaattcacagtaatatttgcagctatagatgaataactaaatactgtcttatgtacatggggatgataagtcaaacaaaatgtatttagggatacaagcatgatttccattgtttcatgattaaattttgttgccaaatggaaaaaatatggaagatttggtttattaacattttcctgctttatatttgttatatgagaaattagtatagagatttcctatctttcaaatgaaatttccttgtattcaagtaattttaggggaaacttaaacttatgtgtgaactctatattcaatcagaatattggtactatgttaagagactggttctcctagtatcttttattgtactttaagataaaatcactgacattgggtgcaaaattttaaaaaaggaaaggttacagacagaagtattaccaaatgtgagtttccagatggataatgaggaaatcttgacttttcatttaatgtcagaaattttatcagttccataaataaattttgctgtcagcacccccgtcttgcactacacttagtgagtgtgctatctattttccagaccccaaaataggtacatattttactcttagtcaatacacttgtagtttgaaggtaaagatttcacgtcaaataaatatctattcaaaaatcaagtctatttattagaaaataattaaaattcataaaatttacaaaattagtttcttccaaaataagaatttaggcttattaatttaagaaaagtaaaaagtagtctatgtcacaaaaatacaaaatattgtatctagggatctattcagtcaaatttataaaacacttacgaagaaaactctgaaactttgctgaggaacatacaaaaagatatgcatacagggagacaaactattctagagtacgaagatttgatattggaaatatgtgaagtcaatttatgaattaaaaaaatttcatttccttgaaactggaaattattctgatattcatttcctagacaaagcaaagaatatttttcagacaaagaataatgaagaagggctagcatactatataaaaaaaactataaagtatcacatattaaaataatagctactggccctaagacagacaaacaggtgaaacaatagagtagaaattgagtaaacagaattatatatgactacttactgtacaaaaaacacagtggctgttaaggatgcattgccaatatatctatataagaatgctagtctccttggaaaaaataaatttagattcttgcctatcatttaacaccaacataaattaggagaaagtggaaacaaaaatttaaaagcttatgaatggaaaggccttttgaggaaagatcccgaaggcagtaagagagagaaaaaagctattttactacataaatgttttaactttcagcattaaaaaggaaacctcaaacaaaagcaagaataagtggcaaatcttgaagggtatagttatactatgtatattacaggaaaaagttaatatcccttagtgatttcatcctgtcctatggctttaaatatcatctaaatgttaacaactctcgaatttatgttttagcttagacttctctcctgaacttcagattcttaactgcctacatgatgtctccacttggatgtatcttagacaaatttaacatgcctagaactgaactcctaattttctacaacctccaccctgtatacctgctccatctgaagtcttccttatctctctgaaggtgactcaattcttttagatgctaagttccaaaattttagattcagtcttaagcatttctttctctcctattccaaatctaattttcttggaaatcttgctggctctaccttcaaaacattttagaatctgaccacttctcaccagctccaatgctattatcctggtctgaataacctccatcttttgcctgggttattctagtgcctcctcatgtttcagcccttgccatagtataatctattctcaccacagcagtcagagagatcttttaagagcacaattgaggtatgttacatttctgctcagaccatgtcatggctccttgttttattcagagtaaaagactaattaagtcctgatagtaacctacaaggccctatgtgatctgccttccttccctttatctctctgatctcctcactactttcattctctttttctttgtaccagttatatatcctcaccagcctttgaatatgtcaggcaaattcctatgttagagattttgcactgtgattgtgtccctctacctaaaaaagtttcttctcccagatattcatatagctagtcctgtgaagtccttcaagttttatcagtgagatcctccctgaccatgctactgaaaatagcaatctctctcctccctggccacactcctgaactccctttatttctttcttatagcatgtagtacctcctagcattctgtttaatttatttactatattattgtctttctctactagaagtgagctccttgagggtggtggagtttgtttcatactttgatccctatcatcagacaagtgcctgaaatactgctgtaagttaaacactgttgaatgaatgaaaaaaaacctacacttaagatcttgtaaaaaagtaacgattgttactaaagatgactatatcagaaaaatgtcaaaggatggctgggcaattcacagaaaaaagactcttcaattaaaaaaaacacactcaactatattagtaatgaatatggaaaaaagatagtatttatgactattggcaaagtatcaacaagcattgccattactttcaatgatgaaaaccacaattacttctgcactaacctaaaaattagcaaagacaaaagtgggagagagtgtccaagattaaaaaagactgcataggcatgaaccttgtttgtatcttaattcaaacaaatcatagaaacaaataatttatgagacaatgggggaaatttgcacacattagatatttgatgacatttttcttttatacacacttgatgtaaatggaagaaatgatgtgctgtctgagatctacttttttttgttttgttttttgaaataaaactttatttattaacactgaaatttgaatgttttataattatcaggtgtcacaaaatatttttctaattttttccagtcatatacaaatgtgaaaaaaaaaatcttggcacatgggacataaaaaacagatagtgaatcagctttggcctgcaagctacaactaaatgaatcctaatctttttttaattttattatttatttattttttaattatactttaagttttagggtacatgtgcacaacgtgcaggttagttacatatgtatacatgtgccatgttggtgtgctgcacccattaacttgtcatttaacattaggtatacctcctaatgctatccctcccccctttcccccaccccataacaggccccagtgtatgatgttccccttcctgtgtccatgtgttctccgagatctactttaaaataatctagtgtaagagctggggtggaggaaagcagatgggttttcaagactagacatgagctgataagttttaaagttaggtgttgggcacatggtactcatagtattctttgtgcttatatacatgtttgtaattgatttactaaaggtagaaattaaaaatattttaacatcaaaagctggtgaggctaaaaaaaaaaaaaaagctccttcatttatactgcttgatgggggttaaatcacttgaaggaggagagttatttggcaatatatatttcaaattttgcataatgggcatttatcttgagccagaaattttacttctaagaatttattgtagggatagtcagatatgagcacaaagataaatatacatatttgcttactaaaacattattggcaataatagtgaaaatttattaaacaacccaaatgcccaacaatatagactatattcattatagttaatctcatcagattttttttttttttttgagacagagtcttgctttgttgcctaggctgaagtgcaatggtgcaatctcggctcactgcagcctcaacttcccaagctgaagtgttcctcccagttcagcctcccaagtagctaggattacaggcatgggccacaatgccttgccaagttttgtattttttgtagagacagggtttcgccctgttgctcaggctggtcttgaactcctgactcaagcgatcggcctgcgtcagtctcccaaaatactgggattgcaggcgtaagccactgtccctggcccagattcttaaaagcactgcatgtgtgtgtatttaagtaaaaagaatatagttaatctagtaaatacttttaaaggtccctaaggcacttgaactcacttataaacaccgttcctattattcctccttgacactattagtcatgtatacaacatacataattcatgaaatggaaaatacataataaataaatagttgatctctaaaatcatttgaatgaaatagtaaagtaacgaaccaagaacaaaacattataaaaacaaaactcaccttctaatatgtgttcaagagaaaacacgttaattataaaatgtaacatggcttcaagtctttccttcaaaaatatcatgagaaggaattaagagtcaagtaaattcttttgttttattgttgacaagtatagtctgtgcaattatactgtatgtgaaatatagatttttaaataaggaataatgagcatcatatagcaggtggtcagtatttctcaatcttggtgttaaaatcctatttcctttctaacatcttgagtacattataaaaaatgaatcatgcccgaaaggtatgagagaatctgaaaaaaacttaagttttgctttccagagatcacaactaggtcaggtctaatgctttattaaaagaaaaagaatacatatgtgtatatacaatatatatgttttaagataatctatccaacttccgtattttcttaatgtaaaggtataatatagggaacttctgctaataataaagatggagataactcagaccaacctttcttctgaaggcaactaaaagtctggacaaaatataaaaaacatctgtttgaaggcactggattattaataagatggtgaaaaattacaggtgagtatcaggaggaagctggaggctcaggaaagtaatttctgcaatttcagctaatttccccttgaggttgtttgcttatgctggaagagctggcagaaaagcatagcaacagtgctaagagctttgggggaatagggtgctggaactggaatttaggatctttgtaaatcttccttgctttgagttgggactctgaacagctatactgtagagggcaaatcagaaataggcaggacctcaggaaactacagtttagtatcaaattacctcagtctctgaaataggactgaggtgattctgaattgctaatgctacctagaatatgaaagaaggaactgtaaatattctctgagatatttaatatcatgatcagatgcatcaaaatattctgcaaataattttgcaaatacctgactggcacacaaaaaatttgcatgcatatgaggaattaagaccacatgcaagaaaatcagtctaaacagatccagtggttacagactttggaattctcagacacagactttaaattatagttgtctctcagtatctgtgagggataggttgcatgaccatactaaaattcatggatgctcatatcctttatatataaaataatacagcatttgcacacaacctatacaatcctcccatatgctttaataatctctagattacttacaatacctaatactatatatatatactatgtaaatagttatgatgcattgttcagggaataatgaccaaaaaaagtctatatgttcagtacagatgaaaccatccatgttttttttttttttgattagttggttgaatccatggatgcagaacccatggaatatggatggctgactataattataccctttgtgcttaataacaatttttgcaaagatatgaaactataaaaaagaataaaatggaaattccagaattggaaatatagcgactgaaattaaaaattttatggatttaaggatcatttgataaagctgaaaaaagaaagtaaactgaagaatgtgagaaacaaaattatccaggaagagaaaaaaacattaaaagcaaaagagtaaagggcatagacaatacaatgggaaagtgccatgcaatataattggagtcccagaagtagaggagagagcaaaagtgacaaaagcaataggtgaggagataatggttaagaattttccaaaacaaacaaaagataatgcagaaacaaattcagaaagtcaggaacccacttaagagaagtaaaaataaagtcaactaggcataacataataaaactattacaagccaaagactgaggaaatatttcaataagccagaagacagattgtattcagaggggcaacacttaacataaaatctgacttcaacagactattattctctgtgaataaggtcttacatattagcccttcactcccaactatttgtctatttgtcttaggtgagctaaatgtgtgctgggacacatttgagccaaatgtcccagcacacatttagctcacataagaaaaatggactctagttgggagtgaggggctaataaacaccagatcccaagaaaattccccataatggaaaatataggcagatttgactatataaaagttaaaagtccaataaattgatcaattattgctcaacatatatataaaaaaaagcagcaccaaatgaacaaagagttattagatacgaagtgccaagtaaaaaaccaagcaaacaaaaagcccctcaaacccataacaaataaacaaaaatttccactagtgtgaatgagcaaaggatacctgacaggggatgaattggcctcatcgataatctgagagttacacattttaaaagactatgcagctgggcgcggtggctcacgcctgtaatcccagcactttgggaggccgaggtggatggatcacgaggtcaggagattgagaccatcctggctaacatggtgaaactctgtctctactaaaaatgcaaaaaattagccaggcatgatggcgggcacctgtagtcccagcttctttggaggctgaggcaggagaatggtgtgaacccaggagacggagcttgcagtgagcagagatcgcgccactgcactccagcctgggtgacagagcgagactcggtctcaaaacaaaacaaaacaaaaaaaacaaaacaaaaaagactatgcattattattgctgttgttactatttttggtttaggtaaagatgaaacagttactctcatgggtactaagtacagaaaaaaattatgaaaagcagtttaacatgatgcattaaaaaaaaaatgatatgtcctttaacctggcaaatcagcttctaggattctgctatgcacgcacaaatgtgaatacacagatgtgttctctgatgtactgttcactaatagaaaaaaggttaattaaattatggtacatctattctctggattctcaaaaacaataaaaaaatgaggtagattcttcatgtatgcaccctaaaaatgttcataataaacaaataaaaccaataaacataataaactaaacaaaaaagttgcaggtcactatgtgtaatgtgctagcattcacgtgaaacctaacaaaaagctttatgtaatacatgtgtattacacacatactgcaaagataaatatttaacagcatttaatctctgagatagggcataaaattgaggttctgagaagagttgaaggtggcagtctacttcttttcttctacattgtctgaatttttttcttaactaagattatatgttagctttgcaattaaaatgtctcacacatacttttatgagagaaagagaaatatcaagtgatttgtcaatagaaaaatagtaaactctacactgttagttgatcttaagaacagattattatcataaaatatgatatgattaattataatttcctgcccttaatagaggttacattttaaagaaggatgggaagcaaaattgttccatcaaaaatagataaaatgggttaggcacagcagctcatgcctataatctcagcaatttgggaggccaggggagacaactgcttgagcccaggagtttgaggtcagcctgggtaacagtgatactttgtttctacaggaaaaaaaaaaaaaaaaaaaaaaaaagccaggagtggtggtgcatgcctgtaattcctagctacttgtggggctgaagtggaaggattgctagagcccaggaggtggaggctgcagcaagctttgattgcaccactggactccaggctgggcaacagggtgagaccctgtcaccaaaaaaaaaaaaaaaaaaaaaaaaaaaaaaaagaaaagagttgtaaaatgaattttactgctagagttatatagaacatcaatgcatacttgttctagatctttggaagatactggttaattgaaatgactataaagctaattttcccattaattttcataaaatagacaaacctattaaaattaactgttctatagattgatatacaaaggttctctttagaaataatgttactatttacttaaaacgaaagttatacttacaacaaggaataggctgaactggtccaagttgtggaaaggtcataagaacagaaatccctggtagatcgttttgctttggttgttcggcagtttggtcagcccaaactaccacttttttcctctctaccaaatgttcgatctctttgaagtcagattcaaaagcagcatttgacaggccggcttcttttaatgtgcaatattgttttcttagggttggaagtaaagcattcaatactctatatacagaagatgagaagaaaggagagataaatatgacattttaaacacattattttagttcttttatgtaacacccaaagcattagcatatcattaaaataatttcccatgtatgaactgagttctagtctcagattgtcttcacttgcttccatatgaccttggacaaggcaaaaactgaagtttttcctctgtaatgtgagggacagataaatagccactacttataataggagattgctttgagggctaaatgagttaagtatatttgtttatttattttttagagacagggtctcactctgtggcccaggctggagtgtagtggcatgatcatctcattgcagcctcgaactcctggcctcacttgatcctcccaccttaggctcccaagttgctgggaccacagatgtgtgccactacactgactaatttttttattttttggtagagatggggtcttgctatgttgtccaggttggtctcaaacacttagcctcaagagatcttcttgcctccacctcccaaagtgctgggattacagggatgagccactgtccctagctaaatgaattaaatgtacataaaagctttttgcaaaacataatgtgatttccaaactacagatgttgttactgctactaattatcataaaattgaaatgaaaaattaatatactatgtgaaatgatgagacatattttaggtatacaacattaaaacacacacacacacacaaacacataacaaaataacaaaacctacctctcctttaagttaaatgacctaaagaaatggcttttggaatggcacttaaagggattttaaggagaaaagttgtggataaggatggggagtataaataatcgttagggcactggattcacggctttaactcaaaatctcccttattagcaacaattacagcagcaccgaatgtaattttttgtgtatcagacctctgtgtaagtgttcattgaagaaatgtcacagctaaacagttataaatttatacactcaatcagtggttcccaaattttctataaagaaatatgtctaacaacatgaatgatcttgaaatgattcagtcctggaatcctgagtccatcaatagatgatgttccagttgactcataacatcgaggagaagagaaccattagaagtatggatagggtagtgggaaaagactaatcgggcattcactttcatacacaaagaaataccaccttttaacctacaagtcttaaatatttcaatagcagcacaagaaattgtgacctttttccttttggtgggggtggctggagggtgctgcatttaagtcaagtaggtaacaagtacaagaaaaagttattaatcaaagtcatttaaacaattacttatttagcatctacactaatcaggtactgtgcaggaaagttatcatagtaggtctgacactgctaagaaaggcttggttataaggttgatacctggctggtattttaaaatatggattttaggagggttcccagcatttccagaactcatatgacaagagtggctcactgtgcttaaactgtttgcacaaacaatgtggtttatgctaaaacaccttctctccttttgaaaagtctggaattttgatacatcctggcagaaagtgcctatgcaaccagctcccaataaaaacctgggacatggaactcaaaagagcttccaaagcagagaacatttcacatgtgctgccacaactttttgctggaggaattgtgcacattctgtgtgattccactggaggaagactcttgaaactctgtgtcagggttcctgcagactttgcgccctgtaccttttccctttgccgaatgtgctttgtagcctcttgctgaaataaagcacagctaagaatacaactatctgatgagtcctgtgagtcctcctaatgaatcactgaacctgaaagtagtcctaggcacctataacacaggaattatatcatgttatttatttttatgtttaatcttttcaaaaactttatatgaaatgtattataatctattcataacctttatatgaactgtattataatctatttttatataagaaaactatagcttagcaaagctaaataatcagcgtaaggttatattgctcatataagctagaattcaagattgattccaaaactcatcctctttcttctatgctacatttcctctgtgatcctctggttcaccagttttctaaattgttgagttagtctttttccccccattttaaggtaaatgtttatcttaagatagtatattcatctggggtctgatatagcaaatgtaatatggttaaaagaaattattgtttcttgatgacttcagctcttgtaagtggtttgagtgttagtaaatgaatattcaatatctgctctaggaatagacacagaaagtataaagagattagtctgaacttccatccacataagagtaactccctgaagtttttcatttttcttttttaaaaaaagctatactaatatgatttcctctaaatcaagataattatcagttatttgagcataccagtagaacatgaatagattcttagagtagcagaaagcacttagagaaataaatatcccatttgcatggaacatcaaagtgatgcctaaggagttgactgacgtatccaatgtgacaaaaaaaagattaagccatactttttattattatgttttaaaaactaaatgactaatgttactactgagctagaagagagaacataaaaggtctgttatagcatcacataaaaatcaggacacctgatatttagcaacttcactgtaatgacagcaaaaaattctggaattatgaataattaacaattgcctacttaaaaattcaagtgatgttaacagcaattgcttctaagtggagaaatggatggctagaagatagaggtagaaggcagatttcttttcaatgtgtgtaggtacttatgaaattttgtattacatagcatatattaagtaaaagtaacatacttttaaagaaaaacactttcaaatttataaaccaatgtaaaaactttgttggaaggaaataactctcttaatgttttctggttcagacagacacttaaaaataatgtatcctgtaacacataaaactcaactttctggcagtggaaaattgaacttctgtgcttcagatatagtgttaaaattggaaaataggtaaatgtatgtactcacgcttcagtctgcctgtgttgctgttcctgaagacaagctagatccatcatatgtctaatttgtgctttcaaatctttgttctggaggtgcaaatggtgacaatgaagatctttcttgagttcctagggcaaacaatacaaaatgtacttcagatcactctaagaatattgaaattaaatcagtttaataattttatattgttgccaatagttttaattctttttagatattaaataatatgtttagaaacaatttgaaaaataagagttctggaaaaggtggtctataatttacaagcaagataagttttcatgtttcctcagaaactgaatgttaggaattcagaagaaattttactatgatgacagcttcttggtctatccaacttgttcacaatgttgtaaaaatacataaatatgaccagtttttgcttaaatgtcacttcctcagaaaggctttccttgagcattgtatttggatagacactctccacccctcaatattgtcatagcatttactctattgatgagtaacaatttcattgatgtattcatttatttataatttctctgttcacaccagaacatatagtttaccaccaggacaggcaccgagtcaggttagttcagtgttaaagccccagtgtacaaagtcttgagttgtgtgcaataaaaatttctgtttttgcatgaaacaaaagatcatatggttgatatcacctttggttggatgacatggttagagcactggcctatgagtcatgtgattttagagatgggcttataaggccttgaaacaaatgctaccttaagttgaatatacagcctttatggagatcagtttactggactaaaatttaagagctggatcagatgatctcttcagtcctttccagttagaaaattaaatgatcctaatagtgtgaacatgtttgccaaatattacgtctgttctcctttaaaagaaaaatatcctttatatacatcctctcataatctcctttatcttcttgaatctttcttcttttaaagagacaagacttgagacccactcttgatattagctccatcctaggcaaaagtgtgaaaatgtttttttgcaggctccattttcctaagcctcatctgtctagggccaggtgtcctgtaatctaccacctaaagttacttgtctttccttaaacctactaaaccttatctacagaatatatcagcgaagggattacaccaaagtataaatggtgataatctagtaacattctcagaaattttattggaaaaatttcaaacaatacaatgaaaaaaatttaaattctttggacattttgaatggctaagacaggaaccccatatgcaggggagctatgcctggaaactggaaacttcctcctagggacaattctcataatccatcttagctttagcgagtggaaacaatgcaaagtattcttcagattcttttaaagaatattaagattaaatcaattttaataaaataattttaataaatcaaatttaataaaactcctttccaatctgcagttgaccaataattttctaaaatgtcaaagttatttttctcctttttagaaagtacttccaatgccagttattgagactatgacagagaacaatgtcactcatcctatttatatattaaagttaggagttatttatatatattaaagttaggatatattaaatttaggagttatttatataattcattatacttcaagaaatatagtatatttatatattaaagttaggagttatttatataattcattatacttcaagaaatatagcaaatgttttagaaaaaaatttatctttatggaggcaggacgatacggctagagtcatgtgattttagagacgggcttagccttgaaacaaatgtcaccttaagccaaatatatgccctttatggagatcagtttacttgactaaaatttaagagctggatcagatgatctcttcagttctttctattactagttagaaaattggatgatcataatagtgtgaacatgtttgccacatattgcatcttttttcctttaaaaaaatctctccttatagcattaggagaaatacctaatgtaaatgatgaattgatgggtgcagcaaaccaatatggtacatgtatacctatgtaacaaacctacatgttgtgctcatgtaccctaaaacttaaagtataaagaaaaaacaatccctcctttatatacatcctctgataatcttattcctttatcttcttgagtcattcttttaaagagacaagacttgattgtataattgtattagtaataatgggtctttggctagcctgttaactttggaaaatcaagtttacaatttaagtctttcttgacatactaatgactaaatctgattggaagaacatggcttattttgagaactataagtaggccagtaatggctaaagtatagagtataatgaaaggtgatatacaagccttgagaagtattcaggtgccagaccatgctaggcttggtaaatcacaatggagaatttattctagggagaaggcagtgaagaacaacaagatcagttttgcgttttaaaaacttgctttggttgcagtggagagaatgaattgcagtagaacatgaacgaaagcagaaggaccaaacgtagcagtccaataatggggcataggagtctgcattaggatagcagcagaggggcataaagagaagtggatagattcagaatattgaagagtaaataatgcaggagagtacagagaaaacagcaccaatttgaaagtcagaagttctgggtatctgtccagagttgtgtgaccttggataagtccctttatcgtaaagttatgtctcaatttgcttatgtaaaaaataatggcttataactagataattttctcagctattttttcagctataaatatctgcacaaaaaagaattgagggttagatttgggtgatggcaatgaaaatggttttaattttatttgctcaagttatttgtgataaaatgcataaaaatcaaagatacaaaaatcaattccaggttcacattcaggtgtacaaaagttcttcgtaagagaaatctagaagacagattgctagaaaaattagatttgaccatattgaaaaagttttaatttattaaaaatccagacaatattgattaacttcataaacactactaagtagacaaaaaagcaaacacaaaatagcgataataatgatatcagaaacaataccataccatttttaaaattggaagcttacggtaatgtgggatagaaaaggcaaactagtaaatttctacaatcgacttttttgcctgcttaatacttcataagttcttagtacttctgtagttggaatgctgattacatggacaaacatataatacaagcataatgtaaattaagttaggatcttgagaggtgaaaatactctagcaatatcatttaaaacttatatacacaaacatatttaaaatttttaatgaatatacttaatgccactgaactgtatatgtacacatggttaagatggtaaattttattttatattttactacacacaaaaaattgtttcaagactcttcttgagaaaatttttgccaactataaggggtaaataatctatgagaatatcacagccataagtaaacaaacaaattaaggaagcttcagtagttaattttaaatgaggtcaaagtttaatactaaccaaaagacttagttttatgtagtttagaaaatgataatcttgagaatatgactgctttagatatacagtctccaaataaactattaaataataagtgaaataaactgaaaagtactgtcaacattccacacctgctaccaatccaagagaatcaactcttgtgtattaaatgagaaacactcatttggccagggttatccagctgttttctttgaagtgattttatcaacaacataaccatatttcagtattcctacaggcactgtgatagagcctgttgcattctatgatgagcctctatggctttatatggaatttcagaagttatatcctctgatcctagctaacttatattatagtttattaatgaatcttgtctttttttgcttactccttttgagtcattatatcatcttaagatggacacaacgtttaaaaaaaacgtgctatagtttccttctgaagtagatattcttaatcttgtgtcaatggaagggctttaggaaacccaatgacttaggaaatatgcaaaattgtataaatttgacctccccaatctcccagctggaagaaaatctacacagctttgaacagattctcaaaggcatctatgacccccaaaaaggtcagaatcattgttctggaagagaaattatattatattatattatattatattatattatattatattatattatatgatgcagttaactctgttgttaactatgtacacttttctttgtacaacttgcttggatattgatgtctatgctattgttgcttagaatagtattaaactattatttatcatccattattatttgttaagtgttgctaggttttgaatgtagcactccacttaagctcataatatcttaaatacatttacagatgacaaattaagacatcaatgttaaacacactgtccaaagtcatataattattgttagagttgttattcaaaacaactactatatgccagaattaccaaacactccaactccaaagtctatccttttcccaatgtttacattatttttgtggggaatattttaaagtgccttacaaactaagttatattattgcaaaatattactagaatgtgctgtattctatgtttaatgtcctaatctacttaactaactgactggtatgcttttatcaagatattttgaggtgcagattaaattgttggctcaataaatatccacaataaggaaatactgtctttggtttccttcttaggaaacaggatatctgctgcttgctcctttcactactttgaactgtcatcattaactactactgctagtcttttctctccagtcatgtctgagcttccatattaacagaataatcatttacagccattgctagcccatcagagaatccattccccagtcataatgcattgactagtatctttgaattataaaaaaattagtacttttgaacatttaaagatctctggcatttgttggtaaatatttcatattttccttcacctgtattttctgtctggtgcctttctaggaattcaaagattttagaagtttccatatcaattagctgatacagagttatagaatttcagaagagcatcaccaaagtagagactgtagatattttttcttttagaaaaagactgaaaagaagttgttcttttaaactattaagtatttgggtgaaaattagttatttcatagcagttgctcctagttgcttagcccacatttactttcttcttctttcttattaagagatctccaatcccattactgggtatatactcaaaggattataaattattctactataaagacacatgcacacatatgtttattgcagtgctatttacaatagcaaagacttgaaaccaacccaaatgcccatcaatgataggctggataaagaaaaggtatattgcacatatataccatgcaatactatgcagccataaaaaagaatgagttcacgtcctttgcagggacatggatgaagctggaagccatcattctcagcaaactaacacaggaacagaaaactaaacactgcacgttctcactcacaagtgggagttgaacaatgagaacacatggacacagggaggggaacatcacatgccagaggctgtcaggggtagggggcaaggggagggagagcattaggacaaatacctaatgcatgccgggcttaaaacatagatgacgagttgataggtgcagcaaaccaccacagcacatctatacctatgtaacaaacctgcacattctgcatatgtatcccagaacttaaagttaaaaaaaaaaaaaagggagctccaatactgttcagagtaattatgcaaaaaaaaaaaaagacaatcttttcagagtgtcttctatctacagatagccatgtgacatatttctagccaatacattagtctgttaggaatttttggaaacatctttgctctcctgatatagacatccctcctcctttctttcctcttcttctggcttaggtgtgtaaacttgagactagatatgaaacagtaatattacaatcatgaaacgacaagcacgataatgacattttaaaggtactgcattagaaatgaactgcatacttccaagacttattgttaagagaacaaaagacagcccacactagttaaggcactaaagttagactactgcaaccttgtagcagaacataattctaatatgatactgtcattacccatcactacacttcaaaaataaaaccttttgccattctaaggtggaatatcttcagttacacacacacaccccctttcaaatcaactttctttaaccataacacatttaatgttgacctcatttaaactatttttgcataaaatatcaagatttcaaacaaattacctacataatgaatcctcaagtgcaggttgaaatagaattgtttattttaacctaaaagctaccttataaaaggatgcaatcacttagaaataacatttaaaagtaaattaaatctcagagttatgaggcaataatagttgtcttatgaaagacatttactcaataagaaaaagaacacattttaggagctcagttcctacaaattaaatctgaacatacagagatatttgtacctttggaatatgaccgttttgacttaagagtcccatttctttttcgacacgatggagccaattagtattctcatcaaattgcttcaattcctcctccctccttttctccaggtaggagcgacgagttttcaacattgcaagtctatgatctcgttttccagtggcctgaaacacgattcatttaacagtaaatctaatttttatgatatttgatgtacatgatattagtaaactagtatattttcttatgtgtactattgtagtaaattaagttaggctacatttttgtatttaaagctatttcccccttgaaatctcagaatactactgcttgcaagaaaataggaaattgagctgtgagtcatttttataaaattacaggttgagagtttatggctttaagaaatacaacgaccaaatcaaagactataactttttttttttttggcgttaaagtacagtcccttcagtatgtttcatttgaaaagttcatacaaggtcagatacattccatgatctccttactcaaaatcaaagcagatgcatatattgctcttgtctttgatgtgaagaaataatgtgttctattgttctgtgtttgcctataaggcctcaaattggctgatataaatcacagaaaaaagagcttatatcagagtgcttatcagtatctgatcttgcctgtagcttggttcaggagccataatgagtattcaaaatatttttggtgttttacctaggcacaaattgaaattaaaattagtctttgatttcttaccctaagggtcttgcacactatcatgaaaacaatgggccagaacatcttgacgtctctggggtatttgcccacagacactaaagtcaataattatcagataggatgggtagacctgaaacagtggatccccaaaggttaaaataagcatttgcagctggttctggaagcaggaaatcaaaaaaaagaagagcagaaactagatttgaatttggtcgaagaactccccctctttgggagataaatatctcttaattttgacaaaagatgtactattgagacttgggcatataatagaaaaatatggttatcttcatctggatagaggatatgagcttataaatgatttaaggtcaggcaaaggtggctatcatgaagtcataaccaaatcttagatattcataaaattttacttaccttcccctcaagggaaatacaagagtcagagagaagcagattccatttcaggctagagtcatagataagggaaaaatagttgatagccaccgtcattctcactaaggttcctgactcttttttatggcttctctggttaattataatatcctagtctctgtctgtcacagtaagacagtaacaaaaacaaaacaaatgtctcaaagaaacaaaaacctggatagtcaagaagagaaaacaaaacaaaatgtaacccatcaaactgaccacagcgctaggatgagaattgtgacagaatgctacaatgcaggctgggatggtggctcatgccagtaattctagcactttgggaggctgaggcaggaggactgcttgaggccagaagtgcaagaccatcctggacaacatagagagaccgtgtctccacacaaaaattaaaaaataaatttaggctggtttcgtggcatgtgcctgtagttccagctacttggtggggtggaggggctgatgtgggagcatcacttgagtccgggaggttgaggctgcagtgcagtgagccatgactgcatgattgtgccactgcactctagcctaggcaacaaagtgagaccctgacagcaaaaactaaaaaaagaatgctatcatgcaaaggttacccaagcatggtacagggacccctaaggattcctgagatcctttcagagagttcacaaggttaaaactatcaaaaccatttttataatatcaggaaaatgttattagtcttttaaaattcttgtttgctcatacagttagttgcacagtagttttctagaggctaaatgatgtgtaacatcacaacagaataacaattcagcagttatgagaatagagctgatatgaaaagccaggcattaaagaaatttgcaaacatgtaaaacaacgccactcttctattttcactatttttgagaaaataaaattatttatgtaaccacacataatttttattttaattattatttaacatttaattatttataatttattgctatttattatattttattaatataatttttattatttacaattaagctattatttaatattatttattttaaaatgaatcaatgcataaatatatttttataaatctgttttaatttctaatatagaaatattggtagagggaatacatataaacaaaagctctttgaagtcctaaataatttttaaaagcagaaaggggtcctgaaacttgaagtttagagccactgctataacagaaggtgggaggtgtgacctattgacttttctctgctattcagcagatgtaaaaatttaggcaaacagattttagtgggtcttcagttacttcgtctgaaaaatgaagatgaaaatattgtacctaactgaaggcaggcagctaattaaatcatctttattttgctttagcatttacagtcgtagggactcttagaaaccagggcatgtgggtctggtggcagcaatatttaatatcagaaaatcctaaatgaaacctcaaaaagtccatttatgactttttacttctaatgtccatcaaatgtatcctctgtgatggttctcagaggacattttccatcagaatactaccaccacccattgctctaaaatggcaagtaagtgtttgggaatacattctaaaatatgcatgttaaaccctgacattagagtgaaatgtttcccattaagttttaaaaaagctttacatatttcattaacaaaatcccctgcacaccacaaaagtcttgcgagagaacaaacataattctacaattttagttgttactactgctttgtgtaaaataattgctaatatggtaaattcatatttttacattaaaaacaatcttacagattatttttatggggtcatgaaattacccaggagcttccctttaatagtaatttcaaaatccatactccagaactatttatagctgaatcgatgttatgagattctttcagcacttctgataacaaacctctattggagagaatccaaaacataaaattccttgccttagaaaagcagtttactcatactacagtttgacacttctgtgtaaaaacaaaacgaatcaacctctagatgtcttcctatgtttttcagtgggttaactacacccaataactcaagagtttttatctccactgcttacctagtctcataagaatagctatgataattgggaacctgaggattgaaatgaccaaaagtaagagatcagaaaaggcaattgggttgaaaaaagtctaagctactgtgcagaaaaataaggatgaaaacagaaaaaaaaaaaacaaaaccataaaaccagtcaaacccattctattactctatgtcttaatctttctagataaccacattcaaaattcaatggaggaattaagacagtaaaataacaattatctaaattactttgggattacatagtccaagctcattaattatactaaataataatattcaagtgtttaaaaaatggaactcaaagttttgataatgctactcccttgcttgaaagactgcattttggctacaaaaatagattttagactcctagcaaccaacaatttcacaatctataccaatatcctaccttcccaatctattgttaataattcctaacatggcaaatatgtttaacatcatgtgccaactccagtccattcattgtggttctggctactttcaaattctgagagaaggtatggccacagatcagtgaaatttgctgtgggtaagggagtgggaattggcacgaagttttgagtatagttatgcatttctacacgtattgtccaacaaatgtgacctgctctggtcaaactcagcagagcaagtcacatttgttggacaatatgtatagaaatgcaaggcccagtacaaatctcatctttccaccaggaagccttcttactaaaaataggaagtcactgcttacttttctgaatagctatagcattcacggtacttagaatatagcagagtgattaagagtgcaggctctgaagccagacagctggggtttaatattggttcagccacattaattaggtaagtaactccagacaacctacttgagttctcagtgcctctgttcctcacaaatggtgataatattagatctatctcataggcttgttataaatactaaattaattaatccatgcaaaaaactttgaaaaatgtctggcaaatagcgttcaatatcagctgtttttattattactatttatatgacaagcatatagtgccttatcatttttattatttttattgctccaactagtatgtaaataattgaaggacaaacacttctctttctttacctccattgtgtcaagcacaagggttttacatgtaaacaagtgttcaataagagtttcgttaaagttgagatgttaagtcaatatcagcttcctatggctgcatttcttgccttttgtcaaaaagtaaatcttagataactgcacattcaaataattgacacttatataatctacacaaaactgagaagaattattacccactgaagattactatgtaatataagtgctaataattgaaataaaccaggcatggtggtggacacctgtagtctcagctacttgggagtctgagctacttgggaggtttgtgtgagttgtggagtgagaccttatctccaaaaataaaataaaataaacataaaaaataaagaaaagaagaaaaacatcttaaaaatttgaaataaaatatataattcttatataatatggtactattccagaattctgttcagcaaccatatgaaagaagttcacaacaacaaattgataaattaatccaatgagatagttatttggaagtcattacatttatcaaagaatattggcaacaatttgaagttcatgaaagtgaaagtacaaatatagcagcaagatataaattcgaagaaataaattgtttcctttctgccttaaaagaatgcaaggaacatcttaaatgcatctcaaaacacaaaattgaaaagaaaatttcattaaggcataaaaagcacacactcaataaatatattactaattttgataatatagtttaatagctactgacaaactggtatcaatgatacattactgaatgctttatgattaatttccggtacagcgtatctttcccatttcataggctaacaaaaatctaaaatgttagctttgaaaagcactctttaaaatctgacagcttctggacattaagaaatgcaaaatttttctatataattattacttctgtgaaagtaggaaaagaagaaaacatgtttggggtggtagtttgagaggaatgggttgttacttataagataaagctttttaataagtagtatttgaacatcatatgtctatgtaattaaattacataaaatacataaaaatacaaataaactcatcaccaatgaatatcgaaactggggagaattgtcagtattttcaaaatatttatttttctgatctttggtgctccgtagtttcacacgtttaggtatatatttatcttatactgcttagaactcatgcttcttgaatttacatcttattagtcctagataattctcaaccacaaatttttgcatattgctctttttcactaaatatctccttctgaaatgcctattagatgcatgtatgacttttcttcttgttacatgaagtctatcctttatacaacatcttgtccacatgtgttatctttatcctgcatttggtgtaattttctcaaatctgtctcccagctcatgtaaaaaagcataaacataagagcacaggctctagatctggcatgctgcagatcaaatcagaactcagcacctaggagctgtgtcaccttggacaaaatgacttgtattttggtttcctcattgattattaggataatattactaaatatttcatggggttttctgagaattaaaaagagtcaatatatgtaaagtgctttaacattttctgatattgtatcaataaggtgagtttttaattttaatgaataaatttttaatttctggaagtttcatgtgttttttacctcatatttgaaagttctttttttgtggtgtctttatctttgattcctttatctttgttcattcttatattttctattagattgctctagtatctaaagatttgggagttctgtcatatttgctaagagtactgaatcttgtctagagtgaattatttctccatctgttttgtaattttgaaagttgaatttattagtggctagagttgagaatctgtttctcttaagaggctgatttttgcattgcttctgccaggttttctagtggtatcattggtttgataatgtgcattgtaattgaccaactttaggggttttctatacaaaatacatgggttgtaacaatttaaacctcaatcattgtgaaaagcaagcctataatggtagtgttcaaatgacttttttttcttaccccaggcatggttacttggggtaagaaaagcaaccatccttgccaacagtgatggttggcaagaattttttttttcctggtatacccctagtataaaggatataggccactgaaatttcaggctttagataggagctctcatttctaactcaccgtctcctttgggtctcttatctaagtagtgatgtaatcaaagttagaagagtgggagtggtctgtgtttggaagggagagggcagtatattattgtattaaaaaaaatagtaaaactgattaaatgtttggtctgcttttcattaggcccctgccctggcacatctaacaatttcagtgataaaattcttctctgacaacaaaaatctcctgttggtctaagttgtaagctactgcctgcatccaggatagactgcttctattgctctacctttagtaactggctaacctgagtgggtaaacttcgtgttaaaagttccataaaatgcccagaaaaacaacagttgtaactgagattctgttgtttttgtttatttcctttttgttactgacatccaggaattaaaatttctttcttgtgcacttaactatgtattttaaaaaatgtttgctacattttctccaacatttccagatgtctgtaaagaatgggtgagtaagtagccttgactataaaatttccagagttagaaccaccgccaccaggccgggcacagtggctcagcctgtaatcccagcactttgggaggctgaggcggatggatcacctgaagtcaggagttcaagactagcatggccaacattgtgaaacatcatctctactaaaaatacaaaaattagctgggcgtagtggtgcacgcttgtaataccagctagctgggaggctgaggcaggagaattgcttgaatccggaaggcagaggttgcagtgagctgaggttgtgccactgcactccagcttgggcaacagaagaagagactccttctgaaaaaaaaaaaaaaaaaaaagcccccatgtttttatactttctaaaatttaattataaaattgttaattaccttttctactttgtcttcagaacacatcatttctatttgtttatggcactgttgttggtagaatgatttaagttcattttctttaagtaacatttccaacttcagatattcttgtctaatttcttctcgattctggaacaagcagttcaggatttcttgaaacctaccaaaattaaaacacattacagaatctcactaattttgtagcaaatcatacaaagtttacttaagagaaatgcataaacaaaggaatatatgtaattcacagaactacactcaatgataaacagcaaataggtaaaaatgactatttcctattataaaaaatgtttagaaaaagttattaactttacttatactacattaggtttctttctcacccaaggaagatcatgttgctgaaagatatgggcatcataaggtgtctggtattgctaacatactgcattttgtctatgcttacatctatatgtaatcctattgcttagcaaaaataaaataatttttgtttgttggaaaaggatgataacctgggcatgcactatccaaattcacaaggccctattagcaacacagcattatgtgttgttacggtgttggctcaccgtgaaggaataagcagtaggaggcataatcatatggtgcctccatggccccctagggaaagtgacagagccagtttttagcttcagaatttgttatagtgaaaacagaccatacggttaggtaccccctacagcctggctaagaatcaactgacacttccaggacttaaaacatccaagtttataatgtggtaaacctagaaaagctgctgcagtgtgcttcctcctaagggcaatgtaggtctagaatatcaaagatgaggagtttgattcatatttccaaatggccacagacttcaagctgattaaattttatgaaaactctggatggaacctgcaatcctgtatccttctgggtcagtagaacactggtcacagtaatattttaattttttaaaatatattcatggttttaagtcatagatcagctctgagctacagtaacagtataaacattcataacagtgaagactttaaaataaagtaaaatcaagtacatgcatttaaaaaaaatgttctaaactggccagaattttagatatactcataaggatcagcggtaaaaacactgccaatttacgtcagtagctcttaatgaaactctattgggaatgaatataagccacaaaacactgactttactgcagttttgtcaaactgataaatatttcataggaaatatgtacagacactgtgatttcattgcattatattacaccaacggtaatgccagtaaatgaattgataagttatctttaggttatacttaaagagataatttcttagtttttctataactttttattcaaggacaaagttaatatttgtcaattttgtgatctagacttattttaggtggtggtgttcactgattaggatgaaaggaagggaattggtatcaaaggtttctagagagctccttaacaacaccaaaaaaagctatcgttttaaaactgaaagagttctaagcaaacattcaagttaaaagtatctgcaccaatatttttaggtaatacatgagtattaaactccacaattttcttggtatagtcataagtgaagttttataattactctttataaaagaacaattgtgttttctttttatgatttccttttgagaattacctttccaaatagctctgatagacagaaaatactgcagattcttccttttatctgtttaaacttcataaaaatgttatttaagttttataacaaaagacactttgaaaattcatggcattaaaaaagtcccatgtaacagtatgatggataaaataaaagatatttcatacatttatttcaaaaggcagtaatttttaggggaaataacaaaagttatttggtacataatttttatcattaacttcttcataaatacaagtaagttacttaacatattgccagaattaaaatcaaagcttttattgttggtcaaaagtcaggttgaaatatgaaaaacatccaaataaatacttatttcatagctcaggcccttccctcctgagaggagggccttaccagaaagaaaggttcaattatttaaagccatcaactccagaaaagtttactaatctgtaaggttttaattggtatagtagacttggttaagcagtgctgagcaattttatggaatcagttactctaaatttgagaagctggagaattctaatagatatccaattacatgaggaccgctcccacatttaactaacaaaatgaaataacaacaactaataatactatatcagagttaaaagttttctttgatgagatgaaaaaccagaaatcatttttacttatttcttaagaaatactgtaaaatattttatgtaaaacttcaacacacaaatgcaattttaaaaaattatagctgaatcaggtctgaaagccacttttgactataatcttgttttccttatagatactgagacactttctactaattctagagctccaaaacagtatgaataaccattggtttaagttgtagttggtttctcatactaataacagataactggctctgcagaatgtcaaataagtttacctttactcaattttaatgaaagttgaacagattcctttactacaggttttccctttaataagctgatgtactttggaagtttcctagattccttatggagtaatttccaaacttgtttatacaggaattccttattgtcctcctagaagtactcaagattaacatcttgatccagtgtctcaatgggtcaatttggaaaactgtcttagctgcttgaggaaaaagtacttttgcatactgtattactaactgctttctttttaaaaatttacctgctaagaagcttagtagaaacctctcaaaattctgtattggtttatatcactgacagatgttcattcaaataattacccattcaggctcaataagtagattagttaagctatagagagttaataaaatgaagaatagagtagatttacatgaaatatacaaataatgattatgtcagaaaagaacaatatgagcgccactaaaattaaactaagtctacagggtgacataactggctttgtctccactatcttatacctaagttctggttctcttcaatatatgtaaaataaaaactaaaattacttgatctacaaagtgtgaaatataaagagaaatcactgacttcttgaattcagttgagaatatatatacaggcataccttgttttattgtgctttgcagacactgttttttttttttttttttaaacaaactgaaggtttgtggcaatcttgctgtgagcaagtctataagcatcatttttcctacagcatgagcattcagtgtcacattttggcaattcttgaaatattgtaaactttttcattattacatatctgtcatggtgatttgtgaccaatgatctttgatgttaccattgtaattgctttagagtgtcacaaattgtgcccatataagacagtaaacttaatcaagtgttgtgtgtgctctgacggctccactgactggctgtttccctacctctcctcagccctccctattccttgaaacacgttgaaatcgggcaaattaataaccctaaaatggcctccaagtgttcaagtgaaagagttagttgcatgtctatcattttaaatcaaaagctggaaattactaagcttctaaggaaggcttgtagaaagccaagataggcgaaaagctaggtctcttgcacaaaacatttagccaagttgtgaatgcaaaggaaaaattcttgaaggaaattaaaagtgctactccagtgaacacacacactaagaaagtgaaacagccttattgctgatatggaaaaagtttcaatggtctggatagattaaactagctatcacatttctttaagtcaaagccttatccagagcaagccttctaaatctgttcaattctacaaaggctgagagaggtgaggaagttgcagaggaaaattctgaaactagcaggtgttggttcataaagtttaaaagtgtaagaaaaaaagccttctctagaacataaaagtctaggtgaagcagcaagtgctgatgtagaagctgcagcaagttatcgagaaaatttaggtaagatcactgatgaaggtggctacactaaacaacagattttcaagatagacaaatcagcttttaattggaacaatatgccatctaggaccttcatatctagagagaagtaaatacctactttcaaagctttaaagaacaggctgactctcttgtcaggggcttttgcagctggtgactttaagttgaagtcgatgttcatttaccatttacaaacatcctagggcccttatgaaatatgctaaatttaactctgcttgtgctctacaaatagaacaacaaagtctagaggacagcacatccgtttacttaataattttaagcccactgttaagacctattgctcagaaaaaaagattcctttcaaaatactaatgctcactgacaatacacctggtcactcaggagttctcatggagatggacgagaagattaatgttgttttcatgtctgctaactcaacacccagtctgcagctcatggatcatgcaattttgacttttgtgtcttcttatttaggaaatacattttgtagggctatagctgctatagatactttctttcatggatcatgcttctctgacggatctaggcaaaataaattgaaaagcatctgaaaagaatttgccattgtagatgccattaagaacatttgtgatatgtgtaaggaggtcaaaatatcaacattaataggaatttggaggaagtagattccaatcctcatggataactatgaggggttcaagatttcagtgaagaaactaactgcagatgtggtggacatgaaagagaactagaattagaagtggagcctgaagacaggactgaattgctgcaatctcatgattaaacttgaagaaatgaagagctgcttctatggataaccaaagaaaatggtttcttgagaaagtttaccacctactccctataaagatgctatctacgttgaaataacaaaggatttagaatatgacataaatatagctgatagagcagtggcaaggtttgaaaagattgtctcaaatttttaaagaagttctaccgtgggtaaaatgctatcaaagagcatcacacactacagataaatctttcatgaaaggacgagtcaatctatgcagtaaacttcattgttgccttattttcagaaattgtcacagccactccaacttgcagcaaccattactcttactagtcagatgccataaacattgagggaagacctccaccagcaaaaagattatgacttgctgaaggttcagattatcattagcatgttttagcaataaagtatgtctaaaattaaagcaaggcattatcttttcagatataatgctattgcacacttaatagactatagtgtagtgtaaacataatttttatatgcaccggaaaaccaaaacatttgtgtgactcattttattatgatatttcatttattgcagtggtctggagaactaaatctgcaatatctctgagtcaagcctgttatgtatgtatgtatgtatgtgtgtatatataaaattataaacatacatatctatgtacatataaaagacataacaaatgataattaacataaaatacttaacaaaattttaaaacaataaattcctcaaaattagatcttaatgtaatgtttacctttcgatttctttttcctgagggtttgaaatcattaactttgcttggtcattttcattagtgaaggctttctgttcttcataggcttttagtttttcttttaacaataaaatctagtagagagaaatcactagttaaaatacaaaagaagtcatttacatggccacagccaaatttagctgtaaaaaataactgaattaaccagattttaattttaatagaaaaatatgttaaatttttggacaattaagattataaaattctataaatgaagaactgcgcaagactagcataaaaatataaacagacatacctctgccttctgctcattacagatctttacatattgagttatatgattattgacattaagaacattgctcttcaactgttgaaagatagaaattatgattgtttatagagagataataatcacagagataaatacatgaataacatgacattgcattttcattgagttccatgaattcagaatttgtgataattatgctcacatttaaaatctatgacaagaggatctgctgagcacattaataatgtaaatgaaattataaagaaattactttattgagaaaaggtatttataagcatgtaattgcaatataataaaattccctactttcttactcctttgacttctaagtaatgcagagatagtttttcatgctaccctttcaggaattcagttggttttcactgagttcttactatattttaacatttttatcatagtatgggccccttgacatttatggtatagtagagaggatattaaacatataaatccaaatacttgatattgagaccttgcctacttctcacttaacctgacttagaattcgaactgtttgatgtttgggttactcatctgtaaagtggagctaagaattcattcctcaaggggctagtgtaagggctacatgaaaatcatgtgttacaaacctttctgtacacccatatagtatgaccttttacaattaattatataatgagcctgggaacatttttcttaagatgacaataacatgctcatcttcagacacttaaggagagtggcagaggggaaagggagaagaggaggagtcatcacaacagcttaactctaggttttttcaaccccgatatattataatttaaacatacatattttgatatacatgtttttaagtacaacacatctttggcaaaaatactaatgtaaccaattattattattatatttctggagaaatacagagattggtacatcaacatcatgaatgtggactataaattatgtagaaaagactttttaaaaaataagctttatacctattttacagatgaaataataggcttggaaagcttaactaacatatccaagattttcaggtgaaagttttggttctcagctaaagcagagcatcatcctgggctgcaacaaatgtgttcaagttttgagtgattactccaagatgccatccaaatacaatacgccatgacatcaaagaggttagggagaaagccactcaagtaccatagtacccaaaagatcgaataatagtatctattatatatatatatatataatcaagatgctttatggtttaaccgtctaaattaatgttcgactaagcaggttagcaaagacccttctaaccctcagctgcctaactcattatgtagaaacataacctgaattaatactttcattacaattataaaaatcatatgcagacaataccttcacaacaaaggcagagtaaacagacttacagaagatttaatgtcctttgcccggttagcatacttaagagtgttatatgtgtcatcgtagaatacagaggaaggactaacagcagctatcattatagtttgacagtttcctccaagagaatcctttaacaagcgagtaagcttactatttctgtaagggatatgctgattctttctctgaaagcacaaaaaagagatcttatgtcattttccacatttaaatgcaaacctgctaccactttagcacttcactgtggggggaggattacctaggtgccgaggcaagagactgaaggtataaactgtttcagtataatgaaaaaaatagctagaataagaatagtcataatacaacttagatatagagatgatcatggacaattatcaatcactattataaacattattaatcattagcttttaatattactctttgttgcattacgaatataacctaggaataaccggcgggtatggggtcagctgctgaagggacattgtgagaagtgatctagaaggcaagaggtgagccttctgttatgccggcacaaacgccgcttgagggctccttggtcaagtggtaatgccagtgtctgggaagacacccattacttagaagaccgcgaaagggagtctcctttccttggaggagtcagggaacactctgctccaccagcttcctgtggaaggctggatattatccaggcctgcccgcagtcatccggaggcctaaacccctccctgtggtgctgtgcttcaatggtcatgctccttgtccactttcatgttcctcctgtactcctggttcctctttgaagttcacagtagacagcaacagaagaaatagtgaaagtcttaaagtctttgatctttcttataagtgcacagaagaaaacacggatgtatgcgccatctctctctctgttatcggctacctaagagggaagggtcccctgtcctatgatcacgtgacttgcctcaccttgtcaatcacttagaagattcaccctccttaccctgcccccttgtcttgtatgcaataaatatcagcgcgcccagctgttcggggccactaccggtctccgtgtcttgatggtagtggtcccctggggcccagctgttttctctttatctctttgtcttgtgtctttatttattacaatctctcgtctccgcacacggggaggacacctgccaagtcccgtagggcttgaccctacacttcactataagaacttaagcgctacccaattttatccagacttataattggtgattgaggtgatgacaaagaatcttacaggattattcagtgaatatatgaaacaaatatcatatttaaaaatatctgcatctgagtaagtatgaactttagttcaaaataaaataatacattaatattggttcattagttgtgatatatgtaccatattaatataaaaaattaacaatagggaaaaccaggtgtggagcatatttaacaaaatctgtatcatcttagtaacttttctgtaaatctaaaactatttgaaaatgtctatttaaaaatatttccaatcattgaaaaaaattaaaacacaaaagtaaacagatgtcctatacaaatatactagaggaataataactgcatggcttagagctacttttctccttaaataaccatctaatatcgatatgaaataaattataattacaatgttacagacatttctcctgaaatcactatagtgttctaaaagaacctaatacttcaagattttcttttacctaaaaagaaaaaagtaaaagtaacaaataaattttgttaagttttgtttctgattttgaagtaacaaataaattttccagaaagtagaatgtttatataagaacagattctttacaatgttaagctaaaagcaacctaatgttatccaatgtatacctttcatattgcaaaacaggaaattgaagctaagcataaagtgaaatgatttgactagggtcacaaagctagtagttcaaagagccagaaataggtcctagttcccccaattctcagtagcaatgtgcttccaccatagcagtctttcccaaacagtgtgaaatatgcactgctggtgctattttaagtgattttacatgtacaagacatatattaaacattgactcacaaatgactcaataggagtcattattaaaaaattatagattagttcatatggcaaaaatcttatgaatggcttaagtttacaaaatccgtctctgtgtcatgctgtctgcttacaagtttgagattttaactcagaatataaaagacatcttgcttttgcaatcttcatgtgatttcagtaatttgcctttttttaaaaaaaaaacctgctccaattattttaatatacttattctaaatcaaaggtaacattttgaacagtttaacaagccaaagcttttttaaaaaaattaaatttaattttttaattatattttaagttctgggatacatgtgcagaacgtgcaggtttcttacataggtatacatgtgccatggtggtttgctgcacccatcaacctgtaatctacattaggtatttctcctaatgctatccctcccctagcccccacaccccctgacaggccctgcctggcgtgtaatgttcccctccctgtgtccatgtgttctcattgttcaactcccacttatgagtgagaacctgctatgcagtgtttggttttctgttcctgtgttagtttgctgaaaatgatggtttctagcttaatccatgtgcctgtaaaggacatgaactcatcctttttcatggctgcatagtattgcatggtgtatatgtgccacattttctttatctagtctatcattgatgggcatttgggttggtttcaagtctttgctactgtgaatagtgctgcaataaacataggtgtgcatgtgtctttctagtaatgatttataattctttgggcgtatatacccagtaatgggatggctgggtcaaatggtatttctggttctagatccttgaggaatcgccacactgtcttccacaatggttcaactaatttacactcccaccaacagtgtaaaagtgttcctatttctccacatcctctccagcatctgttgtttcctgactttttaatgatcgccattctaactggcatgagatggtatctcattgtggttttgatttgcatttttctaatgactaatgatgagcttttcgaacagtttaacaagctaaagctttcgagtatggtttaacttaaattgccatattataatttttttgaaaggatgtcatttttacaaatatctatgttgtgaaatacactgtgttctaggtaagcacttctggcatatggtatatgtaaactatatttaaatatgcccagaagcatctagtaaacattaccttattaggtgatttcatgacattttataacttaaaccccccttaaaaaaaaatcaacactctttaatacttaaccctattttataatatataaaatactaaacaacagaatttagtttttaaaaatatggttagtttgtcttcatgccaatttaattaacatgtttgctgtgtagtgatcactttaaaaaccaagttcaaaataagtgatgagaatgtaccataaaatattaactatcaatctggtattcattactttctatttaaccaattctcattataaaactggaaattaaaaaagaaataaaaatcagtttaatacactaggttttctgaggtctttgatctatatattaacattactgatatacccttgaaatgtgatatatatcagacctttaaaacaaaatcatattaaatagtatctttcttatctatttcaatatttcaaactatttaacaaataaacatataatgcctacctttgaatctgctaaggcattgatgacattcccaagagctaaaagtgatctattaatatttgtgccttctacaaatcgggtccccttagcaccggaagtacttgctcgctcagatcctgccaggtcaatgagtgacatcttggcaatacggacattttgattgatacttgctgttttgtcttgttgtcgcaagtaaatctttttgaaattacaaaatagaaaaaaatgaggataagatttaacaaaattaaaagggaaatatatactttaatagagcacaaaatcaaaattattttcattttaaaaaacaactgaacaaggtataatctaattgatatttaagagcactataagtaatcactatagctgagaaaaatataaaaatgaaaagactttctatatgtgtaccagaggaaaaataactcttgacttgatagaagatcaaaacttccatttctattctggtaccccttgaaccaggacatggaattatcagaggtgatggttagaaacacaggtcctatagaaataacgttcctaagaaaaaaccaaagctactcgttagaatgccttgtatgtatctagagattgcccctcaagggaagctgaattcgaatcttgatccaaactgaagagtaaatttattctaatcccccaaacctgtatatgtcctcataattaaaatatcatatataaaaaagatagtacagtcttgcatcacttaacaacacagctactttctgagaaatgcactgttaggtgatttcctcattgtgaaaatatcataaatcaaacatgagagaaaatgataaaatccacagacttggtaaacagaatgtagcagactgcttttggggtaataagtaggagcatactctaaaataacaataaaaagtaaatacataaaccagtaacacagtcatttattaccaagtctcatgtactgtacataattgtatgtactatattttcatatgactggcagtgcaggttcgtttacaccaacaacaccacaaacatttgagtaatgtgttacgccacgacattatgatgagtatgacatcactagatgaataggaacttttcagctctgttgtaatcttatgggaccactgttgtatatgtggactgttgttgactgaaatgtcgttatgcggcacatgactatacagtaaagtctacttgtcattttcaactgcctagtaaagcataatttattcaaatattttaagctactctgtaaggattatctgaactacattaggtggcaaacaaaagatgaggaaagtaagatattacttaattataaacccatttattaaattgttttataaagcaggatctaagatatttcagagacatctcagtttcacagagaaacatttctctcaagactattaataaccgaagagttagtggtaagaacaaaaaagattaatgaaatttttgttgaccagggactatatgaatggtttgattgccttcaatatcaattctttcaactataacaatttctttcttataagatgtatctcagatggcataatactaaaagaagctttatattatcatttgggtattaaaaggactagaaactataagaaataatagctattaacctatgctttagctctttctggaattgtattcctaagacttactgtgccaactgaatcatacttccttaaggaatgcgactatttacatggtccttatggtgaaatcttttaaagatgaatcagtatgttcccaaattattagttctacaagtctagatttatagagataagttttactgtgaatataagattatttattggaaagattaaatattttcagcaaaaacaaaacttttattactacaactttatcctcctgtatgtttacaaaggtagaaattttgtatttttgttttaagcatctcaattataaatactcttggtgacagaaacttttcaaagctataaaggaaagtactgacaagtgaagtctacagacagatttttttcacatgtatgaaaatgatgattcatgaatttagcttcatttattttaaatccaattttaagagtgatagtctagtggcctcttacttggaaaacagcatgagaacgagaagatgtggcattcatatcagtgggatgttgtgtcctgtttttgtttccattatccaataaatgtaaaatttcttctgaggatttgggctggagaagtttaagtagaagcaaaatttaaaaatcagcaatatatctttaaatttttcaataaatgttcaaaaaacaaaaagatttaaaggtaaaagaaaatgttttctataaattcaatcctattaaatactaactttgtctcatctgcttagtattgtgtgtgtttatatgaagaataaaagataaaatctagtttctcaggagtggagtctattgagtagaataatatgacattaaatgttcaagcagatttttgaaagaaaagctcactcattgggaggctgaggcaggcagatcttgagccccagagtttgagaccagcctgaataacatggtgaactcccgctttctaccaaaaatacaaaaattagccagtctcatagcccggtcacaaaataaataaataaataaataaataaatacatacatacatacatacatacatacatacatacgtagattaaaatttaaaaataaaatttaaaaaatgtttttaaaaagttcactctattttaaaagaaaacaaaataagactaaaaatataccctaatcatttaaattctgtacctcattagttttttttctcaaacaggatggaaagtaagtgcatggctggtgaaaatagcttaatagtcacatttgctattctaacagggaaaaagatgtttatatacatacctggtgtaaagtaagtccatgaacgaccacccctttttgggtatcttcccggacagcaagtggccctgaatttactaagagatcacgaatctgttcattatataccttaaaataaaaagaactgctttagcattgatgtaaaaaaaaaaaatggtgattacatcacacatacactgctaatttaaagttattagaattttatcctaaagacaagcagcacctcacttttattcacctcataccttcgatctcatgttgccattaattttctatcattccagaaaattgaagagtttgattacctaggtttgaaattgaaacttaaagttggagtcatcccctcactcataattcctgccttgatgactatggtgaaacagagataagtaagatagctgaagtgcattttcttcttttctttttttttgagacggagtctcgctgtctcccaggctggagtacagtggcgcaatctcggctcactgcaagctccgcctcccgggttctcaccattctgctgcctcagcctccccagtagctgggactactggcgcctgccaccacgcctggctaattttttgtatttttagtggagacagggtttcaccatgttagccaggatggtcttgatcttctgacctcgtgatccgcccacctcggcctcccaaagtgctgggattacaggcgtgagccaccgcgcccggcccattttcttattttcaggaaatgtatggaacacacggtactgggaaactctgggaaatgcaggttgtatggagatggcatggttcttactgtggaagatatctattatttcacaatgtccaagtagtccaaacttttgaaattttagagcttaaatgttacagaaaacccataccaaggacaggtggtatttgtttcggttatccttcaattacattaattttgacatataattactaataataaatttcaaaaggagacccatttgaatgatgagggaactaagactctgagaggttaaatgatttgtccatggtcataaaggtattagcagagtaggaactggagcctagacagcttcactctactgcatcatactgaatacatatctgtagtttatatctctggccattttttaaaaagtaatttttataagtaacacacatttctatgtagtactagtgacccgattttatgaatttattaaaggtataaagtttattacttgctaaccaaatgacaattacaactattactgacaagatttaagaaaaaaaataacaaaatttagtctctaaaaaattattttgtgttatttccttctgtgttctaataagtagctcatagtgataattcattgctggtacaattctctacaatgaatgataggcattagaatgtaataaacccttaaaagtagtaaaatgataatatgaagaaatatttttaaatcatatctttttgatgagcagcagtcactttactattgcttccccttacccacttcaatcataacaaatcaaaaattagaataattgttctcaagctggtctatgtatgacactcaccaaggagcatttaaaaaacacagatgcctcagccacaccccagacctactaaattaaaatatcccaagtgtgaggcctaggcctatttttaaaaagttctataggcaattctggtgcacagcttgagtagaaaaccatattatacactgccttatcattaataatatgaaagtagattaaaagaaaaaataaaaattgtgaaaataaccaaacttcaaaattctcccagttataaaaaggatctaaggaaaaaataatgaatgctggcaccctcttttcccaattgagttcagaacaattcttttcatagttaaccaaccaaagaaaatgagttttacttcttttttatctataagttctaatgaagaaatttttagcaacatatatgattttaatcaatctttggaagaaattctcagtagggaccaaactacaagatgttaataagagagaaattttatggcaccagaaataaataggcaatgaaatcagggtttttatgtttctgcactggtttctgaaacactatttgaatgcaaagttagagtgtctgccacttagttaccatttgttgactattaagagggtagaaggcctcaaagaatgatggggtatttcaaaaggacacaagagacaacatgaagaagtctcaactagccaaatcttacacaatttgaacatacaaaaaatgattacagaaagggattataatctattcaataaagtaaaaatgcaggaggctgatataataaggggaaaactcttccttatagtagaaagtcaataaagaaatgtacaagaaatgatggaatttaaaaatcaccatcatctaccatcataatagtaattgattcagtcaccaatcatcagtggatactaataacaatagataaaagtttgaggagtaaggaatatttacattgtctacatacatagtacttactaattataaatggtaaaacgtatcttcaaagtggagaaactctacagaccacagcataataaaactgatcaaagttaacattcccagtatggggactaatcaacggcttaagccttttttgatgcagtaagaagtgaacatcactttgcttgtgttactgtcagtgcataatctgaatctaatcatgataaatccaaactaaggatttttatggcagtgttataaaagacaaaaaattacagattaaggagactaagatataacaactgaatacaattttcttttacaataaagggtattaatgaaataactagcaaaatctcactaagatctacagataattatattgtatcattgctaatttcctgattttggtacctatttagcagttatttttaaaaagtcctgtttttacaagtctgcaacctagctcagaaagaaagaaatgtatacacacacatagaaagcaaatgcagtaaaatgctaacatatgggggatctgtatgaagggtatctggaagttctttgtactattgttgcaacttctctataagtctgaaattttgccaaagtaaaaaaaaaaaaaattaacagaaaaagatgcataacaaatattcaagacaaggaattttaatgattttatatattcaccggaaaactcttatataataaaatcatttgtatgaatcatgtcaatgatttcccaaaactattgattaatctaaattcccaacttacctccagatatgaaactgcagtactacatattttctcttctttaatctcatccatgcatttgtaaaggtgtaacattgttagatacatcactccaggttcatcagctgatcctagcatagtgtgggtcttcccagcaccagtggcaccataggcaagtactgagtttttaagaaagggatcaaaactctttgttatgaaatataactctattatatctactatctagtggatagttccatctggatatcctgaacagtatctttaaaccctacaaatccaaaattatagtcttatccttaaaacctgggaatgttaccacaaatctactcttactcctatgttctttactctgataaatggtatcactacccccggccccccactctaacaactactctttcaaagcaatcttaaaatctccctacagattagtcttcatcttccatcttctttatcctttgcaatttgtagtcatctttatgaaaaagaaattggaatacgttaaaccttcttaattgttccaggtttacataagttcaaattcctcactttggaatattataaagcactcagagggtgcctgcttattttgccagcctactgcttccttgctgtgatctagggatacttaattattcaatactctcagcatttgctgctcctttctactctgctgtctttgttctgaggagtccctctacttgatccagctggaaaacttccacctgcttctaaatgctcagcacaaatatcatttttctctgccttccctcattgtcctaggcagacacatccactctttcacttcagcccttagtatatgctgcatatccacatccctctcttctggcatgtatcacactgtatttccaattgagtgctccaccaaggcagaggccatgtatccttatcacctaacaaatcacttgccacaaagcaggtacttgaataaataccagatagatgccatttataagattcctttttaagtttttgttttaggtgaattactttgtttcctagattctattcagtacaagaaagtcatgtgaatttcttccaatgtataaaaaatccactctgtgggccaggctcggtggctcatccctacaatcccagtactttggacagctaaggtgggaggactgcttgagcccaggagtttgagaccagcctgggcaacacagtgagaccctgtatccacaaaaattttcaaaaaattagctgagggcagtggctcattcctgtagtccagctattcaggaggctaagatgggaggattgcttaagcccagcaggttgaggctgcagtgagctgtgattgtaccagcgcactccagcctgggcaacacagcaagacttcatcaaaaaaaaaaaaaagacaaagaaaaagaaaaaaatccactctgtgaattacatatggtaaatacccaatacaataatcagttaaggcaataacttctcatatcattgcagtgagctgtgattgcaccagcgcactccagcctgggcaacacagcaagacttcatcaaaaaaaaaaaaaaaaaaaaaaagacaaagagaaagaaaaaaatccactctgtgaattacatatggtaaacacccaatacaataatcagttaaggcaataacttctcatatcattattggttgaaaatatttttataaattcaatctgtctgctaaaccaaatgttaacaaaccacaaaaatacttttcagtaggatgaattaagatggtgtatattttactttgttctattttatgtaagctggtatataaggaaggtttaaaatggcttattcttaagcttattcttaagctgtcttaaagacagggtaagaatataaagaaagccaaaaagaaaccaaaaattcaatagtagtttgatagagagtctactttttttcttagagttaagaagatttcaaaatctctagagacatatgaagtttcatgaaagaggaaaagaatatcttcttaaaaatgggaatcacagtagtaccaatcaataaaccacactccatccttacttgatcaagagggtatgtcttccatgataaaactgaaatgaatcaggaaaagacagtgaaacatgttatagttgtgagctgtcttggggaaaatatcataaatactaataattacaaaaataagatattaataatggtgtttagtgcctggtaggcactgaaaaactggccattttgcatcttgggtatctctctctctctctctctttttttttttttaaaaaggaaatcaacatggctttcttgaaaggccaaaaaaagcactaaagatgcttcttgggctaacaatgagctaaagggcatgagataattaacatgggatcttaacacttttaccccctttccaatttttattttaggtttgggggtacagtgcaggtttgttacatgggtaaattgcatgctgcggggtttagtgtacagattttttcatcactcaggtaaggagtgtagtaccccataggtagttttttgatcctcagtctcctcctatccttcgccaacttaaacatttttatttcaatgtgccatgaagaaaaactgctgaaaagaaactgtcaaatgttcagaaatgtctgactactctccaggaatagataattacataggtaagctgtaggaaaagttataaatacaggttatgagaaaactgatgttaattgcaataagcatgtggtttaataacataggcaactgaaaggacagatgtctataggggccacagtaaggccctggaagagaaaggtttacaaatgatgtataagttatatatgataactaaaatttcaaagtctatatttacagttcaagtctattgaatttcttaagaatttttagcatacaagctccttaaatttgattatatttagaagggtcaagtagatgtccgtcctaatgcaaaagtgaaatcggatagagaaattaaatcccaaattgaaacaaatacctgtgcaattatatccattcaaaaaactacgaagaattggcttagtagtgtgttcaaaaacttctgactgagttgacgtttcatcaaaaacagcatcaaatacaaatttaagatccttattttgtttctttataacattttgatttgtagttttctttccatggaaaaaactgacttcttcttgtttgggatcaaaaactaggatatgtttatccacaacatgaaccactttatgaaatccagctgctttttctttagtgttttccggacgtacacgaactactactttcatatggtggcacaggtcttcctcagtgacagacattgttgattatcttgattcctatctgtataaatacttgaatacttctctgaaattaaaaaagaaaataaagttttaatatcagtttttactttcatgcaaaacaactggcatgtagtattcattcaaaaaagatgttaataattctgtttcactcactaggtaaacactgggggaaaaatacatacatacgtaggcacacacaacacacacacacaaatatatgcacacatacacacatatgcacttaagtaccctaaaacaaacaattaaatggcacatacgtacacacaactacgataaaaatctctttacctgaaatattagtaggatatttggcttaaactcaaatactatagctgaaccatatataatcacagatatgctatcctaaaacccagatttgattttcacaagccctccagcactatttcagcttaggtcatgggaatgaatagaaaaggatctcccagaaccatatcaaggtatgattggagagccttagcttgtgaactatttttagtaaaagtgatcttggctgcaagccccttggccgtggaaagtgatacacccaggctggtctctacctgtatctcagaagcccatagggttaaaaatagttccaaggatgcattcaactagatagctcaggtctgaaggtgatagagatggcatagactccttggtgtattcagagacctagtctctgaatattgatcctactctctgctttctaattgtgtgcaagtttaaggaatcaatgatagcaggtttaaaaattatggtctgattttatgactggttcacatgcaaagaagaaaagtaaagtaacaaagatcaataatttggaagtctcaactatccagaaaatactctagaatctgcagacccaaaaaccaaaacaaaaaaacaaacaacaacaacaacaaaaagacaaaaaaaaaccaaaccacaaaacaaaataaaacaccccaaaacaacaagatctgagtaaggaccaaaaggtgagacttaggacgatcctgcaatcccacttctgagtatatatccaaaggaattaaaatcagtatgttgaagagatatttgccctgccatgttcgctacaacattattcacaataactaagatatataaacaatctaagtgcctatcaacagataaatagatcgaaaaaatgtggtatatgcactcaatgaaatactattcagccttaaaaaagactaaaattctgtcatttatgacgacatgaatgaatctagggggcattatgctaagtaaaataagccaggtacaaaaagacaaatatcacatgatctcacttttatgcagaatctaaaaatatgaacacagagaagtagagtaatggttaccagaggctgaggagggtggggtgggtggtaaaaggggaaatgttggtcaaagggtacaaagtttcagttatacaagaggaatagttttggtgatctattgcacagcaaggtgaccatggttaattgctaagagtacattttaaatgttctcacaacaaaaaaatggtatgtgaggtgatggatatgttaattaattagcctgatatgctcattccactgtgtacacatgtatcaaaacattacattgtatcctataaacttacacattttttttgtcatttgtaagttaaaataaaagagacaaaaataataaattcacccaaaacaacatttaagtaaggacttgaaggaggtgagggagttagtcatacaggtattgtaggaagcttttccccatagagagaaggacccagggtgctactttatgttggccagaatggagtgagcaaaggggagtgtagtaagacatgagaggagaaatggaggccattaaaagaactttggcttgtactctgagtaagacaaagaacaactggaagtcgttttgagcacaggagtgacataatctatcactctcactgctatgtggataatataatagactagtctattgaagtaatgtagacaagagacaactctgtcagctcagaatagggtagtggcagcagaggtggtaagtggtcagattatgggtgtattctgagggtaaaggtgacagaatttactgacaggtagatgtagagtgaaagagagagacaggattgaagaataactccaaggtttttggcttgagaattagaaggataaagtagtcaccaactgagatggaaataagaagcagggcttttttcagcagggcgggaagtggtgggaggcagggtcagatcagttcagttttggatagcttaagtctcagagttttgacagacatccaggtggaaatgtcagggcaggtggaagttaagaaaataagaaaataaaggctgtcctcagtcttaatgcagtattaagctttaaaaactttcgggaaatcctaaatgaaggaggaaacagcgatctattgtgttaaatactgctaataggtcaagtacttactgagaattaaccatgggatttactaacacagaggccatcgagaaaagcaattttggggcagtagtggggaaaaaacccgaaaggagaagatccatagacaaggtatatggaaaggactgtttcaaagggaggagaaaaatggtgtgagtgaagggggggggtggtgtcaagatgttttgtttgtatttttttttaaatgggagaaatagcaatattcaagctgaaatatgtcacaaaaccacttagccatatactttcggacagaattcaaataaggacagaattcaaaggaatctaaaagcagtagatgctagacatatctttgcctttttaggactctgtctttggccaaaaatagaatactgaaaaggaagggatggtcatttggattctaagtcaggtcccaggtgaattactactttgtttttcatccctgagcttccgtctagccaagtgtcacaaagcacctacacctcacttttatgaccttggacaagttacttaaccccatgagtctcaggtttctatttgtataaaagagataataatacctcaatgtttgttgcaaaggctaaaaggggtaagtattaaggtattcagtaaataccaattctcttcctatctgccccactcctgcttcttcttgccaaagagactgagttcatcttttgagtttttctcaatgggcttatgactagcactttgggtaggccaattcttccttgcttagaactgccctgttatagggcatttagaactgccctattagaggatgtttggcatttctctcccctgtctattaaaggccagcagtgtcccatattccttgtgataacaaaaactatctcctatacacatttctaaaagcttttagaaatattcaacaatattttaacagatatactatttttaaatgatagattaaatatacataaatgaaattgttaacaaagtaaacataaaatactaagtgattatttcaaaataaaccttagaatctcaaggctggaggatatccctccctaattaattagttaattaattcatgacaactattacatgttggagccaggcagcctaggttaaaatctcagctctatgacgtactgactgtgatagtggataagttacttaacctccttgtggctcagcttccccatctgtaaagtaagactaacaacagtacctatctcataaagttgtaaagattacacacttagaacaatactttgtacagagtatgaactacaagtgtttgctattattatttacagtgtagtgagggaaacagatattaataaaatattgacacaaacatgtaaaggtataactgtgacaagtgccattatagggaaaaggttaacagtgctttataagactataatgaggggattatttaagtaacattccagggagaaatttggacaattacctaaaggaatgaggactaaactctgattttttattttgcccaaattcctatctaaggggtctggggagtcacgccctataaatcatggattttcattagatgcgttttatttgaccccgtatattgtgacttgcttttcaattgactctggcataacattatgagacaaagaaaaaatatttaacacccaaaatagatttacttgccaagccttgaaattgccctgcaaagtttcttgttggaaaagtccacgttctatagagaatccccttccctctttgttttcctcttttctttgcagatccagtagataatcaactaaaagccaggcacccttttagctctgcggagaaacattttacaacctgctctctctctctgaaatttgctgagagcttcctctgcaaaataaaacttggtctccacaattatttttaacctgaacattcctttctatcaatcccaggtctttagacaaactcaaccaattgtcaaccagaaaatgtttaaatttacctatagcttggaagcccccgctttgagttgtcccgcctttctgaaccaaaccaacgtatttcttaaatgtatttgattgatgtctcatgcctctctaaaatataaaaaaccacgctgtacactgaccactttgggcacatgctctcaggacctcctgagggctgagtcatgggccatggtcactcatatttggctcagaataaatctcttcaaatattttacagagtttgactctttcagtcaatagaatttaagtataatgtcaaatataactttgtttcacaactagttgcatattttaacaaagacatggatgttaatttttaaagtatcatgtatttccctatcttttaaaaacaaatatgtggatagaattttcttaataaaaatgcctaaggtcaacagaatcatcaaatgtaaaaattaatctatgttcaaatatacttacatctgcaggaattaatgagatcgttcctacattattatgtggtgccttctttgttttctaggatggctgactaaaagaaatgagcatttagttgattctgaataaggcacagagagagtaaattcttcaaccatgtttaatatgaagcagcaaaagaaaagcctaaatatcgttatctgcgcaagaatgtgtggattcacaaattcttttcagacagctgttcagaagatagtattatagcctctgatcaattttgtcctatctacccctaagtggtatttactgaacagaatcaaaagagcaaactggtaggatttcattggtggaactaaaacatgaaagacaaatacttccaacgtgattattcattataatcaggttgagcattccaaatttgaaaatctaaaatctaaaactttgtgagcaccaacatgacactcaaaggaaatgctcattagagtattttggattttcagattagagatcctcaaccagtaagcacaatgcaaaaattaaaaaaaaaaaaaaattccaaaattcaaaaaaacttctggtaccaagcattttatataatagatactaaacctgaagaagaatatttgtctagtctagagagagtagggtatcaaaaaagaagaatgaaggggctaaggaaaggggctttacttgctaatgtataaaacaaaagggttagattatatgatacggaaagtctttttctagctccaaaattttatgattattatgcatgattagaaaagggtacaagagaataagaaatgacagttaatagacttttactttatatccctattctctcatagagagtctagtatatagtaaattttcaaatataccaatgaatgaattaataaactataaaaaatgctcacagaccccaaaaagcaatttactcacttagagtagaaggaaaaagaaaatgtatatctccaagagaaacaacatattagagtacaatagttgacaacaacaggcaatcacaatgagaaccctgaggcatcaaaaagaaaaaaataagaaagaaataatgttcccaatttcttgtttagaaaattaaagaaatggcaagatcatgttttattttcatcattaagtgtggttaaaatactttattacttagcaaagctaatattcaattttctggaagattcatttatgtagaacagctcatgcctctgcaatgtttaatgaggtattaaaatctatttatcctgaatacttgcatggtaataagccaagttgaacaggtcaatttttaacttataacatatgtagctagtgctcatcccagattaaagattcacccctaattatatgcaagtacaataaattttatttaattcattgattgtatgcactgatactttcttcaaaagcattcagatcatgatatgtattgcatgtcatttacctgctccaaactatccaatgacttctcatcataagaataaaatacaaactccttaccatggcctataaattccacatatggccctgcctacctctctgacaatgacttctactgctgtctccccatttcattattctgcttcagctgcaatgactttcttagcattcctaaaacacttcagtcttgttttggcctcctggcttttttatttttgcttactggcaggcattacctccagatatttgcaaattgtaatccctcacttcattattcaggtctcttcaaatgccactttacagaggctttccctaaagaatctatctaagatagtgtcaccctgcctttgcttctccaatcccttactaggtttatttttctttcttgagtttagcactaattggaaagtatatgttatctggttacatatttattgcttgttttccctctagaatgtaagcactgtgagagcacggagttcatctgcacaccagcacctaggacactgcttgtcaaatatttaggacccaataaatacttgttgaataaatgaatgaataaaccagtacagttttaatagacttatagcatgttaccatttgtatttttaaaagatatatatgtatatgcatggtcattcccagaagggtacacaagaaattgataataccactttccctatagaagggaaatatgaaggtctgagtgagagacaggtttattttccatttcatattcttttgtaatatttaaattttaaacaaggtctaggtatttttatttcaaaacagacaaacaacaacaacaaaaccactattaaaatttaccttaatcgcaacatttaatttagtaacaataagttatattttctattgagctcatatcccacaaatagaaatgtactaacaattgacatactcttactaaatcgtcataacaacctatgacataaaccctgtactgtcattcctattttattaaggcagaaagtgtaatacagagaaattaactggctaaagtcacacaactgaggaagcacatgccatgggatttgaactcagactgactgaatctaaaggccaaatacttaatcactactctccctgatgtaaatatataaccctcagaaattttcttatagaagttaaaaatatatatattgcttacttttttagaaattttaaaacatttaattggcaaagattgtataaaacgaagatgtacaatgtgatggtctcatattcttatacacaatggaatgattaccacaattaaattaatacgtctatcaccattgatgctgtacattaaatgccatgaacttatttatcttataacttaaagtgtaccctttgaccaaaatctccctatttcccccaaaactcagcttctggtaccactctcttatatttccttacttttgatagaaacctcagggcctggcacaggagctcttgaatgtgaaaataaaactgattatgatttctctgtacttgtcagatatcacagagtggccattctcaataagtatgctttaacaatgcttagattaggttgatatggtgataattattaataatgaacaacaatgcttttcatttgagtatgtacactctgtctcatgcattatagtaaacacttgacattcgtcattgcattcaatccttataataatcacgtaaggtaggttgtagtagtatttccattttacaaaaacaaagacttagccttaggataagtaacttttccaagatgacgtaacaagtgggcaaccaaatgtcaaatataagtcaattcagtcaaagattcacccctaattttatgaaagtactataataaatttaattcattgattgtattcactgatacgttcatcaaaagcattcagatcatgtgatgcatcacatggcattcacctgctctaaagtctccaatgacttctcaccagaagaataaaacacaaactccttaacgggtacaaatctaaagtccatgttattaacagctaggttctactggaatcagaagagaatgcttccaaacaatgatacagaagtagagtgacagttatgatatattaaatacaaactcaaacctgaattagattcagattcaacaaacactgtataggactgaaaacgatactcaacactatcatctacagtccatcttaaagctctttttggaatatcagtcttgggggcactactcccaccaacttacaaccatcagggaagaataaaaggttcaactggagggaaggccggcactgacagctgagctattttttttcctcttagttttcacagttgtcagcaaaaaaaaaaaaaaaaaaaaaatcatttttatcgtgtctcttaattgatttggggtatttctatgatgggacaagatgcactttcaatataaaaatattatgtatttgtaaatacttttcaatagtagaagaggaagggctgggtgccctgcctcacacctgtaattcctgcactttaggaggccgaggcgggcagatggcttgaggtcaggagttcgagaccagcctggccaacacagtgaatccccgtctctaccaaagatacaaaaattagccgagtgtggtggcaaacgcctgtaatcccagctactcgggaggctgaggcacgagaattgcttgaacctgggaggtggaggttgcagtgagcagcgatcacgtcactgcactccagcttggaccacagagcgagactctgtctcaaataaatgaataaataatagaaaaggagattaattactactattaaaggcagataatcttttaagtttttttctttgtgattgtgctttgtagataatctttgtttattgaaatagttcttacaaactatgaacttaataagaagcactatctagtcattggatatcacattctctagcatcaaagtttataaaaaccttacaaaaacgaaacaggaggctagacgtggtggaatgacactaaaaattaaaatctgtagagtgcctaaatttctacctaattatccataatctaaactatatattataccacaaaatcagttcattctggtcaaatactctcttctcatataataaacagcagtcagtcttggcttctctgtaactgagtgactaatgagctcctaaaaaaaaaaaaaagtcctgcggaaaccaagaacagactataggactgattctaaatgtttacagtttaatttcatctgcagagcatttgccaaacagtatctcttccgtacctcatctgatcctcggcaatcccgttaggcagtcagagcggcttttatctcgatgctacctacaactggggctcaggaggggttcccgattgaccagtgcaagactaacagttaaagctcttcagacaaaaattttatgcagtgatgaacaaaagctcctcgtttgataggtaagcccctgggtacgcttatatgtgagtctgaaacaaaacactcccaatttggagtccggaaaaaagctgacactacaaggtgctgggcagttaaagttctctgccagtctgggaaaggggccaagatgccctttaaaacttaagacttcctctcaggacttaaggagatccctgcccttccagctgagcagtcgaggattacggtcaagcagagctgtgctcttagcaggttctcgaatccctttttcctttcactttctccccaggtaccctgatcccccacccctcaacgcttctgtgtttgtcaagaagcccacctggtcttctagacctgaagcggcaggaccctccgttcccaataatacaagtcaccaaaaggagcgtcccagagtccagttttaccttcatgtgcctctcactcacttcaggcccaggccaggttaccgcaaccacttcactttaatgtccgcctcccagcgcttcagactcaaccacaatattcaaacccaagccactttc

SEQ ID NO: 31 - DNA, 3435 bp, Homo sapiens
gtggttgagtctgaagcgctgggaggcggacattaaagtgaagtggttgcggtaacctggcctgggcctgaagtgagtgagaggcacatgaagagaagtattcaagtatttatacagataggaatcaagataatcaacaatgtctgtcactgaggaagacctgtgccaccatatgaaagtagtagttcgtgtacgtccggaaaacactaaagaaaaagcagctggatttcataaagtggttcatgttgtggataaacatatcctagtttttgatcccaaacaagaagaagtcagttttttccatggaaagaaaactacaaatcaaaatgttataaagaaacaaaataaggatcttaaatttgtatttgatgctgtttttgatgaaacgtcaactcagtcagaagtttttgaacacactactaagccaattcttcgtagttttttgaatggatataattgcacagtacttgcctatggtgccactggtgctgggaagacccacactatgctaggatcagctgatgaacctggagtgatgtatctaacaatgttacacctttacaaatgcatggatgagattaaagaagagaaaatatgtagtactgcagtttcatatctggaggtatataatgaacagattcgtgatctcttagtaaattcagggccacttgctgtccgggaagatacccaaaaaggggtggtcgttcatggacttactttacaccagcccaaatcctcagaagaaattttacatttattggataatggaaacaaaaacaggacacaacatcccactgatatgaatgccacatcttctcgttctcatgctgttttccaaatttacttgcgacaacaagacaaaacagcaagtatcaatcaaaatgtccgtattgccaagatgtcactcattgacctggcaggatctgagcgagcaagtacttccggtgctaaggggacccgatttgtagaaggcacaaatattaatagatcacttttagctcttgggaatgtcatcaatgccttagcagattcaaagagaaagaatcagcatatcccttacagaaatagtaagcttactcgcttgttaaaggattctcttggaggaaactgtcaaactataatgatagctgctgttagtccttcctctgtattctacgatgacacatataacactcttaagtatgctaaccgggcaaaggacattaaatcttctttgaagagcaatgttcttaatgtcaataatcatataactcaatatgtaaagatctgtaatgagcagaaggcagagattttattgttaaaagaaaaactaaaagcctatgaagaacagaaagccttcactaatgaaaatgaccaagcaaagttaatgatttcaaaccctcaggaaaaagaaatcgaaaggtttcaagaaatcctgaactgcttgttccagaatcgagaagaaattagacaagaatatctgaagttggaaatgttacttaaagaaaatgaacttaaatcattctaccaacaacagtgccataaacaaatagaaatgatgtgttctgaagacaaagtagaaaaggccactggaaaacgagatcatagacttgcaatgttgaaaactcgtcgctcctacctggagaaaaggagggaggaggaattgaagcaatttgatgagaatactaattggctccatcgtgtcgaaaaagaaatgggactcttaagtcaaaacggtcatattccaaaggaactcaagaaagatcttcattgtcaccatttgcacctccagaacaaagatttgaaagcacaaattagacatatgatggatctagcttgtcttcaggaacagcaacacaggcagactgaagcagtattgaatgctttacttccaaccctaagaaaacaatattgcacattaaaagaagccggcctgtcaaatgctgcttttgaatctgacttcaaagagatcgaacatttggtagagaggaaaaaagtggtagtttgggctgaccaaactgccgaacaaccaaagcaaaacgatctaccagggatttctgttcttatgacctttccacaacttggaccagttcagcctattccttgttgctcatcttcaggtggaactaatctggttaagattcctacagaaaaaagaactcggagaaaactaatgccatctcccttgaaaggacagcatactctaaagtctccaccatctcaaagtgtgcagctcaatgattctcttagcaaagaacttcagcctattgtatatacaccagaagactgtagaaaagcttttcaaaatccgtctacagtaaccttaatgaaaccatcatcatttactacaagttttcaggctatcagctcaaacataaacagtgataattgtctgaaaatgttgtgtgaagtagctatccctcataatagaagaaaagaatgtggacaggaggacttggactctacatttactatatgtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataaatcacttcaaaaccaagcaaaatgaagttgatcaaatctgcttttcaaagtttatcaataccctttcaaaaatatatttaaaatctttgaaagaagacccatcttaaagctaagtttacccaagtactttcagcaagcagaaaaatgaaactctttgttttcttcttttgtgttctaaaaaaataaaatttcaaaagaaaaggttgtcttttaagttttttaaatatttgttgccttttaaaatccctgagtgtaagttaccatggtggcagcttagttttactatgccacaacaagttgactaggacattttagtaaatggtagtgagttaaattatctttattattttttaaaaataagaatttagaagtggtaaaattatggcccaagatgtatttggttctctattatgttttgatacattattttaatcatatatatgactttccttttcaaaaatactttaatgtacaagtgtaaatatatgtgcccataaaatcattgtaaatattatttagtcatcacaaataaaatattgtcccttgctacttgatatattaaagatgtagattttaaagtggatgtagattttaaagtg

(19) United States
(12) Patent Application Publication
(10) Pub. No.: US 2023/0151432 A1
(43) Pub. Date: May 18, 2023

(54) KIF18A INHIBITORS FOR TREATMENT OF NEOPLASTIC DISEASES

(71) Applicant: AMGEN INC., Thousand Oaks, CA (US)

(72) Inventor: Marc Noel PAYTON, Newbury Park, CA (US)

(73) Assignee: AMGEN INC., Thousand Oaks, CA (US)

(21) Appl. No.: 17/918,209

(22) PCT Filed: Apr. 13, 2021
(86) PCT No.: PCT/US2021/027042

§ 371 Date: Oct. 11, 2022

Related U.S. Application Data
(60) Provisional application No. 63/085,607, filed on Sep. 30, 2020.
(60) Provisional application No. 63/055,111, filed on Jul. 22, 2020.
(60) Provisional application No. 63/009,637, filed on Apr. 14, 2020.

(51) Int. Cl. C12Q 1/6886; A61K 31/438; A61K 31/444; A61K 31/5377; A61K 31/506

(52) U.S. Cl. CPC C12Q 1/6886; A61K 31/438; A61K 31/444; A61K 31/5377; A61K 31/506; C12Q 2600/106; C12Q 2600/156; C12Q 2600/158

(57) ABSTRACT

Provided herein are methods of treating a subject having a neoplastic disease, comprising administering to the subject a KIF18A inhibitor in an amount effective to treat the neoplastic disease a neoplastic disease in a subject. Also provided are methods of inducing or increasing tumor regression in a subject with a tumor and methods of reducing tumor or cancer growth in a subject. In exemplary aspects, the method comprises administering to the subject a KIF18A inhibitor. Methods of inducing or increasing death of tumor or cancer cells in a subject comprising administering to the subject a KIF18A inhibitor are also provided herein. Advantageously, the KIF18A inhibitors selectively treat the neoplastic disease, induce or increase tumor regression, and/or induce or increase death of tumor or cancer cells without overt toxicity to normal somatic cells.

### CROSS REFERENCE TO RELATED APPLICATIONS

This application claims under 35 U.S.C. § 119(e) the benefit of U.S. Provisional Application No. 63/009,637, filed Apr. 14, 2020, U.S. Provisional Application No. 63/055,111, filed Jul. 22, 2020, and U.S. Provisional Application No. 63/085,607, filed Sep. 30, 2020. The entire contents of each application are incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 227,954 byte ASCII (Text) file named “A-2607-WO-PCT_Seqlisting.txt”; created on Mar. 22, 2021.

### BACKGROUND

Cancer is one of the most widespread diseases afflicting mankind, and a leading cause of death worldwide. In the United States alone, cancer is the second most common cause of death, only surpassed by heart disease. In an effort to find an effective treatment or a cure for one or more of the many different cancers, over the last couple of decades, numerous groups have invested a tremendous amount of time, effort and financial resources. However, to date, of the available cancer treatments and therapies, only a few offer any considerable degree of success.

Cancer is often characterized by deregulation of normal cellular processes or unregulated cell proliferation. Cells that have been transformed to cancerous cells proliferate in an uncontrolled and unregulated manner leading to, in some cases, metastasis or the spread of the cancer. Damage to one or more genes, responsible for the cellular pathways, which control progress of proliferation through the cell cycle and centrosome cycle, can cause the loss of normal regulation of cell proliferation. These deregulated genes can code for various tumor suppressor or oncogene proteins, which participate in a cascade of events, leading to unchecked cell-cycling progression and cell proliferation. Various kinase and kinesin proteins have been identified, which play key roles in cell cycle and mitotic regulation and progression of normal dividing cells and cancer cells.

Cancer pathways, phenotypes, differentiation state associated with mitotic and replicative stress may lead to specific vulnerabilities associated with mitotic entry, mitotic spindle formation, centrosome integrity and positioning, MT-kinetochore attachment, sister chromatid cohesion, and SAC control. Thus, a strategy to improve the clinical potential of new antimitotic therapies should exploit a tumor-specific vulnerability while sparing or reducing the collateral damage to normal tissues. KIF18A is an emerging and promising anticancer target as KIF18A inhibition leads to activation of the SAC in mitosis, induction of multipolarity and apoptosis, and inhibits the growth of subset of human cancer cell lines while sparing normal dividing somatic cells.

Mitosis is the process by which a eukaryotic cell segregates its duplicated chromosomes into two identical daughter nuclei. It is generally followed immediately by cytokinesis, which divides the nuclei, cytoplasm, organelles and cell membranes into two daughter cells containing roughly equal shares of these cellular components. Mitosis and cytokinesis together define the mitotic (M) phase of the cell cycle—the division of the mother cell into two daughter cells, genetically identical to each other and to their parent cell. The process of mitosis is complex and highly regulated. The sequence of events is divided into distinct phases, corresponding to the completion of one set of activities and the start of the next. These stages are prophase, prometaphase, metaphase, anaphase and telophase. During the process of mitosis duplicated chromosomes condense and attach to spindle microtubule (MT) fibers that pull the sister chromatids to opposite sides of the cell. Spindle assembly checkpoint (SAC) is active until all sister chromatids are property attached to the spindle kinetochore fibers and spindle tension is achieved during metaphase, if no errors are detected the cells progress to anaphase. The cell then divides in cytokinesis, to produce two identical daughter cells.

Normally, cell-cycle checkpoints are activated if errors are detected (e.g. DNA damage, DNA replication fork stall/collapse, centrosome aberrations, chromosome mis-segregation, micronuclei formation). If these errors to the genome cannot be fixed, the cell normally undergoes cell arrest and apoptosis. However, if the cell is allowed to move through its cell-cycle and progress unchecked, then mutations, chromosome mis-segregation, centrosome aberrations can accumulate overtime. These gene/karyotype/centrosome altemations can accrue and eventually leading cell progeny with pre-malignant or malignant neoplastic characteristics (e.g. uncontrolled proliferation) through adaptation.

Cancers with high intratumor heterogeneity and chromosomal instability (CIN) have complex karyotypes due to continuous chromosomal changes resulting from numerical (gain or loss) and structural alternations. The mechanisms believed to contribute to CIN include defects in kinetochore MT attachment dynamics, centrosome copy number, mitotic checkpoint function, chromosome cohesion, and cell cycle regulation. Chromosome instability (CIN) is associated with an increased level of replicative and mitotic stress enriched for genetic lesions in a subset of tumor suppressor and oncogenes (examples include but not limited to TP53, RB1, BRCA1, BRCA2, homologous recombination deficient (HRD) genes, FBXW7, CCNE1, MYC) that regulate cell-cycle progression/checkpoints, centrosome-cycle, and DNA repair (SL Thompson et al Current Biology. 2010; 20:285-95 and R Nagel et al EMBO Reports 2016; 17:1516-1531).

Mitosis, or cell division, is a validated point-of-intervention in treating cancer. Approved antimitotic drugs are anti-cancer agents that inhibit the function of microtubules. Microtubules are protein polymers formed by α-tubulin and β-tubulin heterodimers that play an important role in the formation of the mitotic spindle apparatus and cytokinesis at the end of mitosis. Anti-cancer agents that target microtubules represent a proven approach for intervening in the proliferation of cancer cells. Taxanes are the most prominent class of antimitotic agent that includes paclitaxel (taxol) and docetaxel (taxotere). The vinca alkaloids are a class of microtubule-destabilizing agents that includes vincristine, vinblastine, and vinorelbine. Other new tubulin binding anti-cancer drugs include ixabepilone and eribulin. These antimitotic agents act to prevent the proliferation of cancer cells by either stabilizing- or destabilizing-microtubules. This direct inhibition of microtubules results in cell arrest and death through apoptosis, mitotic catastrophe, and lethal multipolar division. Paclitaxel was the first compound of the taxane series to be discovered. Docetaxel, a structural analog of paclitaxel, was later discovered. Paclitaxel and docetaxel are commonly used to treat a variety of human malignancies, including ovarian cancer, breast cancer, head and neck cancer, lung cancer, gastric cancer, esophageal cancer, prostate cancer, and AIDS-related Kaposi's sarcoma. The primary side effect of taxanes is myelosuppression, primarily neutropenia, while other side effects include peripheral edema, and neurotoxicity (peripheral neuropathy).

Resistance to anti-mitotic agents such as taxanes is a complicating factor to successful cancer treatment and is often associated with increased expression of the MDR-1 encoded gene and its product, the P-glycoprotein (P-gp). Other documented mechanisms of acquired resistance to taxanes include tubulin mutations, overexpression, amplification, and isotype switching). Mutations in α- or β-tubulin inhibit the binding of taxanes to the correct place on the microtubules; this renders the drug ineffective. Resistance to other anticancer drug classes, including, without limitation to chemotherapeutic agents (e.g. platinum agents, anthracyclines) and targeted therapies (e.g. TKI, PARP inhibitors) has become a major drawback in the effective treatment of cancer and inevitably leads to patient death. Consequently, development of drug resistance remains a problem with all anticancer therapies.

Precision medicine is aimed at improving cancer patient response rates by reducing toxicities to normal tissues and utilizing stratification markers to enrich for patients most likely to benefit from therapy treatment or importantly to exclude patients unlikely to benefit. A biomarker guided approach has the potential to customizing cancer patient treatment to achieve higher response rates and to drive improvement in patient outcomes and quality of life. There is a lack of established stratification markers (biomarkers) available for selecting patients most likely to benefit from current anti-mitotic therapies.

Thus, there is a need for biomarkers to identify patients will benefit from KIF18A inhibitor treatment.

### SUMMARY

Presented herein for the first time are data evidencing biomarkers of sensitivity to KIF18A inhibitor treatment. Cancer cells exhibiting an inactivated TP53 gene and/or at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof, demonstrated sensitivity to treatment with a KIF18A inhibitor. Also provided herein are data demonstrating that KIF18A inhibitor-sensitive cancer cells exhibit a reduced or lost sensitivity or resistance to CDK4/6 inhibitors. The data further support that CDK4/6 inhibitor-sensitive cancer cells exhibit a reduced or lost sensitivity or resistance to KIF18A inhibitors.

The present disclosure provides methods of determining a treatment for a subject with a neoplastic disease (e.g., cancer). In exemplary embodiments, the method comprises assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various aspects, the treatment determined for the subject comprises of a KIF18A inhibitor, when the sample is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In exemplary embodiments, the method comprises determining sensitivity of the neoplastic disease to treatment with a CDK4/6 inhibitor. In various aspects, the treatment for the subject is determined as a treatment comprising a KIF18A inhibitor, when the neoplastic disease is insensitive or resistant to the CDK4/6 inhibitor. In exemplary embodiments, the method comprises determining sensitivity of the neoplastic disease to treatment with a KIF18A inhibitor. In various aspects, the treatment for the subject is determined as a treatment comprising a CDK4/6 inhibitor, when the neoplastic disease is insensitive or resistant to the KIF18A inhibitor.

Methods of identifying a subject with a neoplastic disease as sensitive to treatment with a KIF18A inhibitor are provided herein. In exemplary embodiments, the method comprises assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various instances, the subject is identified as sensitive to treatment with a KIF18A inhibitor, when the sample is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof.

The present disclosure additionally provides a method of identifying a subject with a neoplastic disease as responsive to treatment with a KIF18A inhibitor. In exemplary embodiments, the method comprises determining the sensitivity of the neoplastic disease to treatment with a KIF18A inhibitor. In various aspects, the subject is identified as responsive to treatment with a KIF18A inhibitor, when the cancer cells of the sample are insensitive to the CDK4/6 inhibitor.

Methods of maintaining sensitivity of a neoplastic disease to treatment with a CDK4/6 inhibitor in a subject are provided herein. In exemplary embodiments, the method comprises administering to the subject a KIF18A inhibitor.

Methods of treating a subject with a neoplastic disease, e.g., methods of treating the neoplastic disease in a subject, are provided herein. In exemplary embodiments, the method comprises administering a KIF18A inhibitor to treat the patient. Optionally, the neoplastic disease is resistant to treatment with a CDK4/6 inhibitor. In various instances, the subject is or has been treated with a CDK4/6 inhibitor. In various aspects, the KIF18A inhibitor is co-administered with the CDK4/6 inhibitor. In various instances, the method comprises administering a pharmaceutical combination comprising a CDK4/6 inhibitor and a KIF18A inhibitor. Accordingly, a pharmaceutical combination comprising a KIF18A inhibitor and a CDK4/6 inhibitor is provided herein.

Methods of inducing or increasing tumor regression in a subject with a tumor are additionally provided herein. In exemplary embodiments, the method comprises administering to the subject a KIF18A inhibitor in an amount effective to induce or increase tumor regression. The present disclosure also provides methods of reducing tumor growth or cancer growth in a subject. In exemplary embodiments, the method comprises administering to the subject a KIF18A inhibitor in an amount effective to reduce tumor or cancer growth. Methods of inducing or increasing death of tumor cells or cancer cells in a subject are provided herein. The method in exemplary embodiments comprises administering to the subject a KIF18A inhibitor in an amount effective to induce or increase death of the tumor cells or cancer cells. In various aspects, the neoplastic disease is a cancer, optionally, breast cancer, ovarian cancer, or prostate cancer. In various instances, the neoplastic disease is triple-negative breast cancer (TNBC), non-luminal breast cancer, or high-grade serous ovarian cancer (HGSOC). In exemplary aspects, the neoplastic disease is an endometrial cancer, optionally, serous endometrial cancer. Optionally, the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof. In some aspects, the cancer comprises cells that are positive for a mutant TP53 gene. In various instances, the cancer comprises cells that are positive for an amplified CCNE1 gene, a silenced BRCA1 gene, a deficient Rb1 gene, or a combination thereof. Optionally, the KIF18A inhibitor is administered for oral administration, optionally once a day. In exemplary aspects, the amount of the KIF18A inhibitor is effective to induce at least 50% or at least 75% (e.g., at least 80% or 85% or at least 90% or 95%) tumor regression, compared to a control. In various instances, the KIF18A inhibitor selectively treats the neoplastic disease, selectively induces or increases tumor regression, selectively reduces tumor or cancer growth, and/or selectively induces or increases death of tumor or cancer cells and the KIF18A inhibitor is not toxic to normal somatic cells. In various aspects, the KIF18A inhibitor treats the neoplastic disease, induces or increases tumor regression, reduces tumor or cancer growth, and/or induces or increases death of tumor or cancer cells and the proliferation of the normal somatic cells in the subject is substantially the same as the proliferation of the normal somatic cells of a control subject. In exemplary instances, the KIF18A inhibitor treats the neoplastic disease, induces or increases tumor regression, reduces tumor or cancer growth, and/or induces or increases death of tumor or cancer cells and the level of apoptosis of normal somatic cells is not increased in the subject, relative to the level of apoptosis of normal somatic cells of a control subject, optionally, wherein the level of apoptosis of normal somatic cells is substantially the same as the level of apoptosis of the normal somatic cells of a control subject.

In various aspects of the present disclosure, the KIF18A inhibitor is a compound of formula (I). In exemplary aspects, the KIF18A inhibitor is Compound C1, Compound C2, Compound C3, Compound C4, Compound C5, Compound C6, Compound C7, Compound C8, Compound C9, Compound C10, Compound C11, Compound C12, Compound C13, or Compound C14, or any pharmaceutically-acceptable salt thereof, as described herein.

In alternative aspects, the KIF18A inhibitor is a large molecule KIF18A inhibitor, such as a non-coding RNA, e.g., an siRNA.

### BRIEF DESCRIPTION OF THE DRAWINGS

FIGS. 1A-1C provide tables referenced in Example 1. FIG. 1A provides Table 1A which lists tissue culture growth conditions for the indicated cell line originating from the indicated tissue. FIG. 1B provides Table 1B which lists the seeding density for the NCA for each tested cell line. FIG. 1C provides Table 1C which lists cancer cell lines and their sensitivity to a first KIF18A inhibitor.

FIGS. 2A to 2F are graphs of POC plotted as a function of concentration of KIF18A inhibitor (solid squares) or CDK4/6 inhibitor (open circles). FIGS. 2A-2C demonstrate KIF18A inhibitor-sensitive, CDK4/6 inhibitor-insensitive cells. cells and FIGS. 2D-2F demonstrate KIF18A inhibitor insensitive cells, CDK4/6 inhibitor-sensitive cells.

FIG. 3A is a graph plotting the POC as a function of concentration of KIF18A inhibitor for the OVCAR-8 HGSOC cell line. FIG. 3B is a graph plotting the POC as a function of concentration of KIF18A inhibitor for the MX-1 TNBC cell line. FIG. 3C is a graph plotting the POC as a function of concentration of KIF18A inhibitor for the MAX401NL TNBC cell line. FIG. 3D is a graph plotting the POC as a function of concentration of KIF18A inhibitor for the HCC-1937 TNBC cell line. FIG. 3E is a graph plotting the POC as a function of concentration of KIF18A inhibitor Compound C14, Olaparib, Paclitaxel, Doxorubicin, or Carboplatin for the OVCAR-8 cancer cell line.

FIG. 4A provides Table 2 which lists cancer cell lines and their sensitivity to a second KIF18A inhibitor. FIG. 4B provides Table 3 which lists cancer cell lines and their sensitivity to a second KIF18A inhibitor. FIG. 4C is a graph plotting the POC as a function of concentration of KIF18A inhibitor for breast and ovarian cancer cell lines which demonstrated sensitivity to KIF18A inhibitor treatment. FIG. 4D is a graph plotting the POC as a function of concentration of KIF18A inhibitor for breast and ovarian cancer cell lines which were insensitive to KIF18A inhibitor treatment. FIG. 4E is a graph plotting the POC as a function of concentration of KIF18A inhibitor for a second group of ovarian and breast cancer cell lines which demonstrated sensitivity to KIF18A inhibitor treatment. FIG. 4F is a graph plotting the POC as a function of concentration of KIF18A inhibitor for a second group of ovarian and breast cancer cell lines which were insensitive to KIF18A inhibitor treatment.

FIG. 5A is a graph of the tumor volume of KIF18A inhibitor-treated or vehicle-treated mice plotted as a function of time after cell implantation. FIG. 5B is a graph of the body weight of KIF18A inhibitor-treated or vehicle-treated mice plotted as a function of time after cell implantation.

FIG. 6A is a graph of the tumor volume of KIF18A inhibitor-treated or vehicle-treated mice plotted as a function of time after cell implantation. FIG. 6B is a graph of the body weight of KIF18A inhibitor-treated or vehicle-treated mice plotted as a function of time after cell implantation.

FIG. 7A is an image of DMSO-treated cells or KIF18A inhibitor cells as described herein. FIG. 7B is a pair of graphs demonstrating the % p-Histone or Pericentrin spots plotted as a function of KIF18A inhibitor concentration. FIG. 7C is a table listing the EC50 of the KIF18A inhibitor with respect to p-Histone or Pericentrin spots, as described herein.

FIG. 8A is a series of images of two types of cancer cells stained for centrin-3, percentrin, or centrosome markers upon treatment with KIF18A inhibitor or with DMSO control.

FIG. 8B is a Western blot showing the protein levels of cl-PARP in cells treated with DMSO control, KIF18A inhibitor, or Eg5 Inhibitor. GADPH is a loading control.

FIG. 9 is a series of Western blots showing the levels of the indicated proteins (cl-PARP, Cyclin B1, Mcl-1 Cyclin E1, KIF18A, BubR1) in synchronized or a synchronized cells treated with DMSO control or KIF18A inhibitor. B-actin is a loading control.

FIG. 10A is a series of Western blots showing the levels of the indicated proteins (p-Histone H3, y-H2A.X, cl-PARP, BubR1, Total HEC1, p-Hec1) in cells treated with DMSO control or KIF18A inhibitor. GADPH is a loading control. FIG. 10B is a pair of images showing cells stained for cGAS (green), γH2A.X (red), or DAPI (blue). Cells were treated with DMSO control or KIF18A inhibitor.

FIG. 11 is a pair of images showing cells stained for Centrin-3 (green), KIF18A (red), or DNA (DAPI (blue)). Cells were treated with DMSO control or KIF18A inhibitor.

FIG. 12 is a graph of p-Histone H3 in cells treated with the indicated amount of a KIF18A inhibitor of vehicle control.

FIG. 13A is a graph of the POC plotted as a function of concentration (log concentration) of cells treated with KIF18A inhibitor Compound C14 in the presence (open circles) or absence (closed circles) of a P-gp inhibitor. FIG. 13B is a graph of the POC plotted as a function of concentration (log concentration) of cells treated with paclitaxel (tubulin) in the presence (open circles) or absence (closed circles) of a P-gp inhibitor.

FIG. 14A is a series of FACS plots demonstrating DNA content of human bone marrow mononuclear cells (HBMNC) treated with DMSO, KIF18A inhibitor Compound C9 or Compound C11, ispinesib, paclitaxel or palbociclib. FIG. 14B is a graph demonstrating % human bone marrow mononuclear cells (HBMNC) from Donor 37612 or Donor 37534 stained with anti-BrdU (top graph) or in SubG1 of the cell cycle (bottom graph) wherein the HBMNC were treated with DMSO, KIF18A inhibitor Compound C9 or Compound C11, ispinesib (Eg5), paclitaxel (tubulin) or palbociclib (CDK4/6) for 48 hours. FIG. 14C is a graph of the live cell count (per 1×10⁶) of cells from Donor 37612 or Donor 37534 after treatment with DMSO, KIF18A inhibitor Compound C9 or Compound C11, ispinesib (Eg5), paclitaxel (tubulin) or palbociclib CDK4/6) for 96 hours. FIG. 14D is a series of graphs plotting the % of human Foreskin Fibroblast (hFSF) cells stained positive for BrdU treated with DMSO or different doses of KIF18A inhibitor Compound C11, ispinesib (Eg5), or palbociclib (CDK4/6) for 48 hours.

FIG. 14E is a series of graphs plotting the % of human mammary epithelial cells (HMEC) stained positive for BrdU treated with DMSO or different doses of KIF18A inhibitor Compound C11, ispinesib (Eg5), or palbociclib (CDK4/6) for 48 hours.

FIGS. 15A-15E are heatmaps demonstrating relative total object count (FIG. 15A), BrdU incorporation (FIG. 15B), ci-PARP expression (FIG. 15C), p21 protein expression (FIG. 15D), and yHH2X expression (FIG. 15E) of cells treated with DMSO, KIF18A inhibitor Compound C9 or Compound C11, BI-2536 PLK1, paclitaxel (tubulin), ispinesib (Eg5), GSK923295 (CENP-E), Nutlin-3A (MDM2), or palbociclib (CDK4/6).

FIG. 16 is a series of Western blots of lysates of normal HMEC (Left panel) and BT-549 TNBC cells (right panel) treated with individual KIF18A siRNAs, or non-targeting control (NTC) siRNA, or Positive Control (+ Control; HeLa cells treated with nocodazole (NOC) or Jurkat cells treated with staurosporine). Western blots for cleaved PARP (cl-PARP) apoptosis marker and β-actin to demonstrate equal protein loading in each lane.

FIG. 17A is a series of graphs of the POC of cells treated with KIF18A siRNA, non-targeting control (NTC) siRNA siRNAs, or positive control siRNA (Eg5 siRNA). Dotted-line indicates 50% cell growth inhibition based on NTC siRNAs control. KIF18A siRNAs reduced counts >50% relative to NTC siRNA were considered as KIF18A siRNA sensitive with p-value <0.05. FIG. 17B is a table providing the legend for the graph of FIG. 16A with cell line information (tissue origin, tumor subtype, and genetic status). p-values and cell growth inhibition KIF18A siRNAs relative to NTC. KIF18A siRNAs reduced counts >50% relative to NTC siRNA were considered as KIF18A siRNA sensitive with p-value <0.05. FIG. 17C is a Western blot of lysates of KIF18A siRNA sensitive or insensitive cells showing KIF18A and p-actin protein expression levels.

FIG. 18 is a graph of the sensitivity to KIF18A inhibitor Compound C9 for each of the four groups of breast and ovarian cancer cell lines differing by WGD status and TP53 status.

FIG. 19A provides ADP-Glo concentration-response profiles of KIF18A motor activity presented as MT-ATPase luminescence signal relative to DMSO control (POC), values represent mean±SEM from three independent experiments.

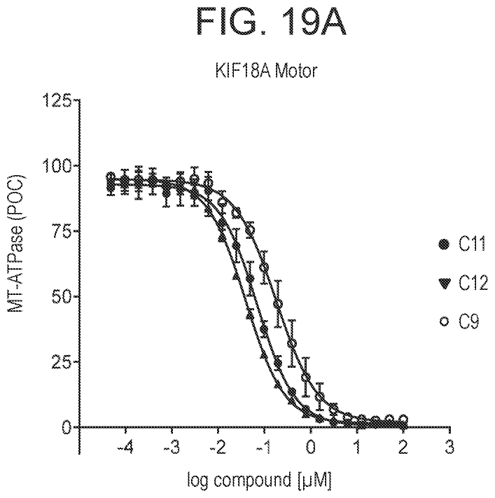

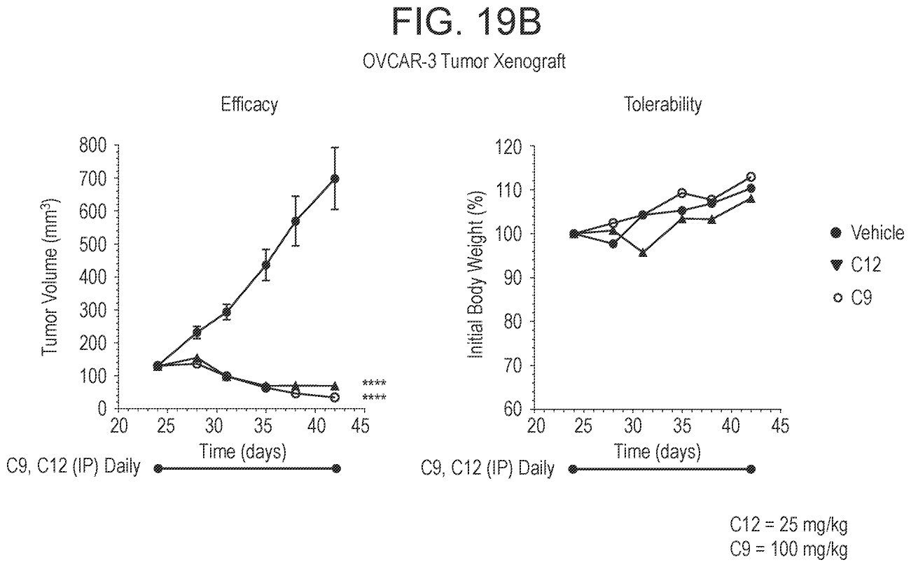

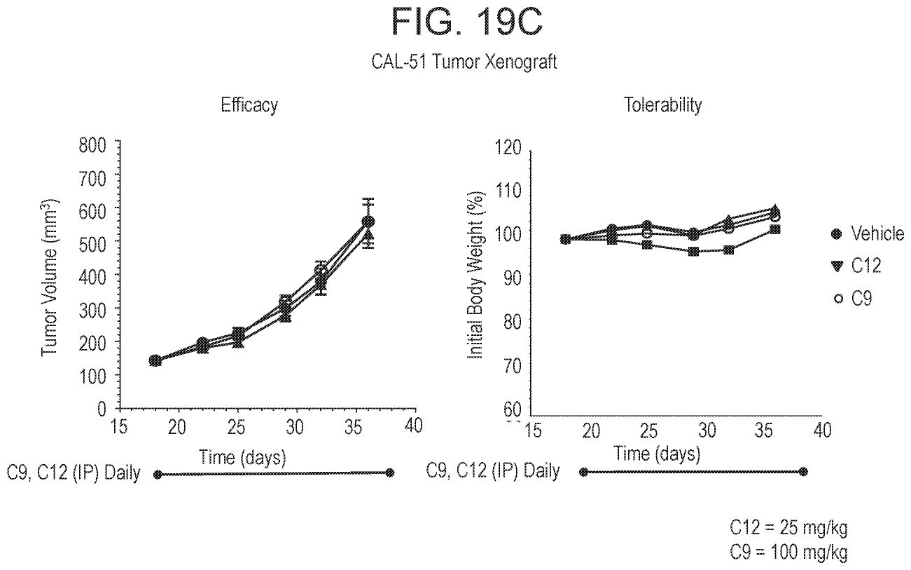

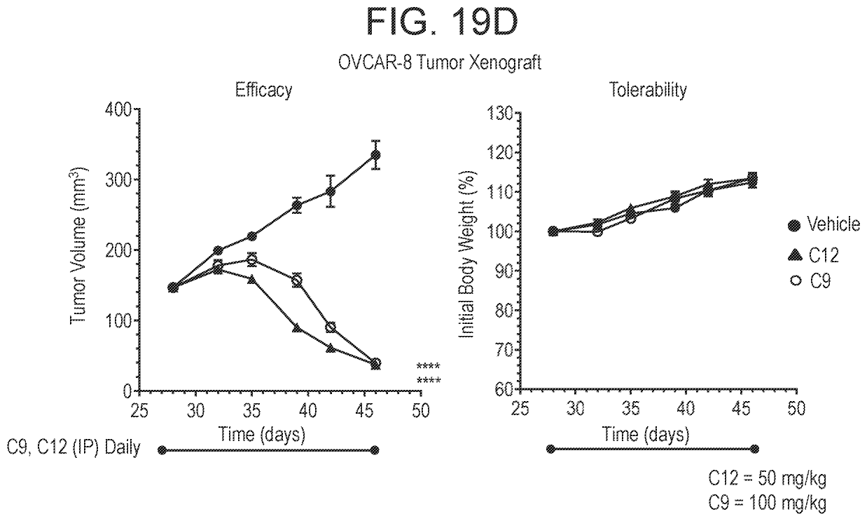

Tumor efficacy and tolerability analysis of Compounds C9 and C12 in OVCAR-3 HGSOC (FIG. 19B) or CAL-51 TNBC tumor xenografts (FIG. 19C). Mice with established tumors administered IP dose of vehicle alone, Compound C9 at 100 mg/kg, or Compound C12 at 25 mg/kg daily for 18 consecutive days. Graphs show tumor volume (left, efficacy) and body weight (right, tolerability) measurements presented as mean t SEM versus time (days) (n=10 per group). Treatment groups compared to vehicle alone group, **p<0.0001 by RMANOVA followed by Dunnett test for multiple comparisons.

Tumor efficacy, tolerability, tumor re-growth analysis of C9 and C12 in OVCAR-8 HGSOC tumor xenografts. Graphs of FIG. 19D show tumor volume (left, efficacy) and body weight (right, tolerability) measurements presented as mean t SEM versus time (days) (n=10 per group). Treatment groups compared to vehicle alone group, **p<0.0001 by RMANOVA followed by Dunnett test for multiple comparisons.

### DETAILED DESCRIPTION

Methods of Determining Treatment, Methods of Identifying Responders to Treatment, and Related Methods

The present disclosure provides methods of determining a treatment for a subject with a neoplastic disease (e.g., cancer). In exemplary embodiments, the method comprises assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various aspects, the treatment determined for the subject comprises a KIF18A inhibitor, when the sample is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof.

In exemplary embodiments, the method comprises determining sensitivity of the neoplastic disease to treatment with a CDK4/6 inhibitor. In various aspects, the treatment for the subject is determined as a treatment comprising a KIF18A inhibitor, when the neoplastic disease is insensitive or resistant to the CDK4/6 inhibitor.

In exemplary embodiments, the method comprises determining sensitivity of the neoplastic disease to treatment with a KIF18A inhibitor. In various aspects, the treatment for the subject is determined as a treatment comprising a CDK4/6 inhibitor, when the neoplastic disease is insensitive or resistant to the KIF18A inhibitor.

Methods of identifying a subject with a neoplastic disease as sensitive to treatment with a KIF18A inhibitor are provided herein. In exemplary embodiments, the method comprises assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various instances, the subject is identified as sensitive to treatment with a KIF18A inhibitor, when the sample is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof.

The present disclosure additionally provides a method of identifying a subject with a neoplastic disease as responsive to treatment with a KIF18A inhibitor. In exemplary embodiments, the method comprises determining the sensitivity of the neoplastic disease to treatment with a KIF18A inhibitor. In various aspects, the subject is identified as responsive to treatment with a KIF18A inhibitor, when the cancer cells of the sample are insensitive to the CDK4/6 inhibitor.

Methods of maintaining sensitivity of a neoplastic disease to treatment with a CDK4/6 inhibitor in a subject are provided herein. In exemplary embodiments, the method comprises administering to the subject a KIF18A inhibitor.

Methods of determining efficacy of treatment with a KIF18A inhibitor in a subject are furthermore provided herein. In exemplary embodiments, the method comprises assaying a sample obtained from the subject after commencement of treatment with the KIF18A inhibitor for one or more of
a) expression level of p-Histone H3
b) centrosome features (centriole and PCM markers include but not limited to centrin-1-3, pericentrin, γ-tubulin; measure spotting pattern or fragmentation, pole-to-pole distance), nsis
c) chromosome features (DNA dyes; measure mitotic chromatin dimensions, lagging chromosomes/anaphase bridges, micronuclei formation),
d) spindle features (tubulin markers, measure multipolarity and spindle geometry), e) KIF18A protein localization (KIF18A marker; measure localization of KIF18A in mitosis,
f) KIF18A protein post-translational modification (KIF18A protein doublet detection by Western analysis),
g) protein or gene expression modulation (such as cyclin B1, securin, p-histone H3 (ser-10), cyclin E1, Mcl-1, BubR1, SAC components, cl-PARP, cl-caspase-3/-7), h) markers of apoptosis (such as TUNEL), DNA damage and repair (such as γ-H2AX (Ser-139).

KIF18A Inhibitors

The present disclosure relates to KIF18A inhibitors. The term “KIF18A inhibitor” means any compound useful for modulating KIF18A protein alone or in a bound complex with microtubules (MT) for treating KIF18A-mediated conditions and/or diseases, including neoplastic diseases (e.g., cancer), inflammation, or ciliopathologies. The KIF18A inhibitor compounds disclosed herein have MT-based KIF18A modulatory activity and, in particular, KIF18A inhibitory activity. To this end, the present disclosure also provides the use of these compounds, as well as pharmaceutically acceptable salts thereof, in the preparation and manufacture of a pharmaceutical composition or medicament for therapeutic, prophylactic, acute or chronic treatment of KIF18A mediated diseases and disorders, including without limitation, cancer. Thus, the compounds of the present disclosure are useful in the manufacture of anti-cancer medicaments.

In various aspects, the term “KIF18A inhibitor” means any compound or molecule that targets KIF18A and reduces or inhibits KIF18A activity. KIF18A gene belongs to Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells (MI Mayr et al, Current Biology 17, 488-98, 2007). KIF18A is overexpressed in various types of cancers, including but not limited to colon, breast, lung, pancreas, prostate, bladder, head, neck, cervix, and ovarian cancers. Overexpression of KIF18A dampens sister chromatid oscillation resulting in tight metaphase plates. Inactivation of KIF18A motor function in KIF18A knockout mice or by mutagenic ethylmethanosulfonate (EMS) treatment in KIF18A^{gcd2/gcd2} mice (missense mutation (R308K) in the motor domain) resulting in viable mice with no gross abnormalities in major organs except for clear testis atrophy and sterility (J Stumpff et al Developmental Cell. 2008; 14:252-262; J Stumpff et al Developmental Cell. 2012; 22:1017-1029; X S Liu et al. Genes & Cancer. 2010; 1:26-39; CL Fonseca et al J Cell Biol. 2019; 1-16; A Czechanski et al Developmental Biology. 2015; 402:253-262. O Rath, F Kozielski. Nature Reviews Cancer. 2012; 12:527-539). Normal human and mouse KIF18A-deficient somatic cells were shown to complete cell division with relatively normal mitotic progression but without proper chromosome alignment resulting in daughter cells with a normal karyotype, some defects in exit from mitosis were noted in a subset of normal cells resulting in micronuclei formation on slower proliferation (CL Fonseca et al J Cell Biol. 2019; 1-16). These genetic studies suggest that normal germ and somatic cells have different dependency on requirements for chromosome alignment and indicate that KIF18A may be dispensable in normal euploidy somatic cell division (XS Liu et al Genes & Cancer. 2010; 1:26-39; A Czechanski et al Developmental Biology. 2015; 402:253-262). In normal human tissues, expression of KIF18A is elevated in tissues with actively cycling cells, with highest expression in the testis (GTEx Portal, GTEx Portal, J Lonsdale et al Nature Genetics. 2013:29; 45:580). In various aspects, the KIF18A inhibitor inhibits ATPase activity. For example, the KIF18A inhibitor inhibits MT-ATPase activity and not basal ATPase activity.

The reduction or inhibition provided by the KIF18A inhibitor may not be a 100% or complete inhibition or abrogation or reduction. Rather, there are varying degrees of reduction or inhibition of which one of ordinary skill in the art recognizes as having a potential benefit or therapeutic effect. In this regard, the KIF18A inhibitor may inhibit the KIF18A protein(s) to any amount or level. In exemplary embodiments, the reduction or inhibition provided by the KIF18A inhibitor is at least or about 10% reduction or inhibition (e.g., at least or about 20% reduction or inhibition, at least or about 30% reduction or inhibition, at least or about 40% reduction or inhibition, at least or about 50% reduction or inhibition, at least or about 60% reduction or inhibition, at least or about 70% reduction or inhibition, at least or about 80% reduction or inhibition, at least or about 90% reduction or inhibition, at least or about 95% reduction or inhibition, at least or about 98% reduction or inhibition).

In exemplary embodiments, the KIF18A inhibitor compounds that can be used in the present methods of the present disclosure is a small molecule compound of formula (I):

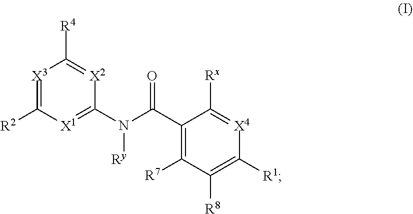

or any pharmaceutically-acceptable salt thereof, wherein:

X¹ is N or —CR⁶;

X² is N or —CR⁵;

X³ is N or —CR³;

X⁴ is N or —CR⁹;

wherein 0, 1, or 2 of X¹, X², X³ and X⁴ is N;

R¹ is —CN, or a group —Z—R¹² wherein Z is —C₀₋₄alk-, —NR¹¹—, —NR¹¹SO₂—, —SO₂NR¹¹—, —NR¹¹—S(═O)(═NH), —S(═O)(═NH)—, —S—, —S(═O)—, —SO₂—, C₀₋₄alk-O—, —(C═O)—, —(C═O)NR¹¹—, —C═N(OH)—, or —NR¹¹(C═O); or

the group —Z—R¹² is —N═S(═O)—(R¹²)₂, wherein the two R¹² pair can alternatively combine with the sulfur atom attached to each of them to form a saturated or partially-saturated 3-, 4-, 5-, or 6-membered monocyclic ring containing 0, 1, 2 or 3 N atoms and 0, 1, or 2 atoms selected from O and S;

R² is halo or a group —Y—R¹³, wherein Y is —C₀₋₄alk-, —N(C₀₋₁alk)-C₀₋₄alk-, —C(═O)NR^{a}R^{a}(C₁₋₄alk), —O—C₀₋₄alk-, S, S═O, S(═O)₂, —SO₂NR¹³, or —S(═O)(═NH)—;

R³ is H, halo, C₁₋₈alk, or C₁₋₄haloalk;

R⁴ is H, halo, R^{4a} or R^{4b};

R⁵ is H, halo, C₁₋₈alk, or C₁₋₄haloalk;

R⁶ is H, halo, C₁₋₈alk, C₁₋₄haloalk, —O—C₁₋₈alk, or —O—R^{6a}; wherein R^{6a} is a saturated or partially-saturated 3-, 4-, 5-, or 6-membered monocyclic ring containing 0, 1, 2 or 3 N atoms and 0, 1, or 2 atoms selected from O and S;

R⁷ is H, halo, C₁₋₈alk, or C₁₋₄haloalk;

R⁸ is H, halo, C₁₋₈alk, C₁₋₄haloalk, —OH, —O—R^{8a}, or —O—R^{8a};

R⁹ is H, halo, C₁₋₈alk, or C₁₋₄haloalk;

R^{x} is selected from the group consisting of

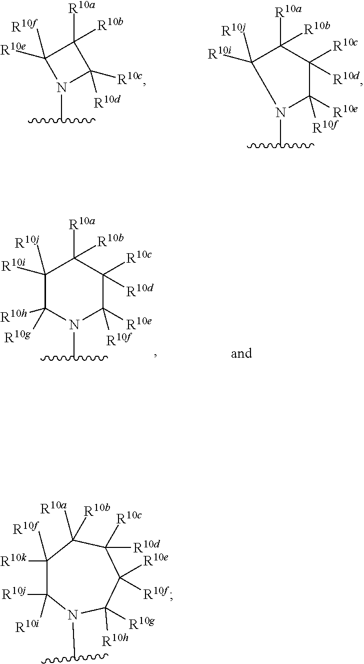

Each of R^{10a}, R^{10b}, R^{10c}, R^{10d}, R^{10e}, R^{10f}, R^{10g}, R^{10h}, R¹⁰ⁱ, and R^{10j} is H, halo, R^{10k} or R^{10l};

or alternatively, each of R^{10a} and R^{10b} pair, R^{10c} and R^{10d} pair, R^{10e} and R^{10f} pair, R^{10g} and R^{10h} pair, or R¹⁰ⁱ and R^{10j} pair, independently, can combine with the carbon atom attached to each of them to form a saturated or partially-saturated 3-, 4-, 5-, 6-membered monocyclic ring spiro to the R^{x} ring; wherein said 3-, 4-, 5-, 6-membered monocyclic ring contains 0, 1, 2 or 3 N atoms and 0, 1, or 2 atoms selected from O and S, and further wherein said 3-, 4-, 5-, 6-membered monocyclic ring is substituted by 0, 1, 2 or 3 group(s) selected from F, Cl, Br, C₁₋₆alk, C₁₋₄haloalk, —OR^{a}, —OC₁₋₄haloalk, CN, —NR^{a}R^{a}, or oxo;

R^{y} is H, C₁₋₄alk, or C₁₋₄haloalk;

R¹¹ is H, R^{11a}, or R^{11b};

R¹² is H, R^{12a}, or R^{12b};

R¹³ is R^{13a} or R^{13b};

R^{4a}, R^{8a}, R^{10k}Ok, R^{11a}, R^{12a}, and R^{13a} is independently, at each instance, selected from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0, 1, or 2 atoms selected from O and S, which is substituted by 0, 1, 2 or 3 group(s) selected from F, Cl, Br, C₁₋₆alk, C₁₋₄haloalk, —OR^{a}, —OC₁₋₄haloalk, CN, —C(═O)R^{b}, —C(═O)OR^{a}, —C(═O)NR^{a}R^{a}, —C(═NR^{a})NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁₋₆alkNR^{a}R⁸, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₆alkC(═O)OR^{a}, R¹⁴, and oxo;

R^{4b}, R^{8b}, R¹⁰ⁱ, R^{11b}, R^{12b}, and R^{13b} is independently, at each instance, selected from the group consisting of C₁₋₆alk substituted by 0, 1, 2, 3, 4, or 5 group(s) selected from F, Cl, Br, —OR^{a}, —OC₁₋₄haloalk, or CN;

R¹⁴ is independently, at each instance, selected from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0 or 1 atoms selected from O and S, which is substituted by 0, 1, 2 or 3 group(s) selected from F, Cl, Br, C₁₋₆alk, C₁₋₄haloalk, —OR^{a}, —OC₁₋₄haloalk, CN, —C(═O)R^{b}, —C(═O)OR^{a}, —C(═O)NR^{a}R^{a}, —C(═NR^{a})NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁₋₆alkNR^{a}R^{a}, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₆alkC(═O)OR^{a}, and oxo;

R^{a} is independently, at each instance, H or R^{b}; and

R^{b} is independently, at each instance, C₁₋₆alk, phenyl, or benzyl, wherein the C₁₋₆alk is being substituted by 0, 1, 2 or 3 substituents selected from halo, —OH, —OC₁₋₄alk, —NH₂, —NHC₁₋₄alk, —OC(═O)C₁₋₄alk, or —N(C₁₋₄alk)C₁₋₄alk; and the phenyl or benzyl is being substituted by 0, 1, 2 or 3 substituents selected from halo, C₁₋₄alk, C₁₋₃haloalk, —OH, —OC₁₋₄alk, —NH₂, —NHC₁₋₄alk, —OC(═O)C₁₋₄alk, or —N(C₁₋₄alk)C₁₋₄alk.

Preparation of the compounds of formula (I) can be found in the previously filed U.S. provisional patent application Ser. Nos. 62/783,061 and 62/783,069; each of which was filed on 20 Dec. 2018; and 62/882,255 and 62/882,268; each of which was filed on 2 Aug. 2019.

In another embodiment, the present invention provides the method of any one of the preceding embodiments, wherein the KIF18A inhibitor is a compound of formula (I), or the pharmaceutically-acceptable salt thereof, wherein 0 of X¹, X², X³ and X⁴ is N.

In another embodiment, the present invention provides the method of any one of the preceding embodiments, wherein the KIF18A inhibitor is a compound of formula (I), or the pharmaceutically-acceptable salt thereof, wherein 1 of X¹, X², X³ and X⁴ is N.

In another embodiment, the present invention provides the method of any one of the preceding embodiments, wherein the KIF18A inhibitor is a compound of formula (I), or the pharmaceutically-acceptable salt thereof, wherein 2 of X¹, X², X³ and X⁴ is N.

In another embodiment, the present invention provides the method of any one of the preceding embodiments, wherein the KIF18A inhibitor is a compound of formula (I), or the pharmaceutically-acceptable salt thereof, wherein each of X¹ and X³ is N; X² is —CR⁵; and X⁴ is —CR⁹; having the formula (Ia):

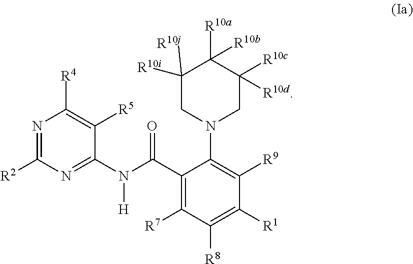

In another embodiment, the present invention provides the method of any one of the preceding embodiment, wherein the KIF18A inhibitor is a compound of formula (I), or the pharmaceutically-acceptable salt thereof, wherein X¹ is —CR⁶; X² is —CR⁵; X³ is N; and X⁴ is —CR⁹; having the formula (Ib):

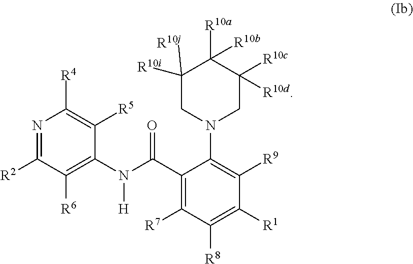

In another embodiment, the present invention provides the method of any one of the preceding embodiment, wherein the KIF18A inhibitor is a compound of formula (I), or the pharmaceutically-acceptable salt thereof, wherein X¹ is N; X² is —CR⁵; X³ is —CR³; and X⁴ is —CR⁹; having the formula (Ic):

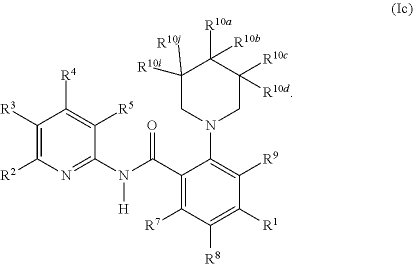

In another embodiment, the present invention provides the method of any one of the preceding embodiment, wherein the KIF18A inhibitor is a compound of formula (I), or the pharmaceutically-acceptable salt thereof, wherein X¹ is —CR⁶; X² is —CR⁵; X³ is —CR³; and X⁴ is —CR⁹; having the formula (Id):

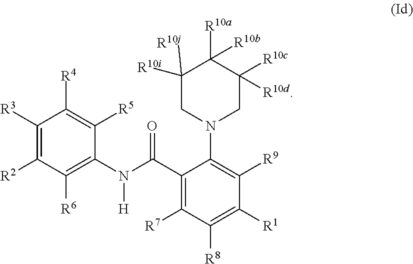

In another embodiment, the present invention provides the method of any one of the preceding embodiment, wherein the KIF18A inhibitor is a compound of formula (I), or the pharmaceutically-acceptable salt thereof, wherein X¹ is —CR⁶; X² is —CR⁵; X³ is —CR³; and X⁴ is —N; having the formula (Ie):

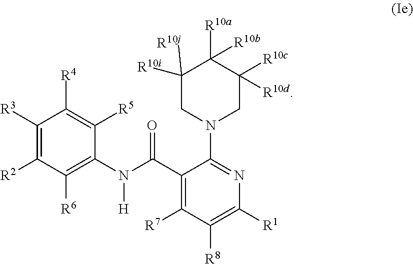

In another embodiment, the present invention provides the method of any one of the preceding embodiment, wherein the KIF18A inhibitor is a compound of formula (I), or the pharmaceutically-acceptable salt thereof, wherein R^{y} is H or methyl. In another sub-embodiment, R^{y} is H.

In another embodiment, the present invention provides the method of any one of the preceding embodiment, wherein the KIF18A inhibitor is a compound of formula (I), or the pharmaceutically-acceptable salt thereof, wherein each of R^{10c}, R^{10d}, R^{10e}, R^{10f}, R^{10g}, R^{10h}, R¹⁰ⁱ, and R^{10j} is H, halo, C₁₋₆alk, or C₁₋₄haloalk; and each of R^{10a} and R^{10b} pair combine with the carbon atom attached to each of them form a saturated 3-, 4-, or 5-membered monocyclic ring spiro to the R^{x} ring; wherein said ring contains 0, 1, 2 or 3 N atoms and 0, 1, or 2 atoms selected from O and S.

In another embodiment, the present invention provides the method of any one of the preceding embodiment, wherein the KIF18A inhibitor is a compound of formula (I), or the pharmaceutically-acceptable salt thereof, wherein each of R^{10c}, R^{10d}, R^{10e}, R^{10f}, R^{10g}, R^{10h}, R¹⁰ⁱ, and R^{10j} is H, methyl, or ethyl; and each of R^{10a} and R^{10b} pair combine with the carbon atom attached to each of them form a cyclopropyl, cyclobutyl, or cyclopentyl ring spiro to the R^{x} ring.

In another embodiment, the present invention provides the method of any one of the preceding embodiment, wherein the KIF18A inhibitor is a compound of formula (I), or the pharmaceutically-acceptable salt thereof, wherein the KIF18A or the pharmaceutically-acceptable salt thereof, wherein the group

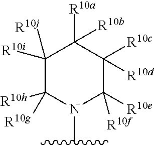

is

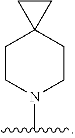 [SMILES: *N1CCC2(CC1)CC2]

In another embodiment, the present invention provides the method of any one of the preceding embodiment, wherein the KIF18A inhibitor is a compound of formula (I), or the pharmaceutically-acceptable salt thereof, wherein R¹ is a group —Z—R¹²; wherein Z is —S(═O)(═NH)—, —NHSO₂—, —SO₂—, —SO₂NH—, or —NH—; and R¹² is cyclopropyl, —CH₂CH₂—OH, —CH(CH₃)CH₂—OH, —C(CH₃)₂CH₂—OH, methyloxetanyl, or tert-butyl.

In another embodiment, the present invention provides the method of any one of the preceding embodiment, wherein the KIF18A inhibitor is a compound of formula (I), or the pharmaceutically-acceptable salt thereof, wherein R¹ is a group —Z—R¹²; wherein Z is —NHSO₂— or —NH—; and R¹² is —CH₂CH₂—OH, —CH(CH₃)CH₂—OH, or —C(CH₃)₂CH₂—OH.

In another embodiment, the present invention provides the method of any one of the preceding embodiment, wherein the KIF18A inhibitor is a compound of formula (I), or the pharmaceutically-acceptable salt thereof, wherein R¹ is a group —Z—R¹²; wherein Z is —NHSO₂— and R¹² is —CH₂CH₂—OH.

In another embodiment, the present invention provides the method of any one of the preceding embodiment, wherein the KIF18A inhibitor is a compound of formula (I), or the pharmaceutically-acceptable salt thereof, wherein R² is a group —Y—R¹³; wherein Y is —C₀₋₄alk-, —O—C₀₋₄alk-, S, S═O, S(═O)₂, or —SO₂NH—; and —R¹³ is 4,4-difluoro-1-piperidinyl; —CH₂CH₂—CF₃, tert-butyl, cyclopentyl, or 2-methylmorpholinyl.

In another embodiment, the present invention provides the method of any one of the preceding embodiment, wherein the KIF18A inhibitor is a compound of formula (I), or the pharmaceutically-acceptable salt thereof, wherein R² is piperidinyl or morpholinyl substituted by 1, 2 or 3 group(s) selected from F, Cl, Br, methyl, or CF₃; or R² is —O—CH₂CH₂— CF₃, —SO₂NH—C(CH₃)₃, or —SO₂-cyclopentyl.

In another embodiment, the present invention provides the method of any one of the preceding embodiment, wherein the KIF18A inhibitor is a compound of formula (I), or the pharmaceutically-acceptable salt thereof, wherein R² is a group —Y—R¹³; wherein Y is —C₀₋₄alk-; and —R¹³ is 4,4-difluoro-1-piperdinyl or 2-methylmorpholinyl.

In another embodiment, the present invention provides the method of any one of the preceding embodiment, wherein the KIF18A inhibitor is a compound of formula (I), or the pharmaceutically-acceptable salt thereof, wherein R⁴ is H or methyl.

In another embodiment, the present invention provides the method of any one of the preceding embodiment, wherein the KIF18A inhibitor is a compound of formula (I), or the pharmaceutically-acceptable salt thereof, wherein R⁵ is H.

In another embodiment, the present invention provides the method of any one of the preceding embodiment, wherein the KIF18A inhibitor is a compound of formula (I), or the pharmaceutically-acceptable salt thereof, wherein R⁶ is H.

In another embodiment, the present invention provides the method of any one of the preceding embodiment, wherein the KIF18A inhibitor is a compound of formula (I), or the pharmaceutically-acceptable salt thereof, wherein R⁷ is H.

In another embodiment, the present invention provides the method of any one of the preceding embodiment, wherein the KIF18A inhibitor is a compound of formula (I), or the pharmaceutically-acceptable salt thereof, wherein R⁸ is H.

In another embodiment, the present invention provides the method of any one of the preceding embodiment, wherein the KIF18A inhibitor is a compound of formula (I), or the pharmaceutically-acceptable salt thereof, wherein R⁹ is H.

In another embodiment, the present invention provides the method of any one of the preceding embodiment, wherein the KIF18A inhibitor is a compound of formula (I), or the pharmaceutically-acceptable salt thereof, wherein said compound is selected from the group consisting of:

| Ex. # * | Chemical Structure | Chemical Name |
|---|---|---|
| C1 | 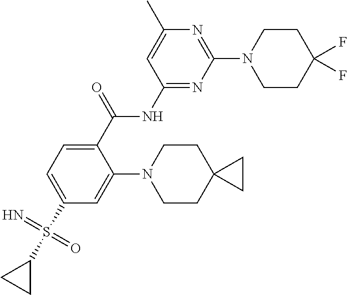 [SMILES: Cc1cc(NC(=O)c2ccc([S@](=N)(=O)C3CC3)cc2N2CCC3(CC2)CC3)nc(N2CCC(F)(F)CC2)n1] | 2-(6-Azaspiro[2.5]octan-6-yl)-4-(R- cyclopropylsulfonimidoyl)-N-(2- (4,4-difluoro-1-piperidinyl)-6- methyl-4-pyrimidinyl)benzamide |
| C2 | 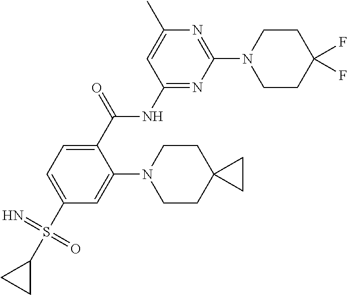 [SMILES: Cc1cc(NC(=O)c2ccc(S(=N)(=O)C3CC3)cc2N2CCC3(CC2)CC3)nc(N2CCC(F)(F)CC2)n1] | 2-(6-azaspiro[2.5]octan-6-yl)-4-(S- cyclopropylsulfonimidoyl)-N-(2- (4,4-difluoro-1-piperidinyl)-6- methyl-4-pyrimidinyl)benzamide |
| C3 | 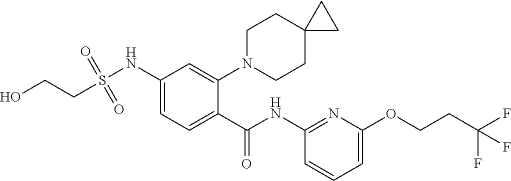 [SMILES: O=C(Nc1cccc(OCCC(F)(F)F)n1)c1ccc(NS(=O)(=O)CCO)cc1N1CCC2(CC1)CC2] | 4-((2-Hydroxyethyl)sulfonamido)-2- (6-azaspiro[2.5]octan-6-yl)-N-(6- (3,3,3-trifluoropropoxy)pyridin-2- yl)benzamide |
| C4 | 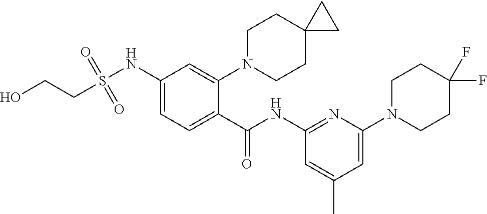 [SMILES: Cc1cc(NC(=O)c2ccc(NS(=O)(=O)CCO)cc2N2CCC3(CC2)CC3)nc(N2CCC(F)(F)CC2)c1] | N-(6-(4,4-difluoropiperidin-1-yl)-4- methylpyridin-2-yl)-4-((2- hydroxyethyl)sulfonamido)-2-(6- azaspiro[2.5]octan-6-yl)benzamide |
| C5 | 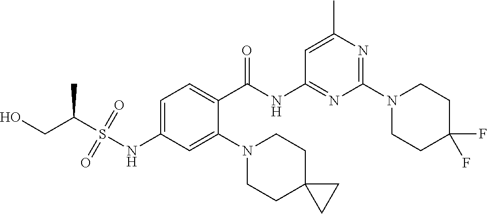 [SMILES: Cc1cc(NC(=O)c2ccc(NS(=O)(=O)[C@H](C)CO)cc2N2CCC3(CC2)CC3)nc(N2CCC(F)(F)CC2)n1] | (R)-N-(2-(4,4-difluoropiperidin-1- yl)-6-methylpyrimidin-4-yl)-4-((2- hydroxy-1- methylethyl)sulfonamido)-2-(6- azaspiro[2.5]octan-6-yl)benzamide |
| C6 | 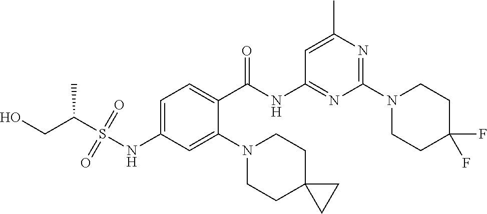 [SMILES: Cc1cc(NC(=O)c2ccc(NS(=O)(=O)[C@@H](C)CO)cc2N2CCC3(CC2)CC3)nc(N2CCC(F)(F)CC2)n1] | (S)-N-(2-(4,4-difluoropiperidin-1- yl)-6-methylpyrimidin-4-yl)-4-((2- hydroxy-1- methylethyl)sulfonamido)-2-(6- azaspiro[2.5]octan-6-yl)benzamide |
| C7 | 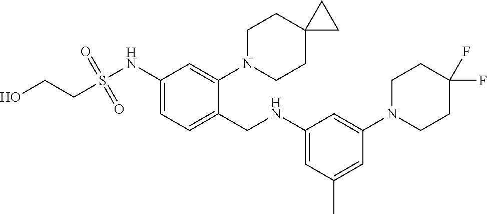 [SMILES: Cc1cc(NCc2ccc(NS(=O)(=O)CCO)cc2N2CCC3(CC2)CC3)cc(N2CCC(F)(F)CC2)c1] | N-(3-(4,4-difluoropiperidin-1-yl)-5- methylphenyl)-4-((2- hydroxyethyl)sulfonamido)-2-(6- azaspiro[2.5]octan-6-yl)benzamide |
| C8 | 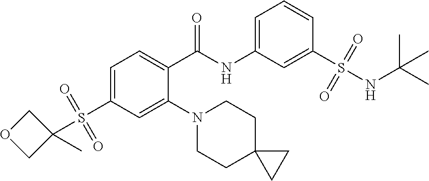 [SMILES: CC(C)(C)NS(=O)(=O)c1cccc(NC(=O)c2ccc(S(=O)(=O)C3(C)COC3)cc2N2CCC3(CC2)CC3)c1] | N-(3-(N-(tert- Butyl)sulfamoyl)pheny)-4-((3- methyloxetan-3-yl)sulfonyl)-2- (6-azaspiro[2.5]octan-6- yl)benzamide |
| C9 | 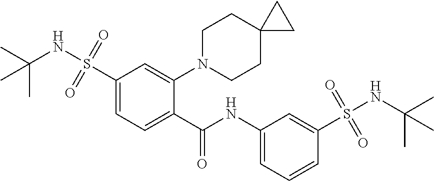 [SMILES: CC(C)(C)NS(=O)(=O)c1cccc(NC(=O)c2ccc(S(=O)(=O)NC(C)(C)C)cc2N2CCC3(CC2)CC3)c1] | 4-(N-(tert-butyl)sulfamoyl)-N-(3- (N-(tert-butyl)sulfamoyl)phenyl)- 2-(6-azaspiro[2.5]octan-6- yl)benzamide |
| C10 | 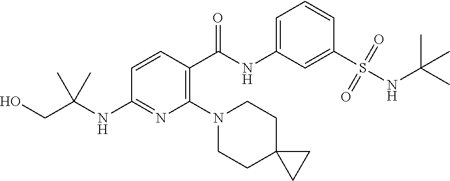 [SMILES: CC(C)(C)NS(=O)(=O)c1cccc(NC(=O)c2ccc(NC(C)(C)CO)nc2N2CCC3(CC2)CC3)c1] | N-(3-(N-(tert- Butyl)sulfamoyl)phenyl)-6-((1- hydroxy-2-methylpropan-2- yl)amino)-2-(6- azaspiro[2.5]octan-6- yl)nicotinamide |
| C11 | 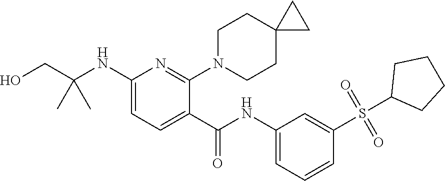 [SMILES: CC(C)(CO)Nc1ccc(C(=O)Nc2cccc(S(=O)(=O)C3CCCC3)c2)c(N2CCC3(CC2)CC3)n1] | N-(3- (cyclopentylsulfonyl)phenyl)-6- ((1-hydroxy-2-methylpropan-2- yl)amino)-2-(6- azaspiro[2.5]octan-6- yl)nicotinamide |
| C12 | 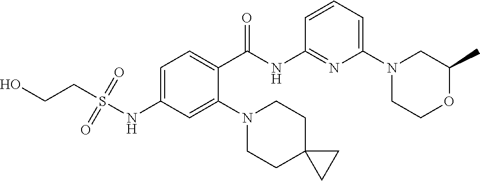 [SMILES: C[C@@H]1CN(c2cccc(NC(=O)c3ccc(NS(=O)(=O)CCO)cc3N3CCC4(CC3)CC4)n2)CCO1] | (R)-4-((2- Hydroxyethyl)sulfonamido)-N- (6-(2-methylmorpholino)pyridin- 2-yl)-2-(6-azaspiro[2.5]octan-6- yl)benzamide |
| C13 | 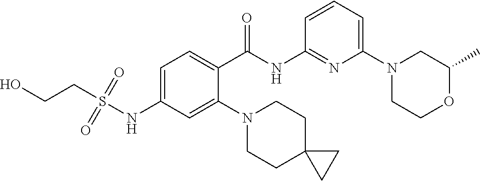 [SMILES: C[C@H]1CN(c2cccc(NC(=O)c3ccc(NS(=O)(=O)CCO)cc3N3CCC4(CC3)CC4)n2)CCO1] | (S)-4-((2- Hydroxyethyl)sulfonamido)-N- (6-(2-methylmorpholino)pyridin- 2-yl)-2-(6-azaspiro[2.5]octan-6- yl)benzamide |
| C14 | 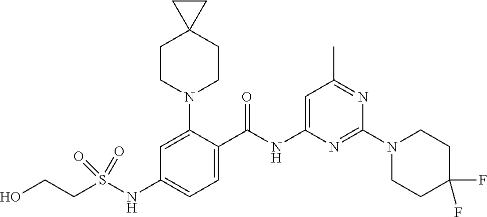 [SMILES: Cc1cc(NC(=O)c2ccc(NS(=O)(=O)CCO)cc2N2CCC3(CC2)CC3)nc(N2CCC(F)(F)CC2)n1] | N-(2-(4,4-Difluoropiperidin-1- yl)-6-methylpyrimidin-4-yl)-4- ((2-hydroxyethyl)sulfonamido)- 2-(6-azaspiro[2.5]octan-6- yl)benzamide |

* Ex. # stands for the example no. as well as the KIF18A Inhibitor Compound's short name used herein, e.g., in EXAMPLES.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

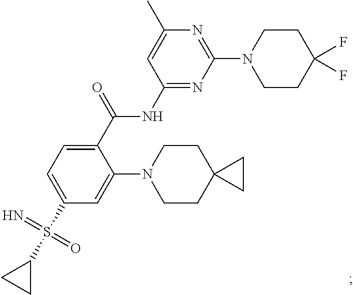 [SMILES: Cc1cc(NC(=O)c2ccc([S@](=N)(=O)C3CC3)cc2N2CCC3(CC2)CC3)nc(N2CCC(F)(F)CC2)n1]

which is named 2-(6-Azaspiro[2.5]octan-6-yl)-4-(R-cyclopropylsulfonimidoyl)-N-(2-(4,4-difluoro-1-piperidinyl)-6-methyl-4-pyrimidinyl)benzamide. In a sub-embodiment, the salt is a HCl salt, wherein said compound is named 2-(6-Azaspiro[2.5]octan-6-yl)-4-(R-cyclopropylsulfonimidoyl)-N-(2-(4,4-difluoro-1-piperdinyl)-6-methyl-4-pyrimidinyl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

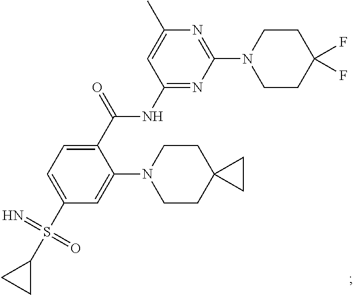 [SMILES: Cc1cc(NC(=O)c2ccc(S(=N)(=O)C3CC3)cc2N2CCC3(CC2)CC3)nc(N2CCC(F)(F)CC2)n1]

which is named 2-(6-azaspiro[2.5]octan-6-yl)-4-(S-cyclopropylsulfonimidoyl)-N-(2-(4,4-difluoro-1-piperidinyl)-6-methyl-4-pyrimidinyl)benzamide. In a sub-embodiment, the salt is a HCl salt, wherein said compound is named 2-(6-azaspiro[2.5]octan-6-yl)-4-(S-cyclopropylsulfonimidoyl)-N-(2-(4,4-difluoro-1-piperdinyl)-6-methyl-4-pyrimidinyl)benzamide hydrochloride.

In another embodiment, the present invention provides the method of any one of the preceding embodiments, wherein the KIF18A or the pharmaceutically-acceptable salt thereof, wherein said compound

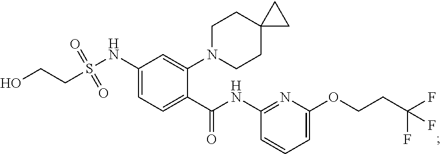 [SMILES: O=C(Nc1cccc(OCCC(F)(F)F)n1)c1ccc(NS(=O)(=O)CCO)cc1N1CCC2(CC1)CC2]

which is named 4-((2-Hydroxyethyl)sulfonamido)-2-(6-azaspiro[2.5]octan-6-yl)-N-(6-(3,3,3-trifluoropropoxy)pyridin-2-yl)benzamide. In a sub-embodiment, the salt is a HCl salt, wherein said compound is named 4-((2-Hydroxyethyl)sulfonamido)-2-(6-azaspiro[2.5]octan-6-yl)-N-(6-(3,3,3-trifluoropropoxy)pyridin-2-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

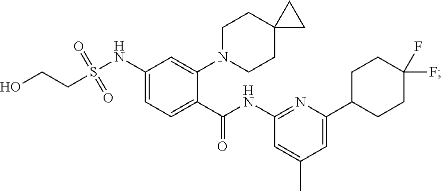 [SMILES: Cc1cc(NC(=O)c2ccc(NS(=O)(=O)CCO)cc2N2CCC3(CC2)CC3)nc(C2CCC(F)(F)CC2)c1]

which is N-(6-(4,4-difluoropiperidin-1-yl)-4-methylpyridin-2-yl)-4-((2-hydroxyethyl)sulfonamido)-2-(6-azaspiro[2.5]octan-6-yl)benzamide. In a sub-embodiment, the salt is a HCl salt, wherein said compound is named N-(6-(4,4-difluoropiperidin-1-yl)-4-methylpyridin-2-yl)-4-((2-hydroxyethyl)sulfonamido)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

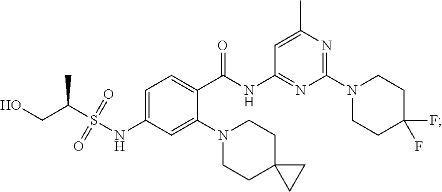 [SMILES: Cc1cc(NC(=O)c2ccc(NS(=O)(=O)[C@H](C)CO)cc2N2CCC3(CC2)CC3)nc(N2CCC(F)(F)CC2)n1]

which is named (R)—N-(2-(4,4-difluoropiperidin-1-yl)-6-methylpyrmidin-4-yl)-4-((2-hydroxy-1-methylethyl)sulfonamido)-2-(6-azaspiro[2.5]octan-6-yl)benzamide. In a sub-embodiment, the salt is a HCl salt, wherein said compound is named (R)—N-(2-(4,4-difluoropiperidin-1-yl)-6-methylpyrmidin-4-yl)-4-((2-hydroxy-1-methylethyl)sulfonamido)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate HCl mesylate tosylate or besylate salt, wherein said compound is

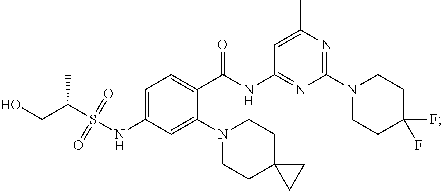 [SMILES: Cc1cc(NC(=O)c2ccc(NS(=O)(=O)[C@@H](C)CO)cc2N2CCC3(CC2)CC3)nc(N2CCC(F)(F)CC2)n1]

which is named (S)—N-(2-(4,4-difluoropiperidin-1-yl)-6-methylpyrimidin-4-yl)-4-((2-hydroxy-1-methylethyl)sulfonamido)-2-(6-azaspiro[2.5]octan-6-yl)benzamide. In a sub-embodiment, the salt is a HCl salt, wherein said compound is named (S)—N-(2-(4,4-difluoropiperidin-1-yl)-6-methylpyrimidin-4-yl)-4-((2-hydroxy-1-methylethyl)sulfonamido)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

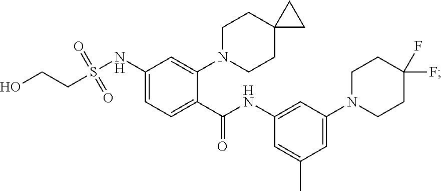 [SMILES: Cc1cc(NC(=O)c2ccc(NS(=O)(=O)CCO)cc2N2CCC3(CC2)CC3)cc(N2CCC(F)(F)CC2)c1]

which is named N-(3-(4,4-difluoropiperidin-1-yl)-5-methylphenyl)-4-((2-hydroxyethyl)sulfonamido)-2-(6-azaspiro[2.5]octan-6-yl)benzamide. In a sub-embodiment, the salt is a HCl salt, wherein said compound is named N-(3-(4,4-difluoropiperidin-1-yl)-5-methylphenyl)-4-((2-hydroxyethyl)sulfonamido)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

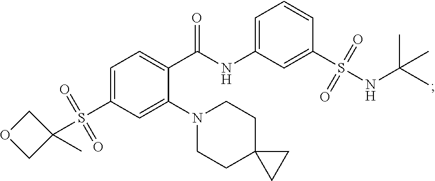 [SMILES: CC(C)(C)NS(=O)(=O)c1cccc(NC(=O)c2ccc(S(=O)(=O)C3(C)COC3)cc2N2CCC3(CC2)CC3)c1]

which is named N-(3-(N-(tert-Butyl)sulfamoyl)phenyl)-4-((3-methyloxetan-3-yl)sulfonyl)-2-(6-azaspiro[2.5]octan-6-yl)benzamide. In a sub-embodiment, the salt is a HCl salt, wherein said compound is named N-(3-(N-(tert-Butyl)sulfamoyl)phenyl)-4-((3-methyloxetan-3-yl)sulfonyl)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

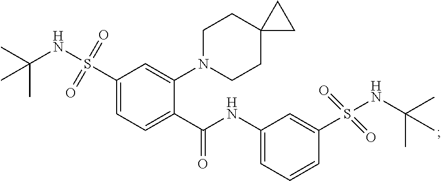 [SMILES: CC(C)(C)NS(=O)(=O)c1cccc(NC(=O)c2ccc(S(=O)(=O)NC(C)(C)C)cc2N2CCC3(CC2)CC3)c1]

which is named 4-(N-(tert-butyl)sulfamoyl)-N-(3-(N-(tert-butyl)sulfamoyl)phenyl)-2-(6-azaspiro[2.5]octan-6-yl)benzamide. In a sub-embodiment, the salt is a HCl salt, wherein said compound is named 4-(N-(tert-butyl)sulfamoyl)-N-(3-(N-(tert-butyl)sulfamoyl)phenyl)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

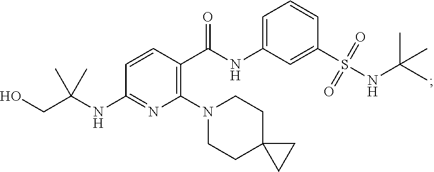 [SMILES: CC(C)(C)NS(=O)(=O)c1cccc(NC(=O)c2ccc(NC(C)(C)CO)nc2N2CCC3(CC2)CC3)c1]

which is named N-(3-(N-(ted-Butyl)sulfamoyl)phenyl)-6-((1-hydroxy-2-methylpropan-2-yl)amino)-2-(6-azaspiro[2.5]octan-6-yl)nicotinamide. In a sub-embodiment, the salt is a HCl salt, wherein said compound is named N-(3-(N-(tert-Butyl)sulfamoyl)phenyl)-6-((1-hydroxy-2-methylpropan-2-yl)amino)-2-(6-azaspiro[2.5]octan-6-yl)nicotinamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

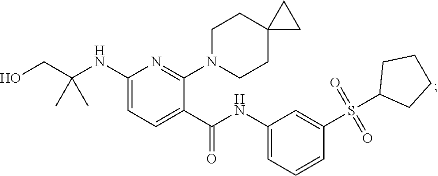 [SMILES: CC(C)(CO)Nc1ccc(C(=O)Nc2cccc(S(=O)(=O)C3CCCC3)c2)c(N2CCC3(CC2)CC3)n1]

which is named 4-(N-(tert-butyl)sulfamoyl)-N-(3-(N-(tert-butyl)sulfamoyl)phenyl)-2-(6-azaspiro[2.5]octan-6-yl)benzamide. In a sub-embodiment, the salt is a HCl salt, wherein said compound is named N-(3-(cyclopentylsulfonyl)phenyl)-6-((1-hydroxy-2-methylpropan-2-yl)amino)-2-(6-azaspiro[2.5]octan-6-yl)nicotinamide hydrochloride.

In another embodiment, the present invention provides the method of any one of the preceding embodiments, wherein the KIF18A or the pharmaceutically-acceptable salt thereof, such as sulfate HCl mesylate tosylate or besylate salt, wherein said compound is

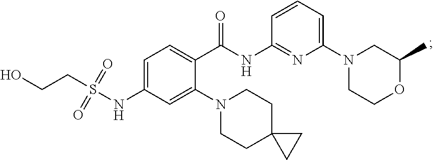 [SMILES: C[C@@H]1CN(c2cccc(NC(=O)c3ccc(NS(=O)(=O)CCO)cc3N3CCC4(CC3)CC4)n2)CCO1]

which is named (R)-4-((2-Hydroxyethyl)sulfonamido)-N-(6-(2-methylmorpholino)pyridin-2-yl)-2-(6-azaspiro[2.5]octan-6-yl)benzamide. In a sub-embodiment, the salt is a HCl salt, wherein said compound is named (R)-4-((2-Hydroxyethyl)sulfonamido)-N-(6-(2-methylmorpholino)pyridin-2-yl)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

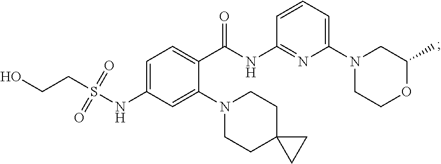 [SMILES: C[C@H]1CN(c2cccc(NC(=O)c3ccc(NS(=O)(=O)CCO)cc3N3CCC4(CC3)CC4)n2)CCO1]

which is named (S)-4-((2-Hydroxyethyl)sulfonamido)-N-(6-(2-methylmorpholino)pyridin-2-yl)-2-(6-azaspiro[2.5]octan-6-yl)benzamide. In a sub-embodiment, the salt is a HCl salt, wherein said compound is named (S)-4-((2-Hydroxyethyl)sulfonamido)-N-(6-(2-methylmorpholino)pyridin-2-yl)-2-(6-azaspiro[2.5]octan-6-yl)benzamide hydrochloride.

In another embodiment, the present invention provides the method of any one of the preceding embodiments, wherein the KIF18A or the pharmaceutically-acceptable salt thereof, such as sulfate, HCl, mesylate, tosylate, or besylate salt, wherein said compound is

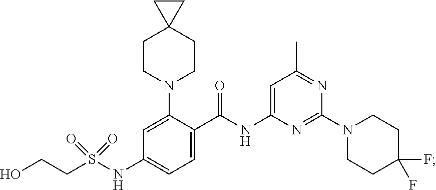 [SMILES: Cc1cc(NC(=O)c2ccc(NS(=O)(=O)CCO)cc2N2CCC3(CC2)CC3)nc(N2CCC(F)(F)CC2)n1]

which is named N-(2-(4,4-Difluoropiperidin-1-yl)-6-methylpyrimidin-4-yl)-4-((2-hydroxyethyl)sulfonamido)-2-(6-azaspiro[2.5]octan-6-yl)benzamide. In a sub-embodiment, the salt is a HCl salt, wherein said compound is named N-(2-(4,4-Difluoropiperidin-1-yl)-6-methylpyrimidin-4-yl)-4-((2-hydroxyethyl)sulfonamido)-2-(6-azaspiro[2.5]octan-6-yl)benzamide hydrochloride.

It is contemplated that the KIF18A inhibitor compounds include all pharmaceutically acceptable isotopically-labelled compounds of the present invention wherein one or more atoms are replaced by atoms having the same atomic number, but an atomic mass or mass number different from the atomic mass or mass number which predominates in nature.

Examples of isotopes suitable for inclusion in the compounds of the invention include, but are not limited to, isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the present invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and 13N, can be useful in Positron Emission Topography (PET) studies for examining substrate receptor occupancy.

Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Specific embodiments of the present invention include the compounds exemplified in the Examples below and their pharmaceutically acceptable salts, complexes, solvates, polymorphs, stereoisomers, metabolites, prodrugs, and other derivatives thereof.

Unless otherwise specified, the following definitions apply to terms found in the specification and claims:

“C_{α-β}alk” means an alkyl group comprising a minimum of α and a maximum of β carbon atoms in a branched or linear relationship or any combination of the three, wherein α and β represent integers. The alkyl groups described in this section may also contain one or two double or triple bonds. A designation of C₀alk indicates a direct bond. Examples of C₁₋₆alkyl include, but are not limited to the following:

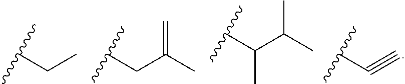

The terms “oxo” and “thioxo” represent the groups ═O (as in carbonyl) and ═S (as in thiocarbonyl), respectively.

“Halo” or “halogen” means a halogen atom selected from F, Cl, Br and I.

“C_{α-β}haloalk” means an alk group, as described above, wherein any number—at least one—of the hydrogen atoms attached to the alk chain are replaced by F, Cl, Br or I.

The group N(R^{a})R^{a} and the like include substituents where the two R^{a} groups together form a ring, optionally including a N, O or S atom, and include groups such as:

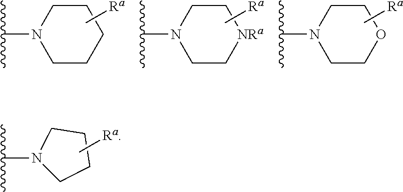

The group N(C_{α-β}alk) C_{α-62} alk, wherein α and β are as defined above, include substituents where the two C_{α-β}alk groups together form a ring, optionally including a N, O or S atom, and include groups such as:

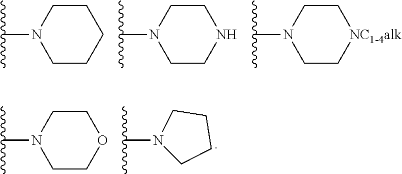

“Bicyclic” structure means a group that features two joined rings. A bicyclic ring can be carbocyclic (all of the ring atoms are carbons), or heterocyclic (the rings atoms consist, for example, 1, 2 or 3 heteroatoms, such as N, O, or S, in addition to carbon atoms). The two rings can both be aliphatic (e.g. decalin and norbornane), or can be aromatic (e.g. naphthalene), or a combination of aliphatic and aromatic (e.g. tetralin). Bicyclic rings include (a) spirocyclic compounds, wherein the two rings share only one single atom, the spiro atom, which is usually a quaternary carbon. Examples of spirocyclic compound include, but are not limited to:

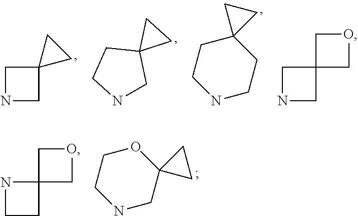

(b) fused bicyclic compounds, wherein two rings share two adjacent atoms. In other words, the rings share one covalent bond, i.e. the bridgehead atoms are directly connected (e.g. α-thujene and decalin). Examples of fused bicyclic rings include, but are not limited to:

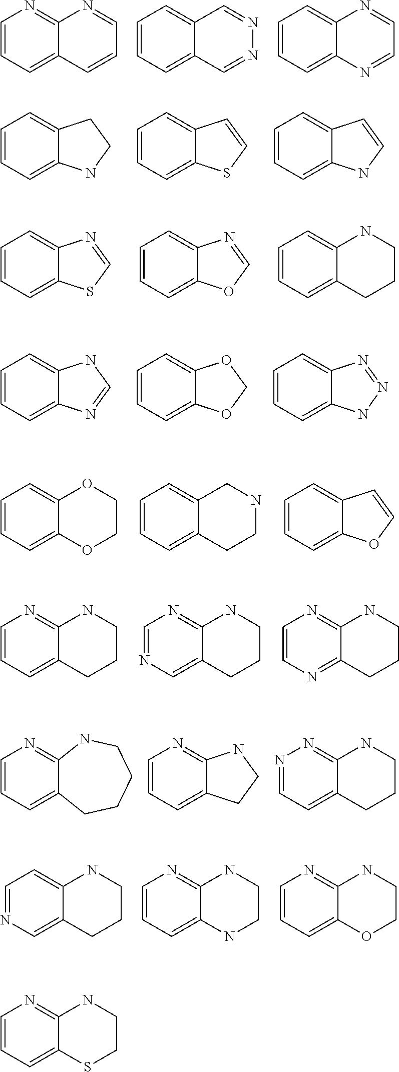

and (c) bridged bicyclic compounds, wherein the two rings share three or more atoms, separating the two bridgehead atoms by a bridge containing at least one atom. For example, norbornane, also known as bicyclo[2.2.1]heptane, can be thought of as a pair of cyclopentane rings each sharing three of their five carbon atoms. Examples of bridged bicyclic rings include, but are not limited to:

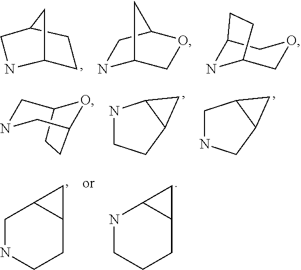

“Carbocycle” or “Carbocyclic” means a ring comprising by itself or in combination with other terms, represents, unless otherwise stated, cyclic version of “C_{α-β}alk”. Examples of carbocycle include cyclopentyl, cyclohexyl, 1-cyclohexenyl, 3-cyclohexenyl, cycloheptyl, cyclobutylene, cyclohexylene and the like.

“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et. al., J. Pharm. Sci. 66:1 (1977).

“Saturated, partially-saturated or unsaturated” includes substituents saturated with hydrogens, substituents completely unsaturated with hydrogens and substituents partially saturated with hydrogens.

It should be noted that compounds of the invention may contain groups that may exist in tautomeric forms, such as cyclic and acyclic amidine and guanidine groups, heteroatom substituted heteroaryl groups (Y′═O, S, NR), and the like, which are illustrated in the following examples:

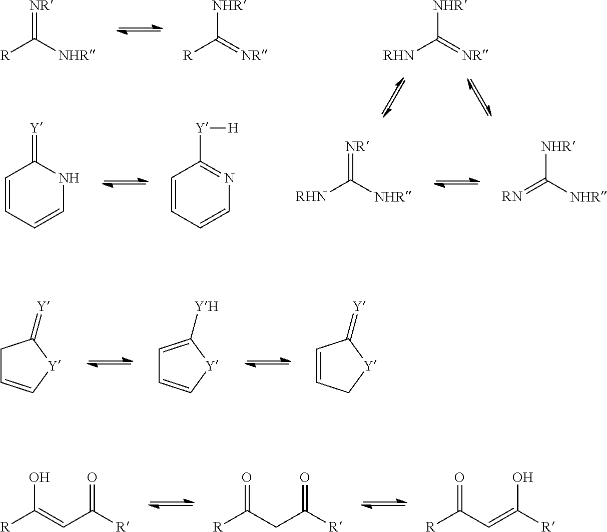

and though one form is named, described, displayed and/or claimed herein, all the tautomeric forms are intended to be inherently included in such name, description, display and/or claim.

Prodrugs of the compounds of this invention are also contemplated to be used in the method of this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, 4/11/81) discloses Mannich-base hydroxamic acid prodrugs, their preparation and use.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

In various aspects, the KIF18A inhibitor is a large molecule compound, e.g., a nucleic acid, oligonucleotide, polynucleotide, polypeptide, protein. In various instances, the KIF18A inhibitor is a molecule that targets and/or binds to a nucleic acid encoding KIF18A. In various aspects, the nucleic acid encoding KIF18A is the human KIF18A gene sequence (provided herein as SEQ ID NO: 30) or the human KIF18A mRNA sequence (provided herein as SEQ ID NO: 31) and the encoded KIF18A protein comprises the amino acid sequence of SEQ ID NO: 11.

Optionally, the KIF18A inhibitor comprises a nucleic acid that targets and/or binds to a nucleic acid encoding KIF18A, optionally, SEQ ID NO: 30 or 31). In exemplary aspects, the KIF18A inhibitor comprises a nucleic acid comprising a nucleotide sequence which is complementary to a portion of a nucleic acid encoding KIF18A (e.g., SEQ ID NO: 30 or 31). Optionally, the KIF18A inhibitor comprises a nucleic acid comprising a nucleotide sequence which binds to a portion of Exon 3, Exon 4, or Exon 7 of the KIF18A gene.

By “nucleic acid” as used herein includes “polynucleotide,” “oligonucleotide,” and “nucleic acid molecule,” and generally means a polymer of DNA or RNA, or modified forms thereof, which can be single-stranded or double-stranded, synthesized or obtained (e.g., isolated and/or purified) from natural sources, which can contain natural, non-natural or altered nucleotides, and which can contain a natural, non-natural or altered inter-nucleotide linkage, such as a phosphoroamidate linkage or a phosphorothioate linkage, instead of the phosphodiester found between the nucleotides of an unmodified oligonucleotide. The nucleic acid, in various aspects, comprises any nucleotide sequence which targets and/or binds to a nucleic acid encoding KIF18A. In some embodiments, the nucleic acid does not comprise any insertions, deletions, inversions, and/or substitutions. In other embodiments, the nucleic acid comprises one or more insertions, deletions, inversions, and/or substitutions. The nucleic acids in some aspects are constructed based on chemical synthesis and/or enzymatic ligation reactions using procedures known in the art. See, for example, Sambrook et. al., supra; and Ausubel et. al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridme, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N-substituted adenine, 7-methylguanine, 5-methylammomethyluracil, 5-methoxyaminomethyl-2-thiouracil, beta-D-mannosyl queosine, 5′-methoxycarboxymethyluracil, 5-methoxyuracil, 2-methylthio-N⁶-isopentenyladenine, uracil-5-oxyacetic acid (v), wybutosine, pseudouratil, queuosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the present disclosure can be purchased from companies, such as Macromolecular Resources (Fort Collins, Colo.) and Synthegen (Houston, Tex.).

In various aspects, the KIF18A inhibitor reduces expression of KIF18A. In various aspects, the KIF18A inhibitor is a non-coding RNA (ncRNA) which reduces expression of KIF18A. In exemplary aspects, the KIF18A inhibitor reduces expression of a KIF18A gene and/or a gene product thereof (e.g., KIF18A mRNA, KIF18A protein). The reduction of expression of a KIF18A gene and/or a gene product thereof (e.g., KIF18A mRNA, KIF18A protein) provided by the KIF18A inhibitor may not be a 100% or complete reduction or inhibition or abrogation. Rather, there are varying degrees of reduction of which one of ordinary skill in the art recognizes as having a potential benefit or therapeutic effect. In this regard, the KIF18A inhibitor may reduce expression of the KIF18A gene and/or gene product to any amount or level. In exemplary embodiments, the reduction provided by the KIF18A inhibitor is at least or about 10% reduction (e.g., at least or about 20% reduction, at least or about 30% reduction, at least or about 40% reduction, at least or about 50% reduction, at least or about 60% reduction, at least or about 70% reduction, at least or about 80% reduction, at least or about 90% reduction, at least or about 95% reduction, at least or about 98% reduction). Suitable methods of determining expression levels of nucleic acids (e.g., KIF18A genes, KIF18A RNA, e.g., mRNA) are known in the art and include but not limited to, quantitative polymerase chain reaction (qPCR) (e.g., quantitative real-time PCR (qRT-PCR)), RNAseq, and Northern blotting. Techniques for measuring gene expression include, for example, gene expression assays with or without the use of gene chips or gene expression microarrays are described in Onken et. al., J Molec Diag 12(4): 461-468 (2010); and Kirby et. al., Adv Clin Chem 44: 247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, Mass.). Suitable methods of determining expression levels of proteins are known in the art and include immunoassays (e.g., Western blotting, an enzyme-linked immunosorbent assay (ELISA), a radioimmunoassay (RIA), immunohistochemical assay and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLoS ONE 3(7): e2535.

In various aspects, the KIF18A inhibitor is an ncRNA which is not translated into a protein. In exemplary aspects, the KIF18A inhibitor is a short ncRNA, e.g., comprising less than about 30 nucleotides). In alternative aspects, the KIF18A inhibitor is a long ncRNA, e.g., comprising greater than about 200 nucleotides), including but not limited to a long non-coding RNA (lncRNA). Optionally, the short ncRNA is a microRNA (miRNA), short interfering RNA (siRNA), or a PIWI-interacting RNA (piRNA). In various aspects, the ncRNA is a small nucleolar RNA (snoRNA), small nuclear RNA (snRNA), extracellular RNA (exRNA), or a small Cajal body-specific RNA (scaRNA). See, e.g., Esteller, Nature Reviews Genetics 12: 861-874 (2011).

In exemplary instances, the KIF18A inhibitor is a molecule which mediates or triggers RNA interference (RNAi). In exemplary aspects, the KIF18A inhibitor is an RNAi trigger. RNAi is a ubiquitous mechanism of gene regulation in plants and animals in which target mRNAs may be degraded in a sequence-specific manner (Setten et. al., Nature Reviews Drug Discovery 18: 421-446 (2019); Sharp, Genes Dev., 15, 485-490 (2001); Hutvagner et. al., Curr. Opin. Genet. Dev., 12, 225-232 (2002); Fire et. al., Nature, 391, 806-811 (1998); Zamore et. al., Cell, 101, 25-33 (2000)). The RNA degradation process is mediated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et. al., Cell. 101, 25-33 (2000); Elbashir et. al., Genes Dev., 15, 188-200 (2001); Hammond et. al., Nature, 404, 293-296 (2000); Bernstein et. al., Nature, 409, 363-366 (2001)). siRNAs are incorporated into a large protein complex that recognizes and cleaves target mRNAs (Nykanen et. al., Cell, 107, 309-321 (2001). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et. al., Nature, 411: 494-498 (2001)).

siRNAs may be engineered and/or synthesized to enter a cell through endocytosis, and, directly interact with RNAi enzymes, Dicer and TAR RNA-binding protein (TRBP) in the cytosol to form the RISC-loading complex (RLC) and undergo strand selection to produce the mature RNA-induced silencing complex (RISC). The mature RISC regulates gene expression by inhibiting mRNA translation, inducing mRNA sequestration into cytoplasmic bodies, promoting mRNA degradation, and directing transcriptional gene silencing. siRNAs usually have full complementarity to a single target mRNA to induce potent and narrowly targeted gene silencing.

In exemplary aspects, the KIF18A inhibitor mediates RNAi and in various instances is a siRNA molecule specific for inhibiting the expression of the nucleic acid (e.g., the mRNA) encoding the KIF18A protein. The term “siRNA” as used herein refers to an RNA (or RNA analog) comprising from about 10 to about 50 nucleotides (or nucleotide analogs) which is capable of directing or mediating RNAi. In exemplary embodiments, a siRNA molecule comprises about 15 to about 30 nucleotides (or nucleotide analogs) or about 18 to about 25 nucleotides (or nucleotide analogs), e.g., 19-21 nucleotides (or nucleotide analogs). The siRNA can be double or single stranded.

In alternative aspects, the KIF18A inhibitor is a short hairpin RNA (shRNA) molecule specific for inhibiting the expression of the nucleic acid (e.g., the mRNA) encoding the KIF18A protein. The term “shRNA” as used herein refers to a molecule of about 20 or more base pairs in which a single-stranded RNA partially contains a palindromic base sequence and forms a double-strand structure therein (i.e., a hairpin structure). An shRNA can be a siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template. Though not wishing to be bound by any theory or mechanism, it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3′-protruding end. The length of the double-stranded portion is not particularly limited, but is preferably about 10 or more nucleotides, and more preferably about 20 or more nucleotides. Here, the 3′-protruding end may be preferably DNA, more preferably DNA of at least 2 nucleotides in length, and even more preferably DNA of 2-4 nucleotides in length.

In exemplary aspects, the KIF18A inhibitor is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011).

In various aspects, the KIF18A inhibitor is an RNA trigger which is a perfectly base-paired dsRNAs or short hairpin RNAs (shRNAs) ranging from 15 to 30 bp in overall length. In various instances, the KIF18A inhibitor is a larger (>21 bp) RNA duplex which interacts with the RNAi pathway enzyme Dicer for cleavage and handoff to the RLC. In alternative aspects, the KIF18A inhibiter is a shorter (<21 bp) siRNA or an analogue thereof that is able to bypass Dicer cleavage and enter the RISC via interactions mediated by the TRBP. This second pathway may still function in Dicer's absence. In various aspects, the KIF18A inhibitor is an ss-siRNA, sshRNA, hydrophobically-modified siRNA, sisiRNA, siRNA (ESC), siRNN, GalXC, DsiRNA, or shRNA, as described in Setten et. al., supra, 2019. Exemplary KIF18A inhibitors which mediate genome editing to cause reduced expression of KIF18A gene or to cause a complete elimination of KIF18A gene function, e.g., a gene knock-out, are described herein. In exemplary aspects, the KIF18A inhibitor comprises a sequence of SEQ ID NO: 12-18.

Pharmaceutical Compositions, Dosing, And Routes of Administration

In various aspects, the KIF18A inhibitor is provided as part of a pharmaceutical composition. Accordingly, pharmaceutical compositions including a compound as disclosed herein, together with a pharmaceutically acceptable excipient, such as, for example, a diluent or carrier, are provided by the present disclosure. Compounds and pharmaceutical compositions suitable for use in the present invention include those wherein the compound can be administered in an effective amount to achieve its intended purpose. Administration of the compound described in more detail below.

Suitable pharmaceutical formulations can be determined by the skilled artisan depending on the route of administration and the desired dosage. See, e.g., Remington's Pharmaceutical Sciences, 1435-712 (18th ed., Mack Publishing Co, Easton, Pa., 1990). Formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data obtainable through animal or human clinical trials.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable e” includes any and all solvents, dispersion media, coatings, antibacterial and antifungal agents, isotonic and absorption delaying agents and the like. The use of such excipients for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-camitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin.

The compound can be present in a pharmaceutical composition as a pharmaceutically acceptable salt. As used herein, “pharmaceutically acceptable salts” include, for example base addition salts and acid addition salts.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Pharmaceutically acceptable acid addition salts include inorganic or organic acid salts. Examples of suitable acid salts include the hydrochlorides, formates, acetates, citrates, salicylates, nitrates, phosphates. Other suitable pharmaceutically acceptable salts are well known to those skilled in the art and include, for example, formic, acetic, citric, oxalic, tartaric, or mandelic acids, hydrochloric acid, hydrobromic acid, sulfuric acid or phosphoric acid; with organic carboxylic, sulfonic, sulfo or phospho acids or N-substituted sulfamic acids, for example acetic acid, trifluoroacetic acid (TFA), propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid (mesylate), toluenesulfonic acids (tosylate), ethanesulfonic acid, 2-hydroxyethanesulfonic acid, ethane 1,2-disulfonic acid, benzenesulfonic acid (besylate), 4-methylbenzenesulfonic acid, naphthalene 2-sulfonic acid, naphthalene 1,5-disulfonic acid, 2- or 3-phosphoglycerate, glucose 6-phosphate, N-cyclohexylsulfamic acid (with the formation of cyclamates), or with other acid organic compounds, such as ascorbic acid.

Pharmaceutical compositions containing the compounds disclosed herein can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen.

For oral administration, suitable compositions can be formulated readily by combining a compound disclosed herein with pharmaceutically acceptable excipients such as carriers well known in the art. Such excipients and carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding a compound as disclosed herein with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Pharmaceutically acceptable ingredients are well known for the various types of formulation and may be for example binders (e.g., natural or synthetic polymers), lubricants, surfactants, sweetening and flavoring agents, coating materials, preservatives, dyes, thickeners, adjuvants, antimicrobial agents, antioxidants and carriers for the various formulation types.

When a therapeutically effective amount of a compound disclosed herein is administered orally, the composition typically is in the form of a solid (e.g., tablet, capsule, pill, powder, or troche) or a liquid formulation (e.g., aqueous suspension, solution, elixir, or syrup).

When administered in tablet form, the composition can additionally contain a functional solid and/or solid carrier, such as a gelatin or an adjuvant. The tablet, capsule, and powder can contain about 1 to about 95% compound, and preferably from about 15 to about 90% compound.

When administered in liquid or suspension form, a functional liquid and/or a liquid carrier such as water, petroleum, or oils of animal or plant origin can be added. The liquid form of the composition can further contain physiological saline solution, sugar alcohol solutions, dextrose or other saccharide solutions, or glycols. When administered in liquid or suspension form, the composition can contain about 0.5 to about 90% by weight of a compound disclosed herein, and preferably about 1 to about 50% of a compound disclosed herein. In one embodiment contemplated, the liquid carrier is non-aqueous or substantially non-aqueous. For administration in liquid form, the composition may be supplied as a rapidly-dissolving solid formulation for dissolution or suspension immediately prior to administration.

When a therapeutically effective amount of a compound disclosed herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound disclosed herein, an isotonic vehicle. Such compositions may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations can optionally contain a preservative to prevent the growth of microorganisms.

Injectable compositions can include sterile aqueous solutions, suspensions, or dispersions and sterile powders for the extemporaneous preparation of sterile injectable solutions, suspensions, or dispersions. In all embodiments the form must be sterile and must be fluid to the extent that easy syringability exists. It must be stable under the conditions of manufacture and storage and must resist the contaminating action of microorganisms, such as bacteria and fungi, by optional inclusion of a preservative. The carrier can be a solvent or dispersion medium containing, for example, water, ethanol, polyol (e.g., glycerol, propylene glycol, and liquid polyethylene glycol, and the like), suitable mixtures thereof, and vegetable oils. In one embodiment contemplated, the carrier is non-aqueous or substantially non-aqueous. The proper fluidity can be maintained, for example, by the use of a coating, such as lecithin, by the maintenance of the required particle size of the compound in the embodiment of dispersion and by the use of surfactants. The prevention of the action of microorganisms can be brought about by various antibacterial and antifungal agents, for example, parabens, chlorobutanol, phenol, sorbic acid, thimerosal, and the like. In many embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the embodiment of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Slow release or sustained release formulations may also be prepared in order to achieve a controlled release of the active compound in contact with the body fluids in the GI tract, and to provide a substantially constant and effective level of the active compound in the blood plasma. For example, release can be controlled by one or more of dissolution, diffusion, and ion-exchange. In addition, the slow release approach may enhance absorption via saturable or limiting pathways within the GI tract. For example, the compound may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

For administration by inhalation, compounds of the present invention are conveniently delivered in the form of an aerosol spray presentation from pressurized packs or a nebulizer, with the use of a suitable propellant. In the embodiment of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

The compounds disclosed herein can be formulated for parenteral administration by injection (e.g., by bolus injection or continuous infusion). Formulations for injection can be presented in unit dosage form (e.g., in ampules or in multidose containers), with an added preservative. The compositions can take such forms as suspensions, solutions, or emulsions in oily or aqueous vehicles, and can contain formulatory agents such as suspending, stabilizing, and/or dispersing agents.

Pharmaceutical formulations for parenteral administration include aqueous solutions of the compounds in water-soluble form. Additionally, suspensions of the compounds can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle (e.g., sterile pyrogen-free water) before use.

Compounds disclosed herein also can be formulated in rectal compositions, such as suppositories or retention enemas (e.g., containing conventional suppository bases). In addition to the formulations described previously, the compounds also can be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In particular, a compound disclosed herein can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or sugar alcohols, such as mannitol, or glucose, to make the solution isotonic with blood.

For veterinary use, a compound disclosed herein is administered as a suitably acceptable formulation in accordance with normal veterinary practice. The veterinarian can readily determine the dosing regimen and route of administration that is most appropriate for a particular animal.

In some embodiments, all the necessary components for the treatment of KIF18A-related disorder using a compound as disclosed herein either alone or in combination with another agent or intervention traditionally used for the treatment of such disease may be packaged into a kit. Specifically, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound disclosed herein, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

A “therapeutically effective amount” means an amount effective to treat or to prevent development of, or to alleviate the existing symptoms of, the subject being treated.

Determination of the effective amounts is well within the capability of those skilled in the art, especially in light of the detailed disclosure provided herein. Generally, a “therapeutically effective dose” refers to that amount of the compound that results in achieving the desired effect. For example, in one preferred embodiment, a therapeutically effective amount of a compound disclosed herein decreases KIF18A activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

The amount of compound administered can be dependent on the subject being treated, on the subject's age, health, sex, and weight, the kind of concurrent treatment (if any), severity of the affliction, the nature of the effect desired, the manner and frequency of treatment, and the judgment of the prescribing physician. The frequency of dosing also can be dependent on pharmacodynamic effects on arterial oxygen pressures. While individual needs vary, determination of optimal ranges of effective amounts of the compound is within the skill of the art. Such doses may be administered in a single dose or it may be divided into multiple doses.

Assaying for Inactivated Genes, Amplified Genes, and Expression Levels

In various embodiments of the methods of the present disclosure, the methods comprise assaying a sample for an inactivated gene (e.g., inactivated TP53 gene, inactivated Rb1 gene, and/or inactivated BRCA). As used herein, the term ‘inactivated’ in the context of a gene refers to a reduction or loss of function of the gene or gene product encoded by the gene. The inactivation of a gene may be caused by one or more known mechanisms. For example, the inactivation of the gene may be caused by a variation in (including, e.g., a loss of) DNA sequence, RNA sequence or protein sequence, relative to the corresponding wild-type gene, RNA, or protein or may be caused by an epigenetic variation that does not involve any alterations in the DNA sequence of the gene.

In various aspects, the assaying step comprises detecting the presence of a variation or anomaly in a gene or a gene product encoded by the gene, which variation or anomaly is relative to the corresponding wild-type gene or gene product, and which presence of the variation leads to or is associated with a silencing of the gene, a reduction or loss of expression of the gene or gene product encoded by the gene, a reduction or loss of function of the gene or gene product encoded by the gene, or a combination thereof. In various instances, the gene product is an RNA transcript or a protein. In various instances, the variation leads to at least a reduction or loss of function of the gene or gene product encoded by the gene. In various instances, the variation leads to at least a reduction or loss of function of the TP53 gene or gene product encoded by the TP53 gene. In various instances, the variation leads to at least a reduction or loss of function of the Rb1 gene or gene product encoded by the Rb1 gene. In various instances, the variation leads to at least a reduction or loss of function of the BRCA gene or gene product encoded by the BRCA gene.

The variation in the gene may be present anywhere in the gene, e.g., within an intron or exon, within a 5′-untranslated region (5′-UTR), or a 3′-untranslated region (3′-UTR). The variation may be present within or at any part of the transcript (e.g., RNA transcript, primary transcript, pre-mRNA, mRNA) encoded by the gene, or may be present within or at any part of the protein encoded by the gene.

In various aspects, the variation is a difference in DNA sequence, RNA sequence or protein sequence, relative to the corresponding wild-type gene, RNA, or protein. In various aspects, the sample is assayed for the inactivated gene by analyzing the nucleotide sequence of the gene, analyzing the nucleotide sequence of an RNA encoded by the gene, or analyzing the amino acid sequence of the protein encoded by the gene and comparing the sequence of gene of the sample to the corresponding wild-type human sequence of the gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein that may result in a gene copy number gain or amplification of the DNA, RNA, or protein. In various aspects, the assaying comprises detecting the presence of a gene mutation in the gene. In various aspects, the assaying comprises detecting the presence of a gene mutation in the gene or loss of nucleotides in the gene. In exemplary instances, the gene mutation is a missense mutation, nonsense mutation, insertion, deletion, duplication, frameshift mutation, truncation, or a repeat expansion. In various instances, the inactivated TP53 gene comprises a mutation, deletion, or truncation, the inactivated Rb1 gene comprises a mutation, deletion, or truncation, and/or the inactivated BRCA gene comprises a mutation, deletion, or truncation. As used herein, the term “BRCA gene” refers to the BRCA1 or the BRCA2 gene. In exemplary instances, the BRCA gene is BRCA1. In exemplary aspects, the BRCA gene is BRCA2.

In various instances, the variation is epigenetic and does not involve any alterations in the DNA sequence of the gene. In exemplary aspects, the inactivated gene is epigenetically silenced and optionally involves a covalent modification of the DNA or histone proteins. The covalent modification of the DNA may be, for example, a cytosine methylation or hydroxymethylation. The covalent modification of the histone protein may be, for example, a lysine acetylation, lysine or arginine methylation, serine or threonine phosphorylation, or lysine ubiquitination or sumoylation. Mechanisms of gene silencing can occur during transcription or translation. Exemplary mechanisms of gene silencing include but are not limited to DNA methylation, histone modification, and RNA interference (RNAi). In various aspects, the inactivated gene is an epigenetically silenced gene having an epigenetically silenced promoter. Optionally, the inactivated TP53 gene has an epigenetically silenced TP53 promoter or the inactivated Rb1 gene has an epigenetically silenced Rb1 promoter or the inactivated BRCA gene has an epigenetically silenced BRCA promoter. Suitable techniques to assay for epigenetic silencing include but are not limited to chromatin immunoprecipitation (ChIP-on chip, ChIP-Seq) fluorescent in situ hybridization (FISH), methylation-sensitive restriction enzymes, DNA adenine methyltransferase identification (DamID) and bisulfite sequencing. See, e.g., Verma et. al., Cancer Epidemiology, Biomarkers, and Prevention 23: 223-233 (2014).

In various aspects, the inactivated gene is inactivated by a virus-induced gene silencing (VIGS). In various instances, the inactivated TP53 gene is inactivated by a viral protein, e.g., human papillomavirus (HPV) E6 protein. Optionally, the HPV E6 protein interacts with the p53 protein encoded by the TP53 gene and renders the p53 protein inactive. In various instances, the inactivated Rb1 gene is inactivated by a viral protein, e.g., HPV E7 protein. Optionally, the HPV E7 protein interacts with the Rb protein encoded by the Rb1 gene and renders the Rb protein inactive. Such modes of silencing are known in the art. See, e.g., Jiang and Milner, Oncogene 21: 6041-6048 (2002).

In various embodiments of the methods of the present disclosure, the methods comprise assaying a sample for a gene amplification, e.g., CCNE1 amplification, or an increase in the number of copies of a gene, e.g., a gene copy number gain of the gene. In various instances, the sample is assayed for the gain or amplified gene by DNA- or RNA-based techniques (gene expression analysis [comparative genomic hybridization, RNA-based hybridization], NGS, PCR, or Southern blot) or by molecular cytogenetic techniques (FISH2 with gene-specific probes, CISH (chromogenic in situ hybridization). In various aspects, competitive or quantitative PCR, genomic hybridization to cDNA microarrays, hybridization and quantification of gene probes to RNA are carried out to detect the gene amplification or gene copy number gain. See., e.g., Harlow and Stewart, Genome Res 3: 163-168 (1993); Heiskanen et. al., Cancer Res 60(4): 799-802 (2000). In various instances, the method comprises assaying a sample for a gene copy number gain or amplification of an MDM2 gene and/or a gene copy number gain or amplification or mutation of an FBXW7 gene. In exemplary aspects, the method comprises assaying a sample for a gene copy number gain or amplification of an MDM2 gene and a reduction in p53 protein levels. In exemplary aspects, the method comprises assaying a sample for a mutation in an FBXW7 gene and an overexpression of a gene product encoded by the CCNE1 gene. Next Generation Sequencing (NGS) may also be employed as a method by which to detect a gene copy number gain or loss or a gene amplification whereby genetic areas are sequenced and sequencing reads are compared to other genes to deduce gain or loss of the gene of interest.

In exemplary aspects, the inactivated TP53 gene (i) comprises a TP53 gene mutation, deletion, truncation, and/or an epigenetically silenced TP53 promoter, (ii) is inactivated by a viral protein or via gene amplification of an MDM2 gene, or (iii) a combination thereof. Optionally, the viral protein is a Human Papillomavirus (HPV) E6 protein. In exemplary aspects, the inactivated Rb1 gene (i) comprises an Rb1 gene mutation, deletion, truncation, and/or an epigenetically silenced Rb1 promoter, (ii) is inactivated by a viral protein or (iii) a combination thereof. Optionally, the viral protein is a Human Papillomavirus (HPV) E7 protein. In exemplary aspects, the inactivated BRCA gene (i) comprises a BRCA gene mutation, deletion, truncation, and/or an epigenetically silenced BRCA promoter. Optionally, the BRCA gene is a BRCA1 gene. Alternatively, the BRCA gene is a BRCA2 gene.

In various aspects, the inactivated TP53 gene, inactivated Rb1 gene, CCNE1 gene copy number gain or amplification and/or inactivated BRCA gene is present in the germline cells of the neoplastic disease (e.g., cancer). In various aspects, the inactivated TP53 gene, inactivated Rb1 gene, CCNE1 gene copy number gain or amplification and/or inactivated BRCA gene is present in the germline cells of the neoplastic disease (e.g., cancer) and absent from somatic cells of the neoplastic disease (e.g., cancer). Optionally, due to somatic mutations of the neoplastic disease, the somatic cells of the neoplastic disease have reverted back to wild-type genotype and thus do not exhibit the inactivated TP53 gene, inactivated Rb1 gene, CCNE1 gene copy number gain or amplification and/or inactivated BRCA gene, though the germline cells of the neoplastic disease still demonstrate inactivated TP53 gene, inactivated Rb1 gene, CCNE1 gene copy number gain or amplification and/or inactivated BRCA gene. For example, the neoplastic disease may be a PARP inhibitor-resistant cancer and only the germline cells of the cancer have an inactivated BRCA1 gene, whereas the somatic cells of the cancer exhibit a restored BRCA1 coding region and function.

In exemplary instances, the assaying step comprises a cytogenetics method and/or molecular method for detecting the presence of an inactivated or amplified gene or gene copy number gain, e.g., an inactivated TP53 gene, inactivated Rb1 gene, amplified CCNE1 gene or inactivated BRCA gene. In exemplary aspects, the assaying step comprises direct DNA sequencing, DNA hybridization and/or restriction enzyme digestion. Optionally, the cytogenetics method comprises karyotyping, fluorescence in situ hybridization (FISH), comparative genomic hybridization (CGH), or a combination thereof. In various instances, the molecular method comprises restriction fragment length polymorphism (RFLP), amplification refractory mutation system (ARMS), polymerase chain reaction (PCR), multiplex ligation dependent probe amplification (MLPA), denaturing gradient gel electrophoresis (DGGE), single strand conformational polymorphism (SSCP), heteroduplex analysis, chemical cleavage of mismatch (CCM), protein truncation test (PTT), oligonucleotide ligation assay (OLA), or a combination thereof. Optionally, the PCR is a multiplex PCR, nested PCR, RT-PCR, or real time quantitative PCR. In various aspects, the assaying step comprises assaying expression levels of RNA or protein encoded by the TP53 gene, Rb1 gene, CCNE1 gene, and/or the BRCA gene. In various aspects, the assaying step comprises ARMS, FISH, IHC, or NGS. Such techniques are described in Su et al., J Experimental Clin Cancer Research 36: 121 (2017) and He et al., Blood 127(24): 3004-3014 (2016). In various instances, the assaying step comprises whole-exome sequencing or whole genome sequencing. In exemplary aspects, the assaying comprises a liquid biopsy. Liquid biopsies are described in detail in the art. See, e.g., Poulet et al., Acta Cytol 63(6): 449-455 (2019), Chen and Zhao, Hum Genomics 13(1): 34 (2019).

In various aspects, the gene copy number gain or amplification leads to overexpressed or increased levels of the gene products (e.g., RNA and/or protein) encoded by the gene. Methods of detecting increased levels in RNA and/or protein are known in the art. In exemplary aspects, the gene copy number gain or amplification of the CCNE1 gene leads to overexpressed or increased levels of the gene products encoded by the CCNE1 gene. In exemplary aspects, the overexpression of the CCNE1 gene product is caused by a mutation in an FBXW7 gene. In various aspects, the sample is positive for overexpression of the CCNE1 gene products and a mutation in an FBXW7 gene.

In various instances, the methods of the present disclosure comprise measuring a level of expression of a gene, via RNA transcripts, e.g., a messenger RNA (mRNA), or a protein, in a sample (e.g., a sample comprising tissue or blood) obtained from a subject. In exemplary aspects of the presently disclosed methods, the method comprises measuring the level of expression of TP53, Rb1, BRCA, CCNE1, or any gene product encoded by the gene, or any combination thereof. Suitable methods of determining expression levels of nucleic acids (e.g., genes, RNA, mRNA) are known in the art and include but not limited to, quantitative polymerase chain reaction (qPCR) (e.g., quantitative real-time PCR (qRT-PCR)), RNAseq, Nanostring, and Northern blotting. Techniques for measuring gene expression also include, for example, gene expression assays with or without the use of gene chips or gene expression microarrays are described in Onken et. al., J Molec Diag 12(4): 461-468 (2010); and Kirby et. al., Adv Clin Chem 44: 247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, Mass.), and Nanostring (Geiss et. al., Nature Biotechnology 26: 317-325 (2008)). Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLoS ONE 3(7): e2535. Proteomic analysis which is the systematic identification and quantification of proteins of a particular biological system are known. Mass spectrometry is typically the technique used for this purpose.

In exemplary aspects, the method comprises measuring the level of a complementary DNA (cDNA) based on the RNA encoded by said gene. Briefly, the method comprises extracting or isolating RNA from the sample (e.g., from the tumor cell(s) of the sample) and synthesizing cDNA based on RNA isolated from the sample. Alternatively or additionally, in some aspects, measuring the expression level comprises isolating RNA from the sample, producing complementary DNA (cDNA) from the RNA, amplifying the cDNA and hybridizing the cDNA to a gene expression microarray. Accordingly, in some aspects, measuring the expression level comprises isolating RNA from the sample and quantifying the RNA by RNA-Seq. In alternative or additional aspects, the level of expression is determined via an immunohistochemical assay. In exemplary aspects, measuring the expression level comprises contacting the sample with a binding agent to TP53, Rb1, BRCA, or CCNE1, or a gene product thereof, or a combination thereof. In some aspects, the binding agent is an antibody, or antigen-binding fragment thereof. In some aspects, the binding agent is a nucleic acid probe specific for TP53, Rb1, BRCA, or CCNE1, or an RNA transcript thereof, or a complement thereof.

Once the expression level of TP53, Rb1, BRCA, or CCNE1, or the gene product thereof, is measured from the sample obtained from the subject, the measured expression level may be compared to a reference level, normalized to a housekeeping gene, mathematically transformed. In exemplary instances, the measured expression level of TP53, Rb1, BRCA, or CCNE1, or the gene product thereof, is centered and scaled. Suitable techniques of centering and scaling biological data are known in the art. See, e.g., van den Berg et. al., BMC Genomics 7: 142 (2006).

The wild-type TP53, Rb1, CCNE1, and BRCA genes, as well as the RNA and proteins encoded by these genes, are known in the art. Exemplary sequences of each are available at the website for the National Center for Biotechnology Information (NCBI) and provided in the sequence listing submitted herewith.

TABLE A

| Gene name | NCBI, | | SEQ | | SEQ |
|---|---|---|---|---|---|
| (abbreviation, | HUGO | mRNA | ID | Protein | ID |
| full) | Gene ID No. | Accession No. | NO: | Accession No. | NO: |
| TP53 | 7157, 11998 | NM_000546.6 | 1 | NP_000537.3 | 2 |
| RB1 | 5925, 9884 | NM_000321.3 | 3 | NP_000312.2 | 4 |
| CCNE1 | 898, 1589 | NM_001238.4 | 5 | NP_001229.1 | 6 |
| BRCA1 | 672, 1100 | NM_007294.4 | 7 | NP_009225.1 | 7 |
| BRCA2 | 675, 1101 | NM_000059.4 | 9 | NP_000050.3 | 10 |

In exemplary embodiments, the methods comprise measuring additional genes, RNA, and/or proteins not listed in Table A. In exemplary embodiments, the methods comprise measuring the expression level of at least one additional gene, RNA, or protein. In exemplary instances, the methods comprise measuring the expression level of at least 2, 3, 4, 5 or more additional genes, at least 2, 3, 4, 5 or more additional RNA, and/or at least 2, 3, 4, 5 or more additional proteins in the sample. In exemplary instances, the methods comprise measuring the expression level of at least 10, 15, 20 or more additional genes, at least 10, 15, 20 or more additional RNA, and/or at least 10, 15, 20 or more additional proteins in the sample. In exemplary instances, the methods comprise measuring the expression level of at least 50, 100, 200 or more additional genes, at least 50, 100, 200 or more additional RNA, and/or at least 50, 100, 200 or more additional proteins in the sample. In exemplary instances, the methods comprise measuring the expression level of a plurality of different genes, a plurality of RNA, and/or a plurality of proteins, in addition to one or more listed in Table A. In exemplary aspects, the methods comprise measuring the expression of one or more homologous recombination deficiency (HRD) genes, including but not limited to BRCA1, BRCA2, ATM, ATRX, BARD1, BLM, BRIP1, CDK12, CHEK1, CHEK2, FANCA, FANCC, FANCD2, FANCE, FANCF, FANCI, FANCL, FANCM, MRE11, NBN, PALB2, RAD50, RAD51, RAD51B, RAD51C, RAD51D, RAD52, RAD54L, and RPA1 (DR Hodgson et al British Journal of Cancer. 2018; 119:1401-9; A L Heeke et al JCO Precis Oncol. 2018; 2:1-3). In exemplary aspects, the methods comprise measuring the expression of one or more kinesin genes, ABC transporter genes, SAC genes, kinetochore genes, EMT genes, PAM50 signature (B Wallden et al BMC Medical Genomics. 2015; 8(1):54), genes of the CIN25/70 gene signatures (SL Carteret al Nature Genetics. 2006; 38(9):1043-8), or a combination thereof.

The assaying step allows for the sample to be identified as “positive” or “negative” for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. As used herein, the term “positive” in the context of a sample means that an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are present in the sample. As used herein, the term “negative” in the context of a sample means that an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are absent from the sample, e.g., the sample does not have an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof is/are present in the sample.

Responsiveness, Sensitivity and Resistance

The present disclosure relates to responsiveness, sensitivity and/or resistance to a drug, e.g., KIF18A inhibitor, CDK4/6 inhibitor. The present disclosure provides a method of identifying a subject with a neoplastic disease as sensitive or responsive to treatment with a KIF18A inhibitor is provided herein. A method of determining a treatment for a subject with a neoplastic disease comprising determining sensitivity of the neoplastic disease to a KIF18A inhibitor or determining sensitivity of the neoplastic disease to a CDK4/6 inhibitor are disclosed herein. The present disclosure also relates to method of treating a subject with a neoplastic disease which is resistant to treatment with a CDK4/6 inhibitor.

As used herein “sensitivity” refers to the way a neoplastic disease (e.g., cancer, tumor) reacts to a drug/compound, e.g., a KIF18A inhibitor, CDK4/6 inhibitor). In exemplary aspects, “sensitivity” means “responsive to treatment” and the concepts of “sensitivity” and “responsiveness” are positively associated in that a neoplastic disease (e.g., tumor or cancer cell) that is responsive to a drug/compound treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a tumor that is resistant to a drug treatment is either not sensitive nor responsive to that drug or was initially sensitive to the drug and is no longer sensitive upon acquiring resistance; that drug is not an effective treatment for that tumor or cancer cell.

The term “responsiveness” as used herein refers to the extent of a therapeutic response or responsiveness of a cancer cell or tumor to a drug/compound (e.g., a KIF18A inhibitor, a CDK4/6 inhibitor) or other treatment (e.g., radiation therapy) as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), in order to provide a baseline for comparison after treatment with a drug (e.g., CDK4/6 inhibitor). The response assessment and evaluation criteria for tumors are published in Eisenhauer et. al., Eur J Cancer 45:228-247 (2009) and Litiére et. al., Journal of Clinical Oncology 37(13): 1102-1110 (2019) DOI: 10.1200/JCO.18.01100. Briefly, Section 4.3 of Eisenhauer et. al., 2009, supra, teaches response criteria to be used to determine objective tumor response for target lesions, as follows:

| Response Type | Signifies that: |
|---|---|
| Complete | Disappearance of all target lesions. Any pathological |
| Response (CR) | lymph nodes (whether target or non-target) must have |
| | reduction in short axis to <10 mm. |
| Partial | At least a 30% decrease in the sum of diameters of |
| Response (PR) | target lesions, taking as reference the baseline sum |
| | diameters. |
| Stable | Neither sufficient shrinkage to qualify for PR nor |
| Disease (SD) | sufficient increase to qualify for PD, taking as |
| | reference the smallest sum diameters while on study |
| Progressive | At least a 20% increase in the sum of diameters of |
| Disease (PD) | target lesions, taking as reference the smallest sum on |
| | study (this includes the baseline sum if that is the |
| | smallest on study). In addition to the relative increase |
| | of 20%, the sum must also demonstrate an absolute |
| | increase of at least 5 mm. (Note: the appearance of |
| | one or more new lesions is also considered |
| | progression). |

In ideal cases, a drug or other treatment results in CR or PR as best over-all response with durable duration of response (DOR). Responses of SD with short DOR or PD in some aspects are used to show that a drug is not an effective treatment for cancer or that a tumor has stopped responding to treatment.

In exemplary aspects, responsiveness accounts for or is based on clinical benefit rate (CBR) which is defined as the proportion of patients in whom the best overall response is determined as complete response (CR), partial response (PR) or stable disease (SD) >16 weeks and 24 weeks. Optionally, the CBR relates to proportion of patients in whom the best overall response is determined as complete response (CR), partial response (PR) or stable disease (SD) >16 weeks and 24 weeks wherein the patients have refractory or relapsed breast cancer or ovarian cancer.

As recognized by one of ordinary skill in the art, such a tumor or cancer cell is understood as one that has lost sensitivity to treatment and/or one that has become resistant to treatment.

Provided herein are methods of identifying a subject with a neoplastic disease as sensitive or responsive to treatment with a KIF18A inhibitor. In exemplary embodiments, the method comprises assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof, wherein the subject is identified as sensitive to treatment with a KIF18A inhibitor, when the sample is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In exemplary embodiments, the method comprises determining the sensitivity of the neoplastic disease to treatment with a CDK4/6. In various aspects, when the neoplastic disease is not sensitive to the CDK4/6 inhibitor, the neoplastic disease is deemed as sensitive to the KIF18A inhibitor. In various aspects, methods of identifying a subject with a neoplastic disease as sensitive or responsive to treatment with a CDK4/6 inhibitor are provided. In exemplary aspects, the method comprises determining the sensitivity of the neoplastic disease to treatment with a KIA18A inhibitor. In various aspects, when the neoplastic disease is not sensitive to the KIF18A inhibitor, the neoplastic disease is deemed as sensitive to the CDK4/6 inhibitor. In various aspects, the methods identify the subject as one who is likely to achieve a complete response upon treatment with the KIF18A inhibitor. In various aspects, the methods identify the subject as one who is likely to achieve at least a partial response upon treatment with the KIF18A inhibitor. In various aspects, the methods identify the subject as one who is likely to not exhibit stable disease or progressive disease upon treatment with the KIF18A inhibitor.

Without being bound to any particular theory, in exemplary embodiments, a neoplastic disease which is sensitive or responsive to a CDK4/6 inhibitor is not sensitive or responsive to a KIF18A inhibitor and a neoplastic disease which is sensitive or responsive to a KIF18A inhibitor is not sensitive or responsive to a CDK4/6 inhibitor. Thus, the present disclosure provides a method of determining a treatment for a subject with a neoplastic disease comprising determining the sensitivity of the neoplastic disease to treatment with a KIF18A inhibitor or a CDK4/6 inhibitor. In various aspects, when the neoplastic disease is insensitive to the CDK4/6 inhibitor, the treatment for the subject is determined as a treatment comprising a KIF18A inhibitor and when the neoplastic disease is insensitive to the KIF18A inhibitor, the treatment for the subject is determined as a treatment comprising a CDK4/6 inhibitor. Accordingly, the present disclosure provides methods of treating a subject with a neoplastic disease resistant to treatment with a CDK4/6 inhibitor, comprising administering a KIF18A inhibitor to treat the patient and methods of treating a neoplastic disease in a subject who is or has been treated with a CDK4/6 inhibitor, comprising administering to the subject a KIF18A inhibitor, optionally, wherein the KIF18A inhibitor is co-administered with the CDK4/6 inhibitor. Also, the present disclosure provides methods of treating a subject with a neoplastic disease resistant to treatment with a KIF18A inhibitor, comprising administering a CDK4/6 inhibitor to treat the patient and methods of treating a neoplastic disease in a subject who is or has been treated with a KIF18A inhibitor, comprising administering to the subject a CDK4/6 inhibitor, optionally, wherein the CDK4/6 inhibitor is co-administered with the KIF18A inhibitor. Pharmaceutical combinations comprising a CDK4/6 inhibitor and a KIF18A inhibitor are provided.

In various instances of the presently disclosed methods, the method further comprises determining the sensitivity to a CDK4/6 or determining the sensitivity to a KIF18A inhibitor. In various instances, the method comprises assaying for the sensitivity to a CDK4/6 inhibitor. In various aspects, assaying the sensitivity comprises measuring or describing quantitatively in terms of the point of intersection of a dose-effect curve with the axis of abscissal values or a line parallel to it; wherein the point corresponds to the dose just required to produce a given degree of effect. In various aspects, assaying the sensitivity comprises carrying out one or more of a nuclear count assay, a centrosome count assay, growth assay, and/or tumor regression assay, such as those described herein. See, e.g., Examples 1-4.

In various instances of the presently disclosed methods, the sensitivity to a CDK4/6 inhibitor is determined by assaying a sample obtained from the subject for the absence of (i) an inactivated Rb1 gene, (ii) an amplified CCNE1 gene, gene copy number gain of the CCNE1 gene, or overexpression of a CCNE1 gene product, or (iii) a combination thereof.

Methods of maintaining sensitivity of a neoplastic disease to treatment with a CDK4/6 inhibitor in a subject are provided herein. In exemplary embodiments, the method comprises administering to the subject a KIF18A inhibitor. In various aspects, at least 50% of the sensitivity to the treatment is maintained. Optionally, at least or about a 50% increase, at least or about a 60% increase, at least or about a 70% increase, at least or about a 80% increase, at least or about a 90% increase, at least or about a 95% increase, or at least or about a 98% increase, at least or about a 100% increase) of the sensitivity to the treatment is maintained.

Additional Steps

With regard to the methods of the invention, the methods may include additional steps. For example, the method may include repeating one or more of the recited step(s) of the method. Accordingly, in exemplary aspects, the method comprises assaying a second sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof, wherein the second sample is obtained from the subject at a different time point, relative to the time at which the first sample was obtained from the subject. In exemplary aspects, the method comprises assaying a sample obtained from the subject every month, every 2 months, every 3 months, every 4 months, or every 6 to 12 months, wherein the assaying is based on a different sample obtained from the same subject.

In exemplary aspects, the presently disclosed method further comprises obtaining a sample from the subject. In various aspects, a sample is obtained by blood draw, apheresis, leukapheresis, biopsy or by collection of urine.

In exemplary aspects, the method further comprises administering a KIF18A inhibitor once the need therefor has been determined. Methods of administering a KIF18A inhibitor to a subject may be the same as or similar to any of the presently disclosed methods of administering a pharmaceutical combination.

In various aspects, the method further comprises assaying the sample for spindle assembly checkpoint (SAC) activation, centrosome aberrations, multipolar spindles or a combination thereof. Suitable methods of assaying the sample for these characteristics/features are described herein. See, Examples 5-10.

Any and all possible combinations of the steps described herein are contemplated for purposes of the inventive methods

Pharmaceutical Combinations

In exemplary embodiments, the KIF18A inhibitor described herein is administered alone, and in alternative embodiments, the KIF18A inhibitor described herein is administered in combination with another therapeutic agent, e.g., another KIF18A inhibitor but a different type (e.g., structure), or another therapeutic which does not inhibit KIF18A. In exemplary aspects, the other therapeutic aims to treat or prevent a neoplastic disease. In exemplary aspects, the other therapeutic is CDK4/6 inhibitor. Accordingly, the present disclose provides pharmaceutical combinations comprising a KIF18A inhibitor. The pharmaceutical combination comprises a KIF18A inhibitor and another active agent. In exemplary instances, the KIF18A inhibitor is formulated with the other active agent and the two active agents are administered simultaneously. In exemplary instances, the KIF18A inhibitor is not formulated with the other active agent and the two active agents may be administered separately or together. In various aspects, the two active agents are administered to the subject sequentially.

In exemplary embodiments, the pharmaceutical combination comprises a KIF18A inhibitor and a CDK4/6 inhibitor. In various aspects, the KIF18A inhibitor is formulated separately from the CDK4/6 inhibitor.

In various aspects, the pharmaceutical combination or KIF18A inhibitor or CDK4/6 inhibitor is formulated with a pharmaceutically acceptable carrier, diluent, or excipient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In various aspects, the pharmaceutical combination or KIF18A inhibitor or CDK4/6 inhibitor is formulated for oral administration or systemic or parenteral administration (e.g., intravenous, subcutaneous, intramuscular administration). In various aspects, the KIF18A inhibitor is formulated for oral administration. In various aspects, the CDK4/6 inhibitor is formulated for oral administration.

CDK4/6 Inhibitors

As used herein, the term “CDK4/6 inhibitor” refers to any compound or molecule that targets the cyclin-dependent kinases, CDK4 and CDK6, and reduces or inhibits their enzyme activity, e.g., kinase activity. In exemplary aspects, the CDK4/6 inhibitor acts on CDK4 and CDK6 to induce cell-cycle arrest. During cell cycle progression, CDK4 and CDK6 target the growth-suppressive protein, retinoblastoma protein (Rb), for phosphorylation, and the Rb protein is inactivated when phosphorylated. When CDK4 and CDK6 are inhibited by CDK4/6 inhibitors, Rb is not phosphorylated (or is less phosphorylated) such that Rb is free to carry out its growth-suppressive function. In exemplary embodiments, the CDK4/6 inhibitor is a serine/threonine kinase inhibitor, a Cytochrome P450 (CYP450) 3A Inhibitor, or both. In various aspects, the CDK4/6 inhibitor inhibits the phosphorylation of retinoblastoma (Rb) protein. In various aspects, the CDK4/6 inhibitor inhibits the function of CYP4503A.

The reduction or inhibition provided by the CDK4/6 inhibitor may not be a 100% or complete inhibition or abrogation or reduction. Rather, there are varying degrees of reduction or inhibition of which one of ordinary skill in the art recognizes as having a potential benefit or therapeutic effect. In this regard, the CDK4/6 inhibitor may inhibit the CDK4 and/or CDK6 protein(s) to any amount or level. In exemplary embodiments, the reduction or inhibition provided by the CDK4/6 inhibitor is at least or about 10% reduction or inhibition (e.g., at least or about 20% reduction or inhibition, at least or about 30% reduction or inhibition, at least or about 40% reduction or inhibition, at least or about 50% reduction or inhibition, at least or about 60% reduction or inhibition, at least or about 70% reduction or inhibition, at least or about 80% reduction or inhibition, at least or about 90% reduction or inhibition, at least or about 95% reduction or inhibition, at least or about 98% reduction or inhibition).

In exemplary aspects, the CDK4/6 inhibitor comprises a structure:

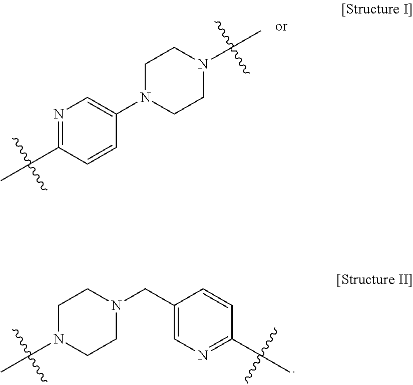 [SMILES: *c1ccc(N2CCN(*)CC2)cn1 ; *c1ccc(CN2CCN(*)CC2)cn1]

In various aspects, the CDK4/6 inhibitor comprises a structure of Structure I or Structure II and further comprises a structure of A-B, wherein A comprises a bicyclic structure and B comprises a monocyclic structure. In exemplary aspects, A-B comprises a structure of Structure III or Structure IV or Structure V:

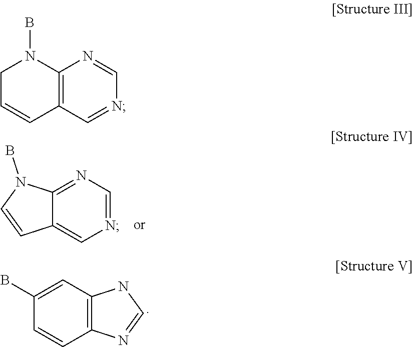

In exemplary aspects, B of Structure III or IV is a cyclopentane. In exemplary aspects, B of Structure V comprises a pyrimidine.

In various aspects, the CDK4/6 inhibitor comprises the structure of

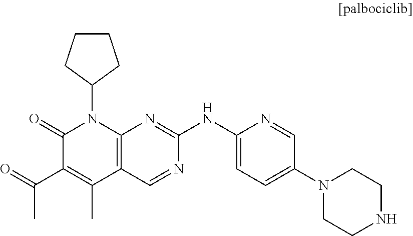 [SMILES: CC(=O)c1c(C)c2cnc(Nc3ccc(N4CCNCC4)cn3)nc2n(C2CCCC2)c1=O]

or a pharmaceutically acceptable salt thereof.

In various aspects, the CDK4/6 inhibitor comprises the structure of

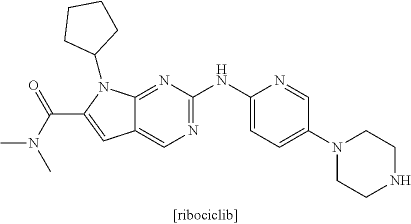 [SMILES: CN(C)C(=O)c1cc2cnc(Nc3ccc(N4CCNCC4)cn3)nc2n1C1CCCC1]

or a pharmaceutically acceptable salt thereof.

In various instances, the CDK4/6 inhibitor comprises the structure of

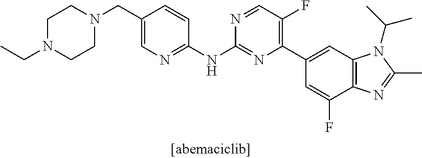 [SMILES: CCN1CCN(Cc2ccc(Nc3ncc(F)c(-c4cc(F)c5nc(C)n(C(C)C)c5c4)n3)nc2)CC1]

or a pharmaceutically acceptable salt thereof.

Methods of Treatment

Additionally provided herein are methods of treating a neoplastic disease in a subject.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a neoplastic disease of the present disclosure can provide any amount or any level of treatment. Furthermore, the treatment provided by the methods of the present disclosure can include treatment of one or more conditions or symptoms or signs of the neoplastic disease being treated. Also, the treatment provided by the methods of the present disclosure can encompass slowing the progression of the neoplastic disease. For example, the methods can treat neoplastic disease by virtue of enhancing the T cell activity or an immune response against the neoplastic disease, reducing tumor or cancer growth or tumor burden, reducing metastasis of tumor cells, increasing cell death of tumor or cancer cells or increasing tumor regression, and the like. In accordance with the foregoing, provided herein are methods of reducing tumor growth or tumor burden or increasing tumor regression in a subject. In exemplary embodiments, the method comprises administering to the subject a KIF18A inhibitor optionally in combination with a CDK4/6 inhibitor. In exemplary embodiments, the subject is or has been treated with a CDK4/6 inhibitor, and the method comprises administering to the subject a KIF18A inhibitor. The terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

In various aspects, the methods treat by way of delaying the onset or recurrence of the neoplastic disease by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject. In exemplary aspects, the methods of the present disclosure provide treatment by way of delaying the occurrence or onset of metastasis. In various instances, the methods provide treatment by way of delaying the occurrence or onset of a new metastasis. Accordingly, provided herein are methods of delaying the occurrence or onset of metastasis in a subject with cancer. In exemplary embodiments, the method comprises administering a KIF18A inhibitor to the subject optionally in combination with a CDK4/6 inhibitor.

In exemplary instances, the treatment provided may be described in terms of or supported by data obtained from a clinical trial wherein the endpoints of the trial are progression-free survival (PFS), overall survival (OS), or time to deterioration of Eastern Cooperative Oncology Group (ECOG) performance status. In various aspects, the present disclosure provides a method of increasing PFS, OS, or time to deterioration of ECOG performance status in a subject with a neoplastic disease. In exemplary embodiments, the neoplastic disease is resistant to or with a reduced sensitivity to a CDK4/6 inhibitor, and the method comprises administering to the subject a KIF18A inhibitor optionally in combination with a CDK4/6 inhibitor. As used herein, the term “progression-free survival” or “PFS” means the time a treated patient experiences without cancer getting worse (by whatever measure is being used to measure worsening). The term “overall survival” means how long the patient lives after treatment. ECOG performance status is a grade or score according to a scale used by doctors and researchers to assess a patient's disease, e.g., how the disease is progressing/regressing, how the disease affects the daily living abilities of the patient, and determine appropriate treatment and prognosis. ECOG performance status is determined according to the following criteria:

| SCORE | ECOG |
|---|---|
| 0 | Fully active, able to carry on all pre-disease |
| | performance without restriction |
| 1 | Restricted in physically strenuous activity |
| | but ambulatory and able to carry out work |
| | of a light or sedentary nature, e.g., light |
| | house work, office work |
| 2 | Ambulatory and capable of all selfcare but |
| | unable to carry out any work activities. |
| | Up and about more than 50% of waking hours |
| 3 | Capable of only limited selfcare, confined to |
| | bed or chair more than 50% of waking hours |
| 4 | Completely disabled. Cannot carry on any selfcare. |
| | Totally confined to bed or chair |
| 5 | Dead |

Oken et. al., Am. J. Clin. Oncol 5: 649-655 (1982)

In exemplary embodiments, the method of treating a subject for a neoplastic disease comprises administering a KIF18A inhibitor to the subject, wherein the subject comprises cells that are positive for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof, said method comprising administering a KIF18A inhibitor to the subject.

In exemplary embodiments, the method of treating a subject with a neoplastic disease comprises (A) assaying a sample obtained from the subject for (a) an inactivated TP53 gene and/or (b) at least one of: (i) an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof, and (B) administering a KIF18A inhibitor to a subject who is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof.

In exemplary embodiments, the neoplastic disease is resistant to treatment with a CDK4/6 inhibitor and the method of treating a subject with such a neoplastic disease comprises administering a KIF18A inhibitor to treat the patient.

In exemplary embodiments, the subject is or has been treated with a CDK4/6 inhibitor and the method treating such a subject comprises administering to the subject a KIF18A inhibitor, optionally, wherein the KIF18A inhibitor is co-administered with the CDK4/6 inhibitor.

In exemplary embodiments, the method of treating a neoplastic disease in a subject comprises administering to the subject a presently disclosed pharmaceutical combination comprising a KIF18A inhibitor. In exemplary instances, the pharmaceutical combination comprises a KIF18A inhibitor and a CDK4/6 inhibitor.

In various aspects, the cancer comprises cells that are positive for an inactivated TP53 gene and/or positive for at least one of an inactivated Rb1 gene, (ii) an amplified CCNE1 gene, gene copy number gain of the CCNE1 gene, or overexpression CCNE1 gene product, (iii) an inactivated BRCA gene or (iv) a combination thereof.

In exemplary aspects, the KIF18A inhibitor is administered to the subject daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various instances, the CDK inhibitor is administered once daily to the subject. Optionally, the KIF18A inhibitor is administered orally once a day.

Methods of inducing or increasing tumor regression in a subject with a tumor are additionally provided herein. In exemplary embodiments, the method comprises administering to the subject a KIF18A inhibitor in an amount effective to induce or increase tumor regression. The present disclosure also provides methods of reducing tumor growth or cancer growth in a subject. In exemplary embodiments, the method comprises administering to the subject a KIF18A inhibitor in an amount effective to reduce tumor or cancer growth. Methods of inducing or increasing death of tumor cells or cancer cells in a subject are provided herein. The method in exemplary embodiments comprises administering to the subject a KIF18A inhibitor in an amount effective to induce or increase death of the tumor cells or cancer cells. In various aspects, the neoplastic disease is a cancer, optionally, breast cancer, ovarian cancer, or prostate cancer. In various instances, the neoplastic disease is triple-negative breast cancer (TNBC), non-luminal breast cancer, or high-grade serous ovarian cancer (HGSOC). In exemplary aspects, the neoplastic disease is an endometrial cancer, optionally, serous endometrial cancer. Optionally, the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof. In some aspects, the cancer comprises cells that are positive for a mutant TP53 gene. In various instances, the cancer comprises cells that are positive for an amplified CCNE1 gene, a silenced BRCA1 gene, a deficient Rb1 gene, or a combination thereof. Optionally, the KIF18A inhibitor is administered for oral administration, optionally once a day. In exemplary aspects, the amount of the KIF18A inhibitor is effective induce at least 50% or at least 75% (e.g., at least 80% or 85% or at least 90% or 95%) tumor regression, compared to a control.

In exemplary embodiments, the methods of the present disclosure are advantageously highly specific to cells of the neoplastic disease. In various aspects, the KIF18A inhibitor effectively treats the neoplastic disease, induces or increases tumor regression, reduces tumor or cancer growth, or induces or increases death of a tumor or cancer cell, with little to no toxicity to normal somatic cells in the subject. In various aspects, the KIF18A inhibitor is administered in an amount effective to treat the neoplastic disease, maintain sensitivity to treatment with a CDK4/6 inhibitor, induce or increase tumor regression, reduce tumor or cancer growth, and/or induce or increase death of a tumor or cancer cell, without a substantial decrease in the proliferation of normal somatic cells in the subject. In exemplary instances, the KIF18A inhibitor is administered in an amount effective to treat the neoplastic disease, maintain sensitivity to treatment with a CDK4/6 inhibitor, induce or increase tumor regression, reduce tumor or cancer growth, or induce or increase death of a tumor or cancer cell, without a substantial increase in the apoptosis of normal somatic cells. As used herein, the term “normal” in reference to cells means cells that are not neoplastic and/or not diseased. In various aspects, the normal somatic cells are human bone marrow mononuclear cells. In various instances, the normal somatic cells are not genetically characterized as TP53^{MUT} or are genetically characterized as TP53^{WT}. In various aspects, the KIF18A inhibitor causes not more than a 25% increase in the apoptosis of normal somatic cells. In various aspects, the KIF18A inhibitor causes not more than a 25% decrease in the proliferation of normal somatic cells in the subject. Optionally, the increase in the apoptosis of normal somatic cells or the decrease in the proliferation of normal somatic cells is less than about 20%, less than about 15%, less than about 10%, less than about 9%, less than about 8%, less than about 7%, less than about 6%, less than about 5%, less than about 4%, less than about 3%, less than about 2%, or less than about 1%. Methods of measuring the proliferation of normal somatic cells and/or apoptosis of normal somatic cells are described herein.

Neoplastic Disease

As used herein, the term “neoplastic disease” refers to any condition that causes growth of a tumor. In exemplary aspects, the tumor is a benign tumor. In exemplary aspects, the tumor is a malignant tumor. In various aspects, the neoplastic disease is cancer. The cancer in various aspects is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, or urinary bladder cancer. In particular aspects, the cancer is head and neck cancer, ovarian cancer, cervical cancer, bladder cancer, oesophageal cancer, pancreatic cancer, gastrointestinal cancer, gastric cancer, breast cancer, endometrial cancer, colorectal cancer, hepatocellular carcinoma, glioblastoma, bladder cancer, lung cancer, e.g., non-small cell lung cancer (NSCLC), or bronchioloalveolar carcinoma. In particular embodiments, the tumor is non-small cell lung cancer (NSCLC), head and neck cancer, renal cancer, triple negative breast cancer, or gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject.

The terms “cancer” and “cancerous” when used herein refer to or describe the physiological condition in mammals that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer, ovarian cancer, and endometrial cancer. While the term “cancer” as used herein is not limited to any one specific form of the disease, it is believed that the methods of the invention will be particularly effective for cancers which are found to be accompanied by unregulated levels of KIF18A or dependent on KIF18A for proper chromosome segregation and survival in the mammal.

In various aspects, the cancer is metastatic, the tumor is unresectable, or a combination thereof. In various aspects, the neoplastic disease is positive for an inactivated TP53 gene and/or positive for at least one of an inactivated Rb1 gene, (ii) an amplified CCNE1 gene, gene copy number gain of the CCNE1 gene, or overexpression of a CCNE1 gene product, (iii) an inactivated BRCA gene or (iv) a combination thereof. In various aspects, the neoplastic disease is triple negative breast cancer (TNBC), nonluminal breast cancer (e.g., basal like mesenchymal), or high grade serous ovarian cancer (HGSOC). In various aspects, the neoplastic disease is resistant or not sensitive (insensitive) to treatment with a CDK4/6 inhibitor. In various aspects, the neoplastic disease is resistant or not sensitive (insensitive) to treatment with a CDK4/6 inhibitor and is Rb1 proficient (vs. Rb1 deficient). In various aspects, the neoplastic disease is resistant to treatment with a KIF18A inhibitor. In various aspects, the neoplastic disease is resistant to treatment with a KIF18A inhibitor and Rb1 deficient (vs. Rb1 proficient).

In exemplary aspects, the neoplastic disease is a breast cancer, optionally, luminal breast cancer or TNBC. In various aspects, the breast cancer has been (a) histologically or cytologically confirmed metastatic or locally recurrent estrogen receptor (ER)-negative (e.g., <1% by immunohistochemistry [IHC]), (b) progesterone receptor (PR)-negative (e.g., <1% IHC) and (c) human epidermal growth factor receptor 2 (Her2)-negative (either fluorescent in situ hybridisation [FISH] negative, 0 or 1+ by IHC, or IHC2+ and FISH negative perASCO/CAP definition). In exemplary aspects, the neoplastic disease is relapsed and/or refractory to at least one line of systemic chemotherapy in the metastatic setting or intolerant of existing therapy(ies) known to provide clinical benefit for the neoplastic disease. In exemplary instances, the cancer has been treated with an immune checkpoint inhibitor. In various instances, the breast cancer is hormone receptor (HR)-positive and/or HER2-negative. In various aspects, the breast cancer is advanced breast cancer and/or metastatic breast cancer. In various aspects, the breast cancer is HR+/HER2− advanced or metastatic breast cancer that has progressed after endocrine therapy. In some aspects, the breast cancer is a hormone receptor-positive (HR+)/HER2-negative (HER2−) advanced or metastatic breast cancer previously treated with endocrine therapy and chemotherapy after the cancer has spread/metastasized. In various instances, the cancer is an HR+/HER2− advanced or metastatic breast cancer that has not been treated with hormonal therapy (Arimidex (chemical name: anastrozole), Aromasin (chemical name: exemestane), and Femara (chemical name: letrozole). In various instances, the breast cancer is HR+/HER2− advanced or metastatic breast cancer that has grown after being treated with hormonal therapy. In various instances, the breast cancer is a HER2-positive breast cancer, including but not limited to those that are similar to the HER2-positive breast cancer cells of Table 2. Optionally, the breast cancer is a HER2-positive, estrogen receptor (ER)-negative breast cancer. In various aspects, the neoplastic disease is ovarian cancer, optionally, high grade serous ovarian cancer (HGSOC). Optionally, the ovarian cancer is platinum-resistant HGSOC. In exemplary aspects, the ovarian cancer is primary peritoneal cancer or fallopian-tube cancer. In various aspects, the neoplastic disease is metastatic or unresectable HGSOC, with platinum-resistance defined as progression during or within 6 months of a platinum-containing regimen. In various aspects, the ovarian cancer has been or is being treated with platinum-resistant recurrence therapy. In various aspects, the neoplastic disease is serous endometrial cancer. Optionally, the neoplastic disease is metastatic or recurrent serous endometrial cancer. In various instances, the endometrial cancer is relapsed and/or refractory to at least one line of systemic therapy in the metastatic/recurrent setting or intolerant of existing therapy(ies) known to provide clinical benefit for the neoplastic disease. In various instances, the neoplastic disease is an advanced or metastatic solid tumor that is unresectable and relapsed and/or refractory to at least one line of systemic chemotherapy or intolerant. Optionally, the advanced or metastatic solid tumor is TP53^{MUT}.

In various instances, the neoplastic disease is resistant to treatment with one or more drugs. In various aspects, the neoplastic disease exhibits reduced sensitivity to treatment with one or more drugs. Optionally, the neoplastic disease is a multidrug resistant neoplastic disease. In exemplary instances, the tumor or cancer cells (e.g., of the neoplastic disease) are multidrug resistant tumor or cancer cells and/or exhibit increased expression of the Multidrug resistance 1 (MDR-1) gene and/or a gene product thereof. In exemplary instances, the tumor or cancer cells (e.g., of the neoplastic disease) exhibit increased expression of a P-glycoprotein (P-gp) encoded by MDR-1 gene. In various aspects, the neoplastic disease exhibits reduced sensitivity or resistance to treatment with an anti-mitotic agent or anthracycline antibiotic, optionally, paclitaxel or doxorubicin. In various aspects, the tumor or cancer cells (e.g., of the neoplastic disease) exhibit mutations in a tubulin gene, overexpression of tubulin, tubulin amplification, and/or isotype switched tubulin expression. In various aspects, the mutations in α- or β-tubulin inhibit the binding of taxanes to the correct place on the microtubules, thereby rendering the taxane ineffective. In exemplary aspects, the neoplastic disease exhibits reduced sensitivity or resistance to treatment with any one or more of a platinum agent an anthracycline, a targeted therapy (e.g. TKI, PARP inhibitors).

In various aspects, the neoplastic disease is a cancer comprising one or more whole genome duplication or whole genome doubling (WGD) events. WGD in the context of cancer is discussed in Lens and Herndema, Nature Reviews Cancer 19: 32-45 (2019); Ganem et. al., Current Opinion in Genetics & Development 17, 157-162, and Davoli et. al., Annual Review of Cell and Developmental Biology 27, 585-610.

Subjects

In exemplary embodiments of the present disclosure, the subject is a mammal, including, but not limited to, mammals of the order Rodentia, such as mice and hamsters, and mammals of the order Logomorpha, such as rabbits, mammals from the order Camivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In various aspects, the subject has a neoplastic disease, e.g., any one of those described herein. The term “patient”, “subject”, or “mammal” as used herein refers to any “patient”, “subject”, or “mammal” including humans, cows, horses, dogs and cats. In one embodiment of the invention, the mammal is a human.

In exemplary aspects, the subject has cancer with a metastasis, an unresectable tumor, or a combination thereof. In various aspects, the cancer or tumor exhibits or has exhibited a resistance or reduced sensitivity to treatment with a CDK4/6 inhibitor. In exemplary aspects, the subject has breast cancer, optionally, luminal breast cancer or triple negative breast cancer (TNBC). In various aspects, the breast cancer has been (a) histologically or cytologically confirmed metastatic or locally recurrent estrogen receptor (ER)-negative (e.g., <1% by immunohistochemistry [IHC]), (b) progesterone receptor (PR)-negative (e.g., <1% IHC) and (c) human epidermal growth factor receptor 2 (Her2)-negative (either fluorescent in situ hybridisation [FISH] negative, 0 or 1+ by IHC, or IHC2+ and FISH negative per ASCO/CAP definition). In exemplary aspects, the subject is relapsed and/or refractory to at least one line of systemic chemotherapy in the metastatic setting or intolerant of existing therapy(ies) known to provide clinical benefit for their condition. In exemplary instances, the subject has prior exposure to an immune checkpoint inhibitor. In various instances, the breast cancer hormone receptor (HR)-positive and/or HER2-negative. In various aspects, the breast cancer is advanced breast cancer and/or metastatic breast cancer. In various aspects, the subject has HR+/HER2− advanced or metastatic breast cancer that has progressed after taking endocrine therapy. In some aspects, the subject is a hormone receptor-positive (HR+)/HER2-negative (HER2−) advanced or metastatic breast cancer patient previously treated with endocrine therapy and chemotherapy after cancer has spread/metastasized. In various instances, the subject has HR+/HER2− advanced or metastatic breast cancer that has not been treated with hormonal therapy before in postmenopausal women (Arimidex (chemical name: anastrozole), Aromasin (chemical name: exemestane), and Femara (chemical name: letrozole). In various instances, the subject is a postmenopausal woman with HR+/HER2− advanced or metastatic breast cancer that has grown after being treated with hormonal therapy. In certain aspects, the subject is a pre/perimenopausal or postmenopausal woman with HR+, human epidermal growth factor receptor 2 (HER2)-negative advanced or metastatic breast cancer, and has received endocrine-based therapy. Optionally, the subject is a postmenopausal woman with HR+, HER2− advanced or metastatic breast cancer, and has received initial endocrine-based therapy or has disease progression upon treatment with the endocrine therapy. In various aspects, the subject has ovarian cancer, optionally, high grade serous ovarian cancer (HGSOC). Optionally, the ovarian cancer is platinum-resistant HGSOC. In exemplary aspects, the subject has primary peritoneal cancer and/or fallopian-tube cancer. In various aspects, the subject has a histologically or cytologically confirmed diagnosis of metastatic or unresectable HGSOC, with platinum-resistance defined as progression during or within 6 months of a platinum-containing regimen. In various aspects, the subject has ovarian cancer and has received or is receiving platinum-resistant recurrence therapy. In various aspects, the subject has serous endometrial cancer. Optionally, the subject has a histologically or cytologically confirmed diagnosis of metastatic or recurrent serous endometrial cancer. In various instances, the subject is relapsed and/or refractory to at least one line of systemic therapy in the metastatic/recurrent setting or intolerant of existing therapy(ies) known to provide clinical benefit for their condition. In various instances, the subject has an advanced or metastatic solid tumor that is unresectable and relapsed and/or refractory to at least one line of systemic chemotherapy or intolerant. Optionally, the advanced or metastatic solid tumor is TP53^{MUT}.

In exemplary aspects, the subject does not have any of the following: (a) active brain metastases, (b) primary central nervous system (CNS) tumor, hematological malignancies or lymphoma, (c) uncontrolled pleural effusions(s), pericardial effusion, or ascites, (d) gastrointestinal (GI) tract disease causing the inability to take oral medication.

Samples

With regard to the methods disclosed herein, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, bone marrow aspirate, peritoneal cavity fluid (e.g., malignant ascites), or urine obtained from the subject. In exemplary aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In various aspects, the sample comprises cancer cells, tumor cells, non-tumor cells, blood, blood cells, or plasma. In exemplary instances, the sample comprises cell-free DNA (cfDNA). In exemplary instances, the sample comprises germline cells of the neoplastic disease (e.g., cancer). In exemplary instances, the sample comprises somatic cells of the neoplastic disease (e.g., cancer).

Controls

In the methods described herein, the level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, BMI, current therapeutic regimen status, medical history, or a combination thereof, but differs from the subject being diagnosed in that the control does not suffer from the disease in question or is not at risk for the disease.

Relative to a control level, the level that is determined may an increased level. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

### Exemplary Embodiments

Exemplary embodiments of the present invention include but are not limited to the following:

E1. A method of determining a treatment for a subject having a neoplastic disease, said method comprising, consisting essentially, or consisting of assaying a sample obtained from the subject for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, wherein the treatment determined for the subject comprises, consists essentially of, or consists of a KIF18A inhibitor, when the sample is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof.
E2. A method of treating a subject having a neoplastic disease, said method comprising, consisting essentially of, or consisting of (A) assaying a sample obtained from the subject for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, and (B) administering a KIF18A inhibitor to a subject that is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof, optionally, wherein the method further comprises obtaining the sample from the subject.
E3. A method of treating a subject having a neoplastic disease, wherein the subject comprises cells that are positive for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, said method comprising, consisting essentially of, or consisting of administering a KIF18A inhibitor to the subject.
E4. A method of identifying a subject having a neoplastic disease as sensitive to treatment with a KIF18A inhibitor, said method comprising, consisting essentially of, or consisting of assaying a sample obtained from the subject for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, wherein the subject is identified as sensitive to treatment with a KIF18A inhibitor, when the sample is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof.
E5. The method of any one of the preceding embodiments, wherein the inactivated TP53 gene (i) comprises a TP53 gene mutation, deletion, truncation, and/or an epigenetically silenced TP53 promoter, (ii) is inactivated by a viral protein or via amplification of an MDM2 gene, or (iii) a combination thereof.
E6. The method of embodiment E5, wherein the viral protein is a Human Papillomavirus (HPV) E6 protein.
E7. The method of any one of the preceding embodiments, wherein the inactivated Rb1 gene (i) comprises an Rb1 gene mutation, deletion, truncation, and/or an epigenetically silenced Rb1 promoter, (ii) is inactivated by a viral protein or (iii) a combination thereof.
E8. The method of embodiment E7, wherein the viral protein is a Human Papillomavirus (HPV) E7 protein.
E9. The method of any one of the preceding embodiments, wherein the overexpression of the CCNE1 gene product is caused by a mutation in an FBXw7 gene.
E10. The method of any one of the preceding embodiments, wherein the inactivated BRCA gene (i) comprises a BRCA gene mutation, deletion, truncation, and/or an epigenetically silenced BRCA promoter.
E11. The method of embodiment E10, wherein the BRCA gene is a BRCA1 gene.
E12. The method of embodiment E10, wherein the BRCA gene is a BRCA2 gene.
E13. The method of any one of the preceding embodiments, further comprising determining the sensitivity to a CDK4/6 inhibitor of cells of the sample, optionally, assaying for the sensitivity to a CDK4/6 inhibitor.
E14. The method of any one of the preceding embodiments, wherein the assaying step comprises a cytogenetics method and/or molecular method for detecting the presence of an inactivated TP53 gene, inactivated Rb1 gene, amplified CCNE1 gene or overexpression of a CCNE1 gene product, or inactivated BRCA gene.
E15. The method of any one of the preceding embodiments, wherein the assaying step comprises direct sequencing, DNA hybridization and/or restriction enzyme digestion.
E16. The method of embodiment E14, wherein the cytogenetics method comprises karyotyping, fluorescence in situ hybridization (FISH), comparative genomic hybridization (CGH), or a combination thereof.
E17. The method of embodiment E14, wherein the molecular method comprises restriction fragment length polymorphism (RFLP), amplification refractory mutation system (ARMS), polymerase chain reaction (PCR), multiplex ligation dependent probe amplification (MLPA), denaturing gradient gel electrophoresis (DGGE), single strand conformational polymorphism (SSCP), heteroduplex analysis, chemical cleavage of mismatch (CCM), protein truncation test (PTT), oligonucleotide ligation assay (OLA), or a combination thereof.
E18. The method of embodiment E17, wherein the PCR is a multiplex PCR, nested PCR, RT-PCR, or real time PCR.
E19. The method of any one of the preceding embodiments, wherein the assaying step comprises assaying expression levels of RNA or protein encoded by the TP53 gene, Rb1 gene, CCNE1 gene, and/or the BRCA gene.
E20. A method of determining a treatment for a subject having a neoplastic disease, said method comprising, consisting essentially of, or consisting of determining sensitivity of the neoplastic disease to treatment with a CDK4/6 inhibitor, wherein the treatment for the subject is determined as a treatment comprising a KIF18A inhibitor, when the neoplastic disease is insensitive to the CDK4/6 inhibitor.
E21. A method of determining a treatment for a subject having a neoplastic disease, said method comprising, consisting essentially of, or consisting of determining sensitivity of the neoplastic disease to treatment with a KIF18A inhibitor, wherein the treatment for the subject is determined as a treatment comprising, consisting essentially of, or consisting of a CDK4/6 inhibitor, when the neoplastic disease is insensitive to the KIF18A inhibitor.
E22. A method of treating a subject having a neoplastic disease resistant to treatment with a CDK4/6 inhibitor, said method comprising, consisting essentially of, or consisting of administering a KIF18A inhibitor to treat the patient.
E23. A method of treating a neoplastic disease in a subject who is or has been treated with a CDK4/6 inhibitor, said method comprising, consisting essentially of, or consisting of administering to the subject a KIF18A inhibitor, optionally, wherein the KIF18A inhibitor is co-administered with the CDK4/6 inhibitor.
E24. A method of maintaining sensitivity of a neoplastic disease to treatment with a CDK4/6 inhibitor in a subject, said method comprising, consisting essentially of, or consisting of administering to the subject a KIF18A inhibitor.
E25. A method of identifying a subject having a cancer as responsive to treatment with a KIF18A inhibitor, said method comprising, consisting essentially of, or consisting of determining the sensitivity of the neoplastic disease to treatment with a KIF18A inhibitor, wherein the subject is identified as responsive to treatment with a KIF18A inhibitor, when the cancer cells of the sample are insensitive to the CDK4/6 inhibitor.
E26. The method of any one of embodiments E20-E25 wherein sensitivity to a CDK4/6 inhibitor is determined by assaying a sample obtained from the subject for the absence of (i) an inactivated Rb1 gene, (ii) an amplified CCNE1 gene or overexpression of a CCNE1 gene product, or (iii) a combination thereof.
E27. The method of any one of the preceding embodiments, wherein the sample comprises cancer cells, tumor cells, non-tumor cells, blood, blood cells, or plasma, optionally, wherein the sample comprises germline cancer cells or somatic cancer cells.
E28. The method of embodiment E27, wherein the sample comprises cell-free DNA (cfDNA).
E29. A pharmaceutical combination comprising, consisting essentially of, or consisting of a CDK4/6 inhibitor and a KIF18A inhibitor.
E30. The method or pharmaceutical combination of any one of embodiments E20-E29 wherein the CDK4/6 inhibitor is palbociclib, ribociclib, and/or abemaciclib.
E31. The method of any one of embodiments E23, E27, E28, and E30, wherein the KIF18A inhibitor and the CDK4/6 inhibitor are separately administered to the subject.
E32. The method of any one of embodiments E23, E27, E28, E30, and E31, wherein the KIF18A inhibitor is formulated and/or packaged separately from the CDK4/6 inhibitor.
E33. The method of any one of the preceding embodiments, wherein the neoplastic disease is a cancer, optionally, breast cancer.
E34. The method of embodiment E33, wherein the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof.
E35. The method of any one of the preceding embodiments, wherein the neoplastic disease is triple-negative breast cancer (TNBC), non-luminal breast cancer, or high-grade serous ovarian cancer (HGSOC).
E36. The method or pharmaceutical combination of any one of the preceding embodiments, wherein the KIF18A inhibitor is administered for oral administration, optionally once a day.
E37. A method of treating a subject having a neoplastic disease, said method comprising, consisting essentially of, or consisting of administering to the subject a KIF18A inhibitor in an amount effective to treat the neoplastic disease.
E38. A method of inducing or increasing tumor regression in a subject with a tumor, said method comprising, consisting essentially of, or consisting of administering to the subject a KIF18A inhibitor in an amount effective to inducing or increasing tumor regression.
E39. A method of reducing tumor or cancer growth in a subject with a tumor, said method comprising, consisting essentially of, or consisting of administering to the subject a KIF18A inhibitor in an amount effective to reducing tumor or cancer growth.
E40. A method of inducing or increasing death of tumor or cancer cells in a subject, said method comprising, consisting essentially of, or consisting of administering to the subject a KIF18A inhibitor in an amount effective to inducing or increasing death of tumor or cancer cells.
E41. The method of any one of the preceding embodiments, wherein the neoplastic disease is a cancer, optionally, breast cancer, ovarian cancer, endometrial cancer, lung cancer, or prostate cancer.
E42. The method of embodiment E41, wherein the neoplastic disease is triple-negative breast cancer (TNBC), non-luminal breast cancer, or high-grade serous ovarian cancer (HGSOC).
E43. The method of embodiment E42, wherein the neoplastic disease is TNBC.
E44. The method of embodiment E42, wherein the neoplastic disease is non-luminal breast cancer.
E45. The method of embodiment E42, wherein the neoplastic disease is HGSOC.
E46. The method of embodiment E41, wherein the neoplastic disease is an endometrial cancer, optionally, serous endometrial cancer.
E47. The method of any one of embodiments E41 to E46, wherein the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof.
E48. The method of embodiment E47, wherein the cancer comprises cells that are positive for a mutant TP53 gene.
E49. The method of embodiment E47 or E48, wherein the cancer comprises cells that are positive for an amplified CCNE1 gene, a silenced BRCA1 gene, a deficient Rb1 gene, or a combination thereof.
E50. The method of any one of the preceding embodiments, wherein the KIF18A inhibitor is administered for oral administration, optionally once a day.
E51. The method of any one of the preceding embodiments, wherein the amount of the KIF18A inhibitor is effective induce at least 50% tumor regression, compared to a control.
E52. The method of any one of the preceding embodiments, wherein the amount of the KIF18A inhibitor is effective induce at least 75% tumor regression, compared to a control.
E53. The method of any one of the preceding embodiments, wherein the amount of the KIF18A inhibitor is effective induce at least 80% or 85% tumor regression, compared to a control.
E54. The method of any one of the preceding embodiments, wherein the amount of the KIF18A inhibitor is effective induce at least 90% or 95% tumor regression, compared to a control.
E55. The method of any one of the preceding embodiments, wherein the KIF18A inhibitor is a compound of formula (I):

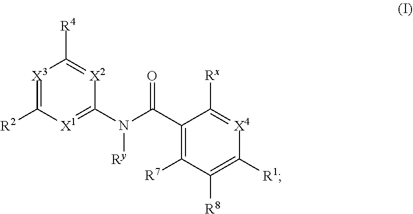

or any pharmaceutically-acceptable salt thereof, wherein:

X¹ is N or —CR⁶;

X² is N or —CR⁵;

X³ is N or —CR³;

X⁴ is N or —CR⁹;

wherein 0, 1, or 2 of X¹, X², X³ and X⁴ is N;

R¹ is —CN, or a group —Z—R¹² wherein Z is —C₀₋₄alk-, —NR¹¹—, —NR¹¹SO₂, —SO₂NR¹¹—, —NR¹¹—S(═O)(═NH), —S(═O)(═NH)—, —S—, —S(═O)—, —SO₂—, C₀₋₄alk-O—, —(C═O)—, —(C═O)NR¹¹—, —C═N(OH)—, or —NR¹¹(C═O); or

the group —Z—R¹² is —N═S(═O)—(R¹²)₂, wherein the two R¹² pair can alternatively combine with the sulfur atom attached to each of them to form a saturated or partially-saturated 3-, 4-, 5-, or 6-membered monocyclic ring containing 0, 1, 2 or 3 N atoms and 0, 1, or 2 atoms selected from O and S:

R² is halo or a group —Y—R¹³, wherein Y is —C₀₋₄alk-, —N(C₀₋₁alk)-C₀₋₄alk-, —C(═O)NR^{a}R^{a}(C₁₋₄alk), —O—C₀₋₄alk-, S, S═O, S(═O)₂, —SO₂NR¹³, or —S(═O)(═NH)—;

R³ is H, halo, C₁₋₈alk, or C₁₋₄haloalk;

R⁴ is H, halo, R^{4a} or R^{4b};

R⁵ is H, halo, C₁₋₈alk, or C₁₋₄haloalk;

R⁶ is H, halo, C₁₋₈alk, C₁₋₄haloalk, —O—C₁₋₈alk, or —O—R^{6a}; wherein R^{6a} is a saturated or partially-saturated 3-, 4-, 5-, or 6-membered monocyclic ring containing 0, 1, 2 or 3 N atoms and 0, 1, or 2 atoms selected from O and S;

R⁷ is H, halo, C₁₋₆alk, or C₁₋₄haloalk;

R⁸ is H, halo, C₁₋₈alk, C₁₋₄haloalk, —OH, —O—Rh, or —O—R^{8b};

R⁹ is H, halo, C₁₋₈alk, or C₁₋₄haloalk;

R^{x} is selected from the group consisting of

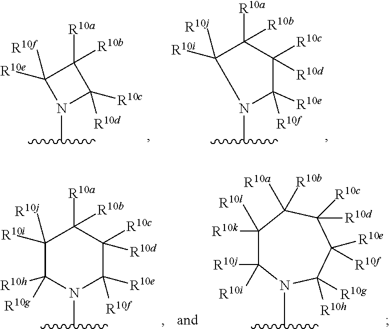

Each of R^{10a}, R^{10b}, R^{10c}, R^{10d}, R^{10e}, R^{10f}, R^{10g}, R^{10h}, R¹⁰ⁱ, and R^{10j} is H, halo, R^{10k}, or R^{10l};

or alternatively, each of R^{10a} and R^{10b} pair, R^{10c} and R^{10d} pair, R^{10e} and R^{10f} pair, R^{10g} and R^{10h} pair, or R¹⁰ⁱ and R^{10j} pair, independently, can combine with the carbon atom attached to each of them to form a saturated or partially-saturated 3-, 4-, 5-, 6-membered monocyclic ring spiro to the R^{x} ring; wherein said 3-, 4-, 5-, 6-membered monocyclic ring contains 0, 1, 2 or 3 N atoms and 0, 1, or 2 atoms selected from O and S, and further wherein said 3-, 4-, 5-, 6-membered monocyclic ring is substituted by 0, 1, 2 or 3 group(s) selected from F, Cl, Br, C₁₋₆alk, C₁₋₄haloalk, —OR⁸, —OC₁₋₄haloalk, CN, —NR^{a}R^{a}, or oxo;

R^{y} is H, C₁₋₄alk, or C₁₋₄haloalk;

R¹¹ is H, R^{11a}, or R^{11b};

R¹² is H, R^{12a}, or R^{12b};

R¹³ is R^{13a} or R^{13b};

R^{4a}, R^{8a}, R^{10k}, R^{11a}, R^{12a}, and R^{13a} is independently, at each instance, selected from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0, 1, or 2 atoms selected from O and S, which is substituted by 0, 1, 2 or 3 group(s) selected from F, Cl, Br, C₁₋₆alk, C₁₋₄haloalk, —OR^{a}, —OC₁₋₄haloalk, CN, —C(═O)R^{b}, —C(═O)OR^{a}, —C(═O)NR^{a}R^{a}, —C(═NR⁸)NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁₋₆alkNR^{a}R^{a}, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₈alkC(═O)OR^{a}, R¹⁴, and oxo;

R^{4b}, R^{8b}, R^{10l}, R^{11b}, R^{12b}, and R^{13b} is independently, at each instance, selected from the group consisting of C₁₋₆alk substituted by 0, 1, 2, 3, 4, or 5 group(s) selected from F, Cl, Br, —OR^{a}, —OC₁₋₄haloalk, or CN;

R¹⁴ is independently, at each instance, selected from the group consisting of a saturated, partially-saturated or unsaturated 3-, 4-, 5-, 6-, or 7-membered monocyclic or 4-, 5-, 6-, 7-, 8-, 9-, 10-, 11-, or 12-membered bicyclic ring containing 0, 1, 2 or 3 N atoms and 0 or 1 atoms selected from O and S, which is substituted by 0, 1, 2 or 3 group(s) selected from F, Cl, Br, C₁₋₆alk, C₁₋₄haloalk, —OR^{a}, —OC₁₋₄haloalk, CN, —C(═O)R^{b}, —C(═O)OR^{a}, —C(═O)NR^{a}R^{a}, —C(═NR^{a})NR^{a}R^{a}, —OC(═O)R^{b}, —OC(═O)NR^{a}R^{a}, —OC₂₋₆alkNR^{a}R^{a}, —OC₂₋₆alkOR^{a}, —SR^{a}, —S(═O)R^{b}, —S(═O)₂R^{b}, —S(═O)₂NR^{a}R^{a}, —NR^{a}R^{a}, —N(R^{a})C(═O)R^{b}, —N(R^{a})C(═O)OR^{b}, —N(R^{a})C(═O)NR^{a}R^{a}, —N(R^{a})C(═NR^{a})NR^{a}R^{a}, —N(R^{a})S(═O)₂R^{b}, —N(R^{a})S(═O)₂NR^{a}R^{a}, —NR^{a}C₂₋₆alkNR^{a}R^{a}, —NR^{a}C₂₋₆alkOR^{a}, —C₁₋₆alkNR^{a}R^{a}, —C₁₋₆alkOR^{a}, —C₁₋₆alkN(R^{a})C(═O)R^{b}, —C₁₋₆alkOC(═O)R^{b}, —C₁₋₆alkC(═O)NR^{a}R^{a}, —C₁₋₆alkC(═O)OR^{a}, and oxo;

R^{a} is independently, at each instance, H or R^{b}; and

R^{b} is independently, at each instance, C₁₋₆alk, phenyl, or benzyl, wherein the C₁₋₆alk is being substituted by 0, 1, 2 or 3 substituents selected from halo, —OH, —OC₁₋₄alk, —NH₂, —NHC₁₋₄alk, —OC(═O)C₁₋₄alk, or —N(C₁₋₄alk)C₁₋₄alk; and the phenyl or benzyl is being substituted by 0, 1, 2 or 3 substituents selected from halo, C₁₋₄alk, C₁₋₃haloalk, —OH, —OC₁₋₄alk, —NH₂, —NHC₁₋₄alk, —OC(═O)C₁₋₄alk, or —N(C₁₋₄alk)C₁₋₄alk.

E56. The method of any one of the preceding embodiments, wherein the KIF18A inhibitor is a compound of formula (I), or the pharmaceutically-acceptable salt thereof, wherein 0 of X¹, X², X³ and X⁴ is N.
E57. The method of any one of the preceding embodiments, wherein the KIF18A inhibitor is a compound of formula (I), or the pharmaceutically-acceptable salt thereof, wherein 1 of X¹, X², X³ and X⁴ is N.
E58. The method of any one of the preceding embodiments, wherein the KIF18A inhibitor is a compound of formula (I), or the pharmaceutically-acceptable salt thereof, wherein 2 of X¹, X², X³ and X⁴ is N.
E59. The method of any one of the preceding embodiments, wherein the KIF18A inhibitor is a compound of formula (I), or the pharmaceutically-acceptable salt thereof, wherein each of X¹ and X³ is N; X² is —CR⁵; and X⁴ is —CR⁹; having the formula (Ia):

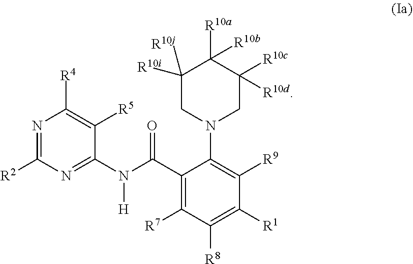

E60. The method of any one of the preceding embodiments, wherein the KIF18A inhibitor is a compound of formula (I), or the pharmaceutically-acceptable salt thereof, wherein X¹ is —CR⁶; X² is —CR⁵; X³ is N; and X⁴ is —CR⁹; having the formula (Ib):

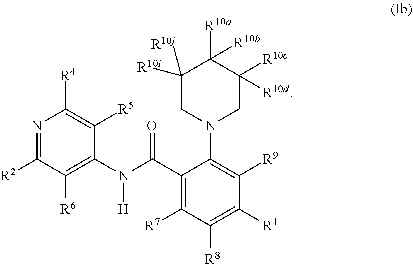

E61. The method of any one of the preceding embodiments, wherein the KIF18A inhibitor is a compound of formula (I), or the pharmaceutically-acceptable salt thereof, wherein X¹ is N; X² is —CR⁵; X³ is —CR³; and X⁴ is —CR⁹; having the formula (Ic):

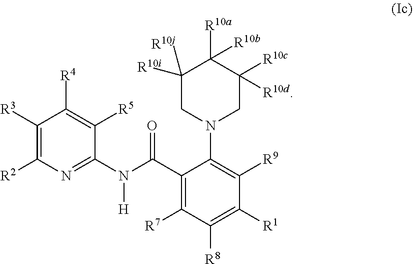

E62. The method of any one of the preceding embodiments, wherein the KIF18A inhibitor is a compound of formula (I), or the pharmaceutically-acceptable salt thereof, wherein X¹ is —CR⁶; X² is —CR⁵; X³ is —CR³; and X⁴ is —CR⁹; having the formula (Id):

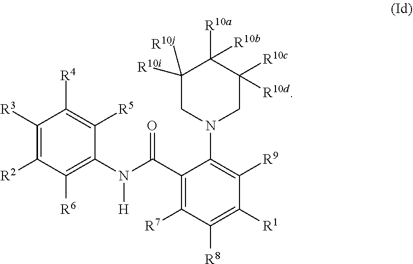

E63. The method of any one of the preceding embodiments, wherein the KIF18A inhibitor is a compound of formula (I), or the pharmaceutically-acceptable salt thereof, wherein X¹ is —CR⁶; X² is —CR⁵; X³ is —CR³; and X⁴ is —N; having the formula (Ie):

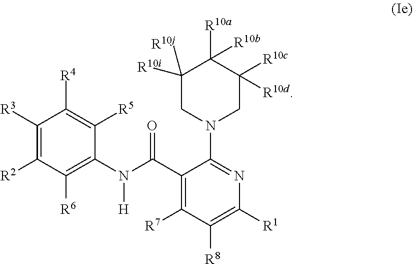

E64. The method of any one of the preceding embodiments, wherein the KIF18A inhibitor is a compound of formula (I), or the pharmaceutically-acceptable salt thereof, wherein R^{y} is H or methyl.
E65. The method of any one of the preceding embodiments, wherein the KIF18A inhibitor is a compound of formula (I), or the pharmaceutically-acceptable salt thereof, wherein each of R^{10c}, R^{10d}, R^{10e}, R^{10f}, R^{10g}, R^{10h}, R¹⁰ⁱ, and R^{10j} is H, halo, C₁₋₆alk, or C₁₋₄haloalk; and each of R^{10a} and R^{10b} pair combine with the carbon atom attached to each of them form a saturated 3-, 4-, or 5-membered monocyclic ring spiro to the R^{x} ring; wherein said ring contains 0, 1, 2 or 3 N atoms and 0, 1, or 2 atoms selected from O and S.
E66. The method of any one of the preceding embodiments, wherein the KIF18A inhibitor is a compound of formula (I), or the pharmaceutically-acceptable salt thereof, wherein each of R^{10c}, R^{10d}, R^{10e}, R^{10f}, R^{10g}, R^{10h}, R¹⁰ⁱ, and R^{10j} is H, methyl, or ethyl; and each of R^{10a} and R^{10b} pair combine with the carbon atom attached to each of them form a cyclopropyl, cyclobutyl, or cyclopentyl ring spiro to the R^{x} ring.
E67. The method of any one of the preceding embodiments, wherein the KIF18A inhibitor is a compound of formula (I), or the pharmaceutically-acceptable salt thereof, wherein the group

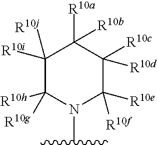

is

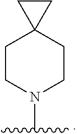 [SMILES: *N1CCC2(CC1)CC2]

E68. The method of any one of the preceding embodiments, wherein the KIF18A inhibitor is a compound of formula (I), or the pharmaceutically-acceptable salt thereof, wherein R¹ is a group —Z—R¹²; wherein Z is —S(═O)(═NH)—, —NHSO₂—, —SO₂—, —SO₂NH—, or —NH—; and R¹² is cyclopropyl, —CH₂CH₂—OH, —CH(CH₃)CH₂—OH, —C(CH₃)₂CH₂—OH, methyloxetanyl, or tert-butyl.
E69. The method of any one of the preceding embodiments, wherein the KIF18A inhibitor is a compound of formula (I), or the pharmaceutically-acceptable salt thereof, wherein R¹ is a group —Z—R¹²; wherein Z is —NHSO₂— or —NH—; and R¹² is —CH₂CH₂—OH, —CH(CH₃)CH₂—OH, or —C(CH₃)₂CH₂—OH.
E70. The method of any one of the preceding embodiments, wherein the KIF18A inhibitor is a compound of formula (I), or the pharmaceutically-acceptable salt thereof, wherein R² is a group —Z—R¹²; wherein Z is —NHSO₂— and R¹² is —CH₂CH₂—OH.
E71. The method of any one of the preceding embodiments, wherein the KIF18A inhibitor is a compound of formula (I), or the pharmaceutically-acceptable salt thereof, wherein R² is a group —Y—R¹³; wherein Y is —C₀₋₄alk-, —O—C₀₋₄alk-, S, S═O, S(═O)₂, or —SO₂NH—; and —R¹³ is 4,4-difluoro-1-piperidinyl; —CH₂CH₂—CF₃, tert-butyl, cyclopentyl, or 2-methylmorpholinyl.
E72. The method of any one of the preceding embodiments, wherein the KIF18A inhibitor is a compound of formula (I), or the pharmaceutically-acceptable salt thereof, wherein R² is piperidinyl or morpholinyl substituted by 1, 2 or 3 group(s) selected from F, Cl, Br, methyl, or CF₃; or R² is —O—CH₂CH₂—CF₃, —SO₂NH—C(CH₃)₃, or —SO₂-cyclopentyl.
E73. The method of any one of the preceding embodiments, wherein the KIF18A inhibitor is a compound of formula (I), or the pharmaceutically-acceptable salt thereof, wherein R² is a group —Y—R¹³; wherein Y is —C₀₋₄alk-; and —R¹³ is 4,4-difluoro-1-piperidinyl or 2-methylmorpholinyl.
E74. The method of any one of the preceding embodiments, wherein the KIF18A inhibitor is a compound of formula (I), or the pharmaceutically-acceptable salt thereof, wherein R⁴ is H or methyl.
E75. The method of any one of the preceding embodiments, wherein the KIF18A inhibitor is a compound of formula (I), or the pharmaceutically-acceptable salt thereof, wherein R⁵ is H.
E76. The method of any one of the preceding embodiments, wherein the KIF18A inhibitor is a compound of formula (I), or the pharmaceutically-acceptable salt thereof, wherein R⁶ is H.
E77. The method of any one of the preceding embodiments, wherein the KIF18A inhibitor is a compound of formula (I), or the pharmaceutically-acceptable salt thereof, wherein R⁷ is H.
E78. The method of any one of the preceding embodiments, wherein the KIF18A inhibitor is a compound of formula (I), or the pharmaceutically-acceptable salt thereof, wherein R^{a} is H.
E79. The method of any one of the preceding embodiments, wherein the KIF18A inhibitor is a compound of formula (I), or the pharmaceutically-acceptable salt thereof, wherein R^{a} is H.
E80. The method of any one of the preceding embodiments, wherein the KIF18A inhibitor is a compound of formula (I), or the pharmaceutically-acceptable salt thereof, wherein said compound is selected from the group consisting of:

| Ex. # | Chemical Structure | Chemical Name |
|---|---|---|
| C1 | 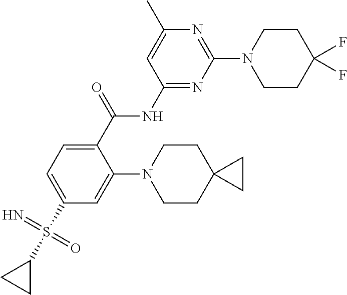 [SMILES: Cc1cc(NC(=O)c2ccc([S@](=N)(=O)C3CC3)cc2N2CCC3(CC2)CC3)nc(N2CCC(F)(F)CC2)n1] | 2-(6-Azaspiro[2.5]octan-6-y)-4-(R- cyclopropylsulfonimidoyl)-N-(2- (4,4-difluoro-1-piperidinyl)-6- methyl-4-pyrimidinyl)benzamide |
| C2 | 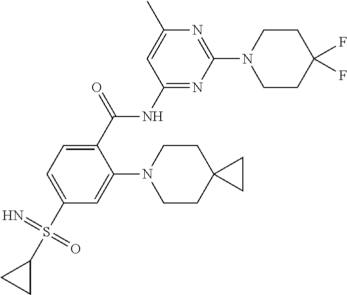 [SMILES: Cc1cc(NC(=O)c2ccc(S(=N)(=O)C3CC3)cc2N2CCC3(CC2)CC3)nc(N2CCC(F)(F)CC2)n1] | 2-(6-azaspiro[2.5]octan-6-yl)-4-(S- cyclopropylsulfonimidoyl)-N-(2- (4,4-difluoro-1-piperidinyl)-6- methyl-4-pyrimidinyl)benzamide |
| C3 | 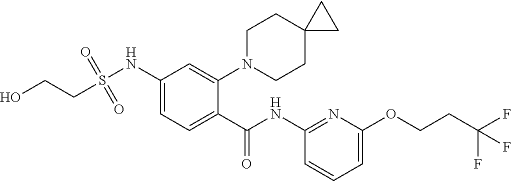 [SMILES: O=C(Nc1cccc(OCCC(F)(F)F)n1)c1ccc(NS(=O)(=O)CCO)cc1N1CCC2(CC1)CC2] | 4-((2-Hydroxyethyl)sulfonamido)-2- (6-azaspiro[2.5]octan-6-yl)-N-(6- (3,3,3-trifluoropropoxy)pyridin-2- yl)benzamide |
| C4 | 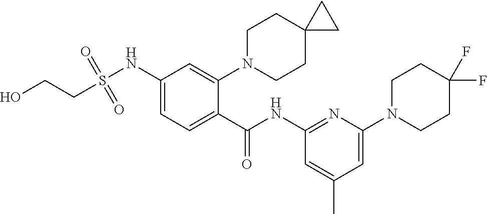 [SMILES: Cc1cc(NC(=O)c2ccc(NS(=O)(=O)CCO)cc2N2CCC3(CC2)CC3)nc(N2CCC(F)(F)CC2)c1] | N-(6-(4,4-difluoropiperidin-1-yl)-4- methylpyridin-2-yl)-4-((2- hydroxyethyl)sulfonamido)-2-(6- azaspiro[2.5]octan-6-yl)benzamide |
| C5 | 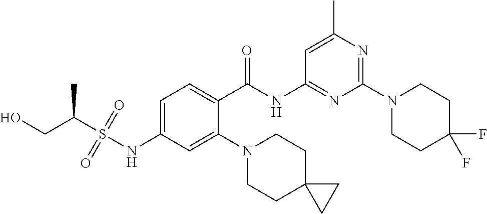 [SMILES: Cc1cc(NC(=O)c2ccc(NS(=O)(=O)[C@H](C)CO)cc2N2CCC3(CC2)CC3)nc(N2CCC(F)(F)CC2)n1] | (R)-N-(2-(4,4-difluoropiperidin-1- yl)-6-methylpyrimidin-4-yl)-4-((2- hydroxy-1- methytethyl)sulfonamido)-2-(6- azaspiro[2.5]octan-6-yl)benzamide |
| C6 | 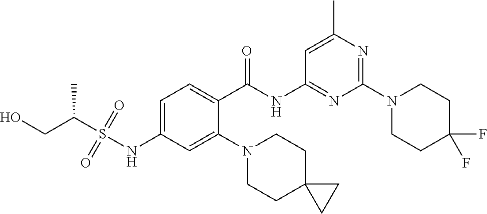 [SMILES: Cc1cc(NC(=O)c2ccc(NS(=O)(=O)[C@@H](C)CO)cc2N2CCC3(CC2)CC3)nc(N2CCC(F)(F)CC2)n1] | (S)-N-(2-(4,4-difluoropiperidin-1- yl)-6-methylpyrimidin-4-yl)-4-((2- hydroxy-1- methylethyl)sulfonamido)-2-(6- azaspiro[2.5]octan-6-yl)benzamide |
| C7 | 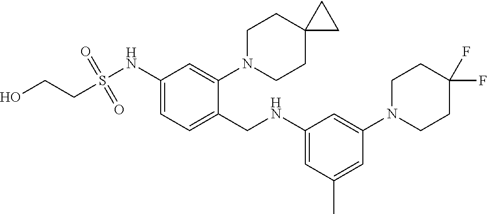 [SMILES: Cc1cc(NCc2ccc(NS(=O)(=O)CCO)cc2N2CCC3(CC2)CC3)cc(N2CCC(F)(F)CC2)c1] | N-(3-(4,4-difluoropiperidin-1-yl)-5- methylphenyl)-4-((2- hydroxyethyl)sulfonamido)-2-(6- azaspiro[2.5]octan-6-yl)benzamide |
| C8 | 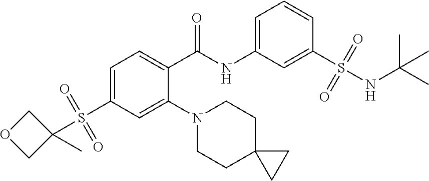 [SMILES: CC(C)(C)NS(=O)(=O)c1cccc(NC(=O)c2ccc(S(=O)(=O)C3(C)COC3)cc2N2CCC3(CC2)CC3)c1] | N-(3-(N-(tert- Butyl)sulfamoyl)phenyl)-4-((3- methyloxetan-3-yl)sulfonyl)-2-(6- azaspiro[2.5]octan-6-yl)benzamide |
| C9 | 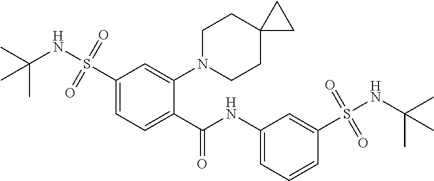 [SMILES: CC(C)(C)NS(=O)(=O)c1cccc(NC(=O)c2ccc(S(=O)(=O)NC(C)(C)C)cc2N2CCC3(CC2)CC3)c1] | 4-(N-(tert-butyl)sulfamoy)-N-(3-(N- (tert-butyl)sulfamoyl)phenyl)-2-(6- azaspiro[2.5]octan-6-yl)benzamide |
| C10 | 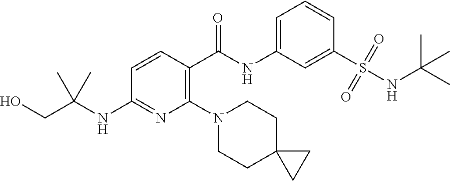 [SMILES: CC(C)(C)NS(=O)(=O)c1cccc(NC(=O)c2ccc(NC(C)(C)CO)nc2N2CCC3(CC2)CC3)c1] | N-(3-(N-(tert- Butyl)sulfamoyl)phenyl-6-((1- hydroxy-2-methylpropan-2- yl)amino)-2-(6-azaspiro[2.5]octan- 6-yl)nicotinamide |
| C11 | 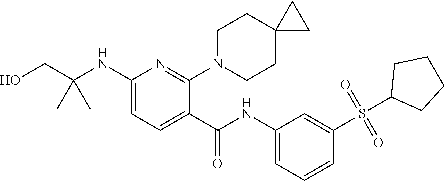 [SMILES: CC(C)(CO)Nc1ccc(C(=O)Nc2cccc(S(=O)(=O)C3CCCC3)c2)c(N2CCC3(CC2)CC3)n1] | N-(3-(cyclopentylsulfonyl)phenyl)- 6-((1-hydroxy-2-methylpropan-2- yl)amino)-2-(6-azaspiro[2.5]octan- 6-yl)nicotinamide |
| C12 | 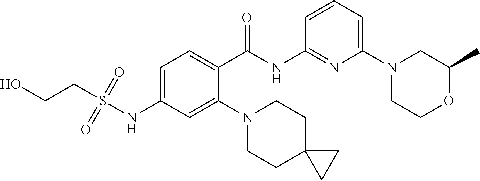 [SMILES: C[C@@H]1CN(c2cccc(NC(=O)c3ccc(NS(=O)(=O)CCO)cc3N3CCC4(CC3)CC4)n2)CCO1] | (R)-4-((2- Hydroxyethyl)sulfonamido)-N-(6- (2-methylmorpholino)pyridin-2-yl)- 2-(6-azaspiro[2.5]octan-6- yl)benzamide |
| C13 | 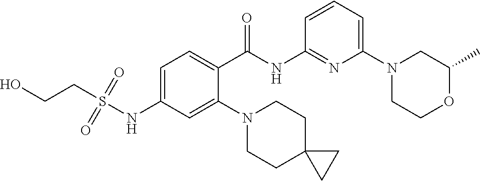 [SMILES: C[C@H]1CN(c2cccc(NC(=O)c3ccc(NS(=O)(=O)CCO)cc3N3CCC4(CC3)CC4)n2)CCO1] | (S)-4-((2- Hydroxyethyl)sulfonamido)-N-(6- (2-methylmorpholino)pyridin-2-yl)- 2-(6-azaspiro[2.5]octan-6- yl)benzamide |
| C14 | 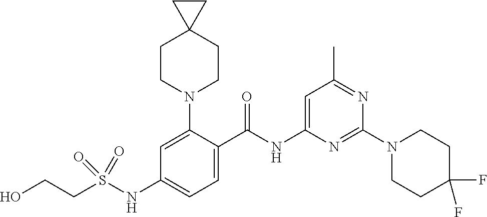 [SMILES: Cc1cc(NC(=O)c2ccc(NS(=O)(=O)CCO)cc2N2CCC3(CC2)CC3)nc(N2CCC(F)(F)CC2)n1] | N-(2-(4,4-Difluoropiperidin-1-yl)-6- methylpyrimidin-4-yl)-4-((2- hydroxyethyl)sulfonamido)-2-(6- azaspiro[2.5]octan-6-yl)benzamide |

* Ex. # stands for the example no. as well as the KIF18A Inhibitor Compound's short name used herein, e.g., in EXAMPLES.

E81. The method of any one of the preceding embodiments, wherein the KIF18A inhibitor is a compound of formula (I), or the pharmaceutically-acceptable salt thereof, wherein said compound is any one of compounds C1, C2, C3, C4, C5, C6, C7, C8, C9, C10, C11, C12, C13, or C14, or the pharmaceutically-acceptable salt thereof.
E82. The method of embodiment E81 wherein said salt is sulfate, HCl, mesylate, tosylate, or besylate salt.
E83. The method of embodiment E82 wherein said salt is HCl salt.
E84. The method of any one of the preceding embodiments, wherein the KIF18A inhibitor selectively treats the neoplastic disease, selectively induces or increases tumor regression, selectively reduces tumor or cancer growth, and/or selectively induces or increases death of tumor or cancer cells and the KIF18A inhibitor is not toxic to normal somatic cells.
E85. The method of any one of the preceding embodiments, wherein the KIF18A inhibitor treats the neoplastic disease, induces or increases tumor regression, reduces tumor or cancer growth, and/or induces or increases death of tumor or cancer cells and the proliferation of the normal somatic cells in the subject is substantially the same as the proliferation of the normal somatic cells of a control subject.
E86. The method of any one of the preceding embodiments, wherein the KIF18A inhibitor treats the neoplastic disease, induces or increases tumor regression, reduces tumor or cancer growth, and/or induces or increases death of tumor or cancer cells and the level of apoptosis of normal somatic cells is not increased in the subject, relative to the level of apoptosis of normal somatic cells of a control subject, optionally, wherein the level of apoptosis of normal somatic cells is substantially the same as the level of apoptosis of the normal somatic cells of a control subject.
E87. The method of any one of embodiments E84-E86, wherein the normal somatic cells are human bone marrow mononuclear cells, human mammary epithelial cells, or human foreskin fibroblast cells.
E88. The method of any one of embodiments E84-E87, wherein the normal somatic cells are not TP53^{MUT} or wherein the normal somatic cells are TP53^{WT}.
E89. The method of any one of the preceding embodiments, wherein the neoplastic disease is a multidrug resistant neoplastic disease.
E90. The method of any one of the preceding embodiments, wherein the tumor or cancer cells are multidrug resistant tumor or cancer cells and/or exhibit increased expression of the Multidrug resistance 1 (MDR-1) gene and/or a gene product thereof.
E91. The method of embodiment E99, wherein the tumor or cancer cells exhibit increased expression of a P-glycoprotein (P-gp).
E92. The method of any one of the preceding embodiments, wherein the neoplastic disease is resistant to treatment with an anti-mitotic agent or anthracycline antibiotic, optionally, paclitaxel or doxorubicin.
E93. A method of treating a neoplastic disease in a subject who is or has been treated with an anti-mitotic agent or anthracycline antibiotic, said method comprising, consisting essentially of, or consisting of administering a KIF18A inhibitor to the subject.
E94. The method of any one of the preceding embodiments, wherein the KIF18A inhibitor reduces expression of a KIF18A gene and/or a KIF18A gene product.
E95. The method of embodiment E94, wherein the KIF18A inhibitor is a non-coding RNA.
E96. The method of embodiments E95, wherein the KIF18A inhibitor mediates RNAi.
E97. The method of any one of the preceding embodiments, wherein the KIF18A inhibitor is an siRNA.
E98. The method of embodiment E97, wherein the siRNA comprises a sequence of any one of SEQ ID NOs: 12-18.
E99. The method of any one of the preceding embodiments, comprising, consisting essentially of or consisting of assaying for an inactivated TP53 gene.
E100. The method of any one of the preceding embodiments, comprising, consisting essentially of or consisting of assaying for an inactivated Rb1 gene.
E101. The method of any one of the preceding embodiments, comprising, consisting essentially of or consisting of assaying for an amplified CCNE1 gene or overexpression of a CCNE1 gene product.
E102. The method of any one of the preceding embodiments, comprising, consisting essentially of or consisting of assaying for an inactivated BRCA gene.
E103. The method of any one of the preceding embodiments, comprising, consisting essentially of or consisting of assaying for an inactivated TP53 gene and an inactivated Rb1 gene.
E104. The method of any one of the preceding embodiments, comprising, consisting essentially of or consisting of assaying for an inactivated TP53 gene and an amplified CCNE1 gene or overexpression of a CCNE1 gene product.
E105. The method of any one of the preceding embodiments, comprising, consisting essentially of or consisting of assaying for an inactivated TP53 gene and an inactivated BRCA gene.
E106. The method of any one of the preceding embodiments, comprising, consisting essentially of or consisting of assaying for an inactivated Rb1 gene and an amplified CCNE1 gene or overexpression of a CCNE1 gene product.
E107. The method of any one of the preceding embodiments, comprising, consisting essentially of or consisting of assaying for an inactivated Rb1 gene and an inactivated BRCA gene.
E108. The method of any one of the preceding embodiments, comprising, consisting essentially of or consisting of assaying for an amplified CCNE1 gene or overexpression of a CCNE1 gene product and an inactivated BRCA gene.
E109. The method of any one of the preceding embodiments, comprising, consisting essentially of or consisting of assaying for an inactivated TP53 gene, an inactivated Rb1 gene, and an amplified CCNE1 gene or overexpression of a CCNE1 gene product.
E110. The method of any one of the preceding embodiments, comprising, consisting essentially of or consisting of assaying for an inactivated TP53 gene, an inactivated Rb1 gene, and an inactivated BRCA gene.
E111. The method of any one of the preceding embodiments, comprising, consisting essentially of or consisting of assaying for an inactivated TP53 gene, an amplified CCNE1 gene or overexpression of a CCNE1 gene product, and an inactivated BRCA gene.
E112. The method of any one of the preceding embodiments, comprising, consisting essentially of or consisting of assaying for an inactivated Rb1 gene, an amplified CCNE1 gene or overexpression of a CCNE1 gene product, and an inactivated BRCA gene.
E113. The method of any one of the preceding embodiments, comprising, consisting essentially of or consisting of assaying for an inactivated TP53 gene, an inactivated Rb1 gene, an amplified CCNE1 gene or overexpression of a CCNE1 gene product, and an inactivated BRCA gene.
E114. The method of any one of the preceding embodiments, wherein the neoplastic disease is a cancer comprising one or more whole genome duplication or whole genome doubling (WGD) events.

The following examples are given merely to illustrate the present invention and not in any way to limit its scope.

### EXAMPLES

### Example 1

This example describes an analysis of cancer cell lines for sensitivity to a KIF18A inhibitor, relative to CDK4/6 inhibitors and an Eg5 inhibitor.

A KIF18A inhibitor, Compound C14, was evaluated in a 4-day image-based nuclear count assay (NCA) using a panel of cancer cell lines including breast cancer cell lines, ovary cancer cell lines, and a prostate cancer cell line. Eg5 motor inhibitor (Ispinesib) was used as a cytotoxic control. A CDK4/6 inhibitor (Palbociclib) was used as a comparator agent active in cell lines with an intact Rb pathway (T VanArsdale et al, Clinical Cancer Research. 2015; 1; 21:2905-2910).

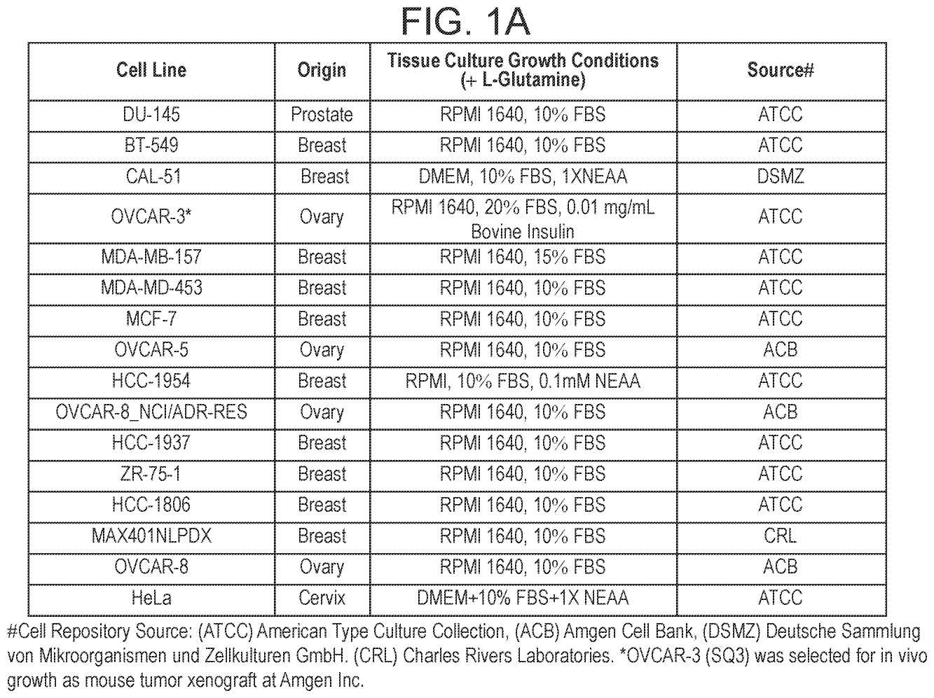

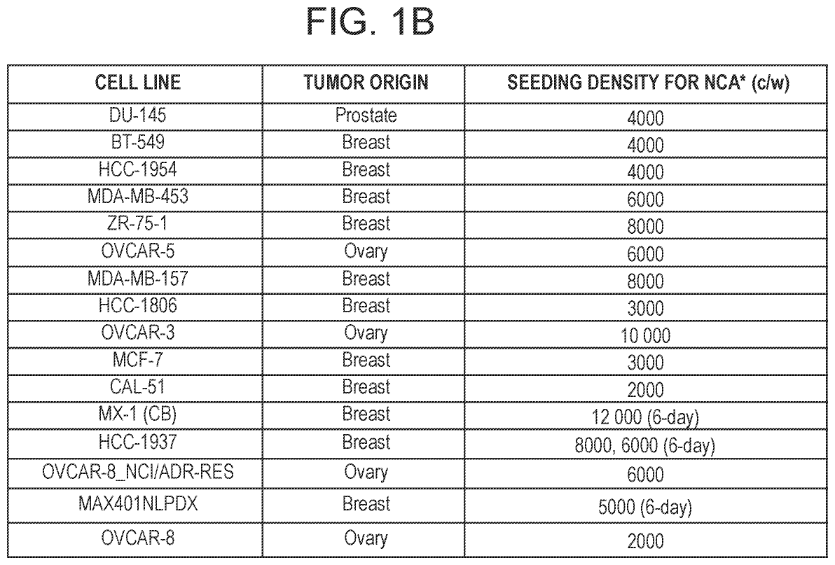

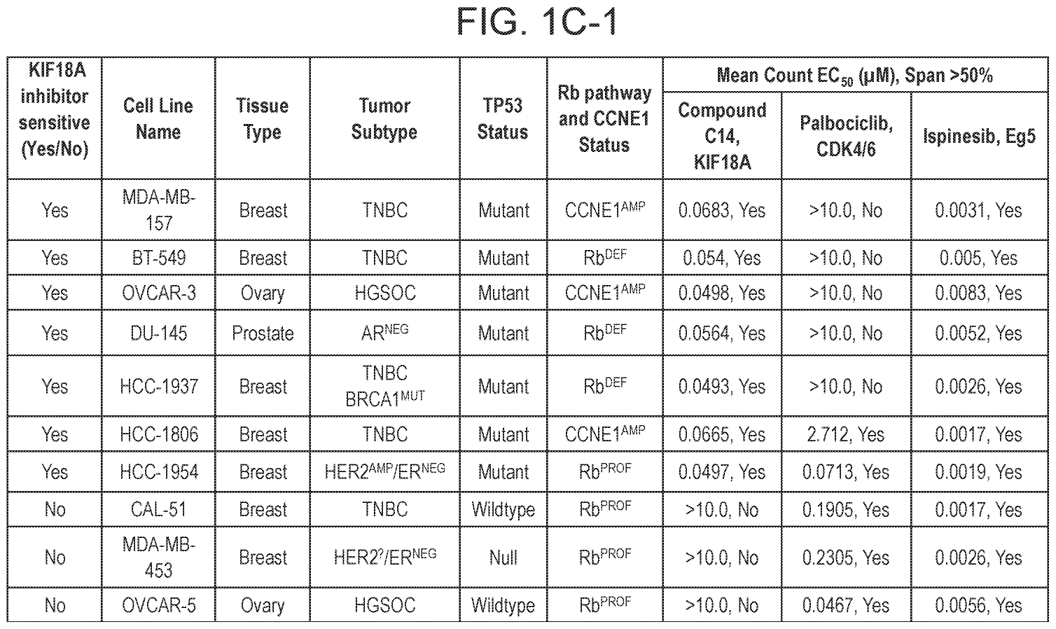

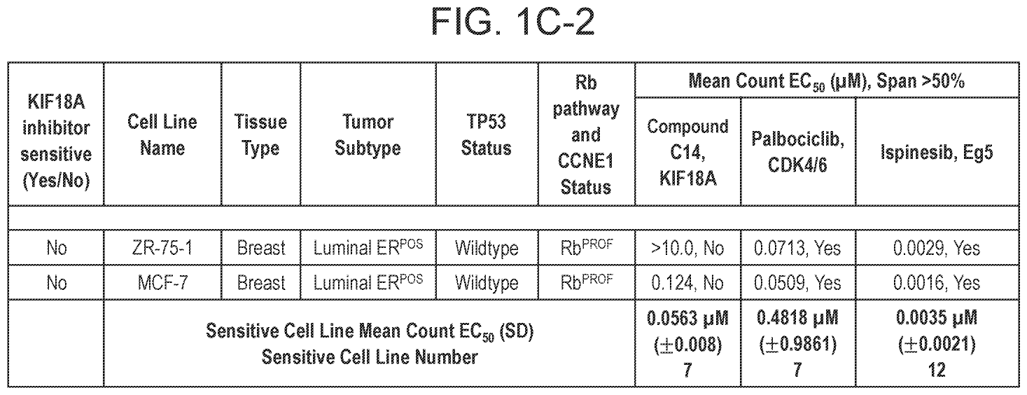

A description of the human cancer cell lines and the cell culture methods for the cell lines are provided in Table 1A of FIG. 1A. All human cancer cell lines were obtained from the American Type Culture Collection (ATCC) (Manassas, Va.) unless otherwise specified. The breast cancer cell line CAL-51 was obtained from DSMZ (GmbH). National Cancer Institute (NCI) Ovarian cancer cell lines OVCAR-8_NCI/ADR-RES expressing P-glycoprotein (P-gp) and OVCAR-5 were obtained from Amgen Cell Bank. MAX401NLPDX cell line was obtained from Charles River Laboratories. Cell lines used in this report were authenticated by ATCC using Short Tandem Repeats (STR) method except for OVCAR-5 cancer cell line originally obtained by ACB from NCI, STR profile obtained from ATCC for OVCAR-5 cells was searched against the ExPasy database and showed 100% match with OVCAR-5. All cell line cultures were maintained in at 37° C. in an atmosphere of 5% CO₂.

Cancer cell lines were seeded in a Corning 96-well Flat Clear Bottom Black Polystyrene plate (Corning, N.Y.) in 100 μL of appropriate complete media at the appropriate density and grown for 24 hours. A description of the cell line seeding densities used in the NCA studies are provided in Table 1B of FIG. 1B.

In one set of experiments, in preparation for cell treatment, a 2× concentration of one of: Compound C14, Pablociclib (CDK4/6 inhibitor), or Ispinesib (Eg5 inhibitor), was serial diluted into 100 μL of complete media using the BRAVO [final 20-point concentration range of 10 μM to 0.0003 μM (for Compound C14 or Palbociclib) and 1 μM to 0.00003 μM (Ispinesib), using staggered dose approach]. The compound was added to the cells with a final volume of 200 μL in complete media containing 0.5% DMSO.

In a second set of experiments, in preparation for cell treatment, 2× concentration of one of: Compound C14, Olaparib (PARP inhibitor), Paclitaxel (taxane), Doxorubicin (anthracycline), and Carboplatin (Platinum) were serial diluted into 100 μL of complete media using the BRAVO [final 20-point concentration range of 10 μM to 0.0003 μM (Compound C14), 100 μM to 0.003 μM (Olaparib, Carboplatin), 1 μM to 0.00003 μM (Paclitaxel), and 2 μM to 0.00006 μM (Doxorubicin) using staggered dose approach]. Compound was added to the cells with a final volume of 200 μL in complete media containing 0.5% DMSO.

After 4 days (96 hours) or 6 days (144 hours) of treatment, the cells were fixed by removing 100 μL of complete media from each well and replacing it with 100 μL of 2×formaldehyde (final 4%) and incubating the plates for 15 minutes at room temperature. After fixation, the cells were permeabilized and stained in 200 μL Wash Buffer (1% BSA, 0.2% Triton X-100, 1×PBS) containing 2 μg/mL Hoechst 33342 DNA dye. The plates were sealed and incubated for 1 hour at room temperature in the dark. Cells were stored at 4° C. in the dark until data acquisition. Imaging data was acquired on Cellomics ArrayScan VTI HCS Reader (SN03090745F, ThermoFisher Scientific) using the Target Activation V4 Assay protocol (Ve 6.6.0 (Build 8153) with a 10×objective, collected 16-fields per well). The Valid Object Count was determined using Hoechst 33342 nuclear object features (area, total and variable intensity in Channel 1) that were within ±3 SD of the DMSO-treated control. The Total Valid Object Count was represented as a Count POC (Percentage of DMSO Control) using the following formula:

Count POC=(Total Valid Object Count in treated well)+(Total Valid Object Count in DMSO treated wells)×100

Compound concentration and Count POC values were plotted using GraphPad Prism software (V7.0.4) and curve-fitting was performed with 4-parameter equation (variable slope). The concentration-response curves and standard deviation represent two independent experiments run in duplicate.

Table 1C (FIG. 1C) indicates the Mean Count EC₅₀ value for each cell line and the Span (POC Top-Bottom), if the Span did not exceed >50%, the cell line was considered insensitive. A Mean Count EC₅₀ value (±SD) across all sensitive cell lines was determined for each test agent.

Consensus information for Table 1C. Cell line characteristics (tissue type, tumor subtype, TP53 mutation status, Rb pathway status) were obtained from the following online databases (Cancer DepMap and CCLE (https://deomap.org/portal/depmap), Broad Institute; Cell Model Passports (https://cellmodelpassoorts.sanger.ac.uk/passports), Sanger Institute IARC (http://p53.iarc.fr/OtherResources.aspx), International Agency for Research on Cancer) and references (O'Brien et al, 2018; Konecny et al, 2012; Finn et al, 2009; Dai et al, 2017; Tilley et al, 1990; Witkiewicz et al, 2018).

Abbreviations for Table 1C. missense mutation (MM), nonsense mutation (NM), frame shift deletion (FSD), frame shift insertion (FSI), stop codon (*), frame shift (fs), amplification (AMP), proficient (PROF), deficient (DEF), amino acid (A.A.), positive (POS), negative (NEG), estrogen receptor (ER), and androgen receptor (AR). Information for MDA-MB-453 HER-2 amplification status uncertain (?).

### REFERENCES FOR TABLE 1C

O'Brien N, Conklin D, Beckmann R, Luo T, Chau K, Thomas J, et al. Preclinical activity of abemaciclib alone or in combination with antimitotic and targeted therapies in breast cancer. Molecular Cancer Therapeutics. 2018; 17(5):897-907.
Konecny G E, et al. Expression of p16 and retinoblastoma determines response to CDK4/6 inhibition in ovarian cancer. Clinical Cancer Research. 2011 Mar. 15; 17(6):1591-1602.
Finn R S et al. PD 0332991, a selective cyclin D kinase 4/6 inhibitor, preferentially inhibits proliferation of luminal estrogen receptor-positive human breast cancer cell lines in vitro. Breast Cancer Research. 2009; 11(5):R77.
Dai X, Cheng H, Bai Z, Li J. Breast cancer cell line classification and its relevance with breast tumor subtyping. Journal of Cancer. 2017; 8(16):3131.
Tilley W D, Wilson C M, Marcelli M, McPhaul M J. Androgen receptor gene expression in human prostate carcinoma cell lines. Cancer Research. 1990; 50(17):5382-5386.
Witkiewicz A K, Chung S, Brough R, Vail P, Franco J, Lord C J, Knudsen E S. Targeting the vulnerability of RB tumor suppressor loss in triple-negative breast cancer. Cell reports. 2018; 22(5):1185-1199.

As shown in Table 1C of FIG. 1C, all cell lines that exhibited sensitivity to the KIA18A inhibitor were mutant TP53 cancer cell lines. Seven of the twelve cell lines were scored as “sensitive” and showed similar 4-day Mean Count EC₅₀ values (0.0563 μM with SD ±0.008). Five Rb-proficient cancer cell lines including four TP53^{WT} lines (CAL-51, ZR-75-1, MCF-7, OVCAR-5) and one TP53^{NULL} line (MDA-MB-453) were Palbociclib-sensitive and KIF18A inhibitor-insensitive. In all KIF18A inhibitor-sensitive cell lines, except for one (HCC-1937 BRCA1 mutant), the cells lines had either a CCNE1 amplification or an Rb^{DEF} status. Together, these data indicate KIF18A inhibitor and Palbociclib have distinct and largely nonoverlapping sensitivity profiles suggesting that Rb pathway status may serve as segmentation biomarker (e.g. RB1 loss and CCE1 amplification). KIF18A inhibitor sensitivity profile is clearly distinct from cytotoxic effects of Ispinesib, with a focal sensitivity profile suggesting only a subset of cancer cell lines exhibit a heightened mitotic-specific vulnerability and KIF18A dependence.

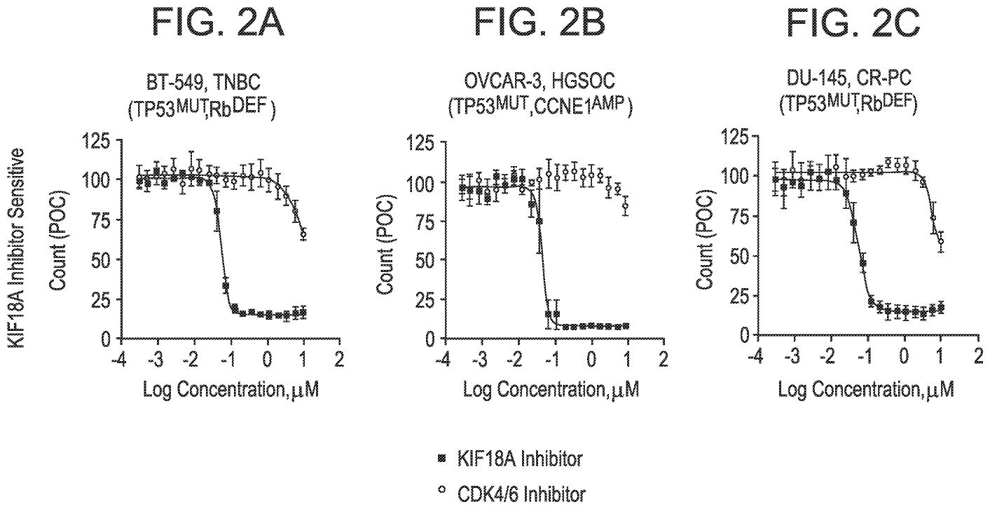

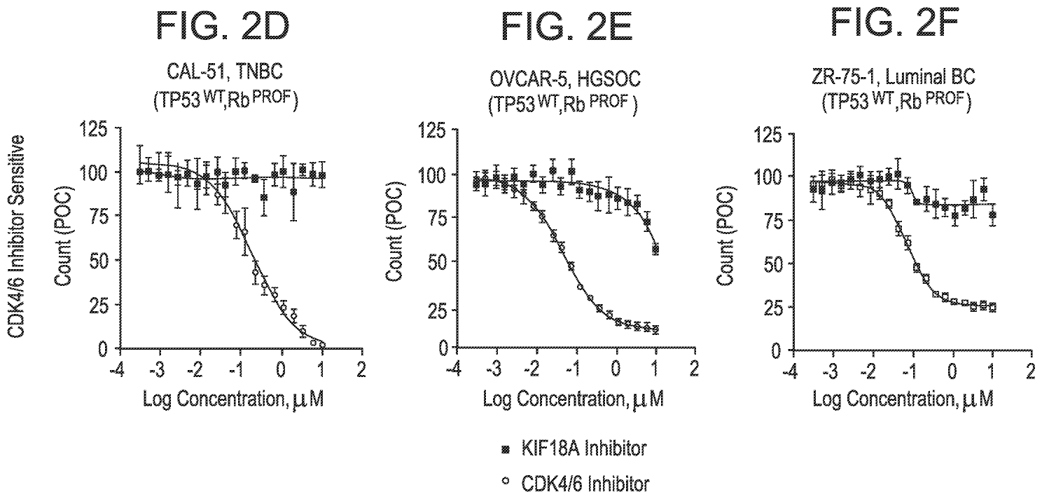

FIGS. 2A-2F are graphs of the Count POC values for six of the tested cell lines plotted as a function of concentration of the KIF18A inhibitor. The six cell lines of FIG. 2A-2F were BT-549 (a TNBC characterized as TP53^{MUT} and RB^{DEF}), OVCAR-3 (an HGSOC characterized as TP53^{MUT} and CCNE1^{AMP}), DU-145, a CR-PC characterized as TP53^{MUT} and RB^{DEF}), CAL-51, a TNBC characterized as TP53′ and RB^{PROF}), OVCAR-5, an HGSOC characterized as TP53^{WT} and RB^{PROF}), and ZR-75, a luminal breast cancer characterized as TP53^{WT} and RB^{PROF}). The concentration-response fitted-curves of FIG. 2A-2F are represented as the mean count based on percentage of control (DMSO) with error bars (SD).

The assay was performed in duplicate in two independent experiments. Interestingly, those cell lines that were sensitive to KIF18A inhibitor were not sensitive to the CDK4-6 inhibitor, palbociclib and vice versa. These data suggest that CDK4/6 inhibitor cancer cell sensitivity may serve as a negative predictor for cancer cell sensitivity to KIF18A inhibitors, and that Rb pathway inactivation (e.g. RB1 loss and CCE1 amplification) may serve as potential response biomarkers to KIF18A inhibitor treatment.

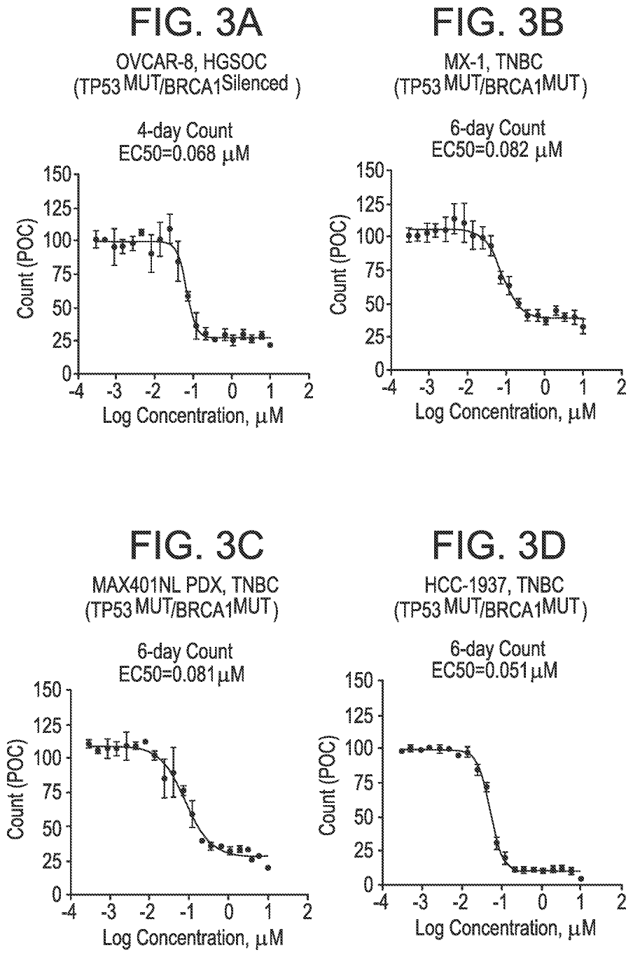

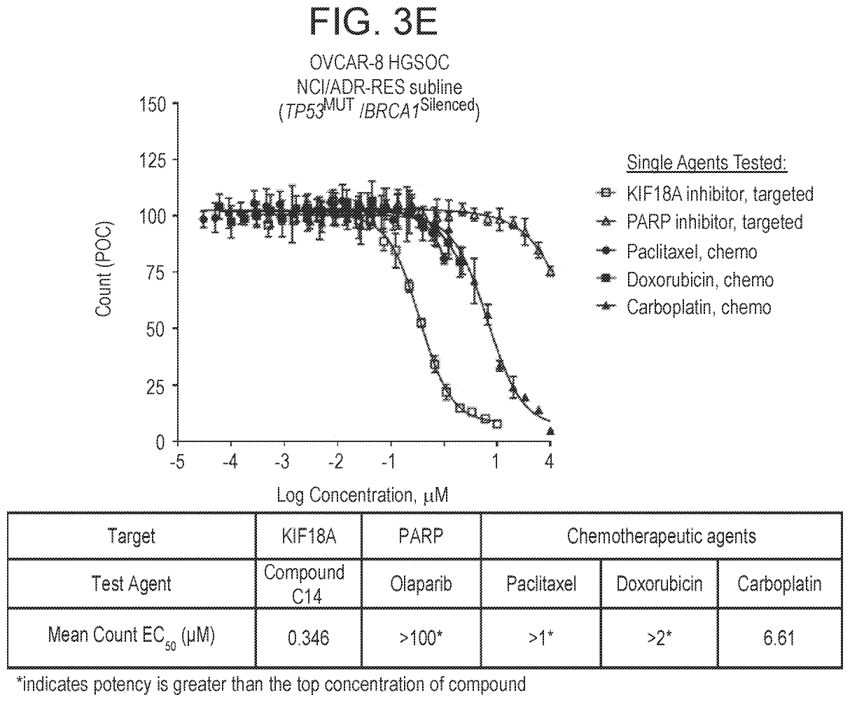

FIGS. 3A-3D represents a series of graphs of the Count POC values for four of the tested cell lines plotted as a function of concentration of KIF18A inhibitor. The four cell lines of FIGS. 3A-3D were OVCAR-8 (an HGSOC characterized as TP53^{MUT} and BRCA1^{Silenced}), MX-1 (a triple negative breast cancer (TNBC characterized as TP53^{MUT} and BRCA1^{MUT}), MAX401NL PDX (a TNBC characterized as TP53^{MUT} and BRCA1^{MUT}), and HCC-1937 (a TNBC characterized as TP53^{MUT} and BRCA1^{MUT}). As shown in each graph of FIGS. 3A-3D, the TP53^{MUT} and BRCA1-deficient cancer cell lines demonstrated sensitivity to treatment with the KIF18A inhibitor with Count EC50 values in 0.051 to 0.082 μM range.

FIG. 3E is a graph of the Count POC values for the OVCAR-8 NCI-ADR RES subline plotted as a function of concentration of the KIF18A inhibitor, PARP inhibitor, paclitaxel, doxorubicin, or carboplatin. As shown in FIG. 3E, the cell line demonstrated the greatest sensitivity to the KIF18A inhibitor. OVCAR-8 NCI-ADR RES cells overexpress drug pump MDR1 or ABCB1 gene (encodes for P-glycoprotein) known to induce multi-drug resistance to anti-cancer agents (A Vert et al OncoTargets and Therapy 2018:11; 221-37), this data suggests that KIF18A inhibitor sensitivity due to drug-efflux is only modestly impacted relative to olaparib, paclitaxel, and doxorubicin. Furthermore, multiple ABCB1 transcriptional fusions have been reported in chemotherapy resistant recurrent ovarian cancer (EL Christie et al Nature Communications. 2019:20; 10:1-10).

### Example 2

This example demonstrates an analysis of cancer cell lines for sensitivity to a KIF18A inhibitor Compound C9.

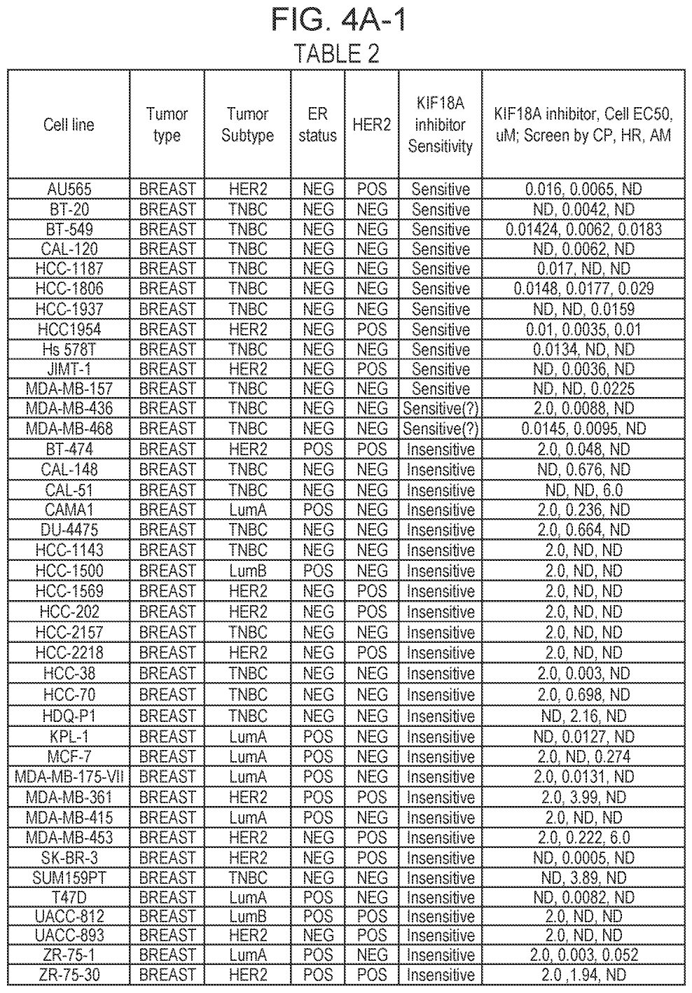

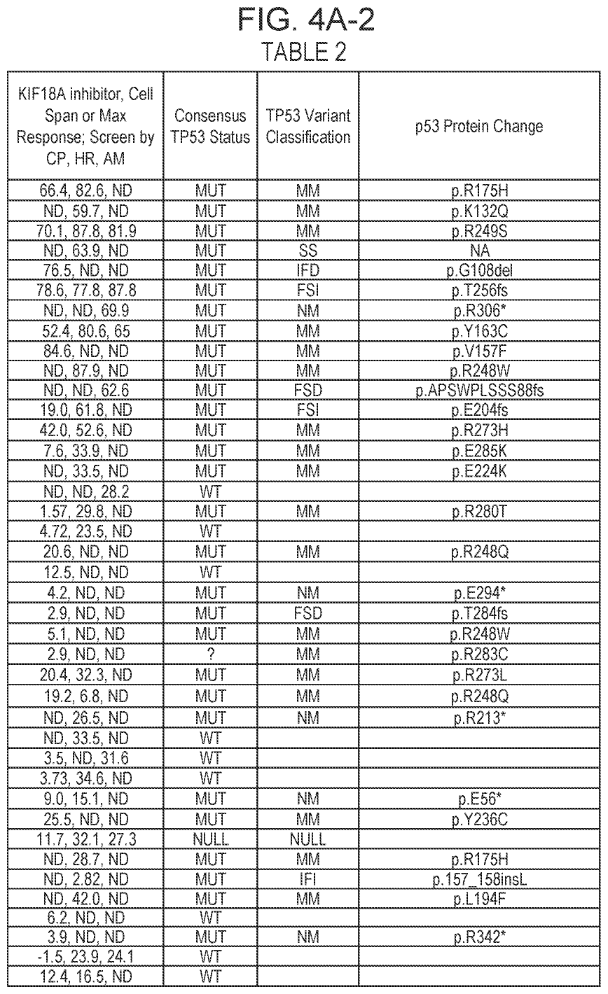

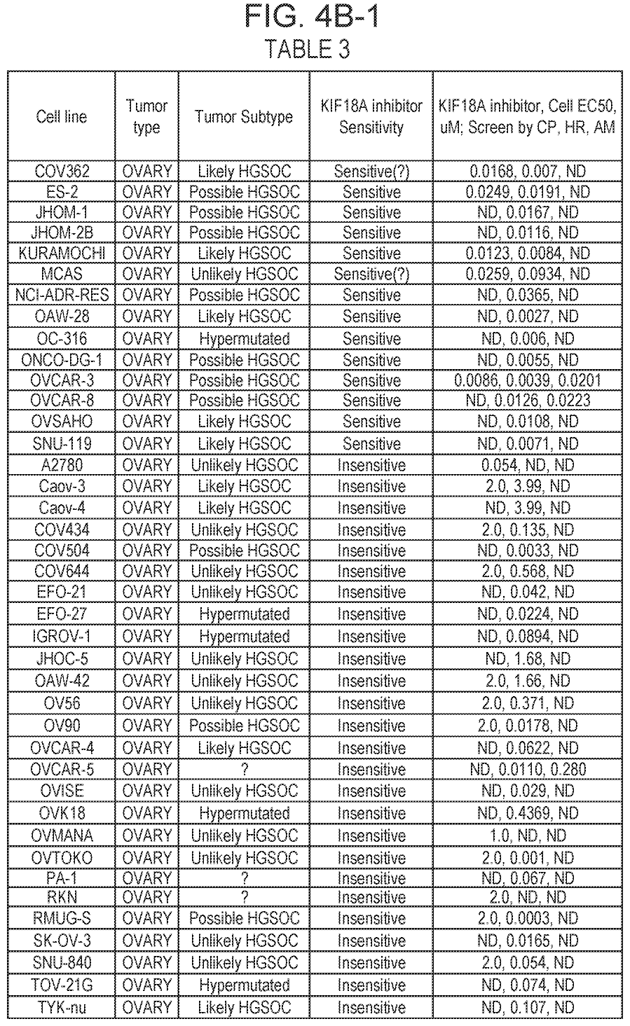

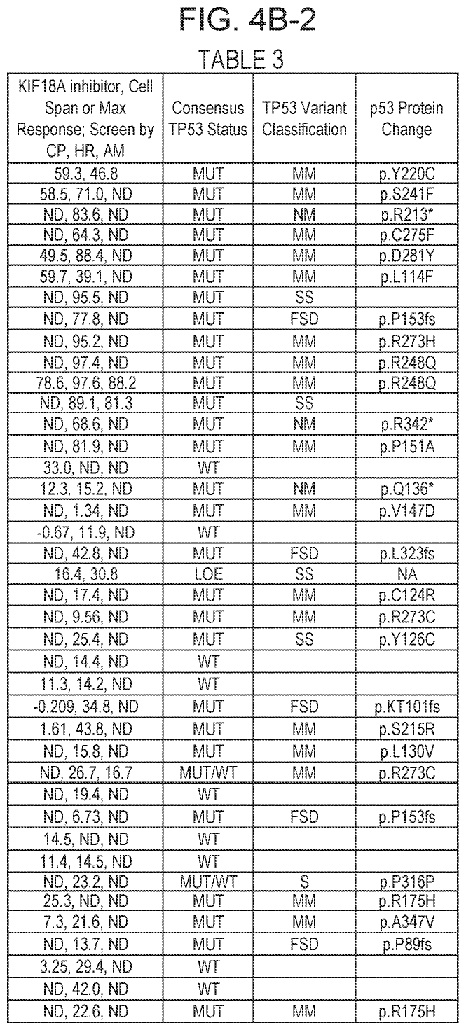

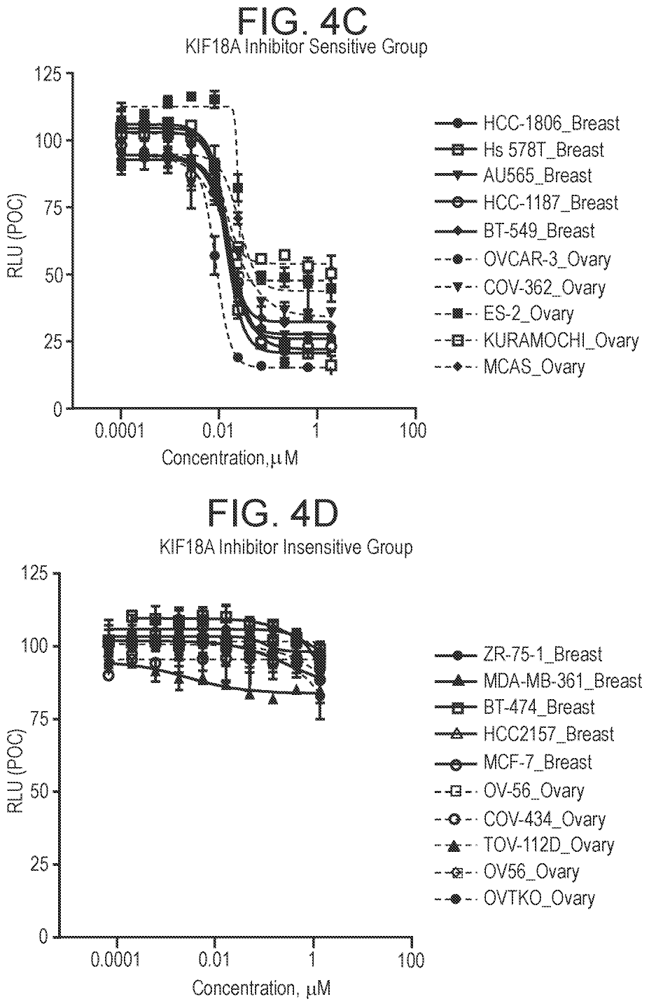

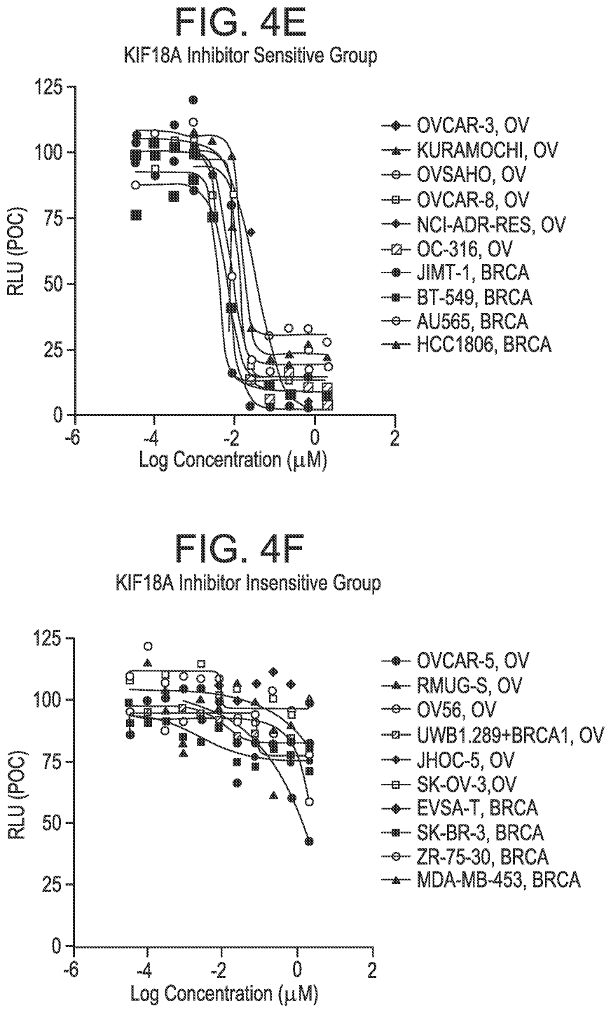

An analysis was carried out to identify cancer cell lines sensitivity profile to the KIF18A inhibitor Compound C9. In this analysis, a panel of different human breast and ovarian cancer cell lines (Table 2 of FIG. 4A and Table 3 of FIG. 4B), were used in either a 4-day or 6-day cancer cell line growth assay screen.

In the 4-day growth assay screen, cancer cell lines were treated in 96-well tissue culture plates at pre-optimized seeding density. Growth media conditions were determined by ChemPartners (Shanghai, China). After 24 hours, cells were treated with KIF18A inhibitor (Compound C9; final 10-point concentration range 2.0 μM to 0.0001 μM, 3-fold dilution) in a 4-day cell growth assay based on quantitation of ATP as an indicator of viable cells using CellTiter-GLO 2.0 readout (CTG, Promega). CTG assay was performed in duplicate for each cell line. Detection was performed using luminescence plate reader and expressed as relative luminescence units (RLU) based on POC (percentage of DMSO control). Raw data was provided to Amgen for curve-fitting analysis with 4-parameter equation (variable slope) using GraphPad Prism software (V7.0.4). The concentration-response curves and standard deviations were graphed (representative curves shown in FIGS. 4C and 4D). Reported values for EC50 (IP) and Span (difference between Max and Min response for fitted points) for each cancer cell line and classified cell lines as sensitive to KIF18A inhibitor compound when EC50 value <0.1 μM and Span ≥40.

An expanded screen was performed using 6-day growth assay, cancer cell lines were treated in black 384-well tissue culture plates at 500-1500 cells per well. Growth media conditions were determined by Horizon Discovery (Cambridge, United Kingdom). After 24 hours, cells were treated with KIF18A inhibitor (Compound C9; final 11-point concentration range 2.0 μM to 0.0000339 μM, 3-fold dilution) in a 6-day cell growth assay based on quantitation of ATP as an indicator of viable cells using CellTiter-GLO 2.0 readout (Promega). Luminescence was detected using Envision plate reader (Perkin Elmer) and expressed as relative luminescence units (RLU) based on POC (percentage of control). Raw data was provided to Amgen for curve-fitting analysis with 4-parameter equation (variable slope) using GraphPad Prism software (V7.0.4). The concentration-response curves and standard deviations were graphed (representative curves shown in FIGS. 4E-4F). Curve-fitting analysis was also performed using Horizon's proprietary software. Horizon Discovery reported values for EC50 (IP) and Max Response (Observed) for each cancer cell line and classified cell lines as sensitive to KIF18A inhibitor compound when EC50 value <0.1 μM and Max Response ≥59.5.

Additionally, a cell count cell line growth assay screen was carried out with the KIF18A inhibitor, as follows: a subset of breast and ovarian cancer cell lines were screened with KIF18A inhibitor Compound C9 as described above using imaging-based Nuclear Count Assay (NCA). Internal data KIF18A inhibitor either 4- or 6-day NCA. Curve-fitting was performed at Amgen using GraphPad Prism 7. Reported values: EC50 value (IP) and Span (difference between Max and Min response for fitted points) for each cancer cell line and classified cell lines as sensitive to KIF18A inhibitor compound when IC50 value <0.1 μM and Span ≥40.

The results of the cell count assays are shown in Table 2 and Table 3 and FIGS. 4C-4F. Table 2 is a summary of the analysis for the panel of human breast cancer cell lines and Table 3 is a summary of the analysis for the panel of human ovarian cancer cell lines.

In Tables 2 and 3, “?” means that there were differences in sensitivity calls between the first screen and the second screen, and “ND” means not determined. The screen was carried out either through a first screen or a second screen. The first screen (ChemPartner, CP) was a 4-day CellTiter-Glo® assay (CTG) assay. Curve-fitting was performed using GraphPad Prism 7. The reported values in Tables 2 and 3 are: EC50 value (IP) and Span or Max Response (difference between Max and Min response for fitted points). The first screen sensitivity scoring [Sensitive cell line group defined as Span ≥40 with EC50 value <0.1 μM]. The second screen (Horizon Discovery, HR) was a 6-day CTG assay. Curve-fitting was performed by HR. The reported values in Tables 2 and 3 are: EC50 value (IP) and Max Response (Observed). The second screen sensitivity scoring [Sensitive cell line group defined as Span ≥59.5 with EC50 value <0.1 μM]. A third screen (Amgen, AM) took place and it was either a 4-day or 6-day nuclear count assay (NCA). Curve-fitting was performed using GraphPad Prism 7. The reported values of Tables 2 and 3 are: EC50 value (IP) and Span (difference between Max and Min response for fitted points). The third screen sensitivity scoring [Sensitive cell line group defined as Span 240 with EC50 value <0.1 μM].

Consensus Information for FIGS. 4A-4F. Cell line characteristics (tissue type, tumor subtype, TP53 mutation status, TP53 variant type, p53 protein change) were obtained from the following online databases (Cancer DepMap and CCLE (https://depmap.org/portal/depmap), Broad Institute; Cell Model Passports (https://cellmodelpassports.sanger.ac.uk/passports), Sanger Institute IARC (http://p53.iarc.fr/OtherResources.aspx), International Agency for Research on Cancer) and references (Dai et al Journal of Cancer, 2017; O'Brien et al Mol. Cancer Ther., 2018; Domcke et al Nature Comm., 2013).

Abbreviations for Table 2 and Table 3. ChemPartner screen (CP), Horizon Discovery screen (HR), Amgen screen (AM), not determined (ND), (TNBC), high grade serous ovary cancer (HGSOC), estrogen receptor (ER), negative (NEG), positive (POS), HER2 receptor positive (HER2), luminal A (LumA), luminal B (LumB), mutant (MUT), wild-type (WT), loss of expression (LOE), missense mutation (MM), nonsense mutation (NM), frame shift deletion (FSD), frame shift insertion (FSI), stop codon (*), frame shift (FS), splice site (SS), in frame deletion (IFD), in frame insertion (IFI), silent (S), amino acid (A.A.), sensitivity column differences in calls between CP and HR screens (?), and tumor subtype and TP53 consensus columns calls uncertain (?).

Tumor Subtypes: Source XDai et al Journal of Cancer 2017, O'Brien et al Mol. Cancer Ther._2018 (Breast), Domcke et al_Nature Comm 2013 (Ovarian Cancer). TNBC=Triple negative breast cancer, HGSOC=high grade serous ovary cancer, ER=Estrogen Receptor, NEG=Negative, POS=Positive, HER2=HER2 receptor positive, LumA=lunimal A, LumB=luminal B, ?=subtype uncertain.

TP53 status: Source CCLE/Sanger/IARC calls: If consensus calls were uncertain either looked in the literature or list as uncertain (?). MUT (mutant), WT (wild-type), LOE (loss of expression). Variant Classification. MM (Missense_Mutation), NM (Nonsense_Mutation), SS (Splice_Site), IFD (In_Frame_Del), FSI (Frame_Shift_Ins), FSD (Frame_Shift_Del), NULL, IFI (In_Frame_Ins), Silent.

### REFERENCES FOR TABLE 2 AND TABLE 3

Dai X, Cheng H, Bai Z, Li J. Breast cancer cell line classification and its relevance with breast tumor subtyping. Journal of Cancer. 2017; 8(16):3131.
O'Brien N, Conklin D, Beckmann R, Luo T, Chau K, Thomas J, et al. Preclinical activity of abemaciclib alone or in combination with antimitotic and targeted therapies in breast cancer. Molecular Cancer Therapeutics. 2018; 17(5):897-907.
Domcke S, Sinha R, Levine D A, Sander C, Schultz, N. Evaluating cell lines as tumour models by comparison of genomic profiles. Nature Communications, 2013; 4(1): 1-10.

As shown in Table 2, breast cancer cell lines sensitive to treatment with the KIF18A inhibitor were positive for a mutant TP53 gene, many of which expressed a mutant TP53 protein due to a missense mutation. None of the nine TP53 wild-type breast cancer cell lines were sensitive to KIF18A inhibitor including two TNBC lines (CAL-51, DU4475). All breast cancer cell lines that were sensitive to treatment with the KIF18A inhibitor had a negative estrogen receptor (ER) status and about three-quarters of these cell lines also had a negative HER2 status. None of the luminal A or luminal B breast cancer cell lines were sensitive to KIF18A inhibitor.

As shown in Table 3, ovarian cancer cell lines that were sensitive to treatment with the KIF18A inhibitor were positive for a mutant TP53 gene, many of which expressed a mutant TP53 protein due to a missense mutation. None of the nine TP53 wild-type ovarian cancer cell lines were sensitive to KIF18A inhibitor. For most of these cancer cell lines, the tumor subtype was “likely” or “possibly” a high grade serous ovarian cancer (HGSOC) based on molecular classification (S. Domcke et al Nature Communications 2013:4:1-10).

Representative concentration-response curves from the first screen (FIGS. 4C-4D) and second screen (FIGS. 4E-4F) are shown in FIGS. 4C-4F, respectively. As shown in FIGS. 4C-4F, the KIF18A inhibitor sensitivity profiles were grouped into “sensitive” (FIGS. 4C, 4E) and “insensitive” (FIGS. 4D, 4F) for breast and ovarian cancer cell lines.

### Example 3

This example demonstrates KIF18A inhibitor Compound C14 induces tumor regressions in human OVCAR-3 HGSOC xenograft model (TP53^{MUT}, CCNE1^{AMP}) in female athymic nude mice.

To demonstrate the effect the KIF18A inhibitor has on tumor regression OVCAR-3 cells were re-selected in vivo and subsequently (OVCAR-3SQ3). Female athymic nude mice were injected with 5×10⁶ cells in 0.1 mL subcutaneously in the right flank. After tumors were established (average tumor volume of 150 mm³), animals were randomized into 4 treatment groups (vehicle alone, KIF18A inhibitor at 10, 30, or 100 mg/kg) with 10 animals per group and treated orally once per day (PO, QD) starting on day 25 post-tumor implantation. Tumor measurement was calculated from the length, width and height of tumors measured with a PRO-MAX electronic digital caliper (Japan Micrometer Mfg. Co. LTD). The tumor volume was calculated as [L×W×H] and expressed in mm³. Tumor volume and animal body weight measurements were determined twice per week (study start day 25, study end day 45). A single mouse was removed from the study (KIF18A inhibitor 30 mg/kg group) due to abdominal distention, a finding likely unrelated to compound.

Tumor Growth Inhibition (TGI) and Tumor Regression formulas.

$$\% TGI compared to vehicle control :$$

$$\% TGI = 100 - [ \frac{( Treated - Initial Volume )}{( Control - Initial Volume )} \times 100 ]$$

$$\% Regression compared final tumor volume to initial tumor volume :$$

$$\% Regression = 100 - [ \frac{( Final Volume )}{( Initial Volume )} \times 100 ]$$

Data was plotted using GraphPad Prism software (V7.0.4), tumor volume and body weight data are expressed as means plus or minus standard error of the mean and plotted as a function of time. Statistical significance of observed differences between growth curves were calculated using SLACR package (v.1.0.3). Statistical significance for tumor regressions were performed by paired Student's t-Test on the initial and final tumor volumes with significant tumor regression p-values (***p≤0.0001) for all three KIF18A inhibitor treatment groups. KIF18A inhibitor dose 10 mg/kg (81% regression, 5 of 10 tumor-free), 30 mg/kg (98% regression, 8 of 9 tumor-free), and 100 mg/kg (97% regression, 7 of 10 tumor-free). No evidence of overt toxicity was observed in the KIF18A inhibitor treated groups as determined by changes in animal body weight relative to vehicle treated group.

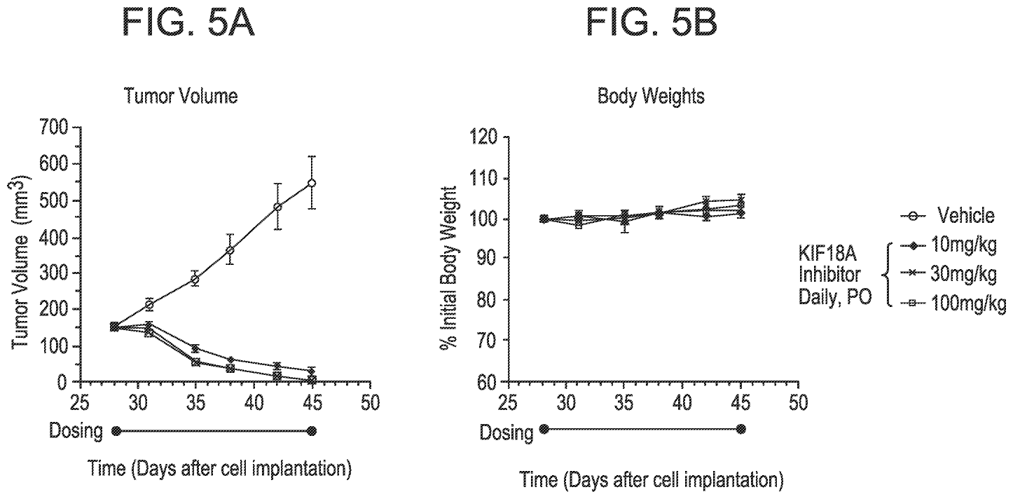

The results are shown in FIGS. 5A-5B. As shown in FIG. 5A, oral daily administration of KIF18A inhibitor significantly inhibited the growth of OVCAR-3 HGSOC tumors (TP53 mutant, CCNE1 amplified) and induced regressions at all three doses. Assessment of tumor re-growth potential after cessation of KIF18A inhibitor showed durable tumor regressions and cures in ≥50% of the animals with no evidence of overt toxicity, indicating KIF18A inhibitor was well-tolerated.

### Example 4

This example demonstrates KIF18A Inhibitor Compound C14 induces tumor regressions in human OVCAR-8 HGSOC xenograft model (TP53^{MUT}, BRCA1^{silenced}) in female athymic nude mice.

Female athymic nude mice were injected with 5×10⁶ OVCAR-8 cells in 0.1 mL subcutaneously in the right flank. Animals with established tumors were randomized (average tumor volumes of ˜134 mm³) into 4 treatment groups (vehicle alone, Compound C14 at 10, 30, or 100 mg/kg) with 10 animals per group and treated orally once per day (PO, QD) starting on day 25 post-tumor implantation. Tumor volume and animal body weight measurements were determined twice per week (study start day 25, study end day 47). Tumor measurements, formulas, and regression analysis as described above for FIGS. 5A-5B. Data was plotted using GraphPad Prism software (V7.0.4), tumor volume and body weight data are expressed as means plus or minus standard error of the mean and plotted as a function of time. Statistical significance of observed differences between growth curves were calculated using SLACR package (v.1.0.3). Statistical significance for tumor growth inhibition was determined for Compound C14 10 mg/kg group (57% TGI, p=0.003) by RMANOVA with a Dunnett's comparison relative to vehicle group. Statistical significance for tumor regressions was performed by paired Student's t-Test based on the initial and final tumor volumes with Compound C14 30 mg/kg (86% regression, p s 0.0001, 4 of 10 tumor-free), and Compound C14 100 mg/kg (98% regression, p s 0.0001, 8 of 10 tumor-free). No evidence of overt toxicity was observed in the Compound C14 treated groups as determined by changes in animal body weight relative to vehicle treated group.

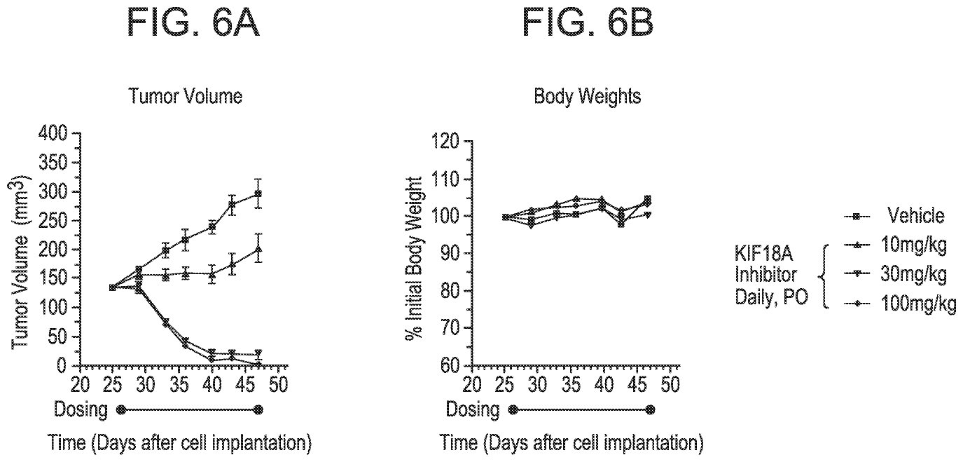

The results are shown in FIG. 6A-6B. As shown in FIGS. 6A-6B, oral daily administration of KIF18A inhibitor significantly inhibited the growth of OVCAR-8 HGSOC tumors (TP53 mutant, BRCA1^{silenced}) and induced regressions at 30 mg/kg and 100 mg/kg doses with no evidence of overt toxicity, indicating KIF18A inhibitor was well-tolerated.

### Example 5

This example describes a study to analyze the effects of a KIF18A inhibitor on the mitotic phenotype of cancer cells.

To analyze the effects of a KIF18A inhibitor on the mitotic phenotype of cancer cells, an imaging-based Centrosome Count Assay (CCA) carried out using a KIF18A inhibitor was carried out. In preparation for plating, MDA-MB-157 TNBC cells were resuspended seven times through a 10 mL syringe with 18-G needle to create a single-cell suspension. Cells were seeded at a density of 30 000 cells per well using the BRAVO Automated Liquid Handling Platform (Agilent Technologies, Santa Clara, Calif.) into Corning 96-well Flat Clear Bottom Black Polystyrene plate (Corning, N.Y.) in 100 μL of complete media and grown for 24 hours. In preparation for cell treatment, a 2× concentration of KIF18A inhibitor Compound C14 was serial diluted using the BRAVO (final 20-point concentration range 5.0 μM to 0.00015 μM, using staggered dose approach) into 100 μL of complete media and then added to the 100 μL of complete media containing the plated cells with a final DMSO concentration of 0.5%. After 24 hours of treatment, 100 μL of complete media was removed from each well and the cells were fixed by adding 100 μL formaldehyde (final 4%) to each well containing remaining 100 μL complete media and incubated for 20 minutes at room temperature. After fixation, the liquid was removed and the cells were washed in 200 μL of Wash Buffer (1% BSA, 0.2% Triton X-100, 1×PBS). Wash Buffer is replaced with 100 μL per well of Blocking Buffer (2 drops of horse serum (Vector Labs, Burlingame, Calif.) per 5 mL Wash Buffer) and incubated overnight at 4° C. The next day, cells were washed with 200 μL per well Wash Buffer. Cells are stained with anti-p-Histone H3 mouse antibody (0.5 μg/mL, 05-806, aka pH3 or p-HH3, Millipore) and anti-pericentrin rabbit antibody (0.5 μg/mL, ab4448-100, Abcam) in 100 μL Wash Buffer for 2 hours at room temperature. Cells were washed twice with 200 μL Wash Buffer. Cells were stained with Invitrogen goat anti-mouse IgG-alexa-647 (A21236) and goat anti-rabbit IgG-alexa-488 (A11034) at 1 μg/mL in Wash Buffer containing Hoechst 33342 DNA dye (2 μg/mL) for 2 hours at room temperature, in the dark. Cells were washed twice with 200 μL per well Wash Buffer. After the last wash, 150 μL of 1×PBS was added to each well and the plates were sealed (Perkin Elmer, Waltham, Mass.). Imaging data was acquired on Cellomics ArrayScan VTI HCS Reader (SN03090745F, ThermoFisher Scientific) using the SpotDetector.V4 Assay protocol (Ver 6.6.0 (Build 8153) with a 20×objective, experiment #1 collected 100-fields per well; experiment #2 collected 67-fields per well). First, acquired mitotic index data (percentage of p-Histone H3 positive objects) was acquired as described above. Next, a virtual scan was conducted with a channel swap (using p-Histone H3 positive objects as the primary object for segmentation, instead of the nuclear object) to enumerate the number of pericentrin spots for each mitotic object. The percentage of mitotic objects with >2 pericentrin spots (proxy for centrosome number) was determined for each well. The minimal number of p-Histone H3 positive objects was set at 250 objects per well for DMSO control. The data outputs include:
(1) Valid Object Count. This represents the total valid nuclear object count per well (based on Object.Area.Ch1 and Object.VarIntensity.Ch1 were used to set range for valid objects, objects outside this range were rejected).
(2) Selected Object Count, pHH3. This represents the total valid p-Histone H3 positive mitotic object count based on the set fluorescence intensity threshold using alexa-647 (channel 3).
(3) % Selected Object pHH3. This represents the percentage of p-Histone H3 positive objects [(Selected Object Count, pHH3+Valid Object Count)×100].
(4) % HIGH_ObjectSpotTotalCountCh2.

The percentage of p-Histone H3 positive mitotic objects for each KIF18A inhibitor concentration was plotted using GraphPad Prism software (V7.0.4) and concentration-response curves were fitted using 4-parameter equation (variable slope). The mean EC50 value and standard deviation were determined from two independent experiments run in duplicate.

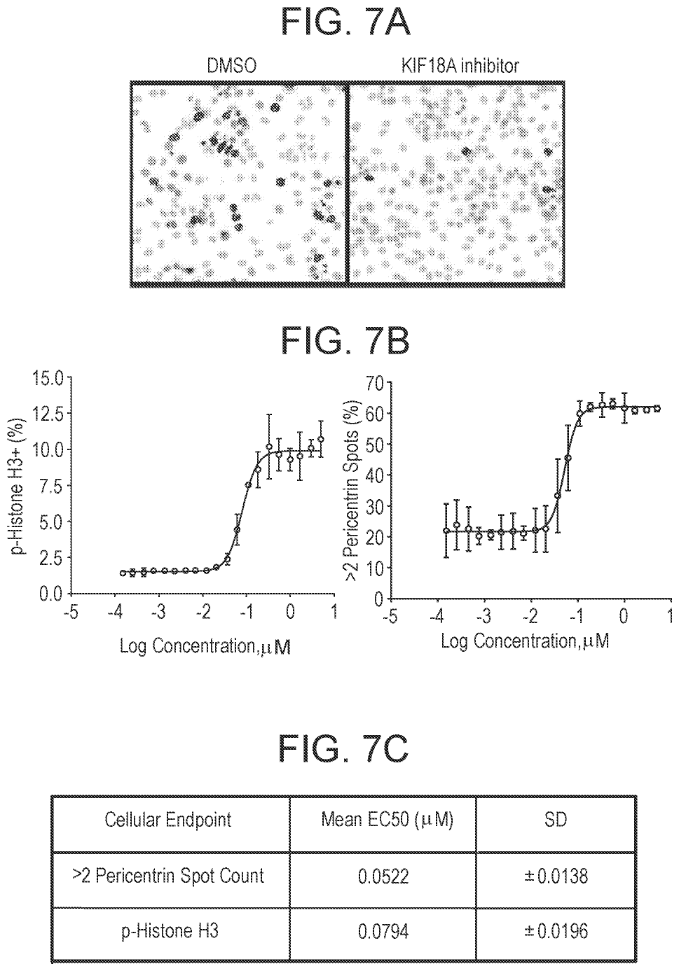

Representative field-level images of DMSO- and KIF18A inhibitor-treated cells are provided in FIG. 7A. KIF18A inhibitor concentration-response fitted-curves showing the mean percentage of mitotic objects with >2 pericentrin spots or the mean percentage of p-Histone H3 positive objects are provided in FIG. 7B. Error bars (SD) are shown. The mean EC50 values for pericentrin spot count and p-Histone H3 are shown in the table of FIG. 7C.

The black objects in FIG. 7A represent p-Histone H3 positive mitotic cells. Enumerated grey spots (pericentrin positive) for each p-Histone H3 positive mitotic cell. As shown in FIGS. 7A to 7C, treatment with KIF18A inhibitor activates the spindle assembly checkpoint (SAC) in mitosis, measured by the increase in p-Histone H3 positive cells with an EC50 value of 0.0794 μM. KIF18A inhibitor induced a concentration-dependent increase in pericentrin spotting (>2 spots per mitotic object) with EC50 value 0.0522 μM was similar to p-Histone H3 assay EC50 value suggesting these mitotic phenotypes are likely coupled in MDA-MB-157 cells. Together, these data suggest that inhibition of KIF18A ATPase motor activity with KIF18A small molecule inhibitors leads to mitotic cell arrest and excessive pericentrin spotting.

### Example 6

This example describes a study to analyze the effects of a KIF18A inhibitor on mitotic centrosome/chromosome features and apoptosis of cancer cells.

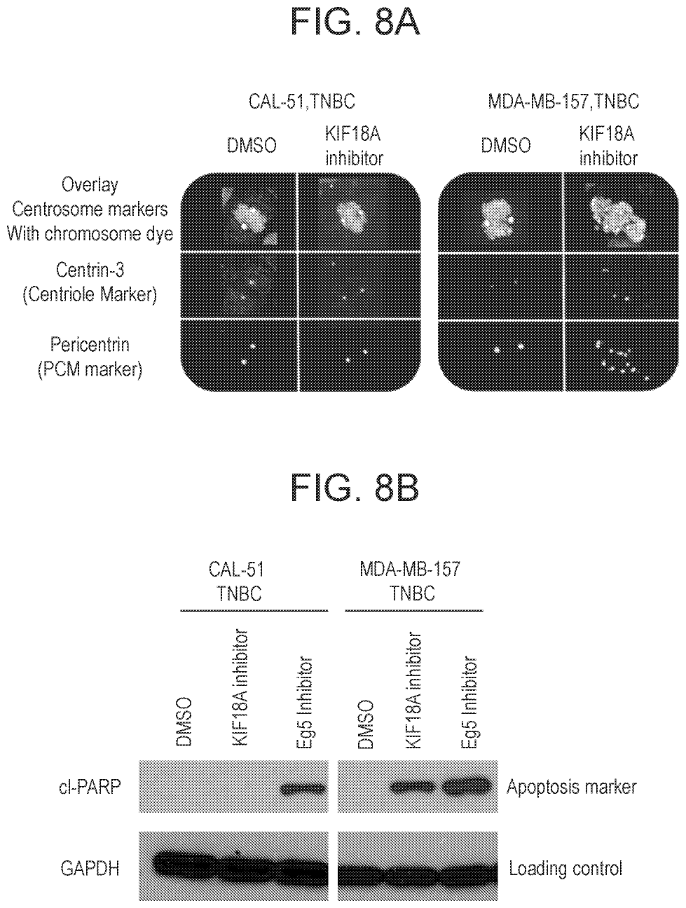

To analyze the effects of a KIF18A inhibitor on mitotic centrosome/chromosome features and apoptosis of cancer cells, centrosome features in CAL-51 and MDA-MB-157 TNBC cells were analyzed by an immunofluorescence imaging analysis. Cells were seeded into Cell Carrier Ultra 96-well Polystyrene plate (PerkinElmer) in 200 μL of complete media and grown. After 24 hours, cells were treated for 24 hours with DMSO (0.05%) or KIF18A inhibitor Compound C11 (0.5 μM). Cells were fixed with 2% formaldehyde for 20 minutes at room temperature followed by permeabilization in 1×PBS with 0.1% Triton X-100 for 20 minutes at room temperature. Cells were washed twice with 200 μL Wash Buffer (1×PBS/0.5% BSA, Rockland Immunochemicals). Cells are stained with 100 μL anti-CETN3 mouse antibody (1:2000, H00001070-M01, Abnova) and anti-pericentrin rabbit antibody (1:2000, ab4448-100, Abcam) in 200 μL of Wash Buffer and incubated overnight at 4° C. Cells were washed twice with 200 μL Wash Buffer. Cells were stained with 100 μL secondary antibodies [goat anti-mouse IgG-alexa-488 (1:1000, A1 1029, Invitrogen) and goat anti-rabbit IgG-alexa-647 (1:1000, A21244, Invitrogen) in Wash Buffer for 2 hours at room temperature protected for light. Cells were washed twice followed by addition of 100 μL Wash Buffer containing Hoechst 33342 DNA dye (2 μg/mL). Cells were imaged on a PerkinElmer Ultraview Vox dual spinning disc confocal microscope using a 60× oil immersion objective with laser excitation wavelengths of 405 nm (Hoechst), 488 nm (alexa-488), and 647 nm (alexa-647). Representative maximal projection images were collected for mitotic objects from each treatment well. The images are shown in FIG. 8A.

In a separate experiment, a Western blotting analysis was carried out. Briefly, CAL-51 and MDA-MB-157 TNBC cell lines were seeded into 6-well plates at a density of 125000, and 150 000, per well, respectively, in complete growth media. The next day, cells were treated with DMSO, KIF18A inhibitor (0.5 μM), or Eg5 inhibitor Ispinesib (0.05 μM) in 3 mL of complete media at a final DMSO concentration of 0.5%. After 48 hours, cell lysates were prepared for each treatment group (combined media and cells from 3 wells). Cells were lysed and Western blotting was carried out as essentially described in Example 5. Primary antibodies included mouse anti-cleaved-PARP (cl-PARP) (#51-900017, BD Pharmingen, 1:500), mouse anti-cyclin B1 (554179, BD Pharmingen, 1:500), and rabbit anti-GAPDH (2118, Cell Signaling, 1:10,000). The results are shown in FIG. 8B.

As shown in FIGS. 8A and 8B, treatment of TNBC cell lines with a KIF18A inhibitor selectively induced alterations in mitotic cell centrosome features (pericentriolar material and centriole numerical changes and fragmentation) and apoptosis only in MDA-MB-157 TP53 mutant and CCNE1 amplified cells, whereas the CAL-51 TP53 wild-type cells showed no changes in centrosome features or apoptosis relative to DMSO treated cells. Together these data suggest that TP53 mutant TNBC cells are dependent on KIF18A motor activity for proper chromosome alignment and segregation, and that KIF18A inhibition leads to SAC activation and/or aberrate centrosome features resulting in multipolar spindles and apoptosis.

### Example 7

This example demonstrates a time course study of cell cycle and apoptosis protein expression in HGSOC cells treated with a KIF18A inhibitor.

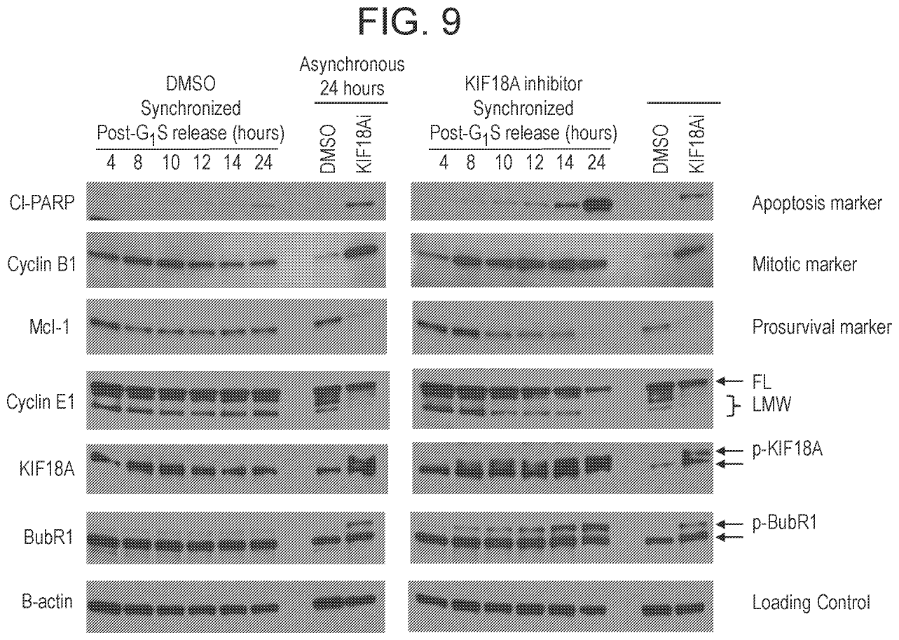

OVCAR-3 HGSOC cells were seeded at a density of 1.4 million cells into 100 mm tissue culture plates in 10 mL of complete growth media. The next day, complete growth media containing 2 mM thymidine was added to the cells and incubated for 16 hours. Cells were washed thrice in 1×PBS before adding complete growth media for 8 hours, followed by and second 2 mM thymidine block for 16 hours. The double thymidine block arrested cells in G1/S phase of the cell cycle. Cells were release from G1/S block by first washing thrice in 1×PBS before adding complete growth media with DMSO or KIF18A inhibitor (Compound C11 at 0.5 μM). Cell lysates were prepared at multiple time points (4, 8, 10, 12, 14, and 24 hours). As controls, asynchronous growing OVCAR-3 cells were treated DMSO or KIF18A inhibitor (Compound C11 at 0.5 μM) and lysates were prepared at 24 hours. Primary antibodies included mouse anti-cleaved-PARP (cl-PARP) (51-900017, BD Pharmingen, 1:500), mouse anti-cyclin B1 (554179, BD Pharmingen, 1:500), rabbit anti-Mcl-1 (5453, Cell Signaling, 1:500), mouse anti-Cyclin E1 (MS-870-P, HE12, NeoMarkers, 1:2000), mouse anti-BubR1 (612503, BD Pharmingen, 1:5000), and rabbit anti-KIF18A (HPA039484, Simga, 1:2000), and mouse anti-β-actin (A5441, Simga, 1:5000). The results are shown in FIG. 9.

An image of the blot is shown in FIG. 9. As shown in FIG. 9, the KIF18A inhibitor-treated cells showed an increase in cyclin B1 and cl-PARP protein levels and a decrease in Mcl-1 and cyclin E1 levels. Also, an increase in KIF18A and BubR1 protein levels are shown. In FIG. 9, “FL” refers to the full length Cyclin E1 protein and “LMW” refers to the low molecular weight form of Cyclin E1. The BubR1 protein blots as a protein doublet due to post translational modification, e.g. phosphorylated, forms and un-modified forms.

The results suggest that KIF18A inhibitor treatment of OVCAR-3 HGSOC cells show marked changes in proteins that regulate cell cycle and mitotic progression (cyclin B1, cyclin E1, BubR1, KIF18A) and apoptosis (Mcl-1, cl-PARP), these changes could serve as markers of target engagement in KIF18A inhibitor sensitive cancers.

### Example 8

This example demonstrates the effects on mitosis, DNA damage and apoptosis in TNBC cells (TP53 mutant, RB1 deficient) treated with two KIF18A inhibitors.

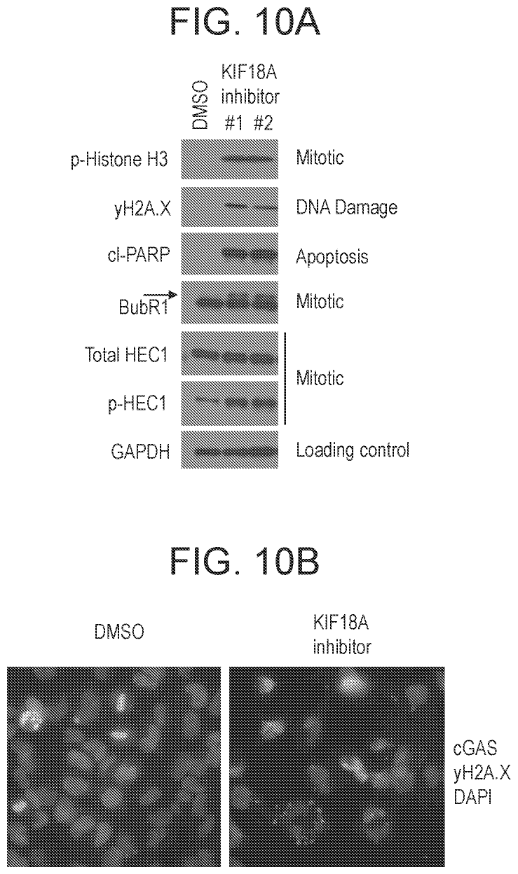

In order to analyze the effects on mitosis, BT-549 TNBC cells were seeded into 6-well plates at a density of 100,000 per well in 3 replicate wells in 4 mL of complete growth media. The next day, cells were treated with DMSO, KIF18A inhibitor #1 (Compound C11, 0.5 μM), or KIF18A inhibitor #2 (Compound C9, 0.01 μM) in 4 mL of complete media at a final DMSO concentration of 0.5%. After 48 hours, cell lysates were prepared for each treatment group (combined media and cells from 3 wells) using Minute™ Total Protein Extraction kit (SD-001, Invent Biotechnologies) lysis conditions according to manufactures protocol, supplemented with protease inhibitor cocktail (cOmplete™, Roche) and phospatase inhibitors (PhosphoStop, Roche). Primary antibodies included rabbit anti-p-Histone H3 (serine-10) (06-570, Millipore, 1: 2000), mouse anti-γH2A.X (serine-139) (05-636, Millipore, 1: 2000), mouse anti-cleaved-PARP (cl-PARP) (#51-900017, BD Pharmingen, 1:500), mouse anti-BubR1 (612503, BD Pharmingen, 1:5000), mouse anti-total HEC1 (ab3613, ABCAM, 1: 1000), rabbit anti-pHEC1(serine-55) (GTX70017, Genetex, 1: 500), and rabbit anti-GAPDH (2118, Cell Signaling, 1:10,000). An image of the Western blot is shown in FIG. 10A.

Also, cGAS and γH2A.X (serine-139) immunostaining in BT-549 TNBC cells was carried out. Cells were seeded into Lab-Tek 2-well chamber slides at a density of 50,000 cells per chamber in complete growth media and grown for 2 days to approximately 50% confluency. Cells were treated for 48 hours with DMSO (0.1%), KIF18A inhibitor (Compound C9, 0.2 μM) in 2 mL of complete media. Cells were fixed in 4% formaldehyde for 15 minutes at room temperature followed by secondary fixation in ice-cold 90% methanol for 10 minutes at 4° C. After fixation, cells were washed in 2 mL of Wash Buffer (1% BSA, 0.2% Triton X-100, 1×PBS) and blocked 1 mL of Blocking Buffer (2 drops of horse serum (Vector Labs, Burlingame, Calif.) per 5 mL Wash Buffer). Cells are stained with anti-cGAS rabbit antibody (1:500, 15102, Cell Signaling) and anti-γH2A.X (serine-139) mouse antibody (1:1000, 05-636, Millipore) in 1 mL of Wash Buffer for 2 hours at room temperature. Cells were washed twice with 2 mL Wash Buffer. Cells were stained with secondary antibodies [goat anti-mouse IgG-alexa-488 (1:2000, A11029. Invitrogen) and goat anti-rabbit IgG-alexa-568 (1:2000, A11036, Invitrogen) in Wash Buffer containing DAPI DNA dye (1:5000, 268298, Millipore) for 1 hour at room temperature protected for light. Cells were washed twice with 2 mL and chambers were removed and a drop of ProLong Anti-Fade (P36934, Invitrogen) was added before mounting coverslips onto glass slides. Wide-field images were captured using 40×objective on upright Nikon Eclipse NI-E epi-fluorescence microscope running Elements software. Representative images taken with a 40×wide field objective are provided in FIG. 10B.

As shown in FIG. 10A, treatment of TP53 mutant and RB1 loss TNBC cells with a KIF18A inhibitors leads to increased expression of mitotic markers (p-Histone H3, p-HEC1, BubR1 doublet), DNA damage marker (γH2A.X), and apoptosis marker (cl-PARP), suggesting KIF18A inhibition leads to SAC activation resulting in an increase in DNA damage and apoptosis, these changes could serve as markers of target engagement in KIF18A inhibitor sensitive cancers.

As shown in FIG. 10B, cytoplasmic micronuclei stained positive for cGAS and/or γH2A.X suggesting that KIF18A inhibitor treatment leads to increase in DNA damage and cGAS positive micronuclei which may serve as a source of immunostimulatory cytoplasmic DNA.

### Example 9

This example demonstrates an experiment to analyze the effect of KIF18A protein localization in mitosis in cancer cells treated with a KIF18A inhibitor.

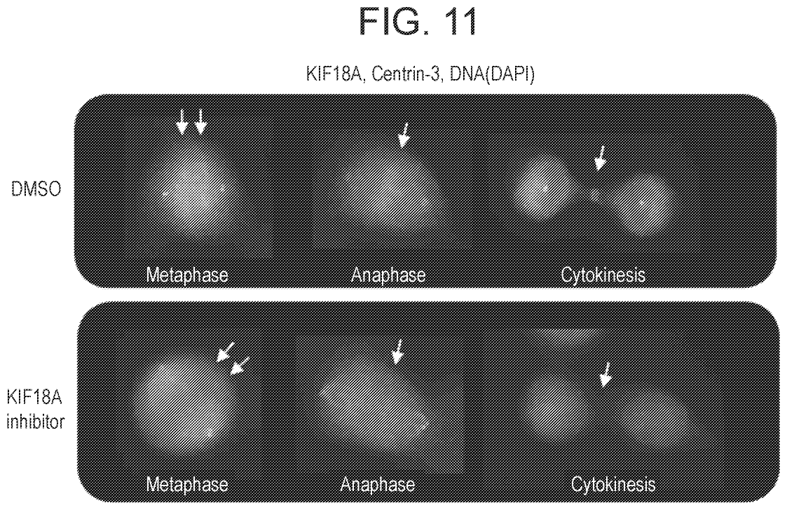

To analyze the effect of KIF18A protein localization in cancer cells treated with a KIF18A inhibitor, KIF18A and Centrin-3 immunostaining in HeLa cells (FIG. 11). Cells were seeded into Lab-Tek 2-well chamber slides at a density of 100,000 cells per chamber in complete growth media and grown for 2 days to approximately 80% confluency. Cells were treated for 6 hours with DMSO, KIF18A inhibitor (Compound C9, 0.05 μM) in 2 mL of complete media. Cells were fixed in 4% formaldehyde for 15 minutes at room temperature followed by secondary fixation in ice-cold 90% methanol for 10 minutes at 4° C. After fixation, cells were washed in 2 mL of Wash Buffer (1% BSA, 0.2% Triton X-100, 1×PBS) and blocked 1 mL of Blocking Buffer (2 drops of horse serum (Vector Labs, Burlingame, Calif.) per 5 mL Wash Buffer). Cells are stained with anti-KIF18A rabbit antibody (1:3000, A301-080A 05-806, Bethyl) and anti-CETN3 mouse antibody (1:1000, H00001070-M01, Abnova) in 1 mL of Wash Buffer for 2 hours at room temperature. Cells were washed twice with 2 mL Wash Buffer. Cells were stained with secondary antibodies [goat anti-mouse IgG-alexa-488 (1:2000, A11029, Invitrogen) and goat anti-rabbit IgG-alexa-568 (1:2000, A11036, Invitrogen) in Wash Buffer containing DAPI DNA dye (1:5000, 268298, Millipore) for 1 hour at room temperature protected for light. Cells were washed twice with 2 mL and chambers were removed and a drop of ProLong Anti-Fade (P36934, Invitrogen) was added before mounting coverslips onto glass slides. Wide-field images were captured using 40×objective on upright Nikon Eclipse NI-E epi-fluorescence microscope running Elements software. Representative images of immunostained mitotic cells are provided in FIG. 11. KIF18A is shown in red and centrin-3 is shown in green.

As shown in FIG. 11, KIF18A inhibitor treatment results in KIF18A protein mis-localization in mitosis, these changes in KIF18A protein localization could serve as marker of target engagement in cancers and surrogate proliferating normal tissues.

### Example 10

This example demonstrates Compound C14 induces p-Histone H3 mitotic marker in Human OVCAR-3 HGSOC tumor xenograft model (TP53^{MUT}, CCNE1^{AMP}) in Female Athymic Nude Mice.

Female athymic nude mice were injected with 5×10⁶ OVCAR-3 cells in 0.1 mL subcutaneously in the right flank. After tumors were established (average tumor volume of 450 to 750 mm³), animals were randomized into 5 treatment groups (vehicle alone, KIF18A inhibitor at 3, 10, 30, or 100 mg/kg) with 3 animals per group. Tumor and blood plasma were collected 24 hours post-treatment for pharmacokinetic analysis and tumor for pharmacodynamic analysis. Preparations of tumor protein lysates from snap frozen, pulverized, and lysed and processed using EpiQuik Total Histone Extraction Kit (OP-0006 Epigentek, Farmingdale, N.Y.) protocol. Protein lysate concentrations were determined using a BCA Protein Assay Kit (23227, Pierce, Rockford, Ill.). Lysates normalized for total protein per well were loaded onto Meso Scale Discovery (MSD) electrochemiluminescent immunoassay plates at 30 μg/well in lysis buffer a processed of pHH3 MSD analysis according to the manufacturer's protocol for the single-plex MSD assays using phospho-Histone H3 (serine-10) antibody (pHH3) and analyzed on a Sector Imager S16000 luminescence detection reader (MSD, Gaithersburg, Md.). Lysates were normalized for total protein and pHH3 fold induction represents the group average raw MSD value divided by the average raw MSD value of the vehicle treated group. Data was plotted using GraphPad Prism software (V7.0.4), the column graph shows the level of pHH3 signal represented as mean RU for each treatment group with standard error of the mean (SEM). The fold induction in pHH3 is noted for each KIF18A inhibitor treatment group relative to mean baseline pHH3 signal for vehicle group. The micromolar concentration of KIF18A inhibitor in plasma and tumor are indicated on the right vertical axis. Statistical significance was determined by one-way ANOVA followed by Dunnett's post hoc analysis (***p=0.0002, ****p≤0.0001).

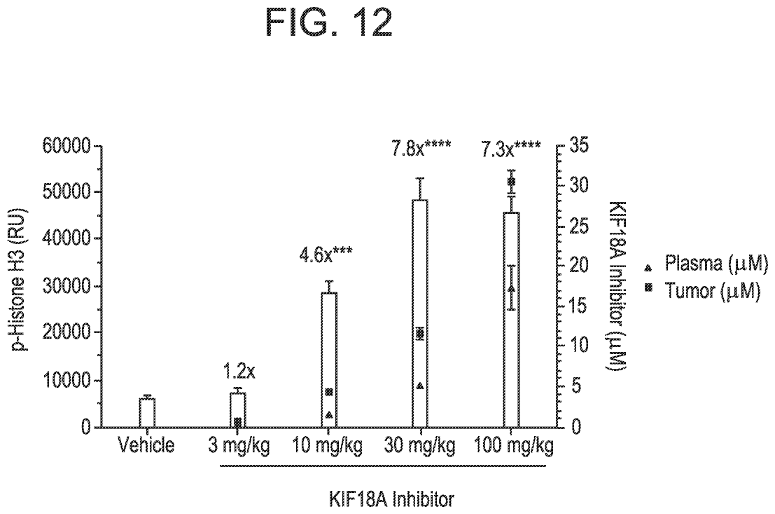

A graph of the luminescence representing p-Histone H3 level was plotted for each dose of KIF18A inhibitor (or vehicle control) is shown in FIG. 12. The graph shows the level of p-Histone H3 signal represented as mean RUs for each treatment group with SEM. The fold induction in p-Histone H3 is noted for each KIF18A inhibitor treatment group relative to mean baseline p-Histone signal for vehicle group. The concentration of KIF18A inhibitor (μM) in plasma (▴) and tumor (▪) are indicated on the right vertical axis. Statistical significance was determined by one-way ANOVA followed by Dunnett's post hoc analysis (***p=0.0002, ****p<0.0001).

As shown in FIG. 12, KIF18A inhibitor induced a dose-dependent increase in p-Histone H3 mitotic marker levels in the OVCAR-3 HGSOC (TP53^{MUT}, CCNE1^{AMP}) tumor xenografts in mice. These data suggest that increased levels of p-Histone H3 was dose- and exposure-dependent indicating p-Histone H3 is a suitable pharmacodynamic marker, where a ≥4.6-fold induction of p-Histone H3 signal correlated with tumor regressions at KIF18A inhibitor doses ≥10 mg/kg.

### Example 11

This example describes exemplary steps for making exemplified KIF18A inhibitors that can be used in the methods of the invention.

The following abbreviations may be used throughout this example:

| | |
|---|---|
| AcOH | acetic acid |
| aq or aq. | aqueous |
| BOC or Boc | tert-butyloxycarbonyl |
| DCE | 1,2-dichloroethane |
| DCM | DCM |
| DMAP | 4-dimethylaminopyridine |
| DMF | N,N-dimethylformamide |
| DMSO | dimethyl sulfoxide |
| ESL or ES | electrospray ionization |
| Et | ethyl |
| Et₂O | diethyl ether |
| EtOH | ethyl alcohol |
| EtOAc | EtOAc |
| G | grams |
| H | hour |
| HPLC | high pressure liquid chromatography |
| iPr | isopropyl |
| iPr₂NEt or DIPEA | N-ethyl diisopropylamine (Hünig’s base) |
| KOAc | potassium acetate |
| LAH | lithium aluminum hydride |
| LDA | lithium diisopropylamide |
| LC MS, LCMS, | liquid chromatography mass spectroscopy |
| LC-MS or LC/MS | |
| m/z | mass divided by charge |
| Me | methyl |
| MeOH | methanol |
| Mg | milligrams |
| Min | minutes |
| mL | milliliters |
| MS | mass spectra |
| NMR | nuclear magnetic resonance |
| RT or rt | room temperature |
| sat. or satd. | saturated |
| SFC | supercritical fluid chromatography |
| TEA or EtN | trimethylamine |
| TFA | trifluoroacetic acid |
| THF | tetrahydrofuran |
| Xantphos | 4,5-bis(diphenylphosphino)-9,9-dimethylxanthene |

Unless otherwise noted, all materials were obtained from commercial suppliers and used without further purification. All parts are by weight and temperatures are in degrees centigrade unless otherwise indicated. All microwave assisted reactions were conducted with a Smith Synthesizer™ from Biotage™. All compounds showed NMR spectra consistent with their assigned structures. Melting points were determined on a Buchi apparatus and are uncorrected. Mass spectral data was determined by electrospray ionization technique. All examples were purified to >90% purity as determined by high-performance liquid chromatography. Unless otherwise stated, reactions were run at room temperature.

In synthesizing compounds of the present invention, it may be desirable to use certain leaving groups. The term “leaving groups” (“LG”) generally refer to groups that are displaceable by a nucleophile. Such leaving groups are known in the art. Examples of leaving groups include, but are not limited to, halides (e.g., I, Br, F, Cl), sulfonates (e.g., mesylate, tosylate), sulfides (e.g., SCH₃), N-hydroxysuccinimide, N-hydroxybenzotriazole, and the like. Examples of nucleophiles include, but are not limited to, amines, thiols, alcohols, Grignard reagents, anionic species (e.g., alkoxides, amides, carbanions) and the like.

The examples presented below illustrate specific embodiments of the present invention. These examples are meant to be representative and are not intended to limit the scope of the claims in any manner.

It is noted that when a percent (%) is used with regard to a liquid, it is a percent by volume with respect to the solution. When used with a solid, it is the percent with regard to the solid composition. Materials obtained from commercial suppliers were typically used without further purification. Reactions involving air or moisture sensitive reagents were typically performed under a nitrogen or argon atmosphere. Purity was measured using high performance liquid chromatography (HPLC) system with UV detection at 254 nm and 215 nm (System A: Agilent Zorbax Eclipse XDB-C8 4.6×150 mm, 5 μm, 5 to 100% CH₃CN in H₂O with 0.1% TFA for 15 min at 1.5 mL/min; System B: Zorbax SB-C8, 4.6×75 mm, 10 to 90% CH₃CN in H₂O with 0.1% formic acid for 12 min at 1.0 mL/min) (Agilent Technologies, Santa Clara, Calif.). Silica gel chromatography was generally performed with prepacked silica gel cartridges (Biotage, Uppsala, Sweden or Teledyne-Isco, Lincoln, Nebr.). ¹H NMR spectra were recorded on a Bruker AV-400 (400 MHz) spectrometer (Bruker Corporation, Madison, Wis.) or a Varian (Agilent Technologies, Santa Clara, Calif.) 400 MHz spectrometer at ambient temperature. All observed protons are reported as parts per million (ppm) downfield from tetramethylsilane (TMS) or other internal reference in the appropriate solvent indicated. Data are reported as follows: chemical shift, multiplicity (s=singlet, d=doublet, t=triplet, q=quartet, br=broad, m=multiplet), coupling constants, and number of protons. Low-resolution mass spectral (MS) data were determined on an Agilent 1100 Series (Agilent Technologies, Santa Clara, Calif.) LC/MS with UV detection at 254 nm and 215 nm and a low resonance electrospray mode (ESI).

Preparation of Intermediate Compounds Intermediates 1-13:

### Intermediate 1: 2-(4,4-Difluoropiperidin-1-yl)-6-methylpyrimidin-4-amine

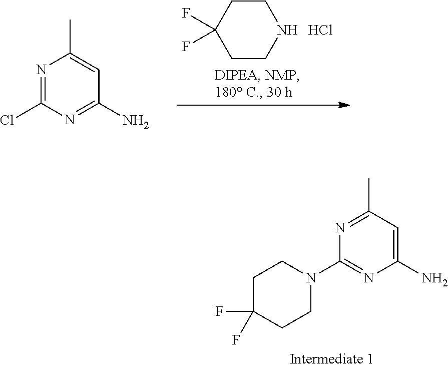

A mixture of 2-chloro-6-methylpyrimidin-4-amine (46 g, 320 mmol, Combi-Blocks, San Diego, Calif.), 4,4-difluoropiperidine hydrochloride (76 g, 481 mmol, Combi-Blocks, San Diego, Calif.) and DIPEA (166 mL, 961 mmol) in NMP (460 mL, 10.00 mL/g) was taken in an autoclave (1 L) and heated at 180° C. for 30 h. The reaction mixture was cooled to room temperature and quenched with water (500 mL), extracted with ethyl acetate (2×1000 mL). The organic layer was washed with brine (500 mL), dried (Na₂SO₄), filtered and concentrated under reduced pressure. The crude material was adsorbed onto a plug of silica gel and purified by column chromatography over silica gel (60-120 mesh), eluting with 50% to 100% E in hexanes as an eluent to give the product. This was re-dissolved in ethyl acetate (500 mL), washed with water (2×500 mL). The organic layer was dried (Na₂SO₄), filtered and concentrated under reduced pressure. The yellow solid was once again suspended in hexanes (400 mL) and stirred for 30 min. The slurry was filtered, washed with hexanes (100 mL), dried under vacuum to provide the title compound (58 g, 79% yield) as a pale yellow solid. ¹H NMR (400 MHz, DMSO-de) 6 ppm 6.33 (s, 2H), 5.63 (s, 1H), 3.80-3.78 (dd, J=6.8, 4.7 Hz, 4H), 2.06 (s, 3H), 1.95-1.85 (tt, J=14.2, 5.7 Hz, 4H). m/z (ESI): 229.2 (M+H)⁺.

### Intermediate 2: 6-(3,3,3-Trifluoropropoxy)pyridin-2-amine

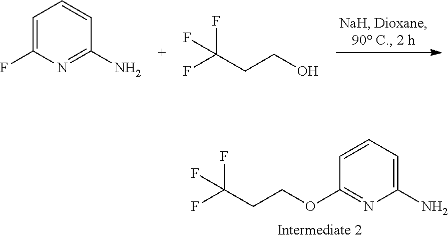 [SMILES: Nc1cccc(F)n1 ; OCCC(F)(F)F ; Nc1cccc(OCCC(F)(F)F)n1]

To a solution of 6-fluoropyridin-2-amine (50 g, 450 mmol, Combi-Blocks) in 1,4-dioxane (500 mL) was added 3,3,3-trifluoropropan-1-ol (102 g, 892 mmol, Apollo) under nitrogen atmosphere and the reaction was cooled to 0° C. NaH (60% in mineral oil, 42.8 g, 1780 mmol) was added to the reaction mixture at 0° C. and the resulting mixture was stirred at 90° C. for 2 h. The reaction mixture was quenched with cold water (500 mL) and extracted with ethyl acetate (2×1000 mL). The combined organic extracts were dried (Na₂SO₄), filtered, and concentrated under reduced pressure. The crude residue was purified by column chromatography over silica gel (60-120 mesh) using 10% ethyl acetate in hexanes to give the title compound (45 g, 50% yield) as a pale brown oil. ¹H NMR (400 MHz, DMSO-d₆): δ 7.30-7.26 (t, J=7.8 Hz, 1H), 6.02-6.00 (dd. J=7.8, 0.8 Hz, 1H), 5.89-5.86 (m, 3H), 4.36-4.33 (t, J=6.2 Hz, 2H), 2.79-2.67 (qt, J=11.5, 6.2 Hz, 2H). m/z (ESI): 207.1 (M+H)+.

### Intermediate 3: 6-(4,4-Difluoropiperidin-1-yl)-4-methylpyridin-2-amine

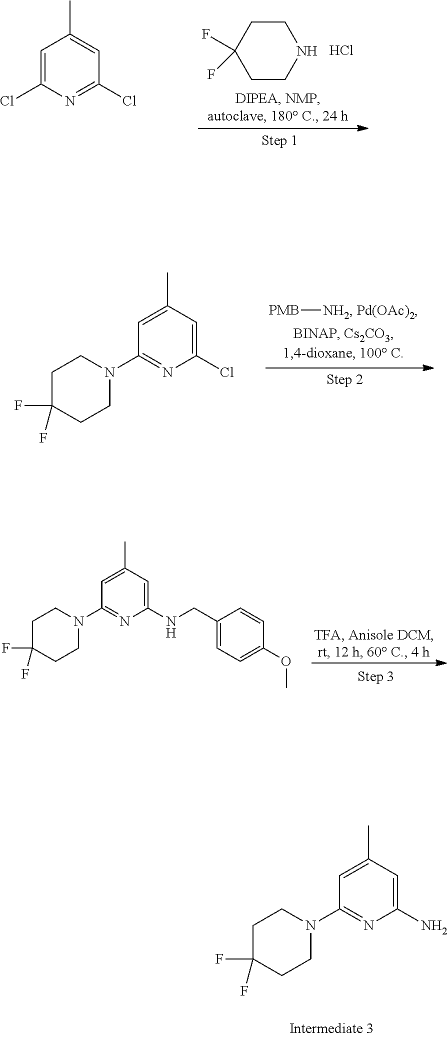

Step 1: To an autoclave was added 2,6-dichloro-4-methylpyridine (80 g, 490 mmol), 4,4-difluoropiperidine hydrochloride (86 g, 540 mmol), and DIPEA (342 mL, 1980 mmol) in NMP (800 mL). The reaction mixture was heated at 180° C. for 24 h. The reaction mixture was cooled to room temperature and basified to pH-9 using 10% aqueous NaHCO₃ solution. The reaction mixture was extracted with ethyl acetate (2×1500 mL), washed with water (1500 mL), dried (Na₂SO₄), filtered, and concentrated under reduced pressure. The crude material was purified by column chromatography over silica gel (60-120 mesh) using 5-10% ethyl acetate in hexanes to give the mixture of 2,6-dichloro-4-methylpyridine and 2-chloro-6-(4,4-difluoropiperidin-1-yl)-4-methylpyridine in 1:3 ratio (102 g) as a pale brown oil. This mixture (102 g) was further purified by reverse phase chromatography using 60% acetonitrile in water as an eluent to give 2-chloro-6-(4,4-difluoropiperidin-1-yl)-4-methylpyridine (70 g, 58% yield) as a pale brown liquid. ¹H NMR (400 MHz, DMSO-d₆): δ 6.76 (s, 1H), 6.57 (s, 1H), 3.66 (t, J=5.6 Hz, 4H), 2.22 (s, 3H), 2.03-1.91 (m, 4H). m/z (ESI): 247.1 (M+H)+.

Step 2: To a solution of 2-chloro-6-(4,4-difluoropiperidin-1-yl)-4-methylpyridine (30.0 g, 122 mmol) in 1,4-dioxane (300 mL) were added (4-methoxyphenyl)methanamine (23.8 mL, 182 mmol) and Cs₂CO₃ (79 g, 240 mmol). The reaction mixture was degassed and purged with nitrogen for 30 min. BINAP (7.57 g, 12.2 mmol) and palladium(II)acetate (2.73 g, 12.2 mmol), were added to the reaction mixture and stirred at 100° C. for 16 h. The reaction mixture was cooled to room temperature, filtered through a CELITE® bed, and washed with ethyl acetate (100 mL). The filtrate was concentrated under reduced pressure. The residue was extracted with EtOAc (2×500 mL), washed with water (500 mL) followed by brine (500 mL). The combined organic extracts were dried (Na₂SO₄), filtered, and concentrated under reduced pressure. The crude residue was purified by column chromatography over silica gel (60-120 mesh) using 5-8% ethyl acetate in hexanes to give 6-(4,4-difluoropiperidin-1-yl)-N-(4-methoxybenzyl)-4-methylpyridin-2-amine (48 g, 76% yield) as a yellow oil. ¹H NMR (400 MHz, DMSO-d₆): δ 7.22 (d, J=7.2 Hz, 2H), 6.85 (d, J=7.2 Hz, 2H), 6.64 (t, J=6.0 Hz, 1H), 5.84 (s, 1H), 5.68 (s, 1H), 4.31 (d, J=6.0 Hz, 2H), 3.71 (s, 3H), 3.56 (t, J=5.6 Hz, 4H), 2.05 (s, 3H), 1.90-1.80 (m, 4H). m/z (ESI): 348.1 (M+H)⁺.

Step 3: To a solution of 6-(4,4-difluoropiperidin-1-yl)-N-(4-methoxybenzyl)-4-methylpyridin-2-amine (48.0 g, 138 mmol) in dry dichloromethane (480 mL) were added anisole (30.2 mL, 276 mmol) and TFA (240 mL, 3120 mmol). The reaction mixture was stirred at 55° C. for 4 h and concentrated under reduced pressure. The residue was dissolved in water (200 mL) and basified with 10% aqueous sodium bicarbonate solution to pH-8 and extracted with ethyl acetate (2×500 mL). The combined organic layers were washed with water (200 mL) followed by brine (200 mL), dried (Na₂SO₄), filtered, and concentrated under reduced pressure. The crude residue was purified by column chromatography over silica gel using 25% to 35% ethyl acetate in hexanes to give 6-(4,4-difluoropiperidin-1-yl)-4-methylpyridin-2-amine (LCMS ˜85%) as a brown oil. This material was further purified by reverse phase chromatography using 50-60% acetonitrile in water to give 6-(4,4-difluoropiperidin-1-yl)-4-methylpyridin-2-amine (16.5 g, 72 mmol, 53% yield) as a brown oil. ¹H NMR (400 MHz, DMSO-d₆): δ 5.86 (s, 1H), 5.65 (s, 1H), 5.48 (s, 2H), 3.56 (t, J=5.2 Hz, 4H), 2.06 (s, 3H), 1.96-1.87 (m, 4H). m/z (ESI): 228.2 (M+H)⁺.

### Intermediate 4:3-(4,4-Difluoropiperidin-1-yl)-5-methylaniline

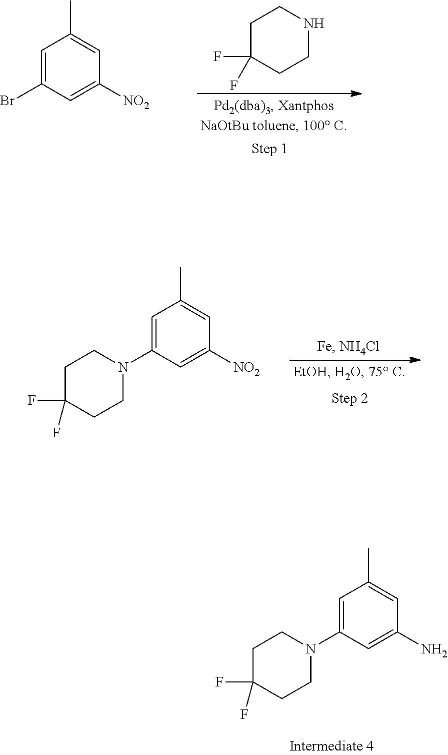 [SMILES: Cc1cc(Br)cc([N+](=O)[O-])c1 ; FC1(F)CCNCC1 ; Cc1cc(N2CCC(F)(F)CC2)cc([N+](=O)[O-])c1 ; Cc1cc(N)cc(N2CCC(F)(F)CC2)c1]

Step 1: A mixture of 1-bromo-3-methyl-5-nitrobenzene (5 g, 23.14 mmol), 4,4-difluoropiperidine (4.21 g, 34.7 mmol), sodium tert-butoxide (6.67 g, 69.4 mmol), Pd₂(dba)₃ (2.12 g, 2.31 mmol) and xantphos (1.34 g, 2.31 mmol) in toluene (50 mL) was stirred at 100° C. for 1.5 h. The reaction mixture was diluted with water and extracted with EtOAc. The organic extract was washed with brine, dried over Na₂SO₄, filtered, concentrated, and purified by silica gel column chromatography using 10% EtOAc in petroleum ether to provide 4,4-difluoro-1-(3-methyl-5-nitrophenyl)piperidine (3.70 g, 14.44 mmol, 62% yield) as a grey solid. ¹H NMR (400 MHz, DMSO-d₆): δ ppm 7.55 (t, J=2.3 Hz, 1H), 7.45 (s, 1H), 7.32 (d, J=2.3 Hz, 1H), 3.46 (t, J=5.8 Hz, 4H), 2.38 (s, 3H), 1.96-2.04 (m, 4H). m/z (ESI): 257.1 (M+H)⁺.

Step 2: A mixture of 4,4-difluoro-1-(3-methyl-5-nitrophenyl)piperidine (3.7 g, 14.44 mmol), iron powder (8.06 g, 144 mmol) and ammonium chloride (7.72 g, 144 mmol) in EtOH (30 mL) and water (7 mL) was stirred at 75° C. for 16 h. The reaction mixture was filtered through a CELITE® pad, washed with methanol, and the filtrate was concentrated. The residue was diluted with water and extracted with EtOAc. The organic extract was washed with brine, dried over Na₂SO₄, filtered, concentrated, and purified by silica gel column chromatography eluting with 30-40% EtOAc in petroleum ether to provide 3-(4,4-difluoropiperidin-1-yl)-5-methylaniline (2.6 g, 11.49 mmol, 80% yield) as a brown solid. ¹H NMR (400 MHz, DMSO-d₆): δ ppm 6.00 (s, 2H), 5.89 (s, 1H), 4.81 (s, 2H), 3.16-3.22 (m, 4H), 2.09 (s, 3H), 1.94-2.04 (m, 4H). m/z (ESI): 227.1 (M+H)⁺.

### Intermediate 5: 4-Methyl-6-morpholinopyridin-2-amine

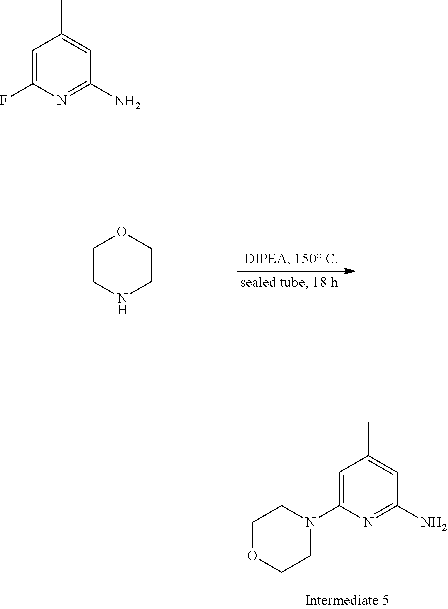

To a 250-mL pressure tube were added 6-fluoro-4-methylpyridin-2-amine (10.0 g, 79 mmol, Sibian chemicals, China), morpholine (8.29 g, 95 mmol), and DIPEA (41.5 mL, 238 mmol). The mixture was heated at 150° C. for 18 h. The reaction mixture was quenched with water (100 mL) and extracted with EtOAc (2×250 mL). The combined organic extracts were washed with brine (200 mL), dried over anhydrous Na₂SO₄, filtered, and concentrated under reduced pressure. The crude residue was absorbed onto a plug of silica gel and purified by flash chromatography through a Redi-Sep pre-packed silica gel column (40 g), eluting with a gradient of 1% to 15% EtOAc in hexanes, to give the title compound (8.5 g, 44.0 mmol, 56% yield) as a brown semisolid. ¹H NMR (400 MHz, DMSO-d₆) δ 5.75 (s, 1H), 5.67 (s, 1H), 5.44 (s, 2H), 3.65 (t, J=8.4 Hz, 4H), 3.30 (t, J=8.4 Hz, 4H), 2.06 (s, 3H). m/z (ESI): 194.2 (M+H)⁺.

### Intermediate 6: 4-Iodo-2-(6-azaspiro[2.5]octan-6-yl)benzoic acid

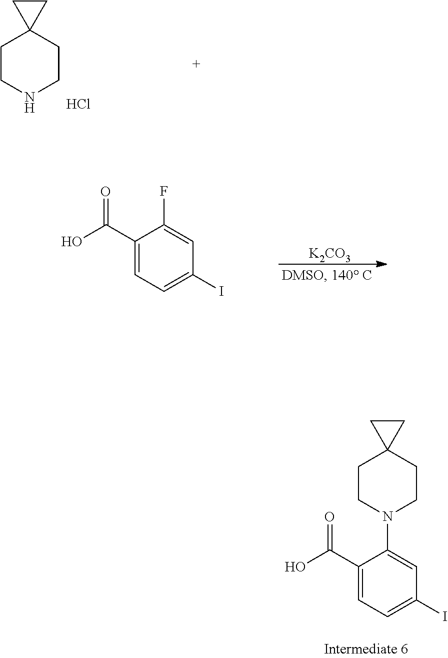 [SMILES: C1CC2(CCN1)CC2 ; Cl ; O=C(O)c1ccc(I)cc1F ; "K2CO3" ; O=C(O)c1ccc(I)cc1N1CCC2(CC1)CC2]

To a solution of 2-fluoro-4-iodobenzoic acid (300 g, 1.13 mol, Combi-Blocks, San Diego, Calif.) in DMSO (2.1 L) was added 6-azaspiro[2.5]octane hydrochloride (216 g, 1.47 mol, Wuxi AppTec) at 20° C. Then K₂CO₃ (468 g, 3.38 mol) was added and the reaction solution was stirred at 140° C. for 48 hours under N₂. The reaction solution was slowly poured into ice water (4.20 L), then extracted with hexanes (2 L×3). The water phase was separated and adjusted to pH=6 with HCl (2 M). Solid was precipitated out and collected. The solid was washed with water (700 mL×3) and filtered. The moist solid was spread out on a large watch glass and dried in the air at 25° C. for 72 hours. 4-Iodo-2-(6-azaspiro[2.5]octan-6-yl)benzoic acid (280 g, 777 mmol, 69% yield) was obtained as a light yellow solid. 400 MHz DMSO-de 6 ppm 8.07 (s, 1H), 7.76-7.66 (m, 2H), 3.10 (t, J=5.2 Hz, 4H), 1.55 (br s, 4H), 0.41 (s, 4H).

### Intermediate 7: 4-((3-Methyloxetan-3-yl)sulfonyl)-2-(6-azaspiro[2.5]octan-6-yl)benzoic acid

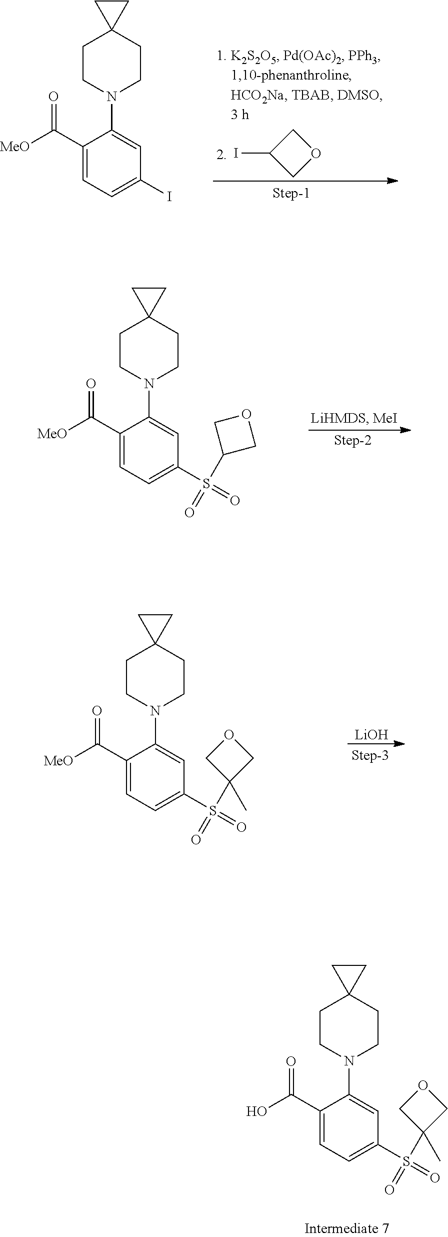 [SMILES: COC(=O)c1ccc(I)cc1N1CCC2(CC1)CC2 ; IC1COC1 ; COC(=O)c1ccc(S(=O)(=O)C2COC2)cc1N1CCC2(CC1)CC2 ; COC(=O)c1ccc(S(=O)(=O)C2(C)COC2)cc1N1CCC2(CC1)CC2 ; "LiOH" ; CC1(S(=O)(=O)c2ccc(C(=O)O)c(N3CCC4(CC3)CC4)c2)COC1]

Step 1: In a glass microwave reaction vessel (20 mL) a solution of methyl 4-iodo-2-(6-azaspiro[2.5]octan-6-yl)benzoate (2.0 g, 5.39 mmol, obtained in a similar way as described for Int. 7) in DMSO (15.0 mL) were added potassium metabisulfite (2.40 g, 10.78 mmol), TBAB (1.91 g, 5.93 mmol), sodium formate (0.81 g, 11.85 mmol), triphenyl phosphine (0.212 g, 0.81 mmol), 1,10-phenanthroline (0.146 g, 0.81 mmol) and palladium acetate (0.060 g, 0.27 mmol) under nitrogen atmosphere. The reaction mixture was degassed and purged with nitrogen for 10 min. The reaction vessel was sealed and heated at 70° C. for 3 h. The reaction mixture was cooled to RT and 3-iodooxetane (2.39 g, 12.97 mmol) was added and stirred at 120° C. for 4 h. The reaction mixture was quenched with water (100 mL) and extracted with EtOAc (2×100 mL). The combined organic extracts were washed with brine (100 mL), dried over Na₂SO₄, filtered, and concentrated under reduced pressure. The crude residue was adsorbed onto a plug of silica gel (60-120 mesh) and purified by silica gel chromatography through a Redi-Sep pre-packed silica gel column (40 g), eluting with a gradient of 1-40% EtOAc in hexanes to give methyl 4-(oxetan-3-ylsulfonyl)-2-(6-azaspiro[2.5]octan-6-yl)benzoate (360 mg, 15% yield) as a yellow solid. ¹H NMR (400 MHz, Chloroform-d): δ 7.79 (dd, J=8.1, 1.6 Hz, 1H), 7.51 (d, J=1.8 Hz, 1H), 7.38 (dd, J=8.0, 1.8 Hz, 1H), 4.98 (dd, J=7.4, 6.2 Hz, 2H), 4.80 (dd, J=8.4, 7.1 Hz, 2H), 4.45 (tt, J=8.4, 6.2 Hz, 1H), 3.94 (s, 3H), 3.22-3.10 (m, 4H), 1.52 (t, J=5.2 Hz, 4H), 0.38 (s, 4H). m/z (ESI): 366.1 [M+1].

Step 2: To a solution of methyl 4-(oxetan-3-ylsulfonyl)-2-(6-azaspiro[2.5]octan-6-yl)benzoate (350 mg, 0.96 mmol) in THF (5 mL) was added LiHMDS (1.0 M solution in hexanes, 1.92 mL, 1.91 mmol) at −78° C. under nitrogen atmosphere and stirred for 1 h. Iodomethane (71.9 μL, 1.15 mmol) was added slowly to the reaction mixture and slowly warmed to RT. The reaction mixture was quenched with satd. aqueous NH₄Cl solution (25 mL) and extracted with EtOAc (2×50 mL). The combined organic extracts were washed with brine (50 mL), dried over Na₂SO₄, filtered, and concentrated under reduced pressure. The crude residue was adsorbed onto a plug of silica gel (60-120 mesh) and purified by silica gel chromatography through a Redi-Sep pre-packed silica gel column (12 g), eluting with a gradient of 1-50% EtOAc in hexanes, to give methyl 4-((3-methyloxetan-3-yl)sulfonyl)-2-(6-azaspiro[2.5]octan-6-yl)benzoate (260 mg, 72% yield) as a pale yellow solid. ¹H NMR (400 MHz, Chloroform-d): δ 7.82 (d, J=8.0 Hz, 1H), 7.53 (d, J=1.6 Hz, 1H), 7.41 (dd, J=8.0, 1.6 Hz, 1H), 5.20 (d, J=6.9 Hz, 2H), 4.43 (d, J=6.9 Hz, 2H), 3.97 (s, 3H), 3.24-3.12 (m, 4H), 1.70 (s, 3H), 1.58 (t, J=5.4 Hz, 4H), 0.40 (s, 4H). m/z (ESI): 380.2 [M+1].

Step 3: To a solution of methyl 4-((3-methyloxetan-3-yl)sulfonyl)-2-(6-azaspiro[2.5]octan-6-yl)benzoate (250 mg, 0.66 mmol) in THF (5 mL), water (5 mL) and methanol (1 mL) was added lithium hydroxide (63 mg, 2.64 mmol) and stirred at RT for 5 h. The reaction mixture was acidified with 1.5N HCl pH ˜4. The aqueous layer was extracted with EtOAc (3×50 mL), washed with brine (25 mL), dried over Na₂SO₄, filtered, and concentrated under reduced pressure to give 4-((3-methyloxetan-3-yl)sulfonyl)-2-(6-azaspiro[2.5]octan-6-yl)benzoic acid (200 mg, 83% yield) as an off-white solid. ¹H NMR (400 MHz, DMSO-d₆): δ 16.01 (s, 1H), 8.05 (d, J=8.1 Hz, 1H), 7.91 (d, J=1.7 Hz, 1H), 7.72 (dd, J=8.0, 1.8 Hz, 1H), 5.01 (d, J=7.4 Hz, 2H), 4.48 (d, J=7.4 Hz, 2H), 3.19 (t, J=5.2 Hz, 4H), 1.60-1.52 (m, 7H), 0.41 (s, 4H). m/z (ESI): 366.2 [M+1].

### Intermediate 8: 6-(4,4-Dimethyl-2-oxooxazolidin-3-yl)-2-(6-azaspiro[2.5]octan-6-yl)nicotinic acid

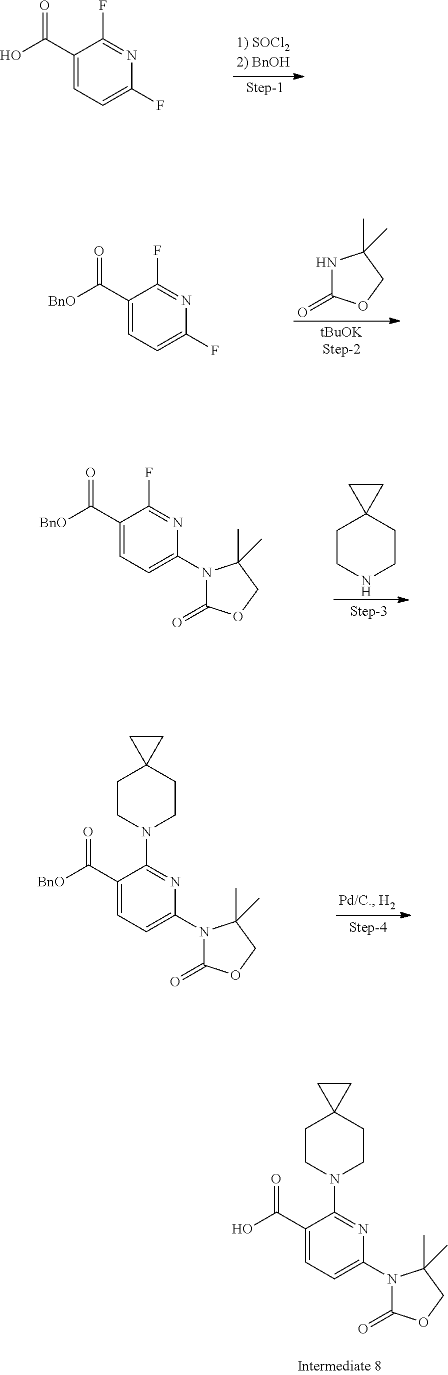

Step 1: 2,6-Difluoronicotinic acid (10.6 g, 66.6 mmol) and thionyl chloride (35 mL, 480 mmol) were combined under nitrogen and heated to gentle reflux for 2 h. The solution was concentrated to dryness under reduced pressure. Toluene (100 mL) was added to the crude and it was evaporated to dryness once more. The crude acid chloride was dissolved in DCM (50 mL) under nitrogen and cooled in an ice bath. A mixture of triethylamine (25 mL, 180 mmol) and benzyl alcohol (7.25 mL, 70.1 mmol) in DCM (50 mL) was added dropwise over 10 min, and the mixture was stirred at rt for 30 min. Then, 0.1 N HCl (100 mL) was added and the phases mixed and separated. The organic phase was taken, dried with magnesium sulfate, and evaporated to dryness under reduced pressure to provide benzyl 2,6-difluoronicotinate which was used without purification. m/z (ESI): 250.0 (M+H)⁺.

Step 2: 4,4-Dimethyloxazolidin-2-one (0.80 g, 6.95 mmol) was dissolved in THF (15 mL) under nitrogen. Potassium t-butoxide (0.75 g, 6.68 mmol) was added and the suspension stirred at RT for 5 min. A solution of benzyl 2,6-difluoronicotinate (1.60 g, 6.42 mmol) in N,N-dimethylacetamide (40 mL) was added and the mixture was stirred at RT for 10 min. Water (75 mL), EtOAc (150 mL), and satd ammonium chloride (25 mL) were added and the phases mixed and separated. The organic phase was taken, washed with brine (50 mL), and evaporated to dryness under reduced pressure. Purification by silica gel chromatography (heptane to EtOAc gradient) gave benzyl 6-(4,4-dimethyl-2-oxooxazolidin-3-yl)-2-fluoronicotinate (1.82 g, 5.29 mmol, 82% yield) as a white solid.

Step 3: Benzyl 6-(4,4-dimethyl-2-oxooxazolidin-3-yl)-2-fluoronicotinate (1.81 g, 5.23 mmol) was dissolved in NMP (20 mL). Cesium carbonate (2.00 g, 6.14 mmol) and 6-azaspiro[2.5]octane (0.60 g, 5.40 mmol) were added and the mixture stirred at RT for 18 h. Water (100 mL) and EtOAc (150 mL) were added and the phases mixed and separated. The organic phase was taken, washed with brine, and evaporated to dryness under reduced pressure. Purification using the silica gel chromatography (0% to 40% EtOAc in heptane) gave benzyl 6-(4,4-dimethyl-2-oxooxazolidin-3-yl)-2-(6-azaspiro[2.5]octan-6-yl)nicotinate (1.77 g, 4.06 mmol, 78% yield) as a milky oil. m/z (ESI): 436.1 (M+H)⁴.

Step 4: Benzyl 6-(4,4-dimethyl-2-oxooxazolidin-3-yl)-2-(6-azaspiro[2.5]octan-6-yl)nicotinate (1.77 g, 4.06 mmol) was dissolved in EtOAc (30 mL) and transferred to a pressure vessel. Ethanol (60 mL) was added followed by 5% palladium on carbon (dry wt., 50% water, 0.250 g, 0.117 mmol). The suspension was stirred under 40 psi hydrogen for 15 min. The mixture was filtered through a pad of CELITE® and the solid washed with EtOAc (50 mL). The combined filtrate was evaporated to dryness under reduced pressure to give 6-(4,4-dimethyl-2-oxooxazolidin-3-yl)-2-(6-azaspiro[2.5]octan-6-yl)nicotinic acid (1.15 g, 3.33 mmol, 82% yield) as a white solid. m/z (ESI): 346.0 (M+H)⁺.

### Intermediate 9: N-(2-(4,4-Difluoropiperidin-1-yl)-6-methylpyrimidin-4-yl)-4-iodo-2-(6-azaspiro[2.5]octan-6-yl)benzamide

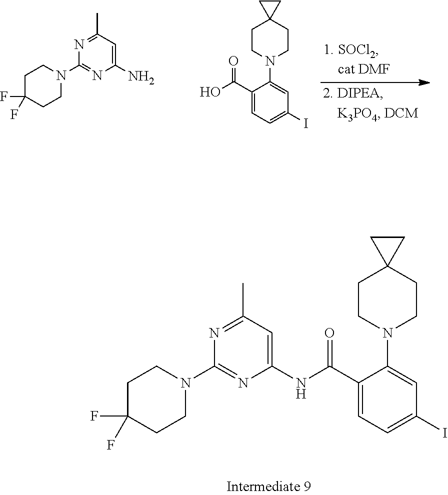 [SMILES: Cc1cc(N)nc(N2CCC(F)(F)CC2)n1 ; O=C(O)c1ccc(I)cc1N1CCC2(CC1)CC2 ; Cc1cc(NC(=O)c2ccc(I)cc2N2CCC3(CC2)CC3)nc(N2CCC(F)(F)CC2)n1]

4-Iodo-2-(6-azaspiro[2.5]octan-6-yl)benzoic acid (150.0 g, 420 mmol, Int. 6) was suspended in dichloromethane (1000 mL) under argon. Catalytic DMF (1.0 mL) was added followed by dropwise addition of a solution of thionyl chloride (54.6 g, 28 mL, 459 mmol, Sigma-Aldrich Corporation) in dichloromethane (500 mL) over 10 minutes. After stirring at ambient temperature for 30 minutes, the mixture was evaporated to dryness under reduced pressure. The crude was azeotroped with toluene (2×300 mL) and suspended in dichloromethane (300 mL) under argon. Tribasic potassium phosphate (267 g, 1.26 mol, Sigma-Aldrich Corporation) was added followed by a solution of 2-(4,4-difluoropiperidin-1-yl)-6-methylpyrimidin-4-amine (100 g, 438 mmol, Int. 4) and N,N-diisopropylethylamine (200 mL, 1.14 mol, Sigma-Aldrich Corporation) in DCM (300 mL, added over 5 minutes). The yellow mixture was stirred at ambient temperature for 3 hours then evaporated to dryness under reduced pressure. The crude solids were suspended in dichloromethane (1 L) and stirred for 10 minutes. The mixture was filtered through a frit and the solids washed with additional dichloromethane (2×100 mL). The solids were discarded, and the filtrate was evaporated to dryness under reduced pressure. The crude residue was suspended in acetonitrile (750 mL) and stirred at ambient temperature for 15 minutes. The suspension was filtered through a glass frit and the solids washed with additional acetonitrile (75 mL). The solids were dried under a stream of nitrogen to give N-(2-(4,4-difluoropiperidin-1-yl)-6-methylpyrimidin-4-yl)-4-iodo-2-(6-azaspiro[2.5]octan-6-yl)benzamide (186 g, 328 mmol, 78% yield). ¹H NMR (400 MHz, DMSO-d) 6 ppm 13.38 (br s, 1H) 7.72-7.87 (m, 3H) 7.39 (s, 1H) 3.91 (br s, 4H) 2.99-3.06 (m, 4H) 2.32 (s, 3H) 1.92-2.07 (m, 4H) 1.62-1.85 (m, 4H) 0.38 (s, 4H). m/z (ESI): 568.0 (M−H)⁺.

Intermediates 10-13 were prepared following a similar procedure as described for intermediate 9.

| | | | LRMS: (ESI + |
|---|---|---|---|
| Int. # | Chemical Structure | Name | ve ion) m/z |
| 10 | 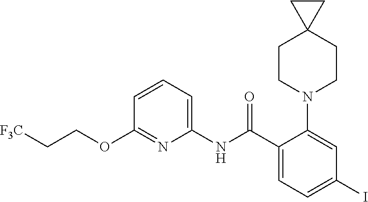 [SMILES: O=C(Nc1cccc(OCCC(F)(F)F)n1)c1ccc(I)cc1N1CCC2(CC1)CC2] | 4-iodo-2-(6-azaspiro[2.5]octan-6-yl)-N- (6-(3,3,3-trifluoropropoxy)pyridin-2- yl)benzamide | 546.1 |
| 11 | 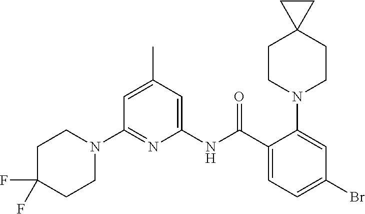 [SMILES: Cc1cc(NC(=O)c2ccc(Br)cc2N2CCC3(CC2)CC3)nc(N2CCC(F)(F)CC2)c1] | 4-bromo-N-(6-(4,4-difluoropiperidin-1- yl)-4-methylpyridin-2-yl)-2-(6- azaspiro[2.5]octan-6-yl)benzamide | 519.2 \| 521.2 |
| 12 | 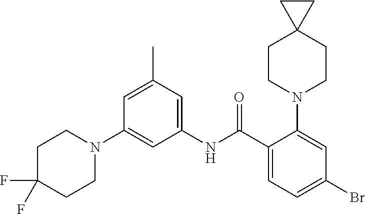 [SMILES: Cc1cc(NC(=O)c2ccc(Br)cc2N2CCC3(CC2)CC3)cc(N2CCC(F)(F)CC2)c1] | 4-bromo-N-(3-(4,4-difluoropiperidin-1- yl)-5-methylphenyl)-2-(6- azaspiro[2.5]octan-6-yl)benzamide | 518.1 \| 520.1 |
| 13 | 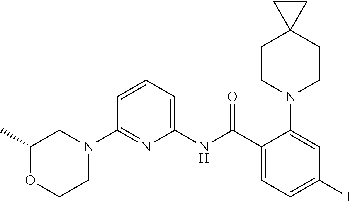 [SMILES: C[C@@H]1CN(c2cccc(NC(=O)c3ccc(I)cc3N3CCC4(CC3)CC4)n2)CCO1] | (R)-4-iodo-N-(6-(2- methylmorpholino)pyridin-2-yl)-2-(6- azaspiro[2.5joctan-6-yl)benzamide | 533.1 |

### Examples C1 and C2: 2-(6-Azaspiro[2.5]octan-6-yl)-4-(R-cyclopropylsulfonimidoyl)-N-(2-(4,4-difluoro-1-piperidinyl)-6-methyl-4-pyrimidinyl)benzamide and 2-(6-azaspiro[2.5]octan-6-yl)-4-(S-cyclopropylsulfonimidoyl)-N-(2-(4,4-difluoro-1-piperidinyl)-6-methyl-4-pyrimidinyl)benzamide

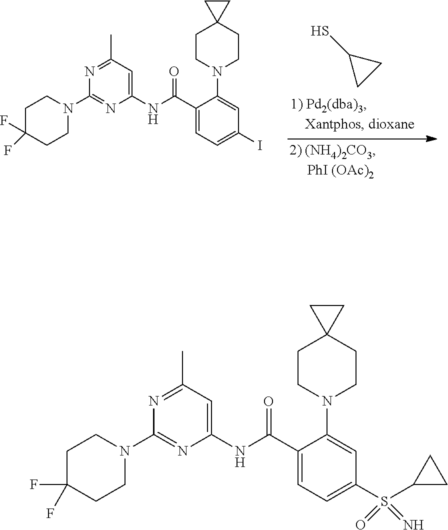

Step 1: Into a 20 mL microwave vessel were placed N-(2-(4,4-difluoropiperdin-1-yl)-6-methylpyrimidin-4-yl)-4-iodo-2-(6-azaspiro[2.5]octan-6-yl)benzamide (1.00 g, 1.762 mmol, Int. 19), tris (dibenzylideneacetone) dipalladium (0) (0.161 g, 0.176 mmol) and 4,5-bis(diphenylphos-phino)-9,9-dimethyl-xanthene (0.102 g, 0.176 mmol) followed by 1,4-dioxane (10 mL). The resulting mixture was stirred and purged with nitrogen for 5 min before 1,1′-dimethyltriethylamine (0.616 mL, 3.52 mmol) was added under nitrogen followed by cyclopropanethiol (0.142 mL, 1.939 mmol). The vessel was sealed and subjected to microwave condition (10 h, 90° C.). The crude mixture was directly loaded onto a silica gel precolumn and subjected to combi-flash column chromatography on a 40 -g ISCO gold column eluting with MeOH/DCM (5 min at 0% and 25 min from 0 to 6%) twice to give 4-(cyclopropylthio)-N-(2-(4,4-difluoropiperidin-1-yl)-6-methylpyrmidin-4-yl)-2-(6-azaspiro[2.5]octan-6-yl)benzamide (0.92 g, 1.791 mmol, 102% yield) as an off-white solid. ¹H NMR (400 MHz, DICHLOROMETHANE-d₂) 6 ppm 13.33 (s, 1H), 8.15 (d, J=8.29 Hz, 1H), 7.48 (s, 1H), 7.22-7.35 (m, 2H), 3.91-4.09 (m, 4H), 3.06 (br t, J=5.18 Hz, 4H), 2.35 (s, 3H), 2.17-2.28 (m, 1H), 1.62-2.10 (m, 6H), 1.52 (s, 2H), 1.13-1.21 (m, 2H), 0.68-0.76 (m, 2H), 0.40 (s, 4H). m/z (ESI): 514.1 (M+H)⁺.

Step 2: To a stirred solution of 4-(cyclopropylthio)-N-(2-(4,4-difluoropiperdin-1-yl)-6-methylpyimidin-4-yl)-2-(6-azaspiro[2.5]octan-6-yl)benzamide (0.89 g, 1.733 mmol) and ammonium carbonate (0.250 g, 2.60 mmol) in MeOH (4.5 mL) and dichloromethane (9.0 mL) was added (acetyloxy)(phenyl)-iodanyl acetate (1.284 g, 3.99 mmol) in one portion as a solid. The resulting mixture was stirred in open air at rt for 18 h. The resulting mixture was directly loaded onto silica gel precolumn (25 g) and subjected to combi-flash column chromatography on a 40 -g ISCO gold column eluting with MeOH/DCM (3 min at 0% and 25 min from 0 to 14%) to give a racemic mixture of 4-(cyclopropanesulfonimidoyl)-N-(2-(4,4-difluoropiperidin-1-yl)-6-methylpyrimidin-4-yl)-2-(6-azaspiro[2.5]octan-6-yl)benzamide (0.95 g, 1.744 mmol, 101% yield) as an off-white solid. The enantiomers were separated via preparative SFC using a Regis (S,S) Whelk-01 (250×21 mm, 5 mm) with a mobile phase of 50% Liquid CO₂ and 50% MeOH using a flow rate of 60 mL/min to generate:

Example C1: 2-(6-Azaspiro[2.5]octan-6-yl)-4-(R-cyclopropylsulfonimidoyl)-N-(2-(4,4-difluoro-1-piperidinyl)-6-methyl-4-pyrimidinyl)benzamide. First eluting peak, ¹H NMR (400 MHz, CHLOROFORM-d) δ ppm 13.20 (br d, J=3.73 Hz, 1H), 8.44 (d, J=8.29 Hz, 1H), 7.96 (d, J=1.45 Hz, 1H), 7.87 (dd, J=1.66, 8.29 Hz, 1H), 7.52 (s, 1H), 4.03 (br s, 4H), 3.14 (t, J=5.29 Hz, 4H), 2.53-2.63 (m, 1H), 2.44 (br s, 3H), 1.95-2.10 (m, 4H), 1.53-1.89 (m, 5H), 1.45 (tdd, J=5.08, 6.92, 10.29 Hz, 1H), 1.20-1.30 (m, 1H), 1.07-1.17 (m, 1H), 0.93-1.03 (m, 1H), 0.44 (s, 4H). m/z (ESI): 545.2 (M+H)⁺.

Example C2: 2-(6-Azaspiro[2.5]octan-6-yl)-4-(R-cyclopropylsulfonimidoyl)-N-(2-(4,4-difluoro-1-piperidinyl-6-methyl-4-pyrimidinyl)benzamide. Second eluting peak. ¹H NMR (400 MHz, CHLOROFORM-d) δ ppm 13.20 (br d, J=3.73 Hz, 1H), 8.44 (d, J=8.29 Hz, 1H), 7.96 (d, J=1.45 Hz, 1H), 7.87 (dd, J=1.66, 8.29 Hz, 1H), 7.52 (s, 1H), 4.03 (br s, 4H), 3.14 (t, J=5.29 Hz, 4H), 2.53-2.63 (m, 1H), 2.44 (br s, 3H), 1.95-2.10 (m, 4H), 1.53-1.89 (m, 5H), 1.45 (tdd, J=5.08, 6.92, 10.29 Hz, 1H), 1.20-1.30 (m, 1H), 1.07-1.17 (m, 1H), 0.93-1.03 (m, 1H), 0.44 (s, 4H). m/z (ESI): 545.2 (M+H)⁺. The stereochemistry assignments were arbitrary.

### Example C3: 4-((2-Hydroxyethyl)sulfonamido)-2-(6-azaspiro[2.5]octan-6-yl)-N-(6-(3,3,3-trifluoropropoxy)pyridin-2-yl)benzamide

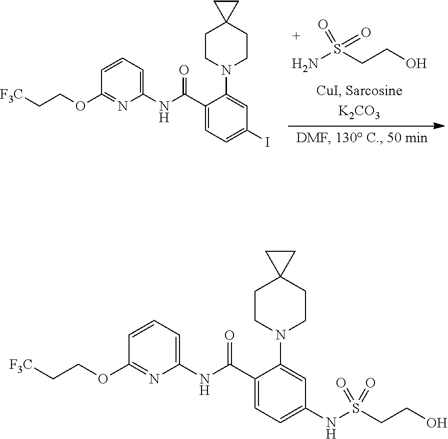 [SMILES: O=C(Nc1cccc(OCCC(F)(F)F)n1)c1ccc(I)cc1N1CCC2(CC1)CC2 ; NS(=O)(=O)CCO ; O=C(Nc1cccc(OCCC(F)(F)F)n1)c1ccc(NS(=O)(=O)CCO)cc1N1CCC2(CC1)CC2]

2-Hydroxyethane-1-sulfonamide (0.741 g, 5.92 mmol, Wuxi AppTec), sarcosine (0.172 g, 1.93 mmol, Ark Pharm, Inc.), copper(I) iodide (0.241 g, 1.26 mmol, Sigma-Aldrich Corporation), potassium carbonate (2.78 g, 20.1 mmol, Thermo Fisher Scientific) and 4-iodo-2-(6-azaspiro[2.5]octan-6-yl)-N-(6-(3,3,3-trifluoropropoxy)pyridin-2-yl)benzamide (2.74 g, 5.02 mmol, Intermediate 38) were combined in degassed dry N,N-dimethylformamide (5 mL) under argon and heated to 130° C. for 50 min. The reaction was cooled to ambient temperature, water (100 mL) and ethyl acetate (150 mL) were added and the phases mixed and separated. The organic layer was washed with satd NH₄Cl: NH₄OH:H₂O (1:1:8, 2×75 mL) and evaporated to dryness under reduced pressure. The crude product was suspended in toluene (30 mL) and heated to 90° C. for 15 min. The mixture was cooled to ambient temperature and the solids were filtered off and dried under a stream of nitrogen. The white solids were suspended in water (100 mL) and heated to 90° C. for 20 minutes. The mixture was cooled to ambient temperature and the solids dried under a stream of nitrogen to give 4-((2-hydroxyethyl)sulfonamido)-2-(6-azaspiro[2.5]octan-6-yl)-N-(6-(3,3,3-trifluoropropoxy)pyridin-2-yl)benzamide (2.41 g, 4.44 mmol, 88% yield). ¹H NMR (500 MHz, DMSO-d₆) δ 13.18 (s, 1H), 10.19 (br s, 1H), 8.08 (d, J=8.72 Hz, 1H), 7.91 (d, J=7.80 Hz, 1H), 7.76 (t, J=7.96 Hz, 1H), 7.29 (d, J=1.99 Hz, 1H), 7.14 (dd, J=2.07, 8.64 Hz, 1H), 6.57 (d, J=7.96 Hz, 1H), 4.93 (br s, 1H), 4.52 (t, J=6.12 Hz, 2H), 3.77 (t, J=6.43 Hz, 2H), 3.37 (t, J=6.43 Hz, 2H), 3.00 (br t, J=4.74 Hz, 4H), 2.80-2.87 (m, 2H), 1.74 (br s, 4H), 0.39 (s, 4H). m/z (ESI): 543.2.2 (M+H)⁺.

### Example C4: N-(6-(4,4-Difluoropiperidin-1-yl)-4-methylpyridin-2-yl)-4-((2-hydroxyethyl)sulfonamido)-2-(6-azaspiro[2.5]octan-6-ylbenzamide

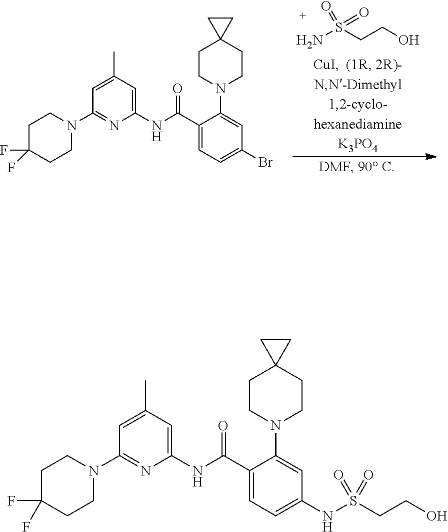 [SMILES: Cc1cc(NC(=O)c2ccc(Br)cc2N2CCC3(CC2)CC3)nc(N2CCC(F)(F)CC2)c1 ; NS(=O)(=O)CCO ; Cc1cc(NC(=O)c2ccc(NS(=O)(=O)CCO)cc2N2CCC3(CC2)CC3)nc(N2CCC(F)(F)CC2)c1]

A mixture of 4-bromo-N-(6-(4,4-difluoropiperdin-1-yl)-4-methylpyridin-2-yl)-2-(6-azaspiro[2.5]octan-6-yl)benzamide (1.0 g, 1.9 mmol, Intermediate 27), methyl 2-sulfamoylacetate (0.361 g, 2.89 mmol, Wuxi AppTec), potassium phosphate (1.23 g, 5.78 mmol), (1R,2R)—N1,N2-dimethylcyclohexane-1,2-diamine (0.137 g, 0.963 mmol) and copper(I) iodide (0.183 g, 0.963 mmol) in DMF (20 mL) was heated at 90° C. for 16 h. Then the reaction mixture was filtered through a plug of CELITE®. The filtrate was diluted with EtOAc, washed with water, brine, dried over Na₂SO₄, filtered, and concentrated. The residue was purified by flash column chromatography eluting with a gradient of 0% to 40% EtOAc in petroleum ether to provide N-(6-(4,4-difluoropiperdin-1-yl)-4-methylpyridin-2-yl)-4-((2-hydroxyethyl)sulfonamido)-2-(6-azaspiro[2.5]octan-6-yl)benzamide (0.580 g, 1.02 mmol, 53% yield) as off-white solid. ¹H NMR (400 MHz, DMSO-d₆) δ ppm 12.85 (s, 1H), 8.04 (d, J=8.6 Hz, 1H), 7.51 (s, 1H), 7.23 (d, J=2.2 Hz, 1H), 7.09 (dd, J=8.7, 2.1 Hz, 1H), 6.56 (s, 1H), 3.74 (dt, J=12.5, 6.2 Hz, 6H), 2.97 (t, J=5.2 Hz, 4H), 2.26 (s, 3H), 1.99 (tt, J=13.6, 5.4 Hz, 3H), 1.79 (s, 4H), 1.60 (br s, 4H), 0.38 (s, 4H). m/z (ESI): 564.2 (M+H)⁺.

### Examples C5 and C6: (R)—N-(2-(4,4-difluoropiperidin-1-yl)-6-methylpyrmidin-4-yl)-4-((2-hydroxy-1-methylethyl)sulfonamido)-2-(6-azaspiro[2.5]octan-6-yl)benzamide and (S)—N-(2-(4,4-difluoropiperidin-1-yl)-6-methylpyrimidin-4-yl)-4-((2-hydroxy-1-methylethyl)sulfonamido)-2-(6-azaspiro[2.5]octan-6-yl)benzamide

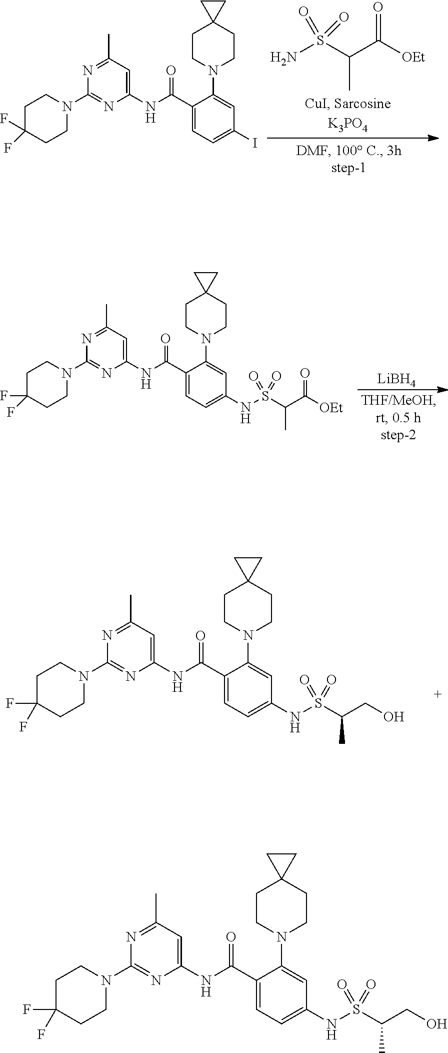 [SMILES: Cc1cc(NC(=O)c2ccc(I)cc2N2CCC3(CC2)CC3)nc(N2CCC(F)(F)CC2)n1 ; CCOC(=O)C(C)S(N)(=O)=O ; CCOC(=O)C(C)S(=O)(=O)Nc1ccc(C(=O)Nc2cc(C)nc(N3CCC(F)(F)CC3)n2)c(N2CCC3(CC2)CC3)c1 ; "LiBH4" ; Cc1cc(NC(=O)c2ccc(NS(=O)(=O)[C@H](C)CO)cc2N2CCC3(CC2)CC3)nc(N2CCC(F)(F)CC2)n1 ; Cc1cc(NC(=O)c2ccc(NS(=O)(=O)[C@@H](C)CO)cc2N2CCC3(CC2)CC3)nc(N2CCC(F)(F)CC2)n1]

Step 1: A mixture of ethyl 2-sulfamoylpropanoate (1.44 g, 7.93 mmol, Int. 22), copper(I) iodide (0.503 g, 2.64 mmol, Strem), sarcosine (0.47 g, 5.29 mmol, Sigma-Aldrich Corporation), and potassium phosphate (4.49 g, 21.2 mmol) in DMF (15 mL) was placed under argon atmosphere and warmed to 50° C. for 5 min. N-(2-(4,4-difluoropiperidin-1-yl)-6-methylpyrimidin-4-yl)-4-iodo-2-(6-azaspiro[2.5]octan-6-yl)benzamide (3.0 g, 5.29 mmol, Int. 19) was added and the mixture was heated to 100° C. for 3 h, then cooled to room temperature. EtOAc (50 mL), IPA (5 mL) and water (50 mL) were added and the mixture was stirred vigorously for 5 min. The resulting biphasic mixture was transferred to a separatory funnel and the layers were separated. The aqueous layer was extracted with EtOAc (2×20 mL) and the combined extracts were then washed with water (2×50 mL), 9:1 NH₄Cl/NH₄OH (1×50 mL), dried over anhydrous MgSO₄, filtered, and concentrated in vacuo to give an oil. The crude oil was purified by silica gel chromatography using a Redi-Sep pre-packed silica gel column (80 g), eluting with 0 to 50% EtOAc/heptane gradient, to provide ethyl 2-(N-(4-((2-(4,4-difluoropiperidin-1-yl)-6-methylpyrimidin-4-yl)carbamoyl)-3-(6-azaspiro[2.5]octan-6-yl)phenyl)sulfamoyl)propanoate (2.76 g, 4.45 mmol, 84% yield) as a white solid. ¹H NMR (400 MHz, DMSO-d₆) δ ppm 13.35 (s, 1H) 10.69 (br s, 1H) 8.07 (d, J=8.71 Hz, 1H) 7.40 (s, 1H) 7.31 (d, J=1.87 Hz, 1H) 7.17 (dd, J=8.60, 1.97 Hz, 1H) 4.06 (qd, J=7.08, 4.87 Hz, 2H) 3.92 (br t, J=5.49 Hz, 4H) 2.98 (br t, J=4.77 Hz, 4H) 2.32 (s, 3H) 1.85-2.06 (m, 5H) 1.73 (br s, 4H) 1.48 (d, J=6.84 Hz, 3H) 1.14 (t, J=7.05 Hz, 3H) 0.39 (s, 4H). ¹⁹F NMR (376 MHz, DMSO-d₆) δ ppm −94.75 (s, 1 F). m/z (ESI): 621.2 (M+H)⁺.

Step 2: To a 250 mL round bottom flask was added ethyl 2-(N-(4-((2-(4,4-difluoropiperidin-1-yl)-6-methylpyrimidin-4-yl)carbamoyl)-3-(6-azaspiro[2.5]octan-6-yl)phenyl)sulfamoyl)propanoate (10.39 g, 16.74 mmol) and lithium borohydride solution, (2.0M in THF, 16.7 mL, 33.5 mmol, Sigma-Aldrich Corporation) in THF (100 mL). Methanol (4.29 mL, 134 mmol) was added slowly over 5 min and the resulting solution was stirred at room temperature for 30 min. 1 N HCl (20 mL) was slowly added followed by EtOAc (20 mL) and the resulting biphasic mixture was transferred to a separatory funnel and the phases were separated. The aqueous layer was extracted with EtOAc (1×25 mL) and the combined extracts were washed with saturated NaHCO₃ (1×50 mL), brine (1×50 mL), dried over anhydrous MgSO₄, filtered, and concentrated to give 8.9 g racemic mixture. This material was separated by preparative SFC using a Chiral Tech AD column (250×30 mm, 5 mm) with a mobile phase of 85% Liquid CO₂ and 15% MeOH with 0.2% TEA using a flowrate of 150 mL/min to give:

Example C5: (R)—N-(2-(4,4-difluoropiperidin-1-yl)-6-methylpyrimidin-4-yl)-4-((2-hydroxy-1-methylethyl)sulfonamido)-2-(6-azaspiro[2.5]octan-6-yl)benzamide. First eluting peak (3.50 g, 6.05 mmol, 36.1% yield, >99% ee). ¹H NMR (400 MHz, DMSO-d₆) δ ppm 13.36 (s, 1H) 8.05 (d, J=8.50 Hz, 1H) 7.40 (s, 1H) 7.31 (d, J=1.87 Hz, 1H) 7.17 (dd, J=8.71, 2.07 Hz, 1H) 3.88-3.97 (m, 4H) 3.84 (dd, J=10.99, 4.35 Hz, 1H) 3.37-3.54 (m, 1H) 3.25-3.30 (m, 1H) 2.97 (br t, J=4.77 Hz, 4H) 2.32 (s, 3H) 1.84-2.06 (m, 4H) 1.57-1.84 (br s, 4H) 1.30 (d, J=6.84 Hz, 3H) 0.39 (s, 4H). 2 exchangeable protons not observed. ¹⁹F NMR (376 MHz, DMSO-d₆) δ ppm −94.74 (s, 1 F). m/z (ESI): 579.2 (M+H)⁺.

Example C6: (S)—N-(2-(4,4-difluoropiperidin-1-yl)-6-methylpyrimidin-4-yl)-4-((2-hydroxy-1-methylethyl)sulfonamido)-2-(6-azaspiro[2.5]octan-6-yl)benzamide. Second eluting peak (2.66 g, 4.60 mmol, 27.5% yield. 98.9% ee). ¹H NMR (400 MHz, DMSO-d₆) δ ppm 13.35 (s, 1H) 8.05 (d, J=8.50 Hz, 1H) 7.40 (s, 1H) 7.31 (d, J=2.07 Hz, 1H) 7.17 (dd. J=8.60, 1.97 Hz, 1H) 3.88-3.97 (m, 4H) 3.84 (dd, J=10.99, 4.35 Hz, 1H) 3.50 (dd, J=10.99, 7.46 Hz, 1H) 3.25-3.32 (m, 1H) 2.97 (br t, J=4.77 Hz, 4H) 2.31 (s, 3H) 1.83-2.06 (m, 4H) 1.73 (br s, 4H) 1.30 (d, J=6.84 Hz, 3H) 0.39 (s, 4H). 2 exchangeable protons not observed. ¹⁹F NMR (376 MHz, DMSO-d₆) δ ppm −94.75 (s, 1 F). m/z (ESI): 579.2 (M+H)⁺. The stereochemistry was arbitrarily assigned.

### Example C7: N-(3-(4,4-difluoropiperidin-1-yl)-5-methylphenyl)-4-((2-hydroxyethyl)sulfonamido)-2-(6-azaspiro[2.5]octan-6-yl)benzamide

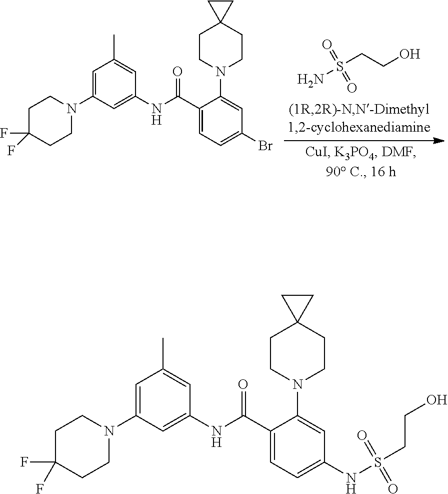 [SMILES: Cc1cc(NC(=O)c2ccc(Br)cc2N2CCC3(CC2)CC3)cc(N2CCC(F)(F)CC2)c1 ; NS(=O)(=O)CCO ; Cc1cc(NC(=O)c2ccc(NS(=O)(=O)CCO)cc2N2CCC3(CC2)CC3)cc(N2CCC(F)(F)CC2)c1]

A mixture of 4-bromo-N-(3-(4,4-difluoropiperidin-1-yl)-5-methylphenyl)-2-(6-azaspiro[2.5]octan-6-yl)benzamide (0.5 g, 0.96 mmol, Intermediate 20), potassium phosphate (0.614 g, 2.89 mmol), 2-hydroxyethane-1-sulfonamide (0.181 g, 1.45 mmol), (1R,2R)—N1,N2-dimethylcyclohexane-1,2-diamine (0.069 g, 0.48 mmol) and copper(I) iodide (0.092 g, 0.48 mmol) in DMF (5 mL) was stirred at 90° C. for 16 h. The reaction mixture was quenched with ice water, filtered through a CELITE® bed, and extracted with EtOAc. The organic extract was washed with brine, dried over Na₂SO₄, filtered, concentrated, and purified by silica gel column chromatography using 40% EtOAc in petroleum ether to provide N-(3-(4,4-difluoropiperidin-1-yl)-5-methylphenyl)-4-((2-hydroxyethyl)sulfonamido)-2-(6-azaspiro[2.5]octan-6-yl)benzamide (0.31 g, 0.54 mmol, 56% yield) as an off-white solid. ¹H NMR (400 MHz, DMSO-d₆): δ ppm 11.55 (s, 1H), 10.09 (s, 1H), 7.83 (d, J=8.5 Hz, 1H), 7.12-7.16 (m, 3H), 7.03 (dd, J=8.5, 2.1 Hz, 1H), 6.60 (s, 1H), 4.97 (br s, 1H), 3.76 (t, J=6.6 Hz, 2H), 3.30-3.34 (m, 6H), 2.97 (t, J=5.3 Hz, 4H), 2.27 (s, 3H), 2.00-2.10 (m, 4H), 1.55 (br s, 4H), 0.36 (s, 4H). m/z (ESI): 563.2 (M+H)⁺.

### Example C8: N-(3-(N-(tert-Butyl)sulfamoyl)phenyl)-4-((3-methyloxetan-3-yl)sulfonyl-2-(6-azaspiro[2.5]octan-6-yl)benzamide

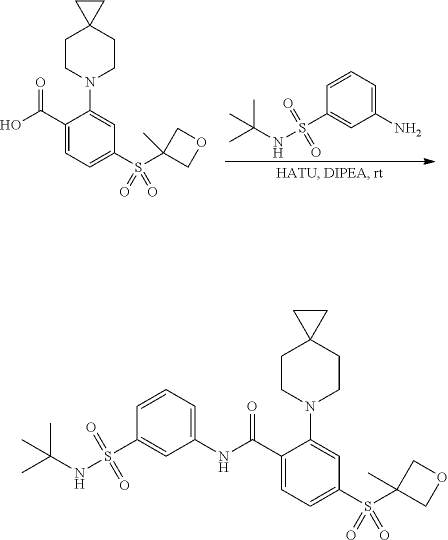

To a solution of 4-((3-methyloxetan-3-yl)sulfonyl)-2-(6-azaspiro[2.5]octan-6-yl)benzoic acid (120 mg, 0.33 mmol, Intermediate 15) in DMF (2 mL) were added HATU (187 mg, 0.49 mmol) and DIPEA (143 μL, 0.821 mmol) at RT and stirred for 10 min. To this reaction mixture, 3-amino-N-(tert-butyl)benzenesulfonamide (82 mg, 0.36 mmol) was added and stirred for 12 h at RT. The reaction mixture was quenched with water (20 mL) and extracted by EtOAc (3×25 mL). The combined organic extracts were washed with brine solution (20 mL), dried over Na₂SO₄, filtered, and concentrated under reduced pressure. The crude residue was purified by silica gel column chromatography using 30% EtOAc in hexanes to give the title compound (110 mg, 58% yield) as an off-white solid. ¹H NMR (400 MHz, Chloroform-d): 612.33 (s, 1H), 8.47 (d, J=8.2 Hz, 1H), 8.31 (d, J=2.1 Hz, 1H), 8.06-7.95 (m, 1H), 7.87 (d, J=1.8 Hz, 1H), 7.79 (dd, J=8.2, 1.7 Hz, 1H), 7.69 (d, J=8.3 Hz, 1H), 7.54 (t, J=8.0 Hz, 1H), 5.19 (d, J=7.0 Hz, 2H), 4.52 (s, 1H), 4.47 (d, J=7.0 Hz, 2H), 3.16 (t, J=5.5 Hz, 4H), 1.73 (s, 3H), 1.70-1.60 (b s, 3H), 1.30 (s, 9H), 0.48 (s, 4H). m/z (ESI): 576.2 [M+1].

### Example C9 was Prepared Analogous to Preparation of Example C8 Above

| | | | LRMS: |
|---|---|---|---|
| | | | (ESI + ve ion) |
| Ex.# | Chemical Structure | Name | m/z |
| C9 | 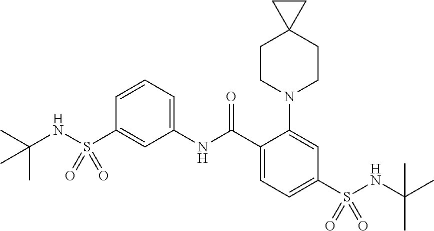 [SMILES: CC(C)(C)NS(=O)(=O)c1cccc(NC(=O)c2ccc(S(=O)(=O)NC(C)(C)C)cc2N2CCC3(CC2)CC3)c1] | 4-(N-(tert-butyl)sulfamoyl)-N- (3-(N-(tert- butyl)sulfamoyl)phenyl)-2-(6- azaspiro[2.5]octan-6- yl)benzamide | 577.2 |

### Example C10: N-(3-(N-(tert-Butyl)sulfamoyl)phenyl)-6-((1-hydroxy-2-methylpropan-2-yl)amino)-2-(6-azaspiro[2.5]octan-6-yl)nicotinamide

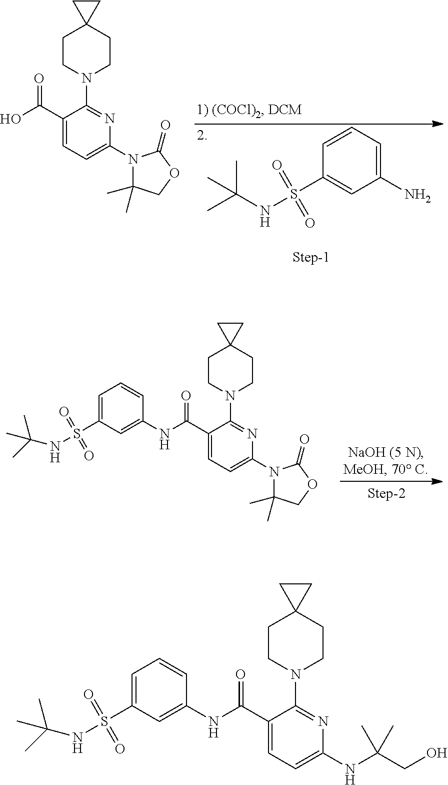

Step 1: A 100-mL round-bottomed flask was charged with 6-(4,4-dimethyl-2-oxooxazolidin-3-yl)-2-(6-azaspiro[2.5]octan-6-yl)nicotinic acid (549 mg, 1.59 mmol, Intermediate 11) and DCM (8 mL). To the reaction mixture at RT was added oxalyl dichloride (1.43 mL, 2.86 mmol, 2M in DCM) was added followed by a couple of drops of DMF. The mixture was stirred at rt for 1 h and the solvent was removed under vacuum. The residue was redissolved in DCM (10 mL) and treated with 3-amino-N-(tert-butyl)benzenesulfonamide (0.38 mL, 1.67 mmol), and DIPEA (1.39 mL, 7.95 mmol). The reaction mixture was stirred at RT for 18 h before it was diluted with water and extracted with EtOAc. The organic extract was washed with brine, dried over Na₂SO₄, filtered and concentrated. The concentrate was purified by flash column chromatography eluting with 0% to 60% EtOAc in heptane to N-(3-(N-(tert-butyl)sulfamoyl)phenyl)-6-(4,4-dimethyl-2-oxooxazolidin-3-yl)-2-(6-azaspiro[2.5]octan-6-yl)nicotinamide (703 mg, 1.26 mmol, 80% yield) as light-yellow solid. MS (ESI, Positive ion) m/z: 556.1 [M+1].

Step 2: A glass vial was charged with N-(3-(N-(tert-butyl)sulfamoyl)phenyl)-6-(4,4-dimethyl-2-oxooxazolidin-3-yl)-2-(6-azaspiro[2.5]octan-6-yl)nicotinamide (703 mg, 1.26 mmol), MeOH (2 mL), and sodium hydroxide (1.26 mL, 6.33 mmol, 5N). Stirred at 70° C. for 1 h, cooled to RT, and the solvent was removed under vacuum. The residue was partitioned between half-saturated NH₄Cl (10 mL) and EtOAc (10 mL). The aqueous phase was extracted with EtOAc (2×10 mL). The combined organic extracts were washed with water (20 mL) and dried over Na₂SO₄. The crude material was absorbed onto a plug of silica gel and purified by chromatography through a Redi-Sep pre-packed silica gel column, eluting with a gradient of 0% to 60% EtOAc in heptane, to provide N-(3-(N-(tert-butyl)sulfamoyl)phenyl)-6-((1-hydroxy-2-methylpropan-2-yl)amino)-2-(6-azaspiro[2.5]octan-6-yl)nicotinamide (485 mg, 0.92 mmol, 72% yield) as white solid. ¹H NMR (400 MHz, DMSO-d₆) δ ppm 11.21 (s, 1H), 8.32 (t, J=1.45 Hz, 1 H), 7.84 (dt, J=7.88, 1.45 Hz. 1H), 7.71 (d, J=8.71 Hz, 1H). 7.50-7.57 (m, 2H), 7.49 (dt, J=7.88, 1.45 Hz, 1H), 6.60 (s, 1H), 6.28 (d, J=8.50 Hz, 1H), 4.81 (t, J=5.70 Hz, 1H), 3.59 (d, J=5.81 Hz, 2H), 3.11-3.17 (m, 4H), 1.44-1.51 (m, 4H), 1.36 (s, 6H), 1.12 (s, 9H), 0.31 (s, 4H). MS (ESI, Positive ion) m/z: 530.2 [M+1].

### Example C11: Was Prepared Analogous to Preparation of Example C10

| | | | LRMS: |
|---|---|---|---|
| | | | (ESI + ve ion) |
| Ex. # | Chemical Structure | Name | m/z |
| C11 | 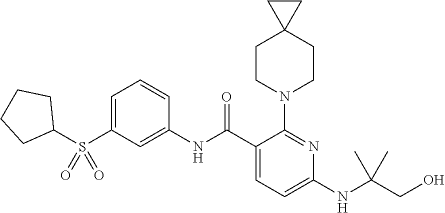 [SMILES: CC(C)(CO)Nc1ccc(C(=O)Nc2cccc(S(=O)(=O)C3CCCC3)c2)c(N2CCC3(CC2)CC3)n1] | N-(3- (cyclopentylsulfonyl) phenyl)-6-((1-hydroxy-2- methylpropan-2- yl)amino)-2-(6- azaspiro[2.5]octan-6- yl)nicotinamide | 527.0 |

### Examples C12-C13: Was Prepared Analogous to Preparation of Examples C3

| | | | LRMS: |
|---|---|---|---|
| | | | (ESI + ve ion) |
| Ex. # | Chemical Structure | Name | m/z |
| C12 | 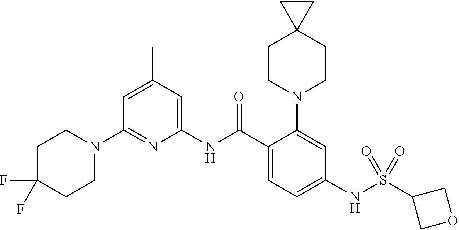 [SMILES: Cc1cc(NC(=O)c2ccc(NS(=O)(=O)C3COC3)cc2N2CCC3(CC2)CC3)nc(N2CCC(F)(F)CC2)c1] | N-(6-(4,4-Difluoropiperidin-1-yl)-4- methylpyridin-2-yl)-4-(oxetane-3- sulfonamido)-2-(6- azaspiro[2.5]octan-6-yl)benzamide | 576.2 |
| C13 | 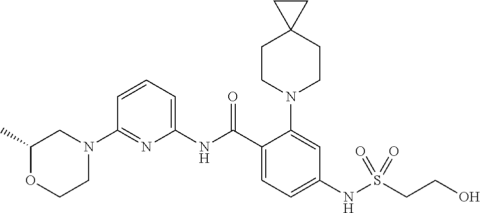 [SMILES: C[C@@H]1CN(c2cccc(NC(=O)c3ccc(NS(=O)(=O)CCO)cc3N3CCC4(CC3)CC4)n2)CCO1] | (R)-4-((2- Hydroxyethyl)sulfonamido)-N-(6-(2- methylmorpholino)pyridin-2-yl)-2- (6-azaspiro[2.5]octan-6- yl)benzamide | 530.2 |

### Example C14: N-(2-(4,4-Difluoropiperidin-1-yl)-6-methylpyrimidin-4-yl)-4-((2-hydroxyethyl)sulfonamido)-2-(6-azaspiro[2.5]octan-6-yl)benzamide

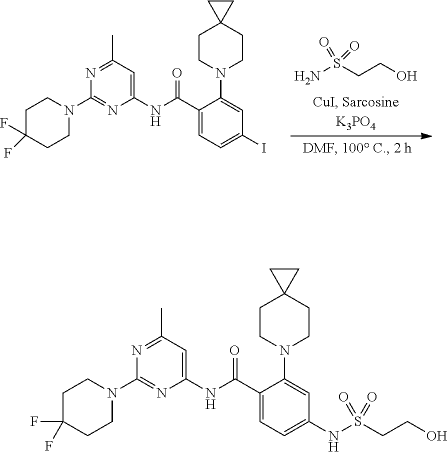 [SMILES: Cc1cc(NC(=O)c2ccc(I)cc2N2CCC3(CC2)CC3)nc(N2CCC(F)(F)CC2)n1 ; NS(=O)(=O)CCO ; Cc1cc(NC(=O)c2ccc(NS(=O)(=O)CCO)cc2N2CCC3(CC2)CC3)nc(N2CCC(F)(F)CC2)n1]

A mixture of 2-hydroxyethane-1-sulfonamide (1.28 g, 10.3 mmol, Wuxi AppTec), copper(I) iodide (0.49 g, 2.56 mmol), potassium phosphate tribasic (5.44 g, 25.6 mmol), and Sarcosine (0.48 g, 5.13 mmol) in a 100 mL round bottom flask was placed under argon atmosphere. Anhydrous DMF (20 mL) was added and the mixture was warmed to 50° C. for 5 minutes. N-(2-(4,4-difluoropiperidin-1-yl)-6-methylpyrimidin-4-yl)-4-iodo-2-(6-azaspiro[2.5]octan-6-yl)benzamide (2.91 g, 5.13 mmol, Int. 19) was added as a solid and the mixture was heated to 100° C. and stirred for 2 h, then cooled to room temperature. EtOAc (20 mL) and water (20 mL) were added, the resulting biphasic mixture was separated, and the aqueous layer was extracted with EtOAc (3×). The combined organic extracts were then washed with water (2×), 9:1 NH₄Cl/NH₄OH (aq), brine, dried over anhydrous MgSO₄, filtered, and concentrated in vacuo to give an oil. The oil was purified by silica gel chromatography, eluting with 0 to 50% EtOAc/heptane gradient, then 50% EtOAc/heptane isocratic elution, to provide an off-white solid. This solid was suspended in methanol, filtered, and dried to give a white solid. This solid was then suspended in water, stirred for 24 h, filtered, and dried in vacuo to provide N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide (1.55 g, 2.75 mmol, 54% yield) as a white solid. ¹H NMR (400 MHz, DMSO-ds) δ ppm 13.37 (s, 1H) 10.03-10.52 (m, 1H) 8.06 (d, J=8.71 Hz, 1H) 7.41 (s, 1H) 7.28 (d, J=1.87 Hz, 1H) 7.15 (dd, J=8.71, 1.87 Hz, 1H) 4.73-5.14 (m, 1H) 3.92 (br t, J=5.39 Hz, 4H) 3.77 (t, J=6.43 Hz, 2H) 3.34-3.40 (m, 2H) 2.98 (br t, J=4.56 Hz, 4H) 2.32 (s, 3H) 1.93-2.07 (m, 4H) 1.58-1.85 (m, 4H) 0.40 (s, 4H). ⁹F NMR (376 MHz, DMSO-d₆) δ ppm −94.74 (s, 1 F). m/z (ESI): 565.2 (M+H)⁺.

Those skilled in the art understand that they can convert the compounds of the invention to their corresponding pharmaceutically acceptable salt thereof by using conventional techniques known in the art. For example, to convert the exemplified compounds C-1 to C-14 to their corresponding HCl salts, those skilled in the art would understand to use the proper equivalent of hydrochloric acid, optionally followed by crystallization step and drying step to isolate the HCl salts.

### Example 12

The following assays were used in testing the exemplary KIF18A compounds that can be used in the methods of the invention. Data for those examples tested in accordance with the procedures described below are presented in Table 4 below.

KIF18A Enzyme Assay: Microtubule-stimulated ATPase activity assay was used to measure KIF18A enzyme activity after treatment with compound. Compounds were 2-fold serially diluted in DMSO (Sigma Inc) over 22-point concentration range. Recombinant human KIF18A (1-467 His-tagged) protein was expressed using a baculovirus system and purified by affinity chromatography by Amgen Inc. Concentrations of KIF18A protein, microtubules (MT), and ATP in the reaction were optimized for standardized homogenous enzyme assay using ADP-Glo™ Kinase/ATPase Assay Kit (Promega Inc). The assay measures ADP formed from the ATPase reaction. Prepare reaction buffer [(15 mM Tris, pH 7.5 (Teknova Inc), 10 mM MgCl2 (JT Baker Inc), 0.01% Pluronic F-68 (Life Technologies Inc), 1 μM Taxol (Cytoskeleton Inc), and 30 μg/mL pig microtubules (Cytoskeleton Inc)]. Add compound and KIF18A protein (30 nM) to prepared reaction buffer and incubated for 15 minutes at RT, next add ATP (at Kₘ, 75 μM) to the reaction mixture and incubated for an additional 15 minutes at RT. Mix 5 μl of ADP-Glo™ Reagent and 2.5 μl of the reaction mixture and incubate for 40 minutes at RT. Add 10 μl ADP-Glo™ Detection Reagent and incubate for 40 minutes at RT. Read luminescence using EnVision microplate reader with ultra-luminescence module (Perkin Elmer Inc).

Concentration-response curve-fitting and IC₅₀ determination was performed using Genedata Screener Software (Standard 15.0.1, Genedata Inc) with a four-parameter logistic regression fit model.

Table 4 provides data for compounds exemplified in the present application as representative KIF18A compounds that can be used in the methods of the present invention, as follows: compound name and biological data. (IC₅₀ in uM, where available. Ex. # refers to Example No.)

TABLE 4
BIOLOGICAL DATA

| | | KIF18A |
|---|---|---|
| | | ATPase |
| | | IC₅₀ |
| Ex. # | Compound Name | (μM) |
| C1 | 2-(6-Azaspiro[2.5]octan-6-yl)-4-(R-cyclopropylsulfonimidoyl)- | 0.064 |
| | N-(2-(4,4-difluoro-1-piperidinyl)-6-methyl-4- | |
| | pyrimidinyl)benzamide | |
| C2 | 2-(6-azaspiro[2.5]octan-6-yl)-4-(S-cyclopropylsulfonimidoyl)- | 0.057 |
| | N-(2-(4,4-difluoro-1-piperidinyl)-6-methyl-4- | |
| | pyrimidinyl)benzamide | |
| C3 | 4-((2-Hydroxyethyl)sulfonamido)-2-(6-azaspiro[2.5]octan-6-yl)- | 0.025 |
| | N-(6-(3,3,3-trifluoropropoxy)pyridin-2-yl)benzamide | |
| C4 | N-(6-(4,4-difluoropiperidin-1-yl)-4-methylpyridin-2-yl)-4-((2- | 0.047 |
| | hydroxyethyl)sulfonamido)-2-(6-azaspiro[2.5]octan-6-yl) | |
| | benzamide | |
| C5 | (R)-N-(2-(4,4-difluoropiperidin-1-yl)-6-methylpyrimidin-4-yl)-4- | 0.062 |
| | ((2-hydroxy-1-methylethyl)sulfonamido)-2-(6-azaspiro[2.5] | |
| | octan-6-yl)benzamide | |
| C6 | (S)-N-(2-(4,4-difluoropiperidin-1-yl)-6-methylpyrimidin-4-yl)-4- | 0.070 |
| | ((2-hydroxy-1-methylethyl)sulfonamido)-2-(6-azaspiro[2.5] | |
| | octan-6-yl)benzamide | |
| C7 | N-(3-(4,4-difluoropiperidin-1-yl)-5-methylphenyl)-4-((2- | 0.034 |
| | hydroxyethyl)sulfonamido)-2-(6-azaspiro[2.5]octan-6-yl) | |
| | benzamide | |
| C8 | N-(3-(N-(tert-Butyl)sulfamoyl)phenyl)-4-((3-methyloxetan-3- | 0.061 |
| | yl)sulfonyl)-2-(6-azaspiro[2.5]octan-6-yl)benzamide | |
| C9 | 4-(N-(tert-butyl)sulfamoyl)-N-(3-(N-(tert-butyl)sulfamoyl) | 0.076 |
| | pheny)-2-(6-azaspiro[2.5]octan-6-yl)benzamide | |
| C10 | N-(3-(N-(tert-Butyl)sulfamoyl)phenyl)-6-((1-hydroxy-2- | 0.070 |
| | methylpropan-2-yl)amino)-2-(6-azaspiro[2.5]octan-6-yl) | |
| | nicotinamide | |
| C11 | N-(3-(cyclopentylsulfonyl)phenyl)-6-((1-hydroxy-2- | 0.041 |
| | methylpropan-2-yl)amino)-2-(6-azaspiro[2.5]octan-6-yl) | |
| | nicotinamide | |
| C12 | (R)-4-((2-Hydroxyethyl)sulfonamido)-N-(6-(2- | 0.030 |
| | methylmorpholino)pyridin-2-yl)-2-(6-azaspiro[2.5]octan-6-yl) | |
| | benzamide | |
| C13 | (S)-4-((2-Hydroxyethyl)sulfonamido)-N-(6-(2- | 0.046 |
| | methylmorpholino)pyridin-2-yl)-2-(6-azaspiro[2.5]octan-6-yl) | |
| | benzamide | |
| C14 | N-(2-(4,4-Difluoropiperidin-1-yl)-6-methylpyrimidin-4-yl)-4- | 0.071 |
| | ((2-hydroxyethyl)sulfonamido)-2-(6-azaspiro[2.5]octan-6-yl) | |
| | benzamide | |

### Example 13

This example demonstrates KIF18A inhibitor activity in multidrug resistant TP53^{MUT} HGSOC cells.

Resistance to anti-mitotic agents, such as taxanes, is a complicating factor to successful cancer treatment and is often associated with increased expression of the MDR-1 encoded gene and its product, P-glycoprotein (P-gp). As shown in Example 1, all cell lines that exhibited sensitivity to a KIA18A inhibitor were mutant TP53 cancer cell lines. Here, sensitivity to KIF18A inhibitor treatment is evaluated in the presence or absence of multidrug resistance in a TP53^{1T} cancer cell line.

A KIF18A inhibitor, Compound C14, was evaluated in a 4-day image-based nuclear count assay (NCA) as essentially described in Example 1, except that OVCAR-8 NCI/ADR cells treated with or without an inhibitor of P-glycoprotein (P-gp), Elacridar (GF120918), were used. OVCAR-8 NCI-ADR cells overexpress drug pump MDR1 orABCB1 gene (encodes for P-glycoprotein) known to induce multi-drug resistance to anti-cancer agents (A Vert et al OncoTargets and Therapy 2018:11; 221-37). For comparison purposes, a 4-day image-based NCA of paclitaxel in the same OVCAR-8 NCI/ADR cells was carried out alongside the Compound C14 NCA. Briefly, OVCAR-8 cells were seeded in duplicate in a Corning 96-well Flat Clear Bottom Black Polystyrene plates (Corning, N.Y.) in 100 μL of appropriate complete media at the appropriate density and grown for 24 hours. In one set of plates, a concentration of Compound C14 or paclitaxel alone was serial diluted into 100 μL of complete media and then added to the cells with a final volume of 200 μL in complete media containing 0.5% DMSO. In a second set of plates, P-gp inhibitor GF120918 (1 μM) was added to culture media along with a concentration of Compound C14 or paclitaxel alone was serial diluted into 100 μL of complete media and then added to the cells with a final volume of 200 μL in complete media containing 0.5% DMSO. After 4 days (96 hours) of treatment, the cells were fixed by removing 100 μL of complete media from each well and replacing it with 100 μL of 2×formaldehyde (final 4%) and incubating the plates for 15 minutes at room temperature. After fixation, the cells were permeabilized and stained in 200 μL Wash Buffer (1% BSA, 0.2% Triton X-100, 1×PBS) containing 2 μg/mL Hoechst 33342 DNA dye. The plates were sealed and incubated for 1 hour at room temperature in the dark. Cells were stored at 4° C. in the dark until data acquisition. Imaging data was acquired on Cellomics ArrayScan VTI HCS Reader (SN03090745F, ThermoFisher Scientific) using the Target Activation V4 Assay protocol (Ve 6.6.0 (Build 8153) with a 10×objective, collected 16-fields per well). The Valid Object Count was determined using Hoechst 33342 nuclear object features (area, total and variable intensity in Channel 1) that were within ±3 SD of the DMSO-treated control. The Total Valid Object Count was represented as a Count POC (Percentage of DMSO Control) using the following formula:

Count POC=(Total Valid Object Count in treated well)+(Total Valid Object Count in DMSO treated wells)×100

Compound concentration and Count POC values were plotted using GraphPad Prism software (V7.0.4) and curve-fitting was performed with 4-parameter equation (variable slope). The concentration-response curves and standard deviation represent two independent experiments run in duplicate.

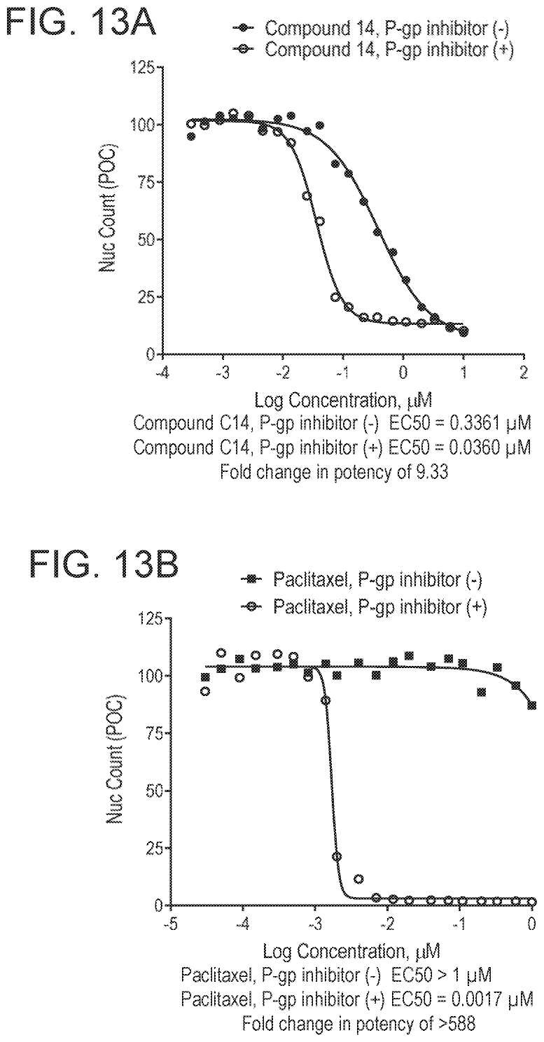

The results of the KIF18A inhibitor NCA and the paclitaxel NCA are shown in FIGS. 13A and 13B, respectively. In the absence of the P-gp inhibitor, the EC50 of the KIF18A inhibitor was about 10-fold higher than the EC50 of the KIF18A inhibitor in the presence of the P-gp inhibitor, whereas the EC50 of paclitaxel in the absence of the P-gp inhibitor was greater than 1 μM and the EC50 of paclitaxel in the present of the P-gp inhibitor was much less (0.0017 μM). The fold change in potency of the KIF18A inhibitor Compound C14 in the presence of the P-gp inhibitor vs in the absence of the P-gp inhibitor was less than 10, whereas the fold change in potency of paclitaxel in the presence of the P-gp inhibitor vs in the absence of the P-gp inhibitor was greater than 500. These results suggest that KIF18A inhibitors are able to effectively treat cancer cells, even multidrug resistant cancer cells.

### Example 14

This example demonstrates that KIF18A inhibitor treatment has minimal effects on normal somatic cells.

The effect KIF18A inhibitor treatment has on proliferation of normal somatic cells (e.g., not neoplastic cells) was tested by assaying proliferation of human bone marrow mononuclear cells (HBMNCs), primary human foreskin fibroblast cells (hFSF) and human mammary epithelial cells via a 5-bromo-2′-deoxyuridine (BrdU) incorporation assay in which BrdU, an analog of the nucleoside thymidine, is used to identify proliferating cells (Payton et. al., Molecular Cancer Therapeutics, 17(12):2575-85 (2018)). Cells were analyzed on BD LSRFortessa flow cytometer using BD FACSDiva software (BD Biosciences), and post-acquisition data analysis was performed using FSC-Express software (De Novo). The percentage of BrdU positive gated events on the stacked DNA content histograms were reported.

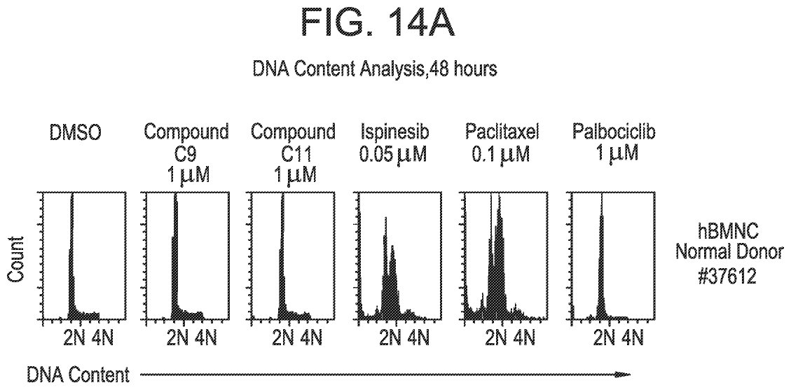

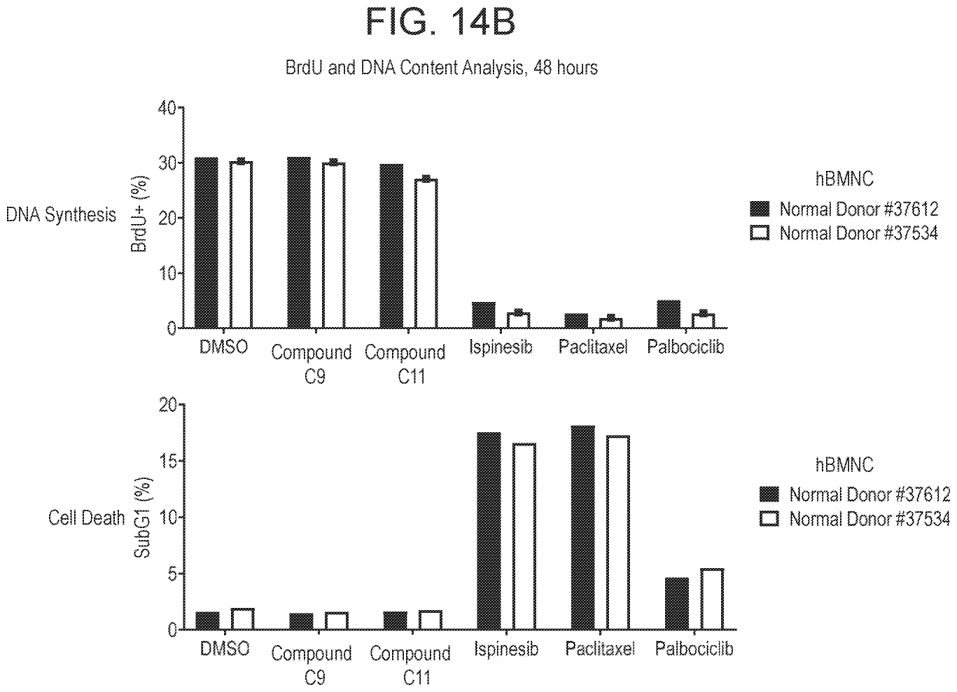

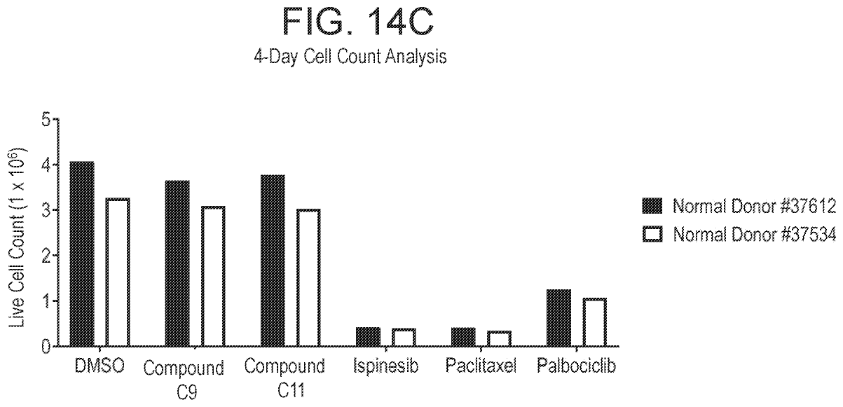

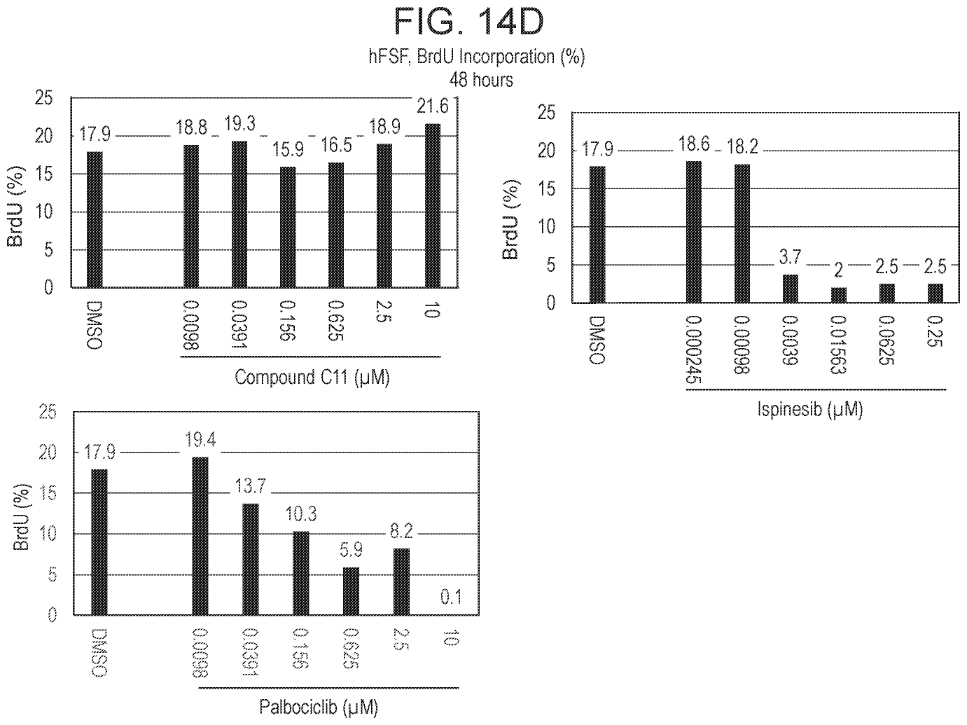

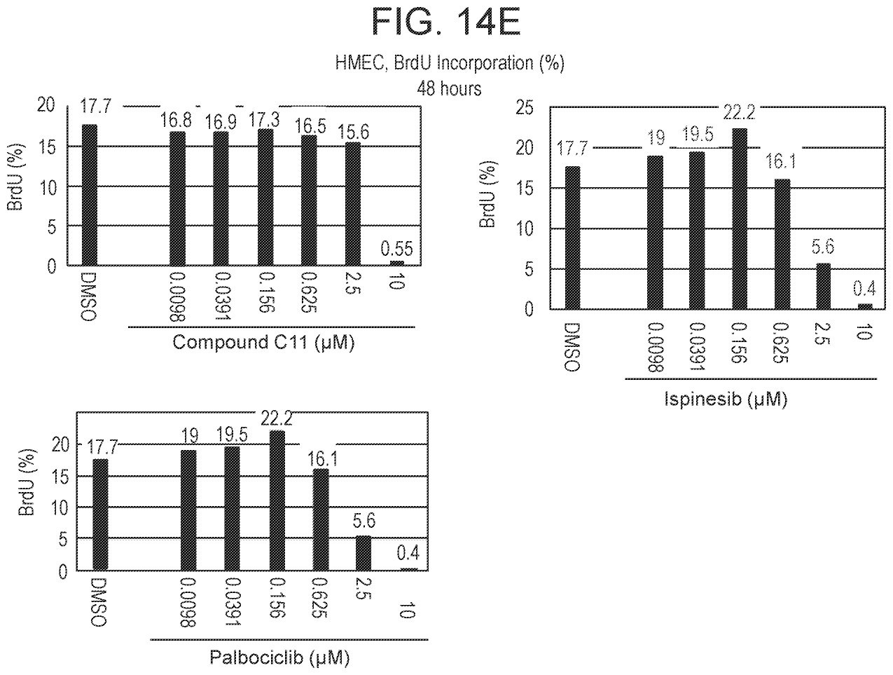

Exemplary flow cytometry results are shown in FIG. 14A. As shown in FIG. 14A, the cell cycle DNA content profiles for either Compound C9 or Compound C11 are similar to DMSO treated cells, in contrast cells treated with Ispinesib (Eg5), an anti-mitotic agent (paclitaxel), or a CDK 4/6 inhibitor (palbociclib) all showed marked effects on cell cycle DNA content profiles including an increase in the Sub-G1 (<2N) population (indicating cell death).

To further examine the effects of KIF18A inhibitor treatment on HBMNC, two normal donors were assessed using the BrdU coupled cell cycle assay and cell count assay (96 hours) was. As shown in FIG. 14B, the effect on proliferation seen in cells from the first donor was repeated in cells from a second donor. In particular, the amount of BrdU-stained proliferating cells treated with Ispinesib, paclitaxel, or a CDK 4/6 inhibitor (palbociclib) was far less than the amount of vehicle control-treated BrdU-stained cells. In contrast, the amount of BrdU incorporated into cells treated with a KIF18A inhibitor, either Compound C9 or Compound C11, was about the same as vehicle control-treated BrdU-stained cells. KIF18A inhibitor treatment was also analyzed for impact on live cell count. After 96 hours, live cells were counted by Vi-CELL XR Cell Viability Analyzer (Beckman Coulter). As shown in FIG. 14C, cells treated with ispinesib (Eg5), an anti-mitotic agent (paclitaxel), or a CDK 4/6 inhibitor (palbociclib) led to a lower cell count, relative to vehicle control treated cells, whereas cells treated with a KIF18A inhibitor (Compound C9, Compound C11) had little to no effect on normal cell counts. As shown in FIGS. 14D and 14E, the effect on BrdU incorporation in hFSF and human mammary epithelial (HMEC) cells were not impacted at concentrations <10 μM KIF18A inhibitor (Compound C11) relative to DMSO treated cells. In contrast, a decrease in BrdU incorporation was observed in cells treated with Ispinesib (Eg5), or a CDK 4/6 inhibitor (palbociclib). These results suggest that, unlike other anti-cancer agents, KIF18A inhibitors do not impact proliferation in normal somatic cells at the concentrations effective against KIF18A inhibitor sensitive cancer cells.

Imaging assays were also carried out to determine the effects of KIF18A inhibitor treatment on normal somatic cells. Arrayscan VTi multiplex imaging assays were carried out with human FSF cells as described below. Briefly, normal human foreskin fibroblast cells were seeded at 6000 cells per well in 96-well imaging plates (Corning) and cultured overnight. The next day, two replicate 96-well plates were treated with DMSO or panel of test agents over a 9-point concentration range using 3-fold dilution with top concentration of 10 μM (KIF18A inhibitor Compound C11, nutlin-3a), 1 μM (KIF18A inhibitor Compound C9, BI-2536, ispinesib, paclitaxel), or 5 μM (palbociclib, GSK923295). After 48 hours of treatment, one plate was pulsed with BrdU (Invitrogen) for 3 hours before fixation. Both 96-well plates fixed with 4% formaldehyde (Thermo Scientific), washed twice with wash buffer [PBS, 1% BSA (Thermo Fisher), 0.2% Triton X-100 (Sigma)]. The first 96-well plate was processed for BrdU epitope detection using acid wash, blocked overnight at 4° C. in wash buffer supplemented with horse serum (4 drops serum per 10 mL) (Vector Labs) and stained with anti-BrdU-AlexaFluor-647 (B35140, Invitrogen, mouse, 3 μg per mL) and anti-p21 (12D1) (2947, Cell Signaling, rabbit, 1:400) antibodies for two hours at room temperature. Cells were washed twice in wash buffer and stained with secondary antibody [anti-rabbit-IgG-AlexaFluor-488 (A1 1034, Invitrogen, 1:2000)] and incubated for one hour at room temperature. The second 96-well plate was blocked overnight at 4° C. in wash buffer supplemented with horse serum (4 drops serum per 10 mL) (Vector Labs) and stained with anti-cl-PARP (214/215) (44-6986, Invitrogen, rabbit, 1:1500) and anti-γH2AX (05-636, Millipore, mouse, 1:1000) antibodies for two hours at room temperature. Cells were washed twice in wash buffer and stained with secondary antibodies [anti-rabbit-IgG-AlexaFluor-647 (A21245, Invitrogen, 1:2000), anti-mouse-IgG-AlexaFluor-488 (A1 1029, Invitrogen, 1:2000)] and incubated for one hour at room temperature. Both 96-well plates were washed twice and counterstained with Hoeschst 33342 (Invitrogen) nuclear dye. Imaging data was collected by widefield imaging on ArrayScan VTi HCS Reader (Thermo Scientific) from 64 fields per well using 20×objective. Valid nuclear object counts were determined for each well as well as the percentage of BrdU, p21, cl-PARP, and γH2AX positive objects for each test agent concentration and DMSO control. Concentration-response heatmaps were generated using GraphPad Prism software (V7.0.4).

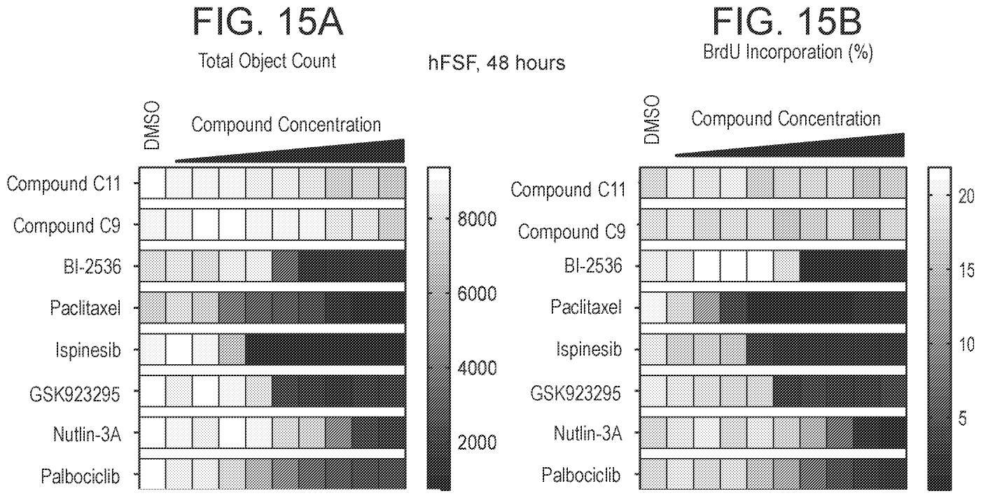

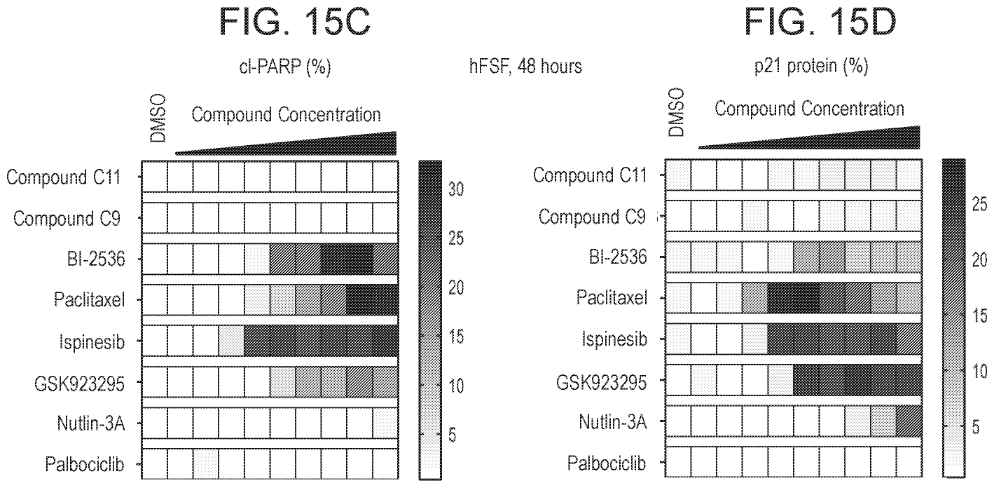

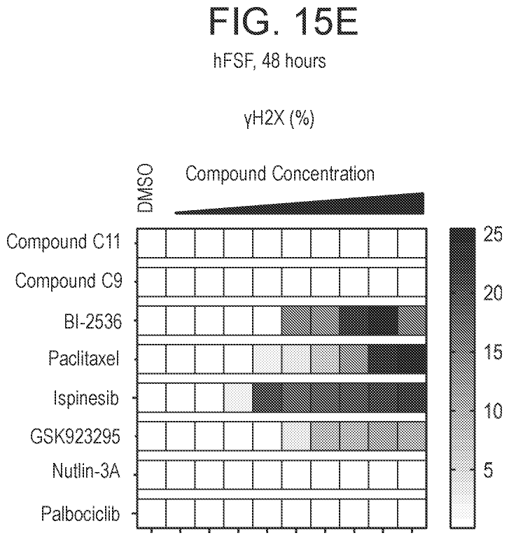

The results are shown in FIGS. 15A-14E. As shown in FIGS. 15A-15B, cells treated with a KIF18A inhibitor (Compound C11 or Compound C9) behaved as vehicle control treated cells in terms of total object count and BrdU incorporation, indicating minimal effects on cell proliferation As shown in FIGS. 15C-15E, cells treated with a KIF18A inhibitor (Compound C11 or Compound C9) showed no induction of apoptosis measured by cl-PARP expression (FIG. 15C), no cell cycle arrest measured by the induction of p21 protein expression (FIG. 15D), or induction of DNA damage measured by increase in γHH2X expression (FIG. 15E). All the comparator agents induced one or more of these markers relative to the DMSO control. These results suggest that, unlike other anti-cancer agents, KIF18A inhibitors do not impact proliferation in normal somatic cells.

Taken together, these results suggest that the effect of KIF18A inhibitors is cancer cell-specific, has little to no toxicity in or on normal somatic cells, and that KIF18A inhibitor treatment is effective for treating the neoplastic disease, maintaining sensitivity to treatment with a CDK4/6 inhibitor, inducing or increasing tumor regression, reducing tumor or cancer growth, and/or inducing or increasing death of a tumor or cancer cell, without overt toxicity to normal somatic cells, as demonstrated by a lack of a substantial decrease in the proliferation of normal somatic cells in the subject and/or lack of a substantial increase in the apoptosis of normal somatic cells.

### Example 15

This example demonstrates RNA-based KIF18A inhibitors that reduce KIF18A gene expression.

A series of seven KIF18A siRNAs was obtained from three different vendors (Qiagen, Dharmacon, Ambion) for use in this study. Non-targeting control (NTC) siRNAs and Eg5 (hKIF11) siRNAs were also obtained to serve as negative controls and positive controls, respectively. The nucleotide sequences of the siRNAs are listed in Table 5.

TABLE 5

| | | | SEQ | | |
|---|---|---|---|---|---|
| | Catalog #, | | ID | Count | Mitotic |
| Gene_SiRNA_ID | Custom ID | Sequence* | NO. | Assay | Assay |
| hKIF18A_1 | S100140224 | ATCCGTCTACAGTAACCTTAA | 12 | Yes | No |
| hKIF18A2 | S00140238 | CAGGTGGAACTAATCTGGTTA | 13 | Yes | Yes |
| hKIF18A_3 | S00140245 | CAGGAGGACTTGGACTCTACA | 14 | Yes | Yes |
| hKIF18A_4 | J-006849-05- | UAAAUUACCCGAACAAGAA | 15 | Yes | Yes |
| | 0005 | | | | |
| hKIF18A_5 | S103090941 | CTCGAAGTGTAAATTACCCGA | 16 | Yes | Yes |
| hKIF18A_6 | J-006849-08- | GGAUAUAAUUGCACAGUAC | 17 | Yes | Yes |
| | 0005 | | | | |
| hKIF18A_7 | 118492 | GCAGCUGGAUUUCAUAAAGTT | 18 | Yes | No |
| hEg5_1 | S102653770 | GCCGATAAGATAGAAGATCAA | 19 | Yes | No |
| (hKIF11) | | | | | |
| hEg5_2 | S03019793 | CTCGGGAAGCTGGAAATATAA | 20 | Yes | No |
| (hKIF11) | | | | | |
| NTC_1 | D-001810-01- | UGGUUUACAUGUCGACUAA | 21 | Yes | Yes |
| | 05 | | | | |
| NTC_2 | D-001810-02- | UGGUUUACAUGUUGUGUGA | 22 | Yes | Yes |
| | 05 | | | | |
| NTC_3 | D-001810-03- | UGGUUUACAUGUUUUCUGA | 23 | Yes | Yes |
| | 05 | | | | |
| NTC_4 | D-001810-04- | UGGUUUACAUGUUUUCCUA | 24 | Yes | Yes |
| | 05 | | | | |
| NTC_5 | Custom | AACGCAGAGTTCGACCGTTTA | 25 | Yes | No |
| | Random 2-4 | | | | |
| NTC_6 | Custom | AAGGCGGGTCCGGCAGTTTTT | 26 | Yes | No |
| | Random 9-1 | | | | |
| NTC_7 | Custom M13-4 | AATGCGCTTCCCTGTTTTTAT | 27 | Yes | No |
| NTC_8 | Custom | AACCACCTTGAACACGTATTT | 28 | Yes | No |
| | Random 10-1 | | | | |
| NTC_9 | Custom | AAGGCCACTTGCGTCAGATTT | 29 | Yes | No |
| | Random 7-1 | | | | |

NTC (non-targeting control);
*as provided by vendor

The KIF18A knockdown efficiency of each siRNA was tested by Western analysis in BT-549, and HMEC cells. Briefly, BT-549, and HMEC cells were seeded in 6-well plates (Thermo Scientific) at 2.0×10⁵ cells per well and cultured overnight. The next day, cells were treated with RNA-lipid complex using RNAiMax Lipofectamine according to manufactures protocol (Invitrogen) with 10 nM individual KIF18A siRNAs (n=7) or NTC siRNA (NTC_2). After 48 hours, cell lysates were prepared using RIPA buffer and processed for Western analysis. The level of β-actin was assayed to demonstrate equal protein loading in each lane. HeLa cells treated with nocodazole overnight were used as a mitotic fraction positive control and Jurkat cells treated with staurosporine as apoptosis positive control.

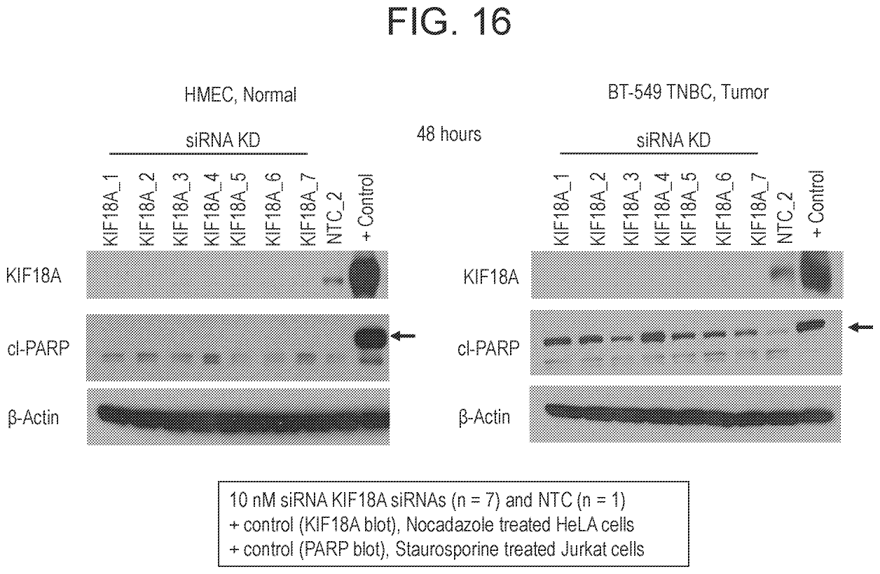

As shown in FIG. 16, each KIF18A siRNA (KIF18A_1 to KIF18A_7) effectively depleted KIF18A protein expression in HMEC and BT-549 cells, whereas cells transfected with control siRNA (NTC_2) showed baseline KIF18A expression, as expected the HeLa cell mitotic fraction exhibited high levels of KIF18A expression. These data show KIF18A inhibitors, such as KIF18A siRNAs, induce apoptosis of BT-549 breast cancer cells without evidence of apoptosis in normal somatic (non-cancerous) breast epithelial cells.

### Example 16

This example explores the effects of RNA-based KIF18A inhibitors on cancer cells.

To determine the pattern of sensitivity and phenotypes induced by siRNA-mediated KIF18A depletion, a panel of eight cancer cell lines (7 breast, 1 ovarian) as well as 1 normal human mammary epithelial cell line (HMEC) was assembled. The cancer cell lines were selected based on tumor subtype and genetic background (TP53, RB1, CCNE1).

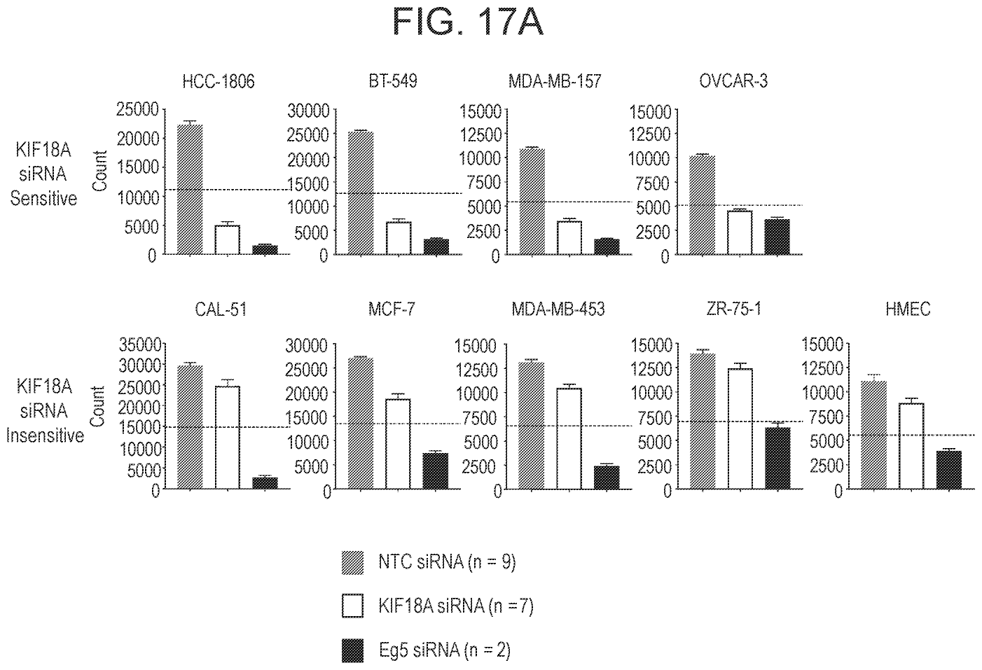

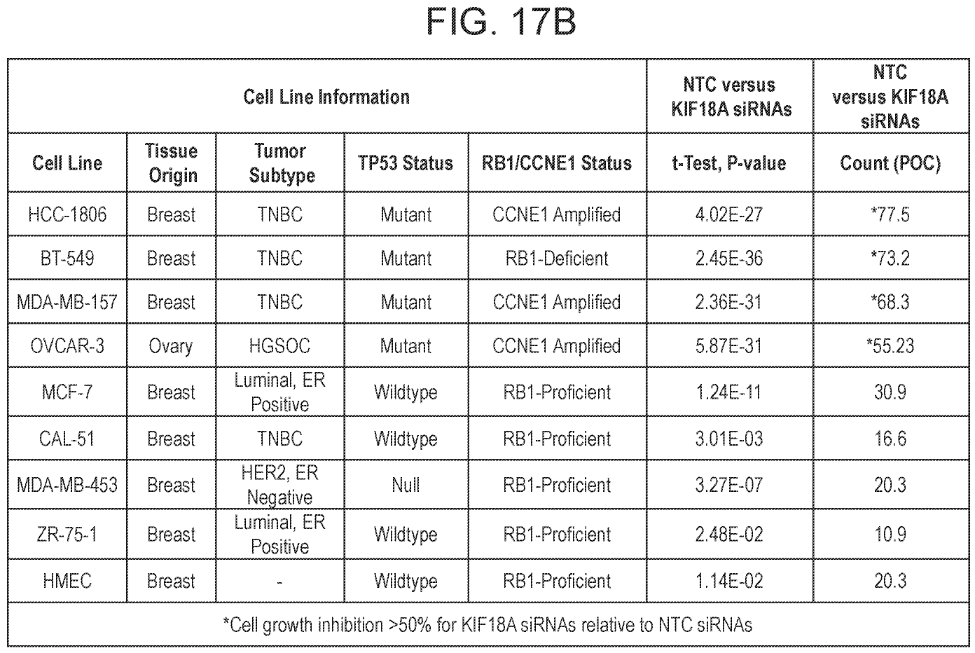

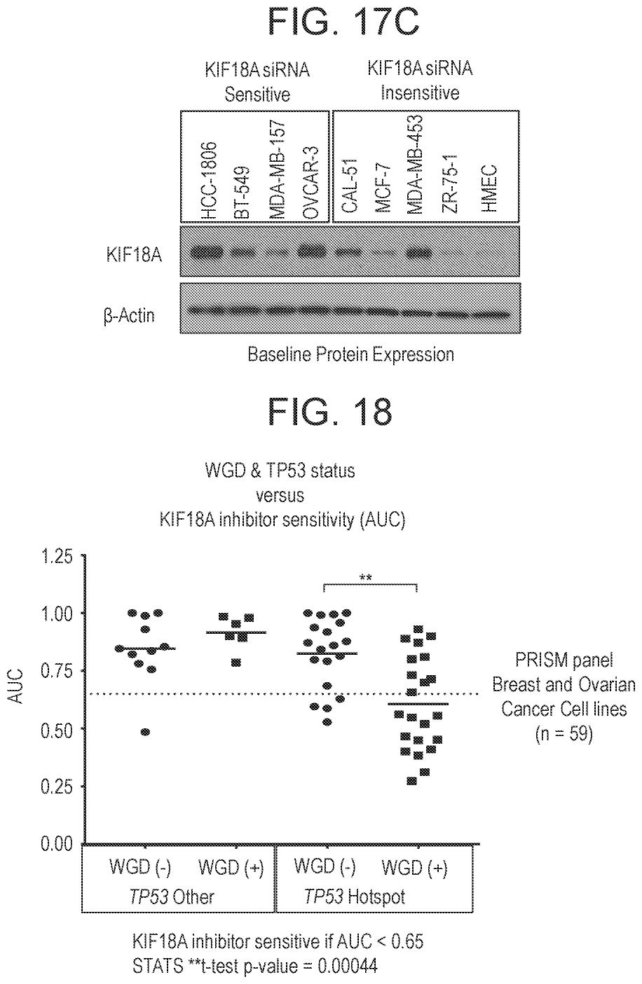

Using an imaging-based nuclear count assay, the panel was used to determine the anti-proliferative effects of individual siRNAs for KIF18A (n=7), compared to the effects of non-targeting controls (NTC, n=9) and cytotoxic controls (KIF11 (Eg5), n=2), on cells treated for four days. Cells were considered as KIF18A siRNA sensitive when >50% inhibition of cell growth was observed. As shown in FIGS. 17A-17B, KIF18A siRNA sensitivity was observed in all three CCNE1 amplified lines HCC-1806 (TNBC), MDA-MB-157 (TNBC), OVCAR-3 (HGSOC) and in RB1-deficient BT-549 TNBC line. KIF18A siRNA insensitive breast cancer cell lines were TP53 wild-type (3 of 4), RB1 proficient (4 of 4), estrogen receptor status (ER positive 2 of 4) and (ER negative 2 of 4). TP53 wild-type CAL-51 TNBC cells with a near normal karyotype were insensitive to KIF18A siRNAs as well as normal HMEC line, consistent with findings with immortalized human retinal pigment epithelial cell line (hTERT-RPE1). In contrast, and as predicted, Eg5 siRNAs were cytotoxic across the cell line panel, demonstrating its essentiality for somatic cell division (FIG. 17A). Results of the KIF18A siRNAs relative to NTC controls are provided in FIG. 17A are summarized in the table (FIG. 17B), summary table contains cell line information, genetic background, and KIF18A vs NTC siRNA groups statistical assessment (t-test) and level of decrease in cell growth. KIF18A protein expression varied across the panel of cell lines and showed no direct correlation with sensitivity (FIG. 17C).

Taken together, these results demonstrate that KIF18A siRNAs which deplete KIF18A expression demonstrate selective anti-proliferative activity on cancer cells of a particular genetic background with respect to TP53, CCNE1, and RB1, which results are consistent with earlier observations (e.g., Examples 1-10). These results also support that KIF18A siRNAs are able to induce apoptosis of cancer cells and inhibit the growth of cancer cells.

### Example 17

This example describes the materials and methods used in Examples 15 and 16.

Cell Lines. All human cancer cell lines were obtained ATCC or DSMZ (GmbH) unless otherwise specified. Cell lines were authenticated by ATCC using short tandem repeat (STR) DNA analysis and referenced against ATCC or ExPasy STR databases. Normal human mammary epithelial cell (HMEC) were purchased from Lonza Inc. All cell line cultures were maintained in at 37° C. in an atmosphere of 5% CO₂.

Imaging Assays

ArrayScan VTI nuclear count assay (siRNAs). A panel of cell lines (HCC-1806, BT-549, MDA-MB-157, OVCAR-3, MCF-7, CAL-51, MDA-MB-453, ZR-75-1, HMEC) were seeded in 96-well imaging plates (Corning) at densities individually optimized for log phase cell growth. The next day, cells were treated with RNA-lipid complex containing individual siRNAs at 10 nM [KIF18A (n=7), Eg5 (n=2), NTC (n=9), siRNA details in (Table 5) and 0.3 μL RNAiMax Lipofectamine (Invitrogen) according to manufactures protocol (Invitrogen). After four days, cells were fixed with 4% formaldehyde (Thermo Scientific), washed with PBS (Invitrogen), and stained with Hoechst 33342 (Invitrogen) nuclear dye in wash buffer [PBS, 1% BSA (Thermo Fisher), 0.2% Triton X-100 (Sigma)]. Valid nuclear objects (within three SD of mean nuclear object area for the control well) were enumerated using Cellomics ArrayScan VTi HCS Reader (Thermo Scientific) equipped with 10×objective using Target Activation BioApplication (Thermo Scientific), valid nuclear object count data was collected for sixteen image fields per well. Graphing and statistical analysis performed using GraphPad Prism 7.04 (GraphPad Software). Data is represented as mean nuclear count and standard error of the mean (SEM) from the aggregated individual siRNA count data from two independent experiments run in duplicate [KIF18A (n=28), Eg5 (n=8), NTC (n=36)]. Significance was computed using unpaired t-test comparing NTC and KIF18A siRNA groups.

Western Analysis:

Western analysis methods. Cell lysates were prepared by combining the non-adherent and adherent cell fractions using either RIPA Buffer (Sigma) or Minute™ Total Protein Extraction Kit (Invent Biotechnologies), supplemented with a cocktail of protease and phosphatase inhibitors (Roche). Total protein concentrations were determined using Bradford dye-binding method (Bio-Rad), lysates were stored at −80° C. Proteins were resolved Tris-Glycine gel (Invitrogen) based on protein size and transferred to PDVF membrane (Bio-Rad). Protein membranes were incubated in 10 mL of blocking buffer [wash buffer (PBS, 0.5% Tween-20), 5% dry milk (Albertsons), 3 drops horse serum (for mouse antibodies, Vector Labs) or goat serum (for rabbit antibodies, Vector Labs)] for 60 minutes at room temperature on an orbital shaker. Primary antibodies were added to blocking buffer and incubated overnight at 4° C. on an orbital shaker. Membranes were washed thrice (15 minutes each) followed by secondary antibody treatment using Vectastain ABC Kit (PK-4002 (mouse), PK-4001 (rabbit), Vector Labs). Protein detection performed with Western Lighting Chemiluminescence reagent (Perkin Elmer) before developing membranes on film (USA Scientific).

Westerns analysis antibodies. Anti-cleaved-PARP (cl-PARP) (51-900017, BD Pharmingen, mouse, 1:500), anti-β-actin (A5441, Sigma, mouse, 1:5000), anti-KIF18A (HPA039484, Sigma, rabbit, 1:2000),

Assessment of baseline KIF18A and Cyclin E1 expression. A panel of cell lines were seeded in 6-well plate (Thermo Scientific) at densities individually optimized for log phase cell growth. At approximately 80% confluency, cells were harvested, and cell lysates were prepared using RIPA buffer and processed for Western analysis as described above.

Assessment of KIF18A siRNA knockdown efficiency. HMEC, and BT-549cells were seeded in 6-well plates (Thermo Scientific) at 2.0×10⁵ cells per well and cultured overnight. The next day, cells were treated with RNA-lipid complex using RNAiMax Lipofectamine according to manufactures protocol (Invitrogen) with 10 nM individual KIF18A siRNAs (n=7) or NTC siRNA (NTC_2). Information on individual siRNAs are shown in Table 5. After 48 hours, cell lysates were prepared using RIPA buffer and processed for Western analysis as describe above. HeLa cells treated with 0.1 μg/mL of nocodazole (Millipore) overnight was used as a mitotic fraction positive control. Jurkat cells were treated with 1 uM of staurosporine for 24 hours as a cl-PARP positive control.

### Example 18

This example demonstrates that TP53^{MUT} human breast and ovarian cancer cell lines comprising one or more whole genome doubling (WGD) events correlate with enrichment to KIF18A inhibitor treatment.

KIF18A inhibitor Compound C9 was screened using PRISM molecular barcoded cancer cell line panel that included 59 breast and ovarian cancer cell lines (Channing Yu et all, Nature Biotech 2016 April; 34(4):419-23, Steven M Corsello et al Nature Cancer 2020 February; 1(2):235-248). Briefly, pool bar-coded cell lines were treated with Compound C9 (8-points, 2.5 μM to 0.001 μM) for 5 days. A curve-fitting analysis was performed and an area under the curve (AUC) viability value was computed for each cell line. WGD status calls for each cancer cell line was obtained from Quinton et al BioRxiv, 2020.06.18.159095; doi: https://doi.org/10.1101/2020.06.18.159095. WGD scores of 0, 1, or 2 were assigned for each cell line according to Quinton et al., 2020, supra. In this correlation analysis, the cancer cell lines were assigned to one of two groups: WGD negative (0 WGD events) or WGD positive (1 or 2 WGD events). TP53 status calls for each cancer cell line was obtained from the Broad Institute cancer dependency map (depmap.org, Mutation DepMap Consortium 20Q2). A “TP53 Hotspot” status call indicated the cell line harbored a TP53 mutation and the “TP53 other” status call indicated the cell line had a wild-type status. Next, KIF18A inhibitor Compound C9 AUC values for each cell line were graphed into four groups: (1) TP53 Other WGD (−), (2) TP53 Other WGD (+), (3) TP53 Hotspot WGD (−), and (4) TP53 Hotspot WGD (+). The lower the Compound C9 AUC viability value was the more sensitive the cell line was to KIF18A inhibitor treatment. An AUC threshold of <0.65 was set to indicate KIF18A inhibitor sensitivity. Graphing of data and statistical test (unpaired t-test, TP53 Hotspot WGD (−) versus WGD (+)) was performed using GraphPad Prism software. Results are shown in FIG. 18.

As shown in FIG. 18, KIF18A inhibitor sensitivity statistically significantly correlated with WGD positive event in TP53^{MUT} cancer cells (p-value=0.00044) suggesting that KIF18A inhibitors reduce the growth and/or induce apoptosis of TP53^{MUT} cancer cells comprising one or more WGD events. These data support that human cancers with TP53^{MUT} plus one or more WGD events are likely to respond to KIF18A inhibitor therapy.

### Example 19

This example describes the characterization of three KI18A inhibitors.

A trio of KIF18A inhibitors (Compound C9, Compound C11, and Compound C12) were synthesized and tested in vitro for KIF18A inhibitory activity. FIG. 19A shows ADP-Glo concentration-response profiles of KIF18A motor activity (presented as MT-ATPase luminescence signal relative to DMSO control (POC)). The values represent mean t SEM from three independent experiments. As shown in FIG. 19A, all three KIF18A inhibitors C9, C11, and C12 exhibited potent human KIF18A inhibitory activity. The IC50s for C9, C11 and C12 were 0.180 μM, 0.07 μM, and 0.04 μM, respectively. As in vivo studies in mice were planned, the mouse KIF18A inhibitory effects of Compound C9 and C12 were assayed. The IC50s for Compounds C9 and C12 in mice were 0.232 μM and 0.039 μM, respectively, and thus demonstrated that the KIF18A inhibitory effects of C9 and C12 were essentially equivalent for mouse and human KIF18A motors.

KIF18A inhibitor cancer cell line sensitivity profiles were determined for KIF18A inhibitor Compounds C9 and C11. Cells of various cancer cell lines were treated with DMSO or increasing concentrations of C9 or C11 for 96 h. Exemplary concentration-response profiles of cancer cell lines for C9 are provided in FIGS. 4C-4F. The concentration-response profiles of some cancer cell lines (including, e.g., BT-549, OVCAR-3) for C11 were similar. Mean Count EC₅₀ values for C9 and C11 in KIF18A inhibitor-sensitive cells (e.g., OVCAR-3 and BT-549) were 0.021 μM and 0.047 μM, respectively. Cancer cell lines CAL-51, MDA-MG-453 and OVCAR-5 were insensitive to C9 and C11. Interestingly, the sensitivity profiles for C9 and C11 were opposite of a CDK 4/6 inhibitor cancer cell lines that were sensitive to KIF18A inhibitors C9 and C11 were insensitive to the CDK 4/6 inhibitor, while those cancer cell lines that were insensitive to KIF18A inhibitors C9 and C11 were sensitive to the CDK 4/6 inhibitor.

To investigate whether KIF18A inhibitory activity of Compounds C9, C11, and C12 would translate to a cellular context, pH3 and PCM spot EC₅₀ values in MDA-MB-157 cells were determined and the results demonstrated a near-perfect potency alignment between mitotic endpoints. Indeed, the cell potency was dramatically improved for Compound C11 (>70-fold), Compound C9 (>450-fold), and Compound C12 (>120-fold) relative to a control compound.

To better understand the durability of response after KIF18A inhibitor treatment, all five KIF18A inhibitor-sensitive cancer cell lines were treated with DMSO or Compound C11 in a 6-day cell growth assay, where the surviving cells were washed, collected, counted, and re-plated in drug-free growth media and cultured for additional 7 to 9 days. MCF-7 cells treated with a CDK 4/6 inhibitor was included as a cytostatic comparator. As expected, treatment with Compound C11 showed a significant decrease in cell growth and colony formation, notably, the cells previously exposed to Compound C11 showed a marked reduction cell growth potential relative to DMSO control, and distinct from the CDK 4/6 inhibitor. Note that HCC-1806 and BT-549 cells showed greater regrowth potential relative to the other cell lines after KIF18A inhibitor withdrawal.

To investigate whether KIF18A inhibitors could circumvent this normal cell toxicity barrier, we examined the effects of Compounds C9 and C11 on a panel of cycling normal somatic cell types. First, we treated human bone marrow mononuclear cells (HBMNC) from two normal donors with DMSO, Compound C9 and C11 at 1 μM, ispinesib at 0.05 μM, paclitaxel at 0.05 μM, or palbociclib at 1 μM for 48 h (cell cycle) or 96 h (cell growth). Remarkably, cell cycle analysis revealed KIF18A inhibitor treatment had minimal diminution in BrdU incorporation (a direct measure of DNA synthesis) relative to DMSO control, distinct from the three comparator agents, ispinesib and paclitaxel were clearly cytotoxic, whereas CDK4/6 inhibitor was largely cytostatic. Cell growth analysis at 96 h showed comparable cell counts for both KIF18A inhibitors and DMSO control, whereas a clear reduction in cell growth was observed for ispinesib and paclitaxel (˜88% reduction), and to a lesser extent with palbociclib (68% reduction).

To establish whether the observed tumor PD effect with both KIF18A inhibitors would result in tumor efficacy, nude mice with OVCAR-3 tumors (130 mm³, n=10) were dosed IP with vehicle alone, Compound C9 at 100 mg/kg, or Compound C12 at 25 mg/kg once daily for 18 consecutive days. Remarkably, both C9 and C12 induced 73% and 46% tumor regressions, respectively (p<0.0001) (FIG. 19B). C9 and C12 treatment was well-tolerated by the mice with no evidence of body weight loss or changes in blood counts (neutrophil, reticulocyte, lymphocyte, monocyte, red blood cells, and white blood cells), expected for decrease on monocytes with C12 (p=0.043). Terminal PK analysis revealed C9 and C12 plasma AUC values of 130 and 53 μM-h, respectively.

To assess anti-tumor activity of KIF18A inhibitors in near-diploid tumor model, nude mice with CAL-51 tumors (140 mm³, n=10) were dosed IP with vehicle alone, C9 at 100 mg/kg, or C12 at 25 mg/kg once daily for 18 consecutive days. CAL-51 was a cancer cell line that was demonstrated in vitro as insensitive to C9 and C11. Both KIF18A inhibitors showed no observable effect on CAL-51 tumor growth relative to vehicle control (FIG. 19C). Consistent with OVCAR-3 study, KIF18A inhibitor treatment was well-tolerated by the mice with no evidence body weight loss (FIG. 19B) and terminal PK analysis showed comparable plasma concentration-time profiles between studies.

To further examine KIF18A inhibitors activity in HGSOC in vivo, mice with OVCAR-8 tumors (145 mm³, n=10) were dosed IP with vehicle alone, C9 at 50 or 100 mg/kg, or C12 at 25 or 50 mg/kg once daily for 18 consecutive days. As shown in FIG. 19D, treatment with C9 or C12 resulted in 16% and 73% tumor regressions at 50 and 100 mg/kg (p<0.0001) or 19% and 75% tumor regressions at 25 and 50 mg/kg (p<0.0001), respectively. As before, inhibition of KIF18A activity was well-tolerated by the mice with no evidence body weight loss (FIG. 19D). At matched doses, the plasma AUC values were 2.8-fold higher for C12 in OVCAR-8 study relative to the other efficacy studies, whereas the C9 PK profiles were comparable across studies.

Collectively, these in vivo data provide the first evidence of anti-cancer activity with small molecule inhibitors of KIF18A with a robust tumor PD response and marked tumor regressions at well-tolerated doses.

### Example 20

The following materials and methods were used in the study of Example 19.

Cell Lines. All human cell lines were procured from ATCC or DSMZ (GmbH) unless otherwise specified. Cell lines were authenticated by ATCC using short tandem repeat (STR) DNA analysis and referenced against ATCC or ExPasy Cellosaurus (Robin et al 2020) STR databases. OVCAR-5, OVCAR-8, and OVCAR-8 NCI/ADR-RES (also known as ADR^{RES}) (Vert et al 2018) cell lines were procured from National Cancer Institute. Human bone marrow mononuclear cells (HBMNCs) and human mammary epithelial cells (HMECs) were procured from Lonza Inc. Human T-cells were isolated from Leukopaks procured from HemaCare Inc. MDA-MB-157 Cas9 cell line was procured from Cellecta Inc. Kyoto HeLa cell line expressing α-tubulin-EGFP and H2B-mCherry proteins was procured from Creative Bioarray Inc. All cell line cultures were maintained in at 37° C. in an atmosphere of 5% CO₂. OVCAR-3, CAL-51, and OVCAR-8 cell lines used for in vivo studies were determined to be free of contamination with mycoplasma and a panel of murine vial pathogens. Cell line information for tumor tissue type, tumor subtype, TP53 mutation status, RB1 and CCNE1 status, and whole genome doubling (WGD) status based on cross referenced public data sources (DepMap Consortium, IARC TP53 database, Sanger Cell Model Passport, Broad Institute CCLE, ATCC, DSMZ) and published studies (Domcke et al 2013, Dai et al 2017, Quinton et al 2020).

Chemistry. The molecular structures have been disclosed for compounds BI-2536 (Steegmaier et al 2007), BTB-1 (Catarinella et al 2009), doxorubicin (Carvalho et al 2009), gemcitabine (Pourquier et al 2002), GF120918 (Hyafil et al 1993), GSK923295 and ispinesib (Rath and Kozielski 2012), nutlin-3a (Vassilev 2004), paclitaxel and docetaxel (Perez 2009), palbociclib (O'Leary et al 2016), KIF18A inhibitor compounds were synthesized by Amgen.

KIF18A Inhibitor Activity

KIF18A compounds C9, C11, C12 were 2-fold serially diluted over a 22-point concentration range in DMSO using 384-well plate (Corning). Recombinant truncated kinesin motor proteins (human KIF18A (residues 1-467, 4 nM), mouse KIF18A (residues 1-467, 4 nM)) were expressed and purified. ADP-Glo luminescence assay (Promega) was used to measure the MT-ATPase motor activity. Porcine brain MTs were procured from Cytoskeleton Inc. Compounds were pre-incubated for <30 min in reaction buffer (15 mM Tris, pH 7.5, 10 mM MgCl₂, 0.01% Pluronic F-68, 2% DMSO, 1 μM paclitaxel, 30 μg/mL MTs) with motor proteins at concentrations indicated above, 30 μM ATP was added to initiate the enzymatic reaction for 15 min at room temperature (RT). ADP-Glo reagents were added according to manufactures protocol, the luminescence intensity proportional to ADP present was measured using an EnVision plate reader (Perkin Elmer). Raw luminescence signal data was normalized to POC (percentage of positive control) and then Activity (%) was computed [POC=100×(sample signal−negative control signal)+(positive control signal−negative control signal). Activity (%)=POC−100]. The positive control (enzyme+substrate) and negative control (substrate alone) had equivalent concentrations of DMSO. Curve-fitting and IC₅₀ values were determined by 4-parameter non-linear regression equation (variable slope) using GraphPad Prism 7.05 (GraphPad Software).

Cell growth assay. A panel of cell lines (HCC-1806, HCC-1937, BT-549, MDA-MB-157, MCF-7, CAL-51, MDA-MB-453, ZR-75-1, OVCAR-3, OVCAR-5) were seeded in 96-well plate at densities optimized for 96 h cell growth assay. The next day, cells were treated with DMSO or one of the following compounds: KIF18A Inhibitor Compounds C9 and C11, or palbociclib, with top concentration of 6 μM (19-point concentration range) using staggered dose method. After 96 h, cells were fixed, stained, imaged, and analyzed as described above. Valid nuclear object counts were determined for each well and Count POC value was computed using the following formula [Count POC=(valid nuclear object count in treated well)+(valid nuclear object count in DMSO treated wells)×100]. Concentration-response curve-fitting was performed by 4-parameter non-linear regression equation using GraphPad Prism 7.05. Mean Count EC₅₀ value was computed for each cell line from two independent experiments run in duplicate. If a maximal response of >50% was not reached at top concentration, cell lines were assigned Count EC₅₀ value of 6 μM and were considered insensitive. A mean Count EC₅₀ value was computed across sensitive cell lines for each test agent.

BrdU and cell cycle analysis. Human bone marrow mononuclear cells (HBMNCs) from two normal donors (#37612, #37534) were cultured for 8 days in defined media as previously described (Payton et al 2018). HBMNCs were seeded in 24-well plate at 1.2×10⁶ cells per well in duplicate plates. Cells were treated with DMSO, Compound C9 (1 μM), Compound C11 (1 μM), ispinesib (0.05 μM), paclitaxel (0.1 μM), or palbociclib (1 μM). Cells were collected at 48 h (cell cycle) and 96 h (cell growth). The first set of plates were pulsed with BrdU for 2 h and processing for BrdU cell cycle analysis as previously described (Payton et al 2018). Cells were analyzed on BD LSRFortessa flow cytometer running BD FACSDiva software and post-acquisition data analysis was performed using FSC-Express software. Data was reported for the percentage of BrdU and subG₁ gated populations. The second set of plates were collected, and cells counted using Vi-CELL XR Cell Viability Analyzer (Beckman Coulter).

Graphing was performed for each donor using GraphPad Prism 7.05.

OVCAR-3 Tumor Xenograft Efficacy Study

Mice were injected with human OVCAR-3 cells (5.0×10⁶) subcutaneously in the right flank. Animals with established tumors were randomized into four groups (n=10 per group) with average tumor volume of 130 mm³. Animals were dosed IP with vehicle alone daily, C9 (100 mg/kg) daily, or C12 (25 mg/kg) daily. Treatment started on day 24 post-tumor implantation and treatment terminal on day 42. Tumor volumes and body weights were recorded twice per week using a digital caliper and analytical lab scale, respectively. After the final dose on day 42, complete blood count analysis (n=6 per treatment group) was performed by IDEXX Inc. Plasma pharmacokinetic analysis was performed for C9 and C12 at 2, 4, 8, 16 and 24 h (n=2 per time point) by standard LC-MS/MS methods. The percentage of tumor growth inhibition (% TGI) was calculated as % TGI relative to vehicle control: % TGI=100−[(Treated−Initial Volume)/(Control−Initial Volume)×100]. The percentage of tumor regression (% TR) was calculated as % TR compared final tumor volume to the initial tumor volume: % TR=100−[(Final Volume)/(Initial Volume)×100]. Data was graphed using GraphPad Prism 7.05. Statistical analysis was performed on treatment groups for tumor growth and blood counts using repeated measures ANOVA and one-way ANOVA, respectively, followed by Dunnett's multiple comparisons test.

CAL-51 Tumor Xenograft Efficacy Study

Mice were injected with human CAL-51 cells (5.0×10⁶) subcutaneously in the right flank. Animals with established tumors were randomized into four groups (n=10 per group) with average tumor volume of 140 mm³. Animals were dosed IP with vehicle alone daily, C9 (100 mg/kg) daily, or C12 (25 mg/kg) daily. Treatment started on day 18 post-tumor implantation and treatment terminal on day 36. Tumor volume and body weight assessment performed as described above. After the final dose on day 36, plasma pharmacokinetic analysis was performed as described above. All data analysis was performed as described above.

OVCAR-8 Tumor Xenograft Efficacy Study

Mice were injected with human OVCAR-8 cells (5.0×10⁶) subcutaneously in the right flank. Animals with established tumors were randomized into four groups (n=10 per group) with average tumor volume of 145 mm³. Animals were dosed IP daily with vehicle alone, C9 (50 or 100 mg/kg), or C12 (25 or 50 mg/kg). Treatment started on day 28 post-tumor implantation and treatment terminal on day 46. Tumor volume and body weight assessment performed as described above. After the final dose on day 46, plasma pharmacokinetic analysis was performed as described above except blood was drawn by retro-orbital bleed method.

### REFERENCES

The following references are cited in this example:
Robin T, Capes-Davis A, Bairoch A. CLASTR: The Cellosaurus STR similarity search tool-A precious help for cell line authentication. International journal of cancer. 2020 Mar. 1; 146(5):1299-306.
Vert A, Castro J, Ribó M, Vilanova M, Benito A. Transcriptional profiling of NCI/ADR-RES cells unveils a complex network of signaling pathways and molecular mechanisms of drug resistance. OncoTargets and therapy. 2018; 11:221.
Domcke S, Sinha R, Levine D A, Sander C, Schultz N. Evaluating cell lines as tumour models by comparison of genomic profiles. Nature communications. 2013 Jul. 9; 4(1):1-0.
Dai X, Cheng H, Bai Z, Li J. Breast cancer cell line classification and its relevance with breast tumor subtyping. Journal of Cancer. 2017; 8(16):3131.
Quinton R J, DiDomizio A, Vittoria M A, Kotýnková K, Ticas C J, Patel S, Koga Y, Vakhshoorzadeh J, Hermance N, Kuroda T S, Parulekar N. Whole-genome doubling confers unique genetic vulnerabilities on tumour cells. Nature. 2021 Jan. 27:1-0.
Steegmaier M, Hoffmann M, Baum A, Lénárt P, Petronczki M, Krššák M, Gürtler U, Garin-Chesa P, Lieb S, Quant J, Grauert M. BI 2536, a potent and selective inhibitor of polo-like kinase 1, inhibits tumor growth in vivo. Current biology. 2007 Feb. 20; 17(4):316-22.
Catarinella M, Grüner T, Strittmatter T, Marx A, Mayer T U. BTB-1: a small molecule inhibitor of the mitotic motor protein Kif18A. Angewandte Chemie International Edition. 2009 Nov. 16; 48(48):9072-6.
Carvalho C, Santos R X, Cardoso S, Correia S, Oliveira P J, Santos M S, Moreira P I. Doxorubicin: the good, the bad and the ugly effect. Current medicinal chemistry. 2009 Sep. 1; 16(25):3267-85.
Pourquier P, Gioffre C, Kohlhagen G, Urasaki Y, Goldwasser F, Hertel L W, Yu S, Pon R T, Gmeiner W H, Pommier Y. Gemcitabine (2′, 2′-difluoro-2′-deoxycytidine), an antimetabolite that poisons topoisomerase I. Clinical Cancer Research. 2002 Aug. 1; 8(8):2499-504.
Hyafil F, Vergely C, Du Vignaud P, Grand-Perret T. In vitro and in vivo reversal of multidrug resistance by GF120918, an acridonecarboxamide derivative. Cancer research. 1993 Oct. 1; 53(19):4595-602.
Rath O, Kozielski F. Kinesins and cancer. Nature reviews cancer. 2012 August; 12(8):527-39.
Vassilev L T, Vu B T, Graves B. Carvajal D, Podlaski F, Filipovic Z, Kong N. Kammlott U, Lukacs C, Klein C, Fotouhi N. In vivo activation of the p53 pathway by small-molecule antagonists of MDM2. Science. 2004 Feb. 6; 303(5659):844-8.
Perez E A. Microtubule inhibitors: Differentiating tubulin-inhibiting agents based on mechanisms of action, clinical activity, and resistance. Molecular cancer therapeutics. 2009 Aug. 1; 8(8):2086-95.
O′leary B, Finn R S, Turner N C. Treating cancer with selective CDK4/6 inhibitors. Nature reviews Clinical oncology. 2016 July; 13(7):417-30.
Payton M, Cheung H K, Ninniri M S, Marinaccio C, Wayne W C, Hanestad K, Crispino J D, Juan G, Coxon A. Dual targeting of Aurora kinases with AMG 900 exhibits potent preclinical activity against acute myeloid leukemia with distinct post-mitotic outcomes. Molecular cancer therapeutics. 2018 Dec. 1; 17(12):2575-85.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of determining a treatment for a subject having a neoplastic disease, comprising assaying a sample obtained from the subject for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, wherein the treatment determined for the subject comprises a KIF18A inhibitor, when the sample is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof.

2. A KIF18A inhibitor for use in treating a subject having a neoplastic disease, wherein the subject comprises cells that are positive for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof.

3. A method of identifying a subject having a neoplastic disease as sensitive to treatment with a KIF18A inhibitor, comprising assaying a sample obtained from the subject for (a) an inactivated TP53 gene and/or (b) at least one of: (i) an inactivated Rb1 gene, (ii) an amplified CCNE1 gene or overexpression of a CCNE1 gene product, (iii) an inactivated BRCA gene or (iv) a combination thereof, wherein the subject is identified as sensitive to treatment with a KIF18A inhibitor, when the sample is positive for an inactivated TP53 gene and/or positive for at least one of an inactivated Rb1 gene, (ii) an amplified CCNE1 gene or overexpression of a CCNE1 gene product, (iii) an inactivated BRCA gene or (iv) a combination thereof.

4. A method of determining a treatment for a subject having a neoplastic disease, comprising determining sensitivity of the neoplastic disease to treatment with a CDK4/6 inhibitor, wherein the treatment for the subject is determined as a treatment comprising a KIF18A inhibitor, when the neoplastic disease is insensitive to the CDK4/6 inhibitor, or determining sensitivity of the neoplastic disease to treatment with a KIF18A inhibitor, wherein the treatment for the subject is determined as a treatment comprising a CDK4/6 inhibitor, when the neoplastic disease is insensitive to the KIF18A inhibitor.

5. A method of identifying a subject having a cancer as responsive to treatment with a KIF18A inhibitor, comprising determining the sensitivity of the neoplastic disease to treatment with a KIF18A inhibitor, wherein the subject is identified as responsive to treatment with a KIF18A inhibitor, when the cancer cells of the sample are insensitive to the CDK4/6 inhibitor.

6. A KIF18A inhibitor for use in (A) treating a subject having a neoplastic disease, wherein the neoplastic disease is resistant to treatment with a CDK4/6 inhibitor or the subject is or has been treated with the CDK4/6 inhibitor or (B) maintaining sensitivity of a neoplastic disease to treatment with a CDK4/6 inhibitor in a subject, optionally, wherein the KIF18A inhibitor is for use together with the CDK4/6 inhibitor.

7. The method of any one of claims 4-6 wherein determining sensitivity comprises assaying a sample obtained from the subject for the absence of (i) an inactivated Rb1 gene, (ii) an amplified CCNE1 gene or overexpression of a CCNE1 gene product, or (iii) a combination thereof.

8. The method of any one of the preceding claims, wherein the sample comprises cancer cells, tumor cells, non-tumor cells, blood, blood cells, or plasma, optionally, wherein the sample comprises germline cancer cells or somatic cancer cells.

9. A pharmaceutical combination comprising a CDK4/6 inhibitor and a KIF18A inhibitor.

10. The method or pharmaceutical combination of any one of claims 4-9 wherein the CDK4/6 inhibitor is palbociclib, ribociclib, and/or abemaciclib.

11. A KIF18A inhibitor for use in treating a subject having a neoplastic disease, inducing or increasing tumor regression in a subject with a tumor, reducing tumor or cancer growth in a subject with a tumor, and/or inducing or increasing death of tumor or cancer cells in a subject.

12. The method or KIF18A inhibitor for use of any one of the preceding claims, wherein the neoplastic disease is a cancer, optionally, breast cancer, ovarian cancer, endometrial cancer, lung cancer, or prostate cancer.

13. The method or KIF18A inhibitor for use of claim 12, wherein the neoplastic disease is triple-negative breast cancer (TNBC), non-luminal breast cancer, or high-grade serous ovarian cancer (HGSOC) or an endometrial cancer, optionally, serous endometrial cancer.

14. The method or KIF18A inhibitor for use of any one of the preceding claims, wherein the cancer comprises cells that are positive for an inactivated TP53 gene and/or positive for at least one of an inactivated Rb gene, (ii) an amplified CCNE1 gene or overexpressed CCNE1 gene product, (iii) an inactivated BRCA gene or (iv) a combination thereof.

15. The method or KIF18A inhibitor for use of claim 14, wherein the cancer comprises cells that are positive for a mutant TP53 gene and/or comprises cells that are positive for an amplified CCNE1 gene, a silenced BRCA1 gene, a deficient Rb1 gene, or a combination thereof.

16. The method or KIF18A inhibitor for use of any one of the preceding claims, wherein the KIF18A inhibitor treats the neoplastic disease, induces or increases tumor regression, reduces tumor or cancer growth, and/or induces or increases death of tumor or cancer cells and (A) the proliferation of the normal somatic cells in the subject is substantially the same as the proliferation of the normal somatic cells of a control subject and/or (B) the level of apoptosis of normal somatic cells is not increased in the subject, relative to the level of apoptosis of normal somatic cells of a control subject, optionally, wherein the level of apoptosis of normal somatic cells is substantially the same as the level of apoptosis of the normal somatic cells of a control subject.

17. The method or KIF18A inhibitor for use of claim 16, wherein the normal somatic cells are human bone marrow mononuclear cells, human mammary epithelial cells, or human foreskin fibroblast cells and/or the normal somatic cells are not TP53^{MUT} or wherein the normal somatic cells are TP53^{WT}.

18. The method or KIF18A inhibitor for use of any one of the preceding claims, wherein (i) the neoplastic disease is a multidrug resistant neoplastic disease, (ii) the tumor or cancer cells are multidrug resistant tumor or cancer cells, (iii) the tumor or cancer cells exhibit increased expression of the Multidrug resistance 1 (MDR-1) gene and/or a gene product thereof, (iv) the tumor or cancer cells exhibit increased expression of a P-glycoprotein (P-gp), or (v) any combination thereof.

19. The method or KIF18A inhibitor for use of any one of the preceding claims, wherein the neoplastic disease is resistant to treatment with an anti-mitotic agent or anthracycline antibiotic, optionally, wherein the anti-mitotic agent or anthracycline antibiotic is paclitaxel or doxorubicin.

20. The method or KIF18A inhibitor for use, or pharmaceutical combination of any one of the preceding claims, wherein the KIF18A inhibitor is administered for oral administration, optionally once a day.

21. The method or KIF18A inhibitor for use of any one of the preceding claims, wherein the KIF18A inhibitor is KIF18A inhibitor Compound C9, also known as 4-(N-(tert-butyl)sulfamoyl)-N-(3-(N-(tert-butyl)sulfamoyl)phenyl)-2-(6-azaspiro[2.5]octan-6-yl)benzamide, having the structure: 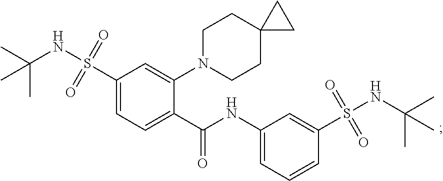 [SMILES: CC(C)(C)NS(=O)(=O)c1cccc(NC(=O)c2ccc(S(=O)(=O)NC(C)(C)C)cc2N2CCC3(CC2)CC3)c1]
or any pharmaceutically acceptable salt thereof.

22. The method or KIF18A inhibitor for use of any one of the preceding claims, wherein the KIF18A inhibitor is KIF18A inhibitor Compound C11, also known as N-(3-(cyclopentylsulfonyl)phenyl)-6-((1-hydroxy-2-methylpropan-2-yl)amino)-2-(6-azaspiro[2.5]octan-6-yl)nicotinamide, having the structure: 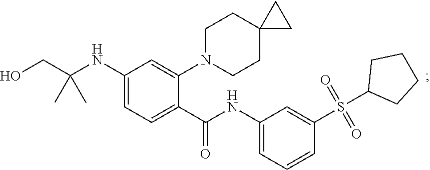 [SMILES: CC(C)(CO)Nc1ccc(C(=O)Nc2cccc(S(=O)(=O)C3CCCC3)c2)c(N2CCC3(CC2)CC3)c1]
or any pharmaceutically acceptable salt thereof.

23. The method or KIF18A inhibitor for use of any one of the preceding claims, wherein the KIF18A inhibitor is KIF18A inhibitor Compound C12, also known as (R)-4-((2-Hydroxyethyl)sulfonamido)-N-(6-(2-methylmorpholino)pyridin-2-yl)-2-(6-azaspiro[2.5]octan-6-yl)benzamide, having the structure: 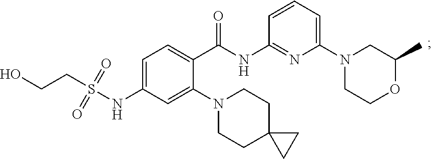 [SMILES: C[C@@H]1CN(c2cccc(NC(=O)c3ccc(NS(=O)(=O)CCO)cc3N3CCC4(CC3)CC4)n2)CCO1]
or any pharmaceutically acceptable salt thereof.

24. The method or KIF18A inhibitor for use of any one of the preceding claims, wherein the KIF18A inhibitor is KIF18A inhibitor Compound C14, also known as N-(2-(4,4-Difluoropiperidin-1-yl)-6-methylpyrmidin-4-yl)-4-((2-hydroxyethyl)sulfonamido)-2-(6-azaspiro[2.5]octan-6-yl)benzamide, having the structure: 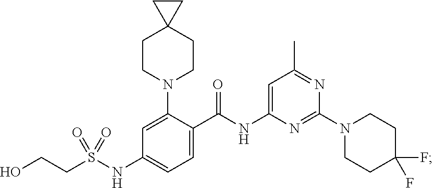 [SMILES: Cc1cc(NC(=O)c2ccc(NS(=O)(=O)CCO)cc2N2CCC3(CC2)CC3)nc(N2CCC(F)(F)CC2)n1]
or any pharmaceutically acceptable salt thereof.

25. A KIF18A inhibitor for use in treating a neoplastic disease in a subject who is or has been treated with an anti-mitotic agent or anthracycline antibiotic, optionally, wherein the KIF18A inhibitor (A) reduces expression of a KIF18A gene and/or a KIF18A gene product (B) is a non-coding RNA, optionally, wherein the KIF18A inhibitor mediates RNAi, and/or (C) is an siRNA, optionally, comprising a sequence of any one of SEQ ID NOs: 12-18.
